(19) United States
(12) Patent Application Publication
(10) Pub. No.: US 2024/0270839 A1
(43) Pub. Date: Aug. 15, 2024

(54) NOVEL ANTI-CLAUDIN18 ANTIBODIES

(71) Applicants: ELPISCIENCE (SUZHOU) BIOPHARMA, LTD., Suzhou, Jiangsu (CN); ELPISCIENCE BIOPHARMA, LTD., Shanghai (CN)

(72) Inventors: Yangsheng QIU, Shanghai (CN); Hongtao LU, Shanghai (CN); Roumei XING, Shanghai (CN); Jingfeng YU, Shanghai (CN); Rui GAO, Shanghai (CN)

(21) Appl. No.: 18/246,516

(22) PCT Filed: Sep. 26, 2021
(86) PCT No.: PCT/CN2021/120683

§ 371 Date: Mar. 24, 2023

(30) Foreign Application Priority Data
Sep. 28, 2020 (WO) PCT/CN2020/118369
Sep. 22, 2021 (CN) 202111107543.X

(51) Int. Cl. C07K 16/28; A61K 38/00; A61K 39/00; A61K 39/395; C07K 14/725; G01N 33/68

(52) U.S. Cl. CPC C07K 16/28; A61K 39/3955; A61K 39/4631; A61K 39/4643; C07K 14/7051; G01N 33/6893; A61K 38/00; C07K 2317/24; C07K 2319/02; C07K 2319/03

(57) ABSTRACT

Provided are anti-CLDN18 (in particular, anti-CLDN18.2) antibodies or antigen-binding fragments thereof, isolated polynucleotides encoding the same, pharmaceutical composition comprising the same and the uses thereof.

### FIELD OF THE INVENTION

The present disclosure generally relates to novel anti-Claudin18 (in particular, anti-Claudin18.2) antibodies and antibody fragments thereof.

### BACKGROUND

Claudin (CLDN) proteins are integral membrane proteins located within the tight junctions of epithelia and endothelia, and are useful for regulating paracellular permeability to ions and solutes. CLDN18 knocked off mice exhibited increased solute permeability and alveolar fluid clearance. The CLDN18 protein is broadly expressed in various cancer types, and at least has two isoforms, CLDN18.1 and CLDN18.2, wherein CLDN18.1 splice variant is expressed in lung, and CLDN18.2 splice variant is expressed in stomach mucosa but not other healthy tissues (Singh et al., Journal of Hematology & Oncology (2017) 10:105). CLDN18.2 provides a highly selective gastric lineage (e.g., gastrocyte-specific) marker with an expression pattern that is restricted to short-lived differentiated epithelial cells and absent from the stem cell zone of gastric glands (Sahin et al., Clin Cancer Res 14(23) 7624-7634, 2008). Sahin et al. also reported that CLDN18.2 is frequently overexpressed in several different types of cancers, including pancreatic, stomach, esophageal, lung and ovarian cancers. Therefore, the published reports suggested that CLDN18.2 may be a diagnostic tool and an attractive target for the development of cancer immunotherapies of diseases associated with epithelial cell-derived tumors. In particular, a monoclonal antibody, Zolbetuximab (also known as IMAB362), generated against CLDN18.2 obtained preliminary results from the clinical trials, which suggests it helpful for advanced gastric cancer.

Needs remain for novel anti-CLDN18 (in particular, anti-CLDN18.2) antibodies.

### SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

In one respect, the present disclosure provides an antibody or an antigen-binding fragment thereof capable of specifically binding to CLDN18, comprising a heavy chain variable region comprising HCDR1, HCDR2 and HCDR3, and/or a light chain variable region comprising LCDR1, LCDR2 and LCDR3, wherein,
a) the HCDR1 comprises an amino acid sequence selected from the group consisting of SEQ ID NOs: 1-28, 201, 202, 332-337; or
b) the HCDR2 comprises an amino acid sequence selected from the group consisting of SEQ ID NOs: 29-67, 203, 338-343, 367; or
c) the HCDR3 comprises an amino acid sequence selected from the group consisting of SEQ ID NOs: 68-94, 344-346; or
d) the LCDR1 comprises an amino acid sequence selected from the group consisting of SEQ ID NOs: 95-113, 205, 347, 348; or
e) the LCDR2 comprises an amino acid sequence selected from the group consisting of SEQ ID NOs: 114-123, 349, 350; or
f) the LCDR3 comprises an amino acid sequence selected from the group consisting of SEQ ID NOs: 124-155, 204, 351-354.

In some embodiments, the antibody or an antigen-binding fragment thereof provided herein comprises a heavy chain variable region comprising HCDR1, HCDR2 and HCDR3, and/or a light chain variable region comprising LCDR1, LCDR2 and LCDR3, wherein,
a) the HCDR1 comprises an amino acid sequence selected from the group consisting of SEQ ID NOs: 1, 4, 11, 15-20, 201, 202, 332-337, and/or
b) the HCDR2 comprises an amino acid sequence selected from the group consisting of SEQ ID NOs: 29, 32, 43, 46-51, 53, 203, 338-343, and/or
c) the HCDR3 comprises an amino acid sequence selected from the group consisting of SEQ ID NOs: 68, 69, 71, 79, 80-85, 344-346, and/or
d) the LCDR1 comprises an amino acid sequence selected from the group consisting of SEQ ID NOs: 95, 96, 101-104, 106, 205, 347, 348, and/or
e) the LCDR2 comprises an amino acid sequence selected from the group consisting of SEQ ID NOs: 114, 115, 117-122, 349, 350, and/or
f) the LCDR3 comprises an amino acid sequence selected from the group consisting of SEQ ID NOs: 124, 127, 135, 137-142, 144, 204, 351-354.

In some embodiments, the antibody or an antigen-binding fragment thereof provided herein comprises a heavy chain variable region comprising HCDR1, HCDR2 and HCDR3, and/or a light chain variable region comprising LCDR1, LCDR2 and LCDR3, wherein,
a) the HCDR1 comprises an amino acid sequence selected from the group consisting of SEQ ID NOs: 1, 4, 11, 15-20, 201, 202, and/or
b) the HCDR2 comprises an amino acid sequence selected from the group consisting of SEQ ID NOs: 29, 32, 43, 46-51, 53, 203, and/or
c) the HCDR3 comprises an amino acid sequence selected from the group consisting of SEQ ID NOs: 68, 69, 71, 79, 80-85, and/or
d) the LCDR1 comprises an amino acid sequence selected from the group consisting of SEQ ID NOs: 95, 96, 101-104, 106, 205, and/or
e) the LCDR2 comprises an amino acid sequence selected from the group consisting of SEQ ID NOs: 114, 115, 117-122, and/or
f) the LCDR3 comprises an amino acid sequence selected from the group consisting of SEQ ID NOs: 124, 127, 135, 137-142, 144, 204.

In some embodiments, the antibody or an antigen-binding fragment thereof provided herein comprises a heavy chain variable region comprising HCDR1, HCDR2 and HCDR3, and/or a light chain variable region comprising LCDR1, LCDR2 and LCDR3, wherein,
a) the HCDR1 comprises an amino acid sequence selected from the group consisting of SEQ ID NOs: 16, 19, 201, 202, and/or
b) the HCDR2 comprises an amino acid sequence selected from the group consisting of SEQ ID NOs: 47, 50, 203 and/or
c) the HCDR3 comprises an amino acid sequence selected from the group consisting of SEQ ID NO: 80, 83, and/or
d) the LCDR1 comprises an amino acid sequence selected from the group consisting of SEQ ID NOs: 96, 103, 205, and/or
e) the LCDR2 comprises an amino acid sequence selected from the group consisting of SEQ ID NO: 118, 120, and/or
f) the LCDR3 comprises an amino acid sequence selected from the group consisting of SEQ ID NOs: 138, 141, 204.

In some embodiments, the antibody or an antigen-binding fragment thereof provided herein comprises a heavy chain variable region comprising:
(1) a HCDR1 comprising the sequence of SEQ ID NO: 1, a HCDR2 comprising the sequence of SEQ ID NO: 29, and a HCDR3 comprising the sequence of SEQ ID NO: 68; or
(2) a HCDR1 comprising the sequence of SEQ ID NO: 2, a HCDR2 comprising the sequence of SEQ ID NO: 30, and a HCDR3 comprising the sequence of SEQ ID NO: 69; or
(3) a HCDR1 comprising the sequence of SEQ ID NO: 3, a HCDR2 comprising the sequence of SEQ ID NO: 31, and a HCDR3 comprising the sequence of SEQ ID NO: 70; or
(4) a HCDR1 comprising the sequence of SEQ ID NO: 4, a HCDR2 comprising the sequence of SEQ ID NO: 32, and a HCDR3 comprising the sequence of SEQ ID NO: 69; or
(5) a HCDR1 comprising the sequence of SEQ ID NO: 5, a HCDR2 comprising the sequence of SEQ ID NO: 33, and a HCDR3 comprising the sequence of SEQ ID NO: 71; or
(6) a HCDR1 comprising the sequence of SEQ ID NO: 4, a HCDR2 comprising the sequence of SEQ ID NO: 34, and a HCDR3 comprising the sequence of SEQ ID NO: 69; or
(7) a HCDR1 comprising the sequence of SEQ ID NO: 6, a HCDR2 comprising the sequence of SEQ ID NO: 32, and a HCDR3 comprising the sequence of SEQ ID NO: 69; or
(8) a HCDR1 comprising the sequence of SEQ ID NO: 7, a HCDR2 comprising the sequence of SEQ ID NO: 35, and a HCDR3 comprising the sequence of SEQ ID NO: 72; or
(9) a HCDR1 comprising the sequence of SEQ ID NO: 8, a HCDR2 comprising the sequence of SEQ ID NO: 36, and a HCDR3 comprising the sequence of SEQ ID NO: 72; or
(10) a HCDR1 comprising the sequence of SEQ ID NO: 6, a HCDR2 comprising the sequence of SEQ ID NO: 37, and a HCDR3 comprising the sequence of SEQ ID NO: 69; or
(11) a HCDR1 comprising the sequence of SEQ ID NO: 5, a HCDR2 comprising the sequence of SEQ ID NO: 38, and a HCDR3 comprising the sequence of SEQ ID NO: 73; or
(12) a HCDR1 comprising the sequence of SEQ ID NO: 8, a HCDR2 comprising the sequence of SEQ ID NO: 35, and a HCDR3 comprising the sequence of SEQ ID NO: 74; or
(13) a HCDR1 comprising the sequence of SEQ ID NO: 2, a HCDR2 comprising the sequence of SEQ ID NO: 39, and a HCDR3 comprising the sequence of SEQ ID NO: 69; or
(14) a HCDR1 comprising the sequence of SEQ ID NO: 9, a HCDR2 comprising the sequence of SEQ ID NO: 40, and a HCDR3 comprising the sequence of SEQ ID NO: 75; or
(15) a HCDR1 comprising the sequence of SEQ ID NO: 9, a HCDR2 comprising the sequence of SEQ ID NO: 41, and a HCDR3 comprising the sequence of SEQ ID NO: 76; or
(16) a HCDR1 comprising the sequence of SEQ ID NO: 10, a HCDR2 comprising the sequence of SEQ ID NO: 42, and a HCDR3 comprising the sequence of SEQ ID NO: 77; or
(17) a HCDR1 comprising the sequence of SEQ ID NO: 11, a HCDR2 comprising the sequence of SEQ ID NO: 43, and a HCDR3 comprising the sequence of SEQ ID NO: 71; or
(18) a HCDR1 comprising the sequence of SEQ ID NO: 12, a HCDR2 comprising the sequence of SEQ ID NO: 44, and a HCDR3 comprising the sequence of SEQ ID NO: 75; or
(19) a HCDR1 comprising the sequence of SEQ ID NO: 13, a HCDR2 comprising the sequence of SEQ ID NO: 40, and a HCDR3 comprising the sequence of SEQ ID NO: 75; or
(20) a HCDR1 comprising the sequence of SEQ ID NO: 14, a HCDR2 comprising the sequence of SEQ ID NO: 45, and a HCDR3 comprising the sequence of SEQ ID NO: 78; or
(21) a HCDR1 comprising the sequence of SEQ ID NO: 8, a HCDR2 comprising the sequence of SEQ ID NO: 35, and a HCDR3 comprising the sequence of SEQ ID NO: 72; or
(22) a HCDR1 comprising the sequence of SEQ ID NO: 15, a HCDR2 comprising the sequence of SEQ ID NO: 46, and a HCDR3 comprising the sequence of SEQ ID NO: 79; or
(23) a HCDR1 comprising the sequence of SEQ ID NO: 16, a HCDR2 comprising the sequence of SEQ ID NO: 47, and a HCDR3 comprising the sequence of SEQ ID NO: 80; or
(24) a HCDR1 comprising the sequence of SEQ ID NO: 201, a HCDR2 comprising the sequence of SEQ ID NO: 47, and a HCDR3 comprising the sequence of SEQ ID NO: 80; or
(25) a HCDR1 comprising the sequence of SEQ ID NO: 17, a HCDR2 comprising the sequence of SEQ ID NO: 48, and a HCDR3 comprising the sequence of SEQ ID NO: 81; or
(26) a HCDR1 comprising the sequence of SEQ ID NO: 18, a HCDR2 comprising the sequence of SEQ ID NO: 49, and a HCDR3 comprising the sequence of SEQ ID NO: 82; or
(27) a HCDR1 comprising the sequence of SEQ ID NO: 19, a HCDR2 comprising the sequence of SEQ ID NO: 50, and a HCDR3 comprising the sequence of SEQ ID NO: 83; or
(28) a HCDR1 comprising the sequence of SEQ ID NO: 202, a HCDR2 comprising the sequence of SEQ ID NO: 50, and a HCDR3 comprising the sequence of SEQ ID NO: 83; or
(29) a HCDR1 comprising the sequence of SEQ ID NO: 202, a HCDR2 comprising the sequence of SEQ ID NO: 203, and a HCDR3 comprising the sequence of SEQ ID NO: 83; or
(30) a HCDR1 comprising the sequence of SEQ ID NO: 17, a HCDR2 comprising the sequence of SEQ ID NO: 51, and a HCDR3 comprising the sequence of SEQ ID NO: 84; or
(31) a HCDR1 comprising the sequence of SEQ ID NO: 2, a HCDR2 comprising the sequence of SEQ ID NO: 52, and a HCDR3 comprising the sequence of SEQ ID NO: 69; or
(32) a HCDR1 comprising the sequence of SEQ ID NO: 20, a HCDR2 comprising the sequence of SEQ ID NO: 53, and a HCDR3 comprising the sequence of SEQ ID NO: 85; or
(33) a HCDR1 comprising the sequence of SEQ ID NO: 21, a HCDR2 comprising the sequence of SEQ ID NO: 54, and a HCDR3 comprising the sequence of SEQ ID NO: 86; or
(34) a HCDR1 comprising the sequence of SEQ ID NO: 22, a HCDR2 comprising the sequence of SEQ ID NO: 55, and a HCDR3 comprising the sequence of SEQ ID NO: 87; or
(35) a HCDR1 comprising the sequence of SEQ ID NO: 2, a HCDR2 comprising the sequence of SEQ ID NO: 56, and a HCDR3 comprising the sequence of SEQ ID NO: 69; or
(36) a HCDR1 comprising the sequence of SEQ ID NO: 1, a HCDR2 comprising the sequence of SEQ ID NO: 57, and a HCDR3 comprising the sequence of SEQ ID NO: 69; or
(37) a HCDR1 comprising the sequence of SEQ ID NO: 11, a HCDR2 comprising the sequence of SEQ ID NO: 43, and a HCDR3 comprising the sequence of SEQ ID NO: 88; or
(38) a HCDR1 comprising the sequence of SEQ ID NO: 23, a HCDR2 comprising the sequence of SEQ ID NO: 58, and a HCDR3 comprising the sequence of SEQ ID NO: 89; or
(39) a HCDR1 comprising the sequence of SEQ ID NO: 24, a HCDR2 comprising the sequence of SEQ ID NO: 59, and a HCDR3 comprising the sequence of SEQ ID NO: 90; or
(40) a HCDR1 comprising the sequence of SEQ ID NO: 25, a HCDR2 comprising the sequence of SEQ ID NO: 60, and a HCDR3 comprising the sequence of SEQ ID NO: 90; or
(41) a HCDR1 comprising the sequence of SEQ ID NO: 26, a HCDR2 comprising the sequence of SEQ ID NO: 61, and a HCDR3 comprising the sequence of SEQ ID NO: 91; or
(42) a HCDR1 comprising the sequence of SEQ ID NO: 26, a HCDR2 comprising the sequence of SEQ ID NO: 62, and a HCDR3 comprising the sequence of SEQ ID NO: 92; or
(43) a HCDR1 comprising the sequence of SEQ ID NO: 27, a HCDR2 comprising the sequence of SEQ ID NO: 367, and a HCDR3 comprising the sequence of SEQ ID NO: 93; or
(44) a HCDR1 comprising the sequence of SEQ ID NO: 26, a HCDR2 comprising the sequence of SEQ ID NO: 63, and a HCDR3 comprising the sequence of SEQ ID NO: 92; or
(45) a HCDR1 comprising the sequence of SEQ ID NO: 26, a HCDR2 comprising the sequence of SEQ ID NO: 64, and a HCDR3 comprising the sequence of SEQ ID NO: 92; or
(46) a HCDR1 comprising the sequence of SEQ ID NO: 28, a HCDR2 comprising the sequence of SEQ ID NO: 65, and a HCDR3 comprising the sequence of SEQ ID NO: 85; or
(47) a HCDR1 comprising the sequence of SEQ ID NO: 28, a HCDR2 comprising the sequence of SEQ ID NO: 66, and a HCDR3 comprising the sequence of SEQ ID NO: 94; or
(48) a HCDR1 comprising the sequence of SEQ ID NO: 28, a HCDR2 comprising the sequence of SEQ ID NO: 66, and a HCDR3 comprising the sequence of SEQ ID NO: 85; or
(49) a HCDR1 comprising the sequence of SEQ ID NO: 26, a HCDR2 comprising the sequence of SEQ ID NO: 67, and a HCDR3 comprising the sequence of SEQ ID NO: 92; or
(50) a HCDR1 comprising the sequence of SEQ ID NO: 11, a HCDR2 comprising the sequence of SEQ ID NO: 61, and a HCDR3 comprising the sequence of SEQ ID NO: 91.

In some embodiments, the antibody or an antigen-binding fragment thereof provided herein comprises a light chain variable region comprising: (1) a LCDR1 comprising the sequence of SEQ ID NO: 95, a LCDR2 comprising the sequence of SEQ ID NO: 114, and a LCDR3 comprising the sequence of SEQ ID NO: 124; or
(2) a LCDR1 comprising the sequence of SEQ ID NO: 96, a LCDR2 comprising the sequence of SEQ ID NO: 114, and a LCDR3 comprising the sequence of SEQ ID NO: 125; or
(3) a LCDR1 comprising the sequence of SEQ ID NO: 96, a LCDR2 comprising the sequence of SEQ ID NO: 115, and a LCDR3 comprising the sequence of SEQ ID NO: 126; or
(4) a LCDR1 comprising the sequence of SEQ ID NO: 96, a LCDR2 comprising the sequence of SEQ ID NO: 115, and a LCDR3 comprising the sequence of SEQ ID NO: 127; or
(5) a LCDR1 comprising the sequence of SEQ ID NO: 96, a LCDR2 comprising the sequence of SEQ ID NO: 116, and a LCDR3 comprising the sequence of SEQ ID NO: 128; or
(6) a LCDR1 comprising the sequence of SEQ ID NO: 97, a LCDR2 comprising the sequence of SEQ ID NO: 115, and a LCDR3 comprising the sequence of SEQ ID NO: 129; or
(7) a LCDR1 comprising the sequence of SEQ ID NO: 96, a LCDR2 comprising the sequence of SEQ ID NO: 115, and a LCDR3 comprising the sequence of SEQ ID NO: 130; or
(8) a LCDR1 comprising the sequence of SEQ ID NO: 98, a LCDR2 comprising the sequence of SEQ ID NO: 115, and a LCDR3 comprising the sequence of SEQ ID NO: 127; or
(9) a LCDR1 comprising the sequence of SEQ ID NO: 96, a LCDR2 comprising the sequence of SEQ ID NO: 115, and a LCDR3 comprising the sequence of SEQ ID NO: 128; or
(10) a LCDR1 comprising the sequence of SEQ ID NO: 99, a LCDR2 comprising the sequence of SEQ ID NO: 115, and a LCDR3 comprising the sequence of SEQ ID NO: 131; or
(11) a LCDR1 comprising the sequence of SEQ ID NO: 99, a LCDR2 comprising the sequence of SEQ ID NO: 115, and a LCDR3 comprising the sequence of SEQ ID NO: 132; or
(12) a LCDR1 comprising the sequence of SEQ ID NO: 99, a LCDR2 comprising the sequence of SEQ ID NO: 115, and a LCDR3 comprising the sequence of SEQ ID NO: 133; or
(13) a LCDR1 comprising the sequence of SEQ ID NO: 100, a LCDR2 comprising the sequence of SEQ ID NO: 115, and a LCDR3 comprising the sequence of SEQ ID NO: 134; or
(14) a LCDR1 comprising the sequence of SEQ ID NO: 101, a LCDR2 comprising the sequence of SEQ ID NO: 115, and a LCDR3 comprising the sequence of SEQ ID NO: 135; or
(15) a LCDR1 comprising the sequence of SEQ ID NO: 96, a LCDR2 comprising the sequence of SEQ ID NO: 115, and a LCDR3 comprising the sequence of SEQ ID NO: 136; or
(16) a LCDR1 comprising the sequence of SEQ ID NO: 102, a LCDR2 comprising the sequence of SEQ ID NO: 117, and a LCDR3 comprising the sequence of SEQ ID NO: 137; or
(17) a LCDR1 comprising the sequence of SEQ ID NO: 103, a LCDR2 comprising the sequence of SEQ ID NO: 118, and a LCDR3 comprising the sequence of SEQ ID NO: 138; or
(18) a LCDR1 comprising the sequence of SEQ ID NO: 103, a LCDR2 comprising the sequence of SEQ ID NO: 118, and a LCDR3 comprising the sequence of SEQ ID NO: 204; or
(19) a LCDR1 comprising the sequence of SEQ ID NO: 96, a LCDR2 comprising the sequence of SEQ ID NO: 119, and a LCDR3 comprising the sequence of SEQ ID NO: 139; or
(20) a LCDR1 comprising the sequence of SEQ ID NO: 104, a LCDR2 comprising the sequence of SEQ ID NO: 118, and a LCDR3 comprising the sequence of SEQ ID NO: 140; or
(21) a LCDR1 comprising the sequence of SEQ ID NO: 96, a LCDR2 comprising the sequence of SEQ ID NO: 120, and a LCDR3 comprising the sequence of SEQ ID NO: 141; or
(22) a LCDR1 comprising the sequence of SEQ ID NO: 205, a LCDR2 comprising the sequence of SEQ ID NO: 120, and a LCDR3 comprising the sequence of SEQ ID NO: 141; or
(23) a LCDR1 comprising the sequence of SEQ ID NO: 96, a LCDR2 comprising the sequence of SEQ ID NO: 121, and a LCDR3 comprising the sequence of SEQ ID NO: 142; or
(24) a LCDR1 comprising the sequence of SEQ ID NO: 105, a LCDR2 comprising the sequence of SEQ ID NO: 115, and a LCDR3 comprising the sequence of SEQ ID NO: 143; or
(25) a LCDR1 comprising the sequence of SEQ ID NO: 106, a LCDR2 comprising the sequence of SEQ ID NO: 122, and a LCDR3 comprising the sequence of SEQ ID NO: 144; or
(26) a LCDR1 comprising the sequence of SEQ ID NO: 95, a LCDR2 comprising the sequence of SEQ ID NO: 115, and a LCDR3 comprising the sequence of SEQ ID NO: 128; or
(27) a LCDR1 comprising the sequence of SEQ ID NO: 98, a LCDR2 comprising the sequence of SEQ ID NO: 115, and a LCDR3 comprising the sequence of SEQ ID NO: 145; or
(28) a LCDR1 comprising the sequence of SEQ ID NO: 96, a LCDR2 comprising the sequence of SEQ ID NO: 115, and a LCDR3 comprising the sequence of SEQ ID NO: 146; or
(29) a LCDR1 comprising the sequence of SEQ ID NO: 96, a LCDR2 comprising the sequence of SEQ ID NO: 115, and a LCDR3 comprising the sequence of SEQ ID NO: 147; or
(30) a LCDR1 comprising the sequence of SEQ ID NO: 96, a LCDR2 comprising the sequence of SEQ ID NO: 115, and a LCDR3 comprising the sequence of SEQ ID NO: 148; or
(31) a LCDR1 comprising the sequence of SEQ ID NO: 107, a LCDR2 comprising the sequence of SEQ ID NO: 115, and a LCDR3 comprising the sequence of SEQ ID NO: 149; or
(32) a LCDR1 comprising the sequence of SEQ ID NO: 108, a LCDR2 comprising the sequence of SEQ ID NO: 115, and a LCDR3 comprising the sequence of SEQ ID NO: 150; or
(33) a LCDR1 comprising the sequence of SEQ ID NO: 108, a LCDR2 comprising the sequence of SEQ ID NO: 115, and a LCDR3 comprising the sequence of SEQ ID NO: 150; or
(34) a LCDR1 comprising the sequence of SEQ ID NO: 107, a LCDR2 comprising the sequence of SEQ ID NO: 115, and a LCDR3 comprising the sequence of SEQ ID NO: 149; or
(35) a LCDR1 comprising the sequence of SEQ ID NO: 109, a LCDR2 comprising the sequence of SEQ ID NO: 123, and a LCDR3 comprising the sequence of SEQ ID NO: 151; or
(36) a LCDR1 comprising the sequence of SEQ ID NO: 110, a LCDR2 comprising the sequence of SEQ ID NO: 115, and a LCDR3 comprising the sequence of SEQ ID NO: 150; or
(37) a LCDR1 comprising the sequence of SEQ ID NO: 111, a LCDR2 comprising the sequence of SEQ ID NO: 115, and a LCDR3 comprising the sequence of SEQ ID NO: 150; or
(38) a LCDR1 comprising the sequence of SEQ ID NO: 101, a LCDR2 comprising the sequence of SEQ ID NO: 120, and a LCDR3 comprising the sequence of SEQ ID NO: 152; or
(39) a LCDR1 comprising the sequence of SEQ ID NO: 101, a LCDR2 comprising the sequence of SEQ ID NO: 120, and a LCDR3 comprising the sequence of SEQ ID NO: 153; or
(40) a LCDR1 comprising the sequence of SEQ ID NO: 112, a LCDR2 comprising the sequence of SEQ ID NO: 120, and a LCDR3 comprising the sequence of SEQ ID NO: 152; or
(41) a LCDR1 comprising the sequence of SEQ ID NO: 101, a LCDR2 comprising the sequence of SEQ ID NO: 120, and a LCDR3 comprising the sequence of SEQ ID NO: 153; or
(42) a LCDR1 comprising the sequence of SEQ ID NO: 107, a LCDR2 comprising the sequence of SEQ ID NO: 115, and a LCDR3 comprising the sequence of SEQ ID NO: 149; or
(43) a LCDR1 comprising the sequence of SEQ ID NO: 101, a LCDR2 comprising the sequence of SEQ ID NO: 120, and a LCDR3 comprising the sequence of SEQ ID NO: 152; or
(44) a LCDR1 comprising the sequence of SEQ ID NO: 101, a LCDR2 comprising the sequence of SEQ ID NO: 120, and a LCDR3 comprising the sequence of SEQ ID NO: 154; or
(45) a LCDR1 comprising the sequence of SEQ ID NO: 96, a LCDR2 comprising the sequence of SEQ ID NO: 115, and a LCDR3 comprising the sequence of SEQ ID NO: 155; or
(46) a LCDR1 comprising the sequence of SEQ ID NO: 108, a LCDR2 comprising the sequence of SEQ ID NO: 115, and a LCDR3 comprising the sequence of SEQ ID NO: 150; or
(47) a LCDR1 comprising the sequence of SEQ ID NO: 107, a LCDR2 comprising the sequence of SEQ ID NO: 115, and a LCDR3 comprising the sequence of SEQ ID NO: 149; or
(48) a LCDR1 comprising the sequence of SEQ ID NO: 113, a LCDR2 comprising the sequence of SEQ ID NO: 115, and a LCDR3 comprising the sequence of SEQ ID NO: 149; or
(49) a LCDR1 comprising the sequence of SEQ ID NO: 108, a LCDR2 comprising the sequence of SEQ ID NO: 115, and a LCDR3 comprising the sequence of SEQ ID NO: 150; or
(50) a LCDR1 comprising the sequence of SEQ ID NO: 107, a LCDR2 comprising the sequence of SEQ ID NO: 115, and a LCDR3 comprising the sequence of SEQ ID NO: 149; or
(51) a LCDR1 comprising the sequence of SEQ ID NO: 101, a LCDR2 comprising the sequence of SEQ ID NO: 120, and a LCDR3 comprising the sequence of SEQ ID NO: 153.

In some embodiments, the antibody or an antigen-binding fragment thereof provided herein further comprises one or more of heavy chain HFR1, HFR2, HFR3 and HFR4, and/or one or more of light chain LFR1, LFR2, LFR3 and LFR4, wherein:
a) the HFR1 comprises the sequence of SEQ ID NO: 355 or 356, or a homologous sequence of at least 80% sequence identity thereof,
b) the HFR2 comprises the sequence of SEQ ID NO: 357 or 358, or a homologous sequence of at least 80% sequence identity thereof,
c) the HFR3 comprises the sequence of SEQ ID NO: 359 or 360, or a homologous sequence of at least 80% sequence identity thereof,
d) the HFR4 comprises the sequence of SEQ ID NO: 361 or 362, or a homologous sequence of at least 80% sequence identity thereof,
e) the LFR1 comprises the sequence of SEQ ID NO: 363 or a homologous sequence of at least 80% sequence identity thereof,
f) the LFR2 comprises the sequence of SEQ ID NO: 364 or a homologous sequence of at least 80% sequence identity thereof,
g) the LFR3 comprises the sequence of SEQ ID NO: 365 or a homologous sequence of at least 80% sequence identity thereof, and
h) the LFR4 comprises the sequence of SEQ ID NO: 366 or a homologous sequence of at least 80% sequence identity thereof.

In some embodiments, the antibody or an antigen-binding fragment thereof provided herein comprises a heavy chain variable region comprising a sequence selected from the group consisting of SEQ ID NOs: 206-259, 311-313, 318-321, and a homologous sequence thereof having at least 80% sequence identity yet retaining specific binding affinity to CLDN18, and a light chain variable region comprising a sequence selected from the group consisting of SEQ ID NOs: 260-310, 314-317, 322-324, and a homologous sequence thereof having at least 80% sequence identity yet retaining specific binding affinity to CLDN18.

In some embodiments, the antibody or an antigen-binding fragment thereof provided herein comprises a heavy chain variable region comprising a sequence selected from the group consisting of SEQ ID NOs: 210, 246, and a homologous sequence thereof having at least 80% sequence identity yet retaining specific binding affinity to CLDN18, and a light chain variable region comprising the sequence selected from the group consisting of SEQ ID NOs: 273, 307, and a homologous sequence thereof having at least 80% sequence identity yet retaining specific binding affinity to CLDN18.

In some embodiments, the antibody or an antigen-binding fragment thereof provided herein further comprises one or more amino acid residue substitutions or modifications yet retains specific binding affinity to CLDN18. In some embodiments, the at least one of the substitutions or modifications is in one or more of the CDR sequences, and/or in one or more of the non-CDR sequences of the heavy chain variable region or light chain variable region.

In some embodiments, the antibody or an antigen-binding fragment thereof further comprises an Fc region, optionally an Fc region of human immunoglobulin (Ig), or optionally an Fc region of human IgG. In some embodiments, the Fc region is derived from human IgG1, IgG2, IgG3, IgG4, IgA1, IgA2 or IgM. In some embodiments, the Fc region derived from human IgG1 comprises one or more mutations selected from the group consisting of L235V, G236A, S239D, F243L, H268F, R292P, Y300L, V305I, S324T, A330L, I332E, and P396L. In some embodiments, the Fc region derived from human IgG1 comprises a mutation selected from the group consisting of: (1) G236A, S239D and I332E; (2) S239D, A330L and I332E; (3) S239D and I332E; (4) S239D, H268F, S324T and I332E; (5) F243L, R292P, Y300L, V305I and P396L; (6) L235V, F243L, R292P, Y300L and P396L.

In some embodiments, the antibody or an antigen-binding fragment thereof provided herein comprises an Fc region comprising an amino acid sequence selected from the group consisting of SEQ ID NOs: 326-331.

In some embodiments, the antibody or an antigen-binding fragment thereof provided herein is humanized. In some embodiments, the antibody or an antigen-binding fragment thereof is a monoclonal antibody, a bispecific antibody, a multi-specific antibody, a recombinant antibody, a chimeric antibody, a labeled antibody, a bivalent antibody, an anti-idiotypic antibody or a fusion protein.

In some embodiments, the antibody or an antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

In some embodiments, the antibody or an antigen-binding fragment thereof provided herein is capable of specifically binding to human CLDN18. In some embodiments, the antibody or an antigen-binding fragment thereof provided herein is capable of specifically binding to human CLDN18.2. In some embodiments, the antibody or an antigen-binding fragment thereof provided herein is capable of binding to both human CLDN18.1 and human CLDN18.2. In some embodiments, the antibody or an antigen-binding fragment thereof provided herein is capable of specifically binding to human CLDN18.2 at an EC₅₀ of no more than 2 nM as measured by FACS assay.

In some embodiments, the antibody or antigen-binding fragment thereof provided herein has one or more properties selected from the group consisting of:
a) specifically binding to human CLDN18.2 but not specifically binding to human CLDN18.1 as measured by FACS assay;
b) specifically binding to both human CLDN18.2 and mouse CLDN18.2 as measured by FACS assay;
c) specifically binding to mouse CLDN18.2 at an EC₅₀ of no more than 4 nM as measured by FACS assay;
d) specifically binding to human CLDN18.2 at a K_{D} value of no more than 10⁻⁸ M as measured by Biacore assay;
e) specifically binding to human CLDN18.2 at a K_{D} value of no more than 10⁻⁸ M as measured by Octet assay.

In another aspect, the present disclosure provides an anti-CLDN18 antibody or an antigen-binding fragment thereof, which competes for binding to human CLDN18 with the antibody or an antigen-binding fragment thereof provided herein.

In some embodiments, the CLDN18 of the present disclosure is a human CLDN18.2 comprising an amino acid sequence of SEQ ID NO: 401.

In some embodiments, the antibody or an antigen-binding fragment thereof provided herein is not Antibody IMAB362, wherein Antibody IMAB362 comprises a heavy chain variable region comprising the sequence of SEQ ID NO: 397, and a light chain variable region comprising the sequence of SEQ ID NO: 398.

In some embodiments, the antibody or an antigen-binding fragment thereof provided herein is bispecific.

In some embodiments, the antibody or an antigen-binding fragment thereof provided herein is capable of specifically binding to a second antigen other than CLDN18, or a second epitope on CLDN18.

In some embodiments, the antibody or an antigen-binding fragment thereof provided herein is linked to one or more conjugate moieties. In some embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binder, a purification moiety or other anticancer drugs.

In another aspect, the present disclosure provides a pharmaceutical composition comprising the antibody or an antigen-binding fragment thereof provided herein, and one or more pharmaceutically acceptable carriers.

In another aspect, the present disclosure provides a chimeric antigen receptor comprising the antibody or an antigen-binding fragment thereof provided herein, a transmembrane region and an intracellular signal region. In some embodiments, the antigen-binding fragment of the chimeric antigen receptor is a scFv. In some embodiments, the transmembrane region of the chimeric antigen receptor comprises a transmembrane region of CD3, CD4, CD8 or CD28. In some embodiments, the intracellular signal region of the chimeric antigen receptor is selected from the group consisting of: an intracellular signal region sequence of CD3, CD27, CD28, CD137, CD134, MyD88, CD40, CD278, TLRs, or a combination thereof.

In another aspect, the present disclosure provides an isolated polynucleotide encoding the antibody or an antigen-binding fragment thereof provided herein, and/or the chimeric antigen receptor provided herein.

In another aspect, the present disclosure provides a vector comprising the isolated polynucleotide of the present disclosure.

In another aspect, the present disclosure provides a host cell comprising the vector of the present disclosure.

In another aspect, the present disclosure provides a kit comprising the antibody or an antigen-binding fragment thereof of the present disclosure, and/or the pharmaceutical composition of the present disclosure, and/or the chimeric antigen receptor of the present disclosure, and a second therapeutic agent.

In another aspect, the present disclosure provides a method of expressing the antibody or antigen-binding fragment thereof of the present disclosure or the chimeric antigen receptor of the present disclosure, comprising culturing the host cell of the present disclosure under the condition at which the vector of the present disclosure is expressed.

In another aspect, the present disclosure provides a method of treating, preventing or alleviating a CLDN18 related disease, disorder or condition in a subject, comprising administering to the subject a therapeutically effective amount of the antibody or an antigen-binding fragment thereof of the present disclosure, and/or the pharmaceutical composition of the present disclosure, and/or the chimeric antigen receptor of the present disclosure.

In some embodiments, the disease, disorder or condition is cancer. In some embodiments, the cancer is an epithelial-cell derived cancer. In some embodiments, the cancer is anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, or adenocarcinoma, or the metastases thereof.

In some embodiments, the cancer is gastric cancer, pancreatic cancer, esophagus cancer, ovarian cancer, or the metastases thereof.

In some embodiments, the subject has been identified as having a cancer cell or tumor infiltrating immune cells expressing CLDN18, optionally at a level significantly higher from the level normally found on non-cancer cells. In some embodiments, the subject is human.

In some embodiments, the administration is via oral, nasal, intravenous, subcutaneous, sublingual, or intramuscular administration.

In some embodiments, the method of treating, preventing or alleviating a CLDN18 related disease, disorder or condition in a subject further comprises administering a therapeutically effective amount of a second therapeutic agent. In some embodiments, the second therapeutic agent is selected from the group consisting of a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

In another aspect, the present disclosure provides a method of modulating CLDN18 activity in a CLDN18-positive cell, comprising exposing the CLDN18-positive cell to the antibody or antigen-binding fragment thereof of the present disclosure, and/or the pharmaceutical composition of the present disclosure and/or the chimeric antigen receptor of the present disclosure.

In another aspect, the present disclosure provides a method of detecting the presence or amount of CLDN18 in a sample, comprising contacting the sample with the antibody or an antigen-binding fragment thereof of the present disclosure, and/or the pharmaceutical composition of the present disclosure and/or the chimeric antigen receptor of the present disclosure, and determining the presence or the amount of CLDN18 in the sample.

In another aspect, the present disclosure provides a method of diagnosing a CLDN18 related disease, disorder or condition in a subject, comprising: a) contacting a sample obtained from the subject with the antibody or an antigen-binding fragment thereof of the present disclosure, and/or the pharmaceutical composition of the present disclosure and/or the chimeric antigen receptor of the present disclosure; b) determining the presence or amount of CLDN18 in the sample; and c) correlating the presence or the amount of CLDN18 to existence or status of the CLDN18 related disease, disorder or condition in the subject.

In another aspect, the present disclosure provides a method of treating, preventing or alleviating a disease, disorder or condition in a subject that would benefit from modulation of CLDN18 activity, comprising administering to the subject a therapeutically effective amount of the antibody or an antigen-binding fragment thereof of the present disclosure, and/or the pharmaceutical composition of the present disclosure and/or the chimeric antigen receptor of the present disclosure.

In another aspect, the present disclosure provides use of the antibody or antigen-binding fragment thereof of the present disclosure, and/or the pharmaceutical composition of the present disclosure and/or the chimeric antigen receptor of the present disclosure in the manufacture of a medicament for treating, preventing or alleviating a CLDN18 related disease, disorder or condition in a subject.

In another aspect, the present disclosure provides use of the antibody or an antigen-binding fragment thereof of the present disclosure, and/or the pharmaceutical composition of the present disclosure and/or the chimeric antigen receptor of the present disclosure in the manufacture of a diagnostic reagent for diagnosing a CLDN18 related disease, disorder or condition in a subject.

In another aspect, the present disclosure provides a kit comprising the antibody or an antigen-binding fragment thereof of the present disclosure, and/or the pharmaceutical composition of the present disclosure and/or the chimeric antigen receptor of the present disclosure, useful in detecting CLDN18.

In some embodiments, the CLDN18 of the present disclosure is human CLDN18.2.

### BRIEF DESCRIPTION OF THE DRAWINGS

FIG. 1 shows the results of ELISA assay against hCLDN18.2 stabilized protein with reference antibody IMAB362.

FIG. 2 shows the results of ELISA assay against hCLDN18.2 stabilized protein with serum of immunized mice.

FIG. 3 shows the results of FACS assay against CHO-K1-hCLDN18.2 stable cell line with serum of immunized mice.

FIG. 4 shows the representative figure of hybridoma screening.

FIG. 5 shows the binding affinities of the purified hybridoma antibodies to HEK293-hCLDN18.2 cells (FIG. 5A), HEK293-hCLDN18.1 cells (FIG. 5B), HEK293-mCLDN18.2 cells (FIG. 5C), and SNU620 cells (FIG. 5D), respectively.

FIG. 6 shows the ADCC study results of 6 chimeric antibodies (i.e., ch99H8, ch97A9, ch60F11, ch35B4, ch22E12, ch33G12) with HEK293/hCLDN18.2 cells as the target cells.

FIG. 7 shows the dose response of the 6 chimeric antibodies (i.e., ch99H8, ch97A9, ch60F11, ch35B4, ch22E12, ch33G12) in ADCC study with NUGC-4/hCLDN18.2 cells as the target cells.

FIG. 8 shows the dose response results of 6 chimeric antibodies (i.e., ch99H8, ch97A9, ch60F11, ch35B4, ch22E12, ch33G12) in ADCC study with HEK293-hCLDN18.1 cells as the target cells.

FIG. 9 shows the CDC study results of 6 chimeric antibodies (i.e., ch99H8, ch97A9, ch60F11, ch35B4, ch22E12, ch33G12) with HEK293/hCLDN18.2 cells as the target cells.

FIG. 10A and FIG. 10B show binding affinities of humanized 22E12 antibodies to MFC/hCLDN18.2 cells.

FIG. 11A and FIG. 11B show binding affinities of humanized 35B4 antibodies to SNU620/hCLDN18.2 cells.

FIG. 12A and FIG. 12B show the ADCC study results of humanized 22E12 and 35B4 antibodies on NK92-CD16a and HEK293/hCLDN18.2 cells (FIG. 12A) or NUGC4 cells (FIG. 12B).

FIG. 13 shows the ADCC study results of Fc engineered humanized 35B4 (FIG. 13A) and 22E12 (FIG. 13B) antibodies on NK92-CD16a and HEK293/hCLDN18.2 cells.

FIG. 14 shows the representative image of immunohistochemistry (IHC) staining of the antibodies provided herein.

FIG. 15A to 15E show the kinetics study results using surface plasmon resonance (SPR) for antibody hu35B4.H1L2 (FIG. 15A), antibody hu22E12.H1L2 (FIG. 15B), antibody ch99H8 (FIG. 15C), antibody ch97A9 (FIG. 15D), and reference antibody IMAB362 (FIG. 15E), respectively.

FIG. 16 shows the internalization rates of exemplary antibodies on SNU620 cells.

FIG. 17A and FIG. 17B show the tumor volume changes (FIG. 17A; * indicates p<0.05; *** indicates p<0.001) and body weight changes (FIG. 17B) in mice over the days post treatments of vehicle, human IgG1 isotype, hu22E12.H1L2 alone, anti-SIRPα antibody alone, and hu22E12.H1L2+anti-SIRPα antibody combo, respectively.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### Definitions

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain comprises a variable region (VH) and a first, second, third, and optionally fourth constant region (CH1, CH2, CH3, CH4 respectively); mammalian light chains are classified as λ or κ, while each light chain comprises a variable region (VL) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding fragments disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27: 55-77 (2003); Marie-Paule Lefranc et al., Immunome Research, 1(3), (2005); Marie-Paule Lefranc, Molecular Biology of B cells (second edition), chapter 26, 481-514, (2015)). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragments include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multispecific antibody, a camelized single domain antibody, a nanobody, a domain antibody, and a bivalent domain antibody. An antigen-binding fragment is capable of binding to the same antigen to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody (e.g. of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879 (1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J. Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a V_{L} domain in the same polypeptide chain (V_{H}-V_{L} or V_{L}-VH) (see, e.g. Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two binding sites, four binding sites, and six binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

In certain embodiments, an “scFv dimer” is a bivalent diabody or bispecific scFv (BsFv) comprising V_{H}-V_{L} (linked by a peptide linker) dimerized with another V_{H}-VL moiety such that V_{H}'s of one moiety coordinate with the V_{L}'s of the other moiety and form two binding sites which can target the same antigens (or epitopes) or different antigens (or epitopes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv)₂” or “(dsFv-dsFv′)” comprises three peptide chains: two V_{H} moieties linked by a peptide linker (e.g. a long flexible linker) and bound to two V_{L} moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse.

In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human.

The CDRs of humanized antibodies provided in the present disclosure may contain mutation(s) compared to the CDRs of their parent antibodies.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. Specific binding can be characterized in binding affinity, for example, represented by K_{D} value, i.e., the ratio of dissociation rate to association rate (k_{off}/kₒₙ) when the binding between the antigen and antigen-binding molecule reaches equilibrium. K_{D} may be determined by using any conventional method known in the art, including but are not limited to surface plasmon resonance method, Octet method, microscale thermophoresis method, HPLC-MS method and Fluorescence Activated Cell Sorting (FACS) assay method. A K_{D} value of ≤10⁻⁶ M (e.g. ≤5×10⁻⁷M, 2×10⁻⁷ M, ≤10⁻⁷ M, ≤5×10⁻⁸M, ≤2×10⁻⁸M, ≤10⁻⁸M, ≤5×10⁻⁹M, ≤4×10⁻⁹M, ≤3×10⁻⁹M, ≤2×10⁻⁹M, ≤10⁻⁹M, ≤5×10⁻¹⁰M, ≤4×10⁻¹⁰ M, ≤3×10⁻¹⁰ M, ≤2×10⁻¹⁰ M, ≤10⁻¹⁰ M) can indicate specific binding between an antibody or antigen binding fragments thereof and CLDN18.2 (e.g. human CLDN18.2).

The ability to “compete for binding to CLDN18.2” as used herein refers to the ability of a first antibody or antigen-binding fragment to inhibit the binding interaction between CLDN18.2 and a second anti-CLDN18.2 antibody to any detectable degree. In certain embodiments, an antibody or antigen-binding fragment that compete for binding to CLDN18.2 inhibits the binding interaction between CLDN18.2 and a second anti-CLDN18.2 antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e. including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among amino acid residues with neutral hydrophilic side chains (e.g. Cys, Ser, Thr, Asn and Gln), among amino acid residues with acidic side chains (e.g. Asp, Glu), among amino acid residues with basic side chains (e.g. His, Lys, and Arg), or among amino acid residues with aromatic side chains (e.g. Trp, Tyr, and Phe). As known in the art, conservative substitution usually does not cause significant change in the protein conformational structure, and therefore could retain the biological activity of a protein.

The term “homologous” as used herein refers to nucleic acid sequences (or its complementary strand) or amino acid sequences that have sequence identity of at least 60% (e.g. at least 65%, 70%, 75%, 80%, 85%, 88%, 90%, 91%, 92%, 93%, 94%, 95%, 96%, 97%, 98%, 99%) to another sequences when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as C1 complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

“Antibody-dependent cell-mediated cytotoxicity” or “ADCC” as used herein refers to a cell-mediated reaction in which effector cells that express Fc receptors (FcRs) recognize bound antibody or antigen-binding fragment on a target cell and subsequently cause lysis of the target cell. “ADCC activity” or “ADCC effect” refers to the ability of the antibody or antigen-binding fragment which is bound on the target cell to elicit an ADCC reaction as described above.

“Complement dependent cytotoxicity” or “CDC” as used herein refers to a mechanism by which antibodies can mediate specific target cell lysis through activation of an organism's complement system. In CDC, the C1q binds the antibody and this binding triggers the complement cascade which leads to the formation of the membrane attack complex (MAC) (C5b to C9) at the surface of the target cell, as a result of the classical pathway complement activation. “CDC activity” or “CDC effect” refers to the ability of the antibody or antigen-binding fragment which is bound on the target cell to elicit a CDC reaction as described above.

“Target cells” as used herein refer to cells to which antibodies comprising an Fc region specifically bind, generally via the protein part that is C-terminal to the Fc region. “Effector cells” are leukocytes which express one or more Fc receptors and perform effector functions. Examples of human leukocytes which mediate ADCC include peripheral blood mononuclear cells (PBMCs), natural killer (NK) cells, monocytes, cytotoxic T cells and neutrophils; with PBMCs and NK cells being preferred. The effector cells may be isolated from a native source thereof, e.g., from blood or PBMCs as is known in the art.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or an antigen-binding fragment thereof, at least one promoter (e.g. SV40, CMV, EF-1α) operably linked to the nucleic acid sequence, and at least one selection marker.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient”, “subject” or “individual” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a CLDN18 (in particular, CLDN18.2) related disease, or refer to identification of a subject with a CLDN18 (in particular, CLDN18.2) related disease who may benefit from a particular treatment regimen. In some embodiments, diagnosis contains the identification of abnormal amount or activity of CLDN18.2. In some embodiments, diagnosis refers to the identification of a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample.

In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from stomach, heart, liver, spleen, lung, kidney, skin or blood vessels of the subject.

“CLDN18” as used herein refers to Claudin18 and includes any variants thereof, including CLDN18.1 and CLDN18.2, conformations, isoforms and species homologs of CLDN18 which are naturally expressed by cells or are expressed by cells transfected with the CLDN18 gene. In certain embodiments, the CLDN18 is human CLDN18. CLDN18 as used herein may be from other animal species, such as from human, mouse, and cynomolgus, among others. The terms “CLDN18”, “CLDN-18”, “CLDN 18”, “Claudin18”, “Claudin-18”, or “Claudin 18” may be used interchangeably in the present disclosure.

“CLDN18.1” is a splice variant of CLDN18, and includes post-translationally modified variants, isoforms and species homologs of CLDN18.1 which are naturally expressed by cells or are expressed on cells transfected with the CLDN18.1 gene. The terms “CLDN18.1”, “CLDN-18.1”, “CLDN 18.1”, “Claudin18.1”, “Claudin-18.1”, or “Claudin 18.1” may be used interchangeably in the present disclosure. Exemplary sequence of human CLDN18.1 protein is disclosed in NCBI Ref Seq No. NP_057453.1.

“CLDN18.2” is a splice variant of CLDN18, and includes post-translationally modified variants, isoforms and species homologs of CLDN18.2 which are naturally expressed by cells or are expressed on cells transfected with the CLDN18.2 gene. The terms “CLDN18.2”, “CLDN-18.2”, “CLDN 18.2”, “Claudin18.2”, “Claudin-18.2”, or “Claudin 18.2” may be used interchangeably in the present disclosure. Exemplary sequence of human CLDN18.2 protein is disclosed in NCBI Ref Seq No. NP_001002026.1.

The term “anti-CLDN18 antibody” refers to an antibody that is capable of specific binding to CLDN18 (e.g. human CLDN18). The term “anti-human CLDN18 antibody” refers to an antibody that is capable of specific binding to human CLDN18. In some embodiments, the anti-CLDN18 antibody provided herein is capable of binding to both CLDN18.1 and CLDN18.2. In some embodiments, the anti-CLDN18 antibody provided herein is capable of specifically binding to CLDN18.2, but not binding to CLDN18.1 or binding less well to CLDN18.1 (e.g. the binding affinity to CLDN18.1 is at least 10-fold lower than that to CLDN18.2, or at least 50-fold lower, or at least 100-fold lower, or at least 200-fold lower than that to CLDN18.2). In some embodiments, the anti-CLDN18 antibody provided herein does not have detectable binding affinity to CLDN18.1. In some embodiments, the binding affinity is determined by FACS assay. In some embodiments, the binding affinity is determined by Mean Fluorescence Intensity (MFI) detected by FACS assay.

A “CLDN18 related” disease, disorder or condition as used herein refers to any disease or condition caused by, exacerbated by, or otherwise linked to increased or decreased expression or activities of CLDN18. In some embodiments, the CLDN18 related disease, disorder or condition is a disorder related to excessive cell proliferation, such as, for example, cancer. In certain embodiments, the CLDN18 related disease or condition is characterized in expressing or over-expressing of CLDN18 and/or CLDN18 related genes such as CLDN18.1 gene or CLDN18.2 gene.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “CLDN18-positive cell” as used herein refer to a cell (e.g. epithelial cell) that expresses CLDN18 on the surface of the cell.

### Anti-CLDN18 Antibodies

The present disclosure provides anti-CLDN18 (in particular, anti-CLDN18.2) antibodies and antigen-binding fragments thereof. The anti-CLDN18 antibodies and antigen-binding fragments provided herein are capable of specific binding to CLDN18 (in particular, CLDN18.2).

In certain embodiments, the antibodies and antigen-binding fragments thereof provided herein specifically bind to human CLDN18 (in particular, human CLDN18.2). In certain embodiments, the antibodies and antigen-binding fragments thereof provided herein is capable of binding to both human CLDN18.1 and human CLDN18.2. In certain embodiments, the antibodies and antigen-binding fragments thereof provided herein specifically bind to human CLDN18.2 at a K_{D} value of no more than 10⁻⁷ M, no more than 8×10⁻⁸ M, no more than 5×10⁻⁸ M, no more than 2×10⁻⁸ M, no more than 10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, no more than 6×10⁻¹⁰ M, no more than 5×10⁻¹⁰ M, no more than 4×10⁻¹⁰ M, no more than 3×10⁻¹⁰ M, no more than 2×10⁻¹⁰ M as measured by Biacore assay. Biacore assay is based on surface plasmon resonance technology, see, for example, Murphy, M. et al., Current protocols in protein science, Chapter 19, unit 19.14, 2006.

In certain embodiments, the antibodies and the antigen-binding fragments thereof provided herein specifically bind to human CLDN18 (in particular, CLDN18.2) at a K_{D} value of no more than 10⁻⁸ M, no more than 8×10⁻⁹ M, no more than 5×10⁻⁹ M, no more than 1×10⁻⁹ M, no more than 8×10⁻¹⁰ M, no more than 5×10⁻¹⁰ M, no more than 1×10⁻¹⁰ M, no more than 8×10⁻¹¹ M, no more than 5×10⁻¹¹ M, no more than 1×10⁻¹¹ M, no more than 8×10⁻¹² M, no more than 5×10⁻¹² M, no more than 1×10⁻¹² M as measured by Octet assay. Octet assay is based on bio-layer interferometry technology, see, for example, Abdiche, Yasmina N., et al. Analytical biochemistry 386.2 (2009): 172-180, and Sun Y S., Instrumentation Science & Technology, 2014, 42(2): 109-127.

Binding of the antibodies or the antigen-binding fragments thereof provided herein to CLDN18 can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), FACS assay, and other binding assay. In certain embodiments, the antibodies and antigen-binding fragments thereof provided herein specifically bind to human or mouse CLDN18 (in particular, human or mouse CLDN18.2) at an EC₅₀ value (i.e. 50% binding concentration) of no more than 10⁻⁹ M, no more than 8×10⁻¹⁰ M, no more than 7×10⁻¹⁰ M, no more than 6×10⁻¹⁰ M, no more than 5×10⁻¹⁰ M, no more than 4×10⁻¹⁰ M, no more than 3×10⁻¹⁰ M, no more than 2×10⁻¹⁰ M by FACS assay. In certain embodiments, the binding is measured by ELISA or FACS assay. In certain embodiments, the EC₅₀ value is measured by the method as described in Example 2.3 of the present disclosure.

In some embodiments, the antibody or an antigen-binding fragment thereof provided herein specifically binds to CLDN18.2. In some embodiments, the antibody or an antigen-binding fragment thereof provided herein specifically binds to human CLDN18.2. In some embodiments, the antibody or an antigen-binding fragment thereof provided herein does not bind to other members of CLDN family (for example, CLDN18.1). In some embodiments, the antibody or an antigen-binding fragment thereof provided herein specifically binds to human CLDN18.2, but does not specifically bind to human CLDN18.1, for example, as measured by FACS assay.

In certain embodiments, the antibodies and antigen-binding fragments thereof provided herein specifically bind to mouse CLDN18.2 at an EC₅₀ value of no more than 10⁻⁸ M, no more than 8×10⁻⁹ M, no more than 5×10⁻⁹ M, no more than 2×10⁻⁹ M, no more than 10⁻⁹ M, no more than 8×10⁻¹⁰ M, no more than 7×10⁻¹⁰ M, no more than 6×10⁻¹⁰ M, no more than 5×10⁻¹⁰ M, or no more than 4×10⁻¹⁰ M by FACS assay.

### Illustrative Anti-CLDN18 Antibodies

In certain embodiments, the present disclosure provides anti-CLDN18 antibodies (e.g. anti-CLDN18.2 antibodies) and antigen-binding fragments thereof comprising one or more (e.g. 1, 2, 3, 4, 5, or 6) CDRs comprising the sequences selected from the group consisting of SEQ ID NOs: 1-155, 201-205, 332-354, and 367. In certain embodiments, the present disclosure further encompass antibodies and antigen binding fragments thereof having no more than one, two or three amino acid residue substitutions to any of SEQ ID NOs: 1-155, 201-205, 332-354, and 367. The specific amino acid sequence of each CDR as described above is shown in Table 2 below.

Antibody “99H8” as used herein refers to a monoclonal antibody comprising a heavy chain variable region having the sequence of SEQ ID NO: 254, and a light chain variable region having the sequence of SEQ ID NO: 302.

Antibody “99G8” as used herein refers to a monoclonal antibody comprising a heavy chain variable region having the sequence of SEQ ID NO: 229, and a light chain variable region having the sequence of SEQ ID NO: 282.

Antibody “99A7” as used herein refers to a monoclonal antibody comprising a heavy chain variable region having the sequence of SEQ ID NO: 235, and a light chain variable region having the sequence of SEQ ID NO: 288.

Antibody “97A9” as used herein refers to a monoclonal antibody comprising a heavy chain variable region having the sequence of SEQ ID NO: 250, and a light chain variable region having the sequence of SEQ ID NO: 290.

Antibody “84E8” as used herein refers to a monoclonal antibody comprising a heavy chain variable region having the sequence of SEQ ID NO: 236, and a light chain variable region having the sequence of SEQ ID NO: 293.

Antibody “83H3” as used herein refers to a monoclonal antibody comprising a heavy chain variable region having the sequence of SEQ ID NO: 256, and a light chain variable region having the sequence of SEQ ID NO: 268.

Antibody “80F10” as used herein refers to a monoclonal antibody comprising a heavy chain variable region having the sequence of SEQ ID NO: 251, and a light chain variable region having the sequence of SEQ ID NO: 291.

Antibody “79C3” as used herein refers to a monoclonal antibody comprising a heavy chain variable region having the sequence of SEQ ID NO: 220, and a light chain variable region having the sequence of SEQ ID NO: 284.

Antibody “78H6” as used herein refers to a monoclonal antibody comprising a heavy chain variable region having the sequence of SEQ ID NO: 222, and a light chain variable region having the sequence of SEQ ID NO: 260.

Antibody “73E4” as used herein refers to a monoclonal antibody comprising a heavy chain variable region having the sequence of SEQ ID NO: 253, and a light chain variable region having the sequence of SEQ ID NO: 270.

Antibody “69B2” as used herein refers to a monoclonal antibody comprising a heavy chain variable region having the sequence of SEQ ID NO: 232, and a light chain variable region having the sequence of SEQ ID NO: 287.

Antibody “68E9” as used herein refers to a monoclonal antibody comprising a heavy chain variable region having the sequence of SEQ ID NO: 223, and a light chain variable region having the sequence of SEQ ID NO: 285.

Antibody “68D1” as used herein refers to a monoclonal antibody comprising a heavy chain variable region having the sequence of SEQ ID NO: 252, and a light chain variable region having the sequence of SEQ ID NO: 272.

Antibody “66E6” as used herein refers to a monoclonal antibody comprising a heavy chain variable region having the sequence of SEQ ID NO: 255, and a light chain variable region having the sequence of SEQ ID NO: 299.

Antibody “66E12” as used herein refers to a monoclonal antibody comprising a heavy chain variable region having the sequence of SEQ ID NO: 233, and a light chain variable region having the sequence of SEQ ID NO: 292.

Antibody “64C1” as used herein refers to a monoclonal antibody comprising a heavy chain variable region having the sequence of SEQ ID NO: 217, and a light chain variable region having the sequence of SEQ ID NO: 262.

Antibody “64C10” as used herein refers to a monoclonal antibody comprising a heavy chain variable region having the sequence of SEQ ID NO: 219, and a light chain variable region having the sequence of SEQ ID NO: 264.

Antibody “61A5” as used herein refers to a monoclonal antibody comprising a heavy chain variable region having the sequence of SEQ ID NO: 211, and a light chain variable region having the sequence of SEQ ID NO: 261.

Antibody “60F11” as used herein refers to a monoclonal antibody comprising a heavy chain variable region having the sequence of SEQ ID NO: 234, and a light chain variable region having the sequence of SEQ ID NO: 274.

Antibody “59G12” as used herein refers to a monoclonal antibody comprising a heavy chain variable region having the sequence of SEQ ID NO: 216, and a light chain variable region having the sequence of SEQ ID NO: 263.

Antibody “59F5” as used herein refers to a monoclonal antibody comprising a heavy chain variable region having the sequence of SEQ ID NO: 218, and a light chain variable region having the sequence of SEQ ID NO: 262.

Antibody “59E7” as used herein refers to a monoclonal antibody comprising a heavy chain variable region having the sequence of SEQ ID NO: 215, and a light chain variable region having the sequence of SEQ ID NO: 263.

Antibody “56B2” as used herein refers to a monoclonal antibody comprising a heavy chain variable region having the sequence of SEQ ID NO: 227, and a light chain variable region having the sequence of SEQ ID NO: 286.

Antibody “54F5” as used herein refers to a monoclonal antibody comprising a heavy chain variable region having the sequence of SEQ ID NO: 221, and a light chain variable region having the sequence of SEQ ID NO: 281.

Antibody “38B9” as used herein refers to a monoclonal antibody comprising a heavy chain variable region having the sequence of SEQ ID NO: 213, and a light chain variable region having the sequence of SEQ ID NO: 308.

Antibody “35B4” as used herein refers to a monoclonal antibody comprising a heavy chain variable region having the sequence of SEQ ID NO: 210, and a light chain variable region having the sequence of SEQ ID NO: 307.

Antibody “35A10” as used herein refers to a monoclonal antibody comprising a heavy chain variable region having the sequence of SEQ ID NO: 207, and a light chain variable region having the sequence of SEQ ID NO: 280.

Antibody “33G12” as used herein refers to a monoclonal antibody comprising a heavy chain variable region having the sequence of SEQ ID NO: 212, and a light chain variable region having the sequence of SEQ ID NO: 309.

Antibody “22E12” as used herein refers to a monoclonal antibody comprising a heavy chain variable region having the sequence of SEQ ID NO: 246, and a light chain variable region having the sequence of SEQ ID NO: 273.

Antibody “15E10” as used herein refers to a monoclonal antibody comprising a heavy chain variable region having the sequence of SEQ ID NO: 208, and a light chain variable region having the sequence of SEQ ID NO: 278.

Antibody “100F4” as used herein refers to a monoclonal antibody comprising a heavy chain variable region having the sequence of SEQ ID NO: 249, and a light chain variable region having the sequence of SEQ ID NO: 294.

Antibody “40C1” as used herein refers to a monoclonal antibody comprising a heavy chain variable region having the sequence of SEQ ID NO: 214, and a light chain variable region having the sequence of SEQ ID NO: 269.

Antibody “41B3” as used herein refers to a monoclonal antibody comprising a heavy chain variable region having the sequence of SEQ ID NO: 206, and a light chain variable region having the sequence of SEQ ID NO: 305.

Antibody “66D7-1” as used herein refers to a monoclonal antibody comprising a heavy chain variable region having the sequence of SEQ ID NO: 230, and a light chain variable region having the sequence of SEQ ID NO: 279.

Antibody “66D7-2” as used herein refers to a monoclonal antibody comprising a heavy chain variable region having the sequence of SEQ ID NO: 257, and a light chain variable region having the sequence of SEQ ID NO: 271.

Antibody “51G10” as used herein refers to a monoclonal antibody comprising a heavy chain variable region having the sequence of SEQ ID NO: 259, and a light chain variable region having the sequence of SEQ ID NO: 271.

Antibody “365F6” as used herein refers to a monoclonal antibody comprising a heavy chain variable region having the sequence of SEQ ID NO: 241, and a light chain variable region having the sequence of SEQ ID NO: 283.

Antibody “360C2” as used herein refers to a monoclonal antibody comprising a heavy chain variable region having the sequence of SEQ ID NO: 231, and a light chain variable region having the sequence of SEQ ID NO: 306.

Antibody “319F2” as used herein refers to a monoclonal antibody comprising a heavy chain variable region having the sequence of SEQ ID NO: 230, and a light chain variable region having the sequence of SEQ ID NO: 303.

Antibody “317A7” as used herein refers to a monoclonal antibody comprising a heavy chain variable region having the sequence of SEQ ID NO: 248, and a light chain variable region having the sequence of SEQ ID NO: 289.

Antibody “315F10” as used herein refers to a monoclonal antibody comprising a heavy chain variable region having the sequence of SEQ ID NO: 258, and a light chain variable region having the sequence of SEQ ID NO: 289.

Antibody “314D7” as used herein refers to a monoclonal antibody comprising a heavy chain variable region having the sequence of SEQ ID NO: 245, and a light chain variable region having the sequence of SEQ ID NO: 266.

Antibody “310H5” as used herein refers to a monoclonal antibody comprising a heavy chain variable region having the sequence of SEQ ID NO: 228, and a light chain variable region having the sequence of SEQ ID NO: 300.

Antibody “308E8” as used herein refers to a monoclonal antibody comprising a heavy chain variable region having the sequence of SEQ ID NO: 247, and a light chain variable region having the sequence of SEQ ID NO: 289.

Antibody “305G8” as used herein refers to a monoclonal antibody comprising a heavy chain variable region having the sequence of SEQ ID NO: 244, and a light chain variable region having the sequence of SEQ ID NO: 266.

Antibody “256C10⁻¹” as used herein refers to a monoclonal antibody comprising a heavy chain variable region having the sequence of SEQ ID NO: 258, and a light chain variable region having the sequence of SEQ ID NO: 301.

Antibody “256C10⁻²” as used herein refers to a monoclonal antibody comprising a heavy chain variable region having the sequence of SEQ ID NO: 209, and a light chain variable region having the sequence of SEQ ID NO: 301.

Antibody “248D9” as used herein refers to a monoclonal antibody comprising a heavy chain variable region having the sequence of SEQ ID NO: 238, and a light chain variable region having the sequence of SEQ ID NO: 275.

Antibody “246B8” as used herein refers to a monoclonal antibody comprising a heavy chain variable region having the sequence of SEQ ID NO: 240, and a light chain variable region having the sequence of SEQ ID NO: 276.

Antibody “243A8” as used herein refers to a monoclonal antibody comprising a heavy chain variable region having the sequence of SEQ ID NO: 226, and a light chain variable region having the sequence of SEQ ID NO: 297.

Antibody “242G5” as used herein refers to a monoclonal antibody comprising a heavy chain variable region having the sequence of SEQ ID NO: 224, and a light chain variable region having the sequence of SEQ ID NO: 296.

Antibody “237E3” as used herein refers to a monoclonal antibody comprising a heavy chain variable region having the sequence of SEQ ID NO: 226, and a light chain variable region having the sequence of SEQ ID NO: 298.

Antibody “226E9” as used herein refers to a monoclonal antibody comprising a heavy chain variable region having the sequence of SEQ ID NO: 225, and a light chain variable region having the sequence of SEQ ID NO: 310.

Antibody “226D5” as used herein refers to a monoclonal antibody comprising a heavy chain variable region having the sequence of SEQ ID NO: 239, and a light chain variable region having the sequence of SEQ ID NO: 277.

Antibody “217B5” as used herein refers to a monoclonal antibody comprising a heavy chain variable region having the sequence of SEQ ID NO: 237, and a light chain variable region having the sequence of SEQ ID NO: 304.

Antibody “214E4” as used herein refers to a monoclonal antibody comprising a heavy chain variable region having the sequence of SEQ ID NO: 243, and a light chain variable region having the sequence of SEQ ID NO: 267.

Antibody “213A9” as used herein refers to a monoclonal antibody comprising a heavy chain variable region having the sequence of SEQ ID NO: 242, and a light chain variable region having the sequence of SEQ ID NO: 295.

Antibody “206C7” as used herein refers to a monoclonal antibody comprising a heavy chain variable region having the sequence of SEQ ID NO: 244, and a light chain variable region having the sequence of SEQ ID NO: 265.

Antibody “203D12” as used herein refers to a monoclonal antibody comprising a heavy chain variable region having the sequence of SEQ ID NO: 242, and a light chain variable region having the sequence of SEQ ID NO: 289.

Antibody “203A5” as used herein refers to a monoclonal antibody comprising a heavy chain variable region having the sequence of SEQ ID NO: 225, and a light chain variable region having the sequence of SEQ ID NO: 296.

The specific amino acid sequences of the heavy chain variable region and light chain variable region of each exemplary antibody as described above are shown in Table 3 below.

In certain embodiments, the present disclosure provides anti-CLDN18 (in particular, anti-CLDN18.2) antibodies and antigen-binding fragments thereof comprising one or more (e.g. 1, 2, 3, 4, 5, or 6) CDR sequences of Antibody 99H8, 99G8, 99A7, 97A9, 84E8, 83H3, 80F10, 79C3, 78H6, 73E4, 69B2, 68E9, 68D1, 66E6, 66E12, 64C1, 64C10, 61A5, 60F11, 59G12, 59F5, 59E7, 56B2, 54F5, 38B9, 35B4, 35A10, 33G12, 22E12, 15E10, 100F4, 40C1, 41B3, 66D7-1, 66D7-2, 51G10, 365F6, 360C2, 319F2, 317A7, 315F10, 314D7, 310H5, 308E8, 305G8, 256C10⁻¹, 256C10⁻², 248D9, 246B8, 243A8, 242G5, 237E3, 226E9, 226D5, 217B5, 214E4, 213A9, 206C7, 203D12 or 203A5.

In certain embodiments, the present disclosure provides anti-CLDN18 (in particular, anti-CLDN18.2) antibodies and antigen-binding fragments thereof comprising a HCDR1 comprising an amino acid sequence selected from the group consisting of SEQ ID NOs: 1-28, 201, 202, 332-337, a HCDR2 comprising an amino acid sequence selected from the group consisting of SEQ ID NOs: 29-67, 203, 338-343, 367, and a HCDR3 comprising an amino acid sequence selected from the group consisting of SEQ ID NOs: 68-94, 344-346, and/or a LCDR1 comprising an amino acid sequence selected from the group consisting of SEQ ID NOs: 95-113, 205, 347, 348, a LCDR2 comprising an amino acid sequence selected from the group consisting of SEQ ID NOs: 114-123, 349, 350, and a LCDR3 comprising an amino acid sequence selected from the group consisting of SEQ ID NOs: 124-155, 204, 351-354.

In certain embodiments, the present disclosure provides anti-CLDN18 (in particular, anti-CLDN18.2) antibodies and antigen-binding fragments thereof comprising a HCDR1 comprising an amino acid sequence selected from the group consisting of SEQ ID NOs: 1, 4, 11, 15-20, 201, 202, 332-337, a HCDR2 comprising an amino acid sequence selected from the group consisting of SEQ ID NOs: 29, 32, 43, 46-51, 53, 203, 338-343, a HCDR3 comprising an amino acid sequence selected from the group consisting of SEQ ID NOs: 68, 69, 71, 79, 80-85, 344-346, and/or a LCDR1 comprising an amino acid sequence selected from the group consisting of SEQ ID NOs: 95, 96, 101-104, 106, 205, 347, 348, a LCDR2 comprising an amino acid sequence selected from the group consisting of SEQ ID NOs: 114, 115, 117-122, 349, 350, and a LCDR3 comprising an amino acid sequence selected from the group consisting of SEQ ID NOs: 124, 127, 135, 137-142, 144, 204, 351-354.

In certain embodiments, the present disclosure provides anti-CLDN18 (in particular, anti-CLDN18.2) antibodies and antigen-binding fragments thereof comprising a HCDR1 comprising an amino acid sequence selected from the group consisting of SEQ ID NOs: 1, 4, 11, 15-20, 201, 202, a HCDR2 comprising an amino acid sequence selected from the group consisting of SEQ ID NOs: 29, 32, 43, 46-51, 53, 203, a HCDR3 comprising an amino acid sequence selected from the group consisting of SEQ ID NOs: 68, 69, 71, 79, 80-85, and/or a LCDR1 comprising an amino acid sequence selected from the group consisting of SEQ ID NOs: 95, 96, 101-104, 106, 205, a LCDR2 comprising an amino acid sequence selected from the group consisting of SEQ ID NOs: 114, 115, 117-122, and a LCDR3 comprising an amino acid sequence selected from the group consisting of SEQ ID NOs: 124, 127, 135, 137-142, 144, 204.

In certain embodiments, the present disclosure provides anti-CLDN18 (in particular, anti-CLDN18.2) antibodies and antigen-binding fragments thereof comprising a HCDR1 comprising an amino acid sequence selected from the group consisting of SEQ ID NOs: 16, 19, 201, 202, a HCDR2 comprising an amino acid sequence selected from the group consisting of SEQ ID NOs: 47, 50, 203, a HCDR3 comprising an amino acid sequence selected from the group consisting of SEQ ID NO: 80, 83, and/or a LCDR1 comprising an amino acid sequence selected from the group consisting of SEQ ID NOs: 96, 103, 205, a LCDR2 comprising an amino acid sequence selected from the group consisting of SEQ ID NO: 118, 120, and a LCDR3 comprising an amino acid sequence selected from the group consisting of SEQ ID NOs: 138, 141, 204.

In certain embodiments, the present disclosure provides anti-CLDN18 (in particular, anti-CLDN18.2) antibodies and antigen-binding fragments thereof comprising a HCDR1 comprising the sequence of SEQ ID NO: 1, a HCDR2 comprising the sequence of SEQ ID NO: 29, and a HCDR3 comprising the sequence of SEQ ID NO: 68, and/or a LCDR1 comprises the sequence of SEQ ID NO: 95, a LCDR2 comprises the sequence of SEQ ID NO: 114, and a LCDR3 comprises the sequence of SEQ ID NO: 124.

In certain embodiments, the present disclosure provides anti-CLDN18 (in particular, anti-CLDN18.2) antibodies and antigen-binding fragments thereof comprising a HCDR1 comprising the sequence of SEQ ID NO: 2, a HCDR2 comprising the sequence of SEQ ID NO: 30, a HCDR3 comprising the sequence of SEQ ID NO: 69, and/or a LCDR1 comprising the sequence of SEQ ID NO: 96, a LCDR2 comprising the sequence of SEQ ID NO: 114, and a LCDR3 comprising the sequence of SEQ ID NO: 125.

In certain embodiments, the present disclosure provides anti-CLDN18 (in particular, anti-CLDN18.2) antibodies and antigen-binding fragments thereof comprising a HCDR1 comprising the sequence of SEQ ID NO: 3, a HCDR2 comprising the sequence of SEQ ID NO: 31, a HCDR3 comprising the sequence of SEQ ID NO: 70, and/or a LCDR1 comprising the sequence of SEQ ID NO: 96, a LCDR2 comprising the sequence of SEQ ID NO: 115, and a LCDR3 comprising the sequence of SEQ ID NO: 126.

In certain embodiments, the present disclosure provides anti-CLDN18 (in particular, anti-CLDN18.2) antibodies and antigen-binding fragments thereof comprising a HCDR1 comprising the sequence of SEQ ID NO: 4, a HCDR2 comprising the sequence of SEQ ID NO: 32, a HCDR3 comprising the sequence of SEQ ID NO: 69, and/or a LCDR1 comprising the sequence of SEQ ID NO: 96, a LCDR2 comprising the sequence of SEQ ID NO: 115, and a LCDR3 comprising the sequence of SEQ ID NO: 127.

In certain embodiments, the present disclosure provides anti-CLDN18 (in particular, anti-CLDN18.2) antibodies and antigen-binding fragments thereof comprising a HCDR1 comprising the sequence of SEQ ID NO: 5, a HCDR2 comprising the sequence of SEQ ID NO: 33, a HCDR3 comprising the sequence of SEQ ID NO: 71, and/or a LCDR1 comprising the sequence of SEQ ID NO: 96, a LCDR2 comprising the sequence of SEQ ID NO: 116, and a LCDR3 comprising the sequence of SEQ ID NO: 128.

In certain embodiments, the present disclosure provides anti-CLDN18 (in particular, anti-CLDN18.2) antibodies and antigen-binding fragments thereof comprising a HCDR1 comprising the sequence of SEQ ID NO: 4, a HCDR2 comprising the sequence of SEQ ID NO: 34, a HCDR3 comprising the sequence of SEQ ID NO: 69, and/or a LCDR1 comprising the sequence of SEQ ID NO: 97, a LCDR2 comprising the sequence of SEQ ID NO: 115, and a LCDR3 comprising the sequence of SEQ ID NO: 129.

In certain embodiments, the present disclosure provides anti-CLDN18 (in particular, anti-CLDN18.2) antibodies and antigen-binding fragments thereof comprising a HCDR1 comprising the sequence of SEQ ID NO: 6, a HCDR2 comprising the sequence of SEQ ID NO: 32, a HCDR3 comprising the sequence of SEQ ID NO: 69, and/or a LCDR1 comprising the sequence of SEQ ID NO: 96, a LCDR2 comprising the sequence of SEQ ID NO: 115, and a LCDR3 comprising the sequence of SEQ ID NO: 127.

In certain embodiments, the present disclosure provides anti-CLDN18 (in particular, anti-CLDN18.2) antibodies and antigen-binding fragments thereof comprising a HCDR1 comprising the sequence of SEQ ID NO: 7, a HCDR2 comprising the sequence of SEQ ID NO: 35, a HCDR3 comprising the sequence of SEQ ID NO: 72, and/or a LCDR1 comprising the sequence of SEQ ID NO: 96, a LCDR2 comprising the sequence of SEQ ID NO: 115, and a LCDR3 comprising the sequence of SEQ ID NO: 130.

In certain embodiments, the present disclosure provides anti-CLDN18 (in particular, anti-CLDN18.2) antibodies and antigen-binding fragments thereof comprising a HCDR1 comprising the sequence of SEQ ID NO: 8, a HCDR2 comprising the sequence of SEQ ID NO: 36, a HCDR3 comprising the sequence of SEQ ID NO: 72, and/or a LCDR1 comprising the sequence of SEQ ID NO: 96, a LCDR2 comprising the sequence of SEQ ID NO: 115, and a LCDR3 comprising the sequence of SEQ ID NO: 130.

In certain embodiments, the present disclosure provides anti-CLDN18 (in particular, anti-CLDN18.2) antibodies and antigen-binding fragments thereof comprising a HCDR1 comprising the sequence of SEQ ID NO: 6, a HCDR2 comprising the sequence of SEQ ID NO: 37, a HCDR3 comprising the sequence of SEQ ID NO: 69, and/or a LCDR1 comprising the sequence of SEQ ID NO: 98, a LCDR2 comprising the sequence of SEQ ID NO: 115, and a LCDR3 comprising the sequence of SEQ ID NO: 127.

In certain embodiments, the present disclosure provides anti-CLDN18 (in particular, anti-CLDN18.2) antibodies and antigen-binding fragments thereof comprising a HCDR1 comprising the sequence of SEQ ID NO: 5, a HCDR2 comprising the sequence of SEQ ID NO: 38, a HCDR3 comprising the sequence of SEQ ID NO: 73, and/or a LCDR1 comprising the sequence of SEQ ID NO: 96, a LCDR2 comprising the sequence of SEQ ID NO: 115, and a LCDR3 comprising the sequence of SEQ ID NO: 128.

In certain embodiments, the present disclosure provides anti-CLDN18 (in particular, anti-CLDN18.2) antibodies and antigen-binding fragments thereof comprising a HCDR1 comprising the sequence of SEQ ID NO: 8, a HCDR2 comprising the sequence of SEQ ID NO: 35, a HCDR3 comprising the sequence of SEQ ID NO: 74, and/or a LCDR1 comprising the sequence of SEQ ID NO: 96, a LCDR2 comprising the sequence of SEQ ID NO: 115, and a LCDR3 comprising the sequence of SEQ ID NO: 130.

In certain embodiments, the present disclosure provides anti-CLDN18 (in particular, anti-CLDN18.2) antibodies and antigen-binding fragments thereof comprising a HCDR1 comprising the sequence of SEQ ID NO: 6, a HCDR2 comprising the sequence of SEQ ID NO: 32, a HCDR3 comprising the sequence of SEQ ID NO: 69, and/or a LCDR1 comprising the sequence of SEQ ID NO: 96, a LCDR2 comprising the sequence of SEQ ID NO: 115, and a LCDR3 comprising the sequence of SEQ ID NO: 127.

In certain embodiments, the present disclosure provides anti-CLDN18 (in particular, anti-CLDN18.2) antibodies and antigen-binding fragments thereof comprising a HCDR1 comprising the sequence of SEQ ID NO: 2, a HCDR2 comprising the sequence of SEQ ID NO: 39, a HCDR3 comprising the sequence of SEQ ID NO: 69, and/or a LCDR1 comprising the sequence of SEQ ID NO: 99, a LCDR2 comprising the sequence of SEQ ID NO: 115, and a LCDR3 comprising the sequence of SEQ ID NO: 131.

In certain embodiments, the present disclosure provides anti-CLDN18 (in particular, anti-CLDN18.2) antibodies and antigen-binding fragments thereof comprising a HCDR1 comprising the sequence of SEQ ID NO: 5, a HCDR2 comprising the sequence of SEQ ID NO: 33, a HCDR3 comprising the sequence of SEQ ID NO: 71, and/or a LCDR1 comprising the sequence of SEQ ID NO: 96, a LCDR2 comprising the sequence of SEQ ID NO: 115, and a LCDR3 comprising the sequence of SEQ ID NO: 128.

In certain embodiments, the present disclosure provides anti-CLDN18 (in particular, anti-CLDN18.2) antibodies and antigen-binding fragments thereof comprising a HCDR1 comprising the sequence of SEQ ID NO: 9, the HCDR2 comprising the sequence of SEQ ID NO: 40, the HCDR3 comprising the sequence of SEQ ID NO: 75, and/or a LCDR1 comprising the sequence of SEQ ID NO: 99, a LCDR2 comprising the sequence of SEQ ID NO: 115, and a LCDR3 comprising the sequence of SEQ ID NO: 132.

In certain embodiments, the present disclosure provides anti-CLDN18 (in particular, anti-CLDN18.2) antibodies and antigen-binding fragments thereof comprising a HCDR1 comprising the sequence of SEQ ID NO: 9, the HCDR2 comprising the sequence of SEQ ID NO: 41, the HCDR3 comprising the sequence of SEQ ID NO: 76, and/or a LCDR1 comprising the sequence of SEQ ID NO: 99, a LCDR2 comprising the sequence of SEQ ID NO: 115, and a LCDR3 comprising the sequence of SEQ ID NO: 133.

In certain embodiments, the present disclosure provides anti-CLDN18 (in particular, anti-CLDN18.2) antibodies and antigen-binding fragments thereof comprising a HCDR1 comprising the sequence of SEQ ID NO: 10, the HCDR2 comprising the sequence of SEQ ID NO: 42, the HCDR3 comprising the sequence of SEQ ID NO: 77, and/or a LCDR1 comprising the sequence of SEQ ID NO: 100, a LCDR2 comprising the sequence of SEQ ID NO: 115, and a LCDR3 comprising the sequence of SEQ ID NO: 134.

In certain embodiments, the present disclosure provides anti-CLDN18 (in particular, anti-CLDN18.2) antibodies and antigen-binding fragments thereof comprising a HCDR1 comprising the sequence of SEQ ID NO: 11, the HCDR2 comprising the sequence of SEQ ID NO: 43, the HCDR3 comprising the sequence of SEQ ID NO: 71, and/or a LCDR1 comprising the sequence of SEQ ID NO: 101, a LCDR2 comprising the sequence of SEQ ID NO: 115, and a LCDR3 comprising the sequence of SEQ ID NO: 135.

In certain embodiments, the present disclosure provides anti-CLDN18 (in particular, anti-CLDN18.2) antibodies and antigen-binding fragments thereof comprising a HCDR1 comprising the sequence of SEQ ID NO: 12, the HCDR2 comprising the sequence of SEQ ID NO: 44, the HCDR3 comprising the sequence of SEQ ID NO: 75, and/or a LCDR1 comprising the sequence of SEQ ID NO: 99, a LCDR2 comprising the sequence of SEQ ID NO: 115, and a LCDR3 comprising the sequence of SEQ ID NO: 132.

In certain embodiments, the present disclosure provides anti-CLDN18 (in particular, anti-CLDN18.2) antibodies and antigen-binding fragments thereof comprising a HCDR1 comprising the sequence of SEQ ID NO: 13, the HCDR2 comprising the sequence of SEQ ID NO: 40, the HCDR3 comprising the sequence of SEQ ID NO: 75, and/or a LCDR1 comprising the sequence of SEQ ID NO: 99, a LCDR2 comprising the sequence of SEQ ID NO: 115, and a LCDR3 comprising the sequence of SEQ ID NO: 132.

In certain embodiments, the present disclosure provides anti-CLDN18 (in particular, anti-CLDN18.2) antibodies and antigen-binding fragments thereof comprising a HCDR1 comprising the sequence of SEQ ID NO: 12, the HCDR2 comprising the sequence of SEQ ID NO: 44, the HCDR3 comprising the sequence of SEQ ID NO: 75, and/or a LCDR1 comprising the sequence of SEQ ID NO: 99, a LCDR2 comprising the sequence of SEQ ID NO: 115, and a LCDR3 comprising the sequence of SEQ ID NO: 132.

In certain embodiments, the present disclosure provides anti-CLDN18 (in particular, anti-CLDN18.2) antibodies and antigen-binding fragments thereof comprising a HCDR1 comprising the sequence of SEQ ID NO: 14, the HCDR2 comprising the sequence of SEQ ID NO: 45, the HCDR3 comprising the sequence of SEQ ID NO: 78, and/or a LCDR1 comprising the sequence of SEQ ID NO: 96, a LCDR2 comprising the sequence of SEQ ID NO: 115, and a LCDR3 comprising the sequence of SEQ ID NO: 136.

In certain embodiments, the present disclosure provides anti-CLDN18 (in particular, anti-CLDN18.2) antibodies and antigen-binding fragments thereof comprising a HCDR1 comprising the sequence of SEQ ID NO: 8, the HCDR2 comprising the sequence of SEQ ID NO: 35, the HCDR3 comprising the sequence of SEQ ID NO: 72, and/or a LCDR1 comprising the sequence of SEQ ID NO: 96, a LCDR2 comprising the sequence of SEQ ID NO: 115, and a LCDR3 comprising the sequence of SEQ ID NO: 130.

In certain embodiments, the present disclosure provides anti-CLDN18 (in particular, anti-CLDN18.2) antibodies and antigen-binding fragments thereof comprising a HCDR1 comprising the sequence of SEQ ID NO: 15, the HCDR2 comprising the sequence of SEQ ID NO: 46, the HCDR3 comprising the sequence of SEQ ID NO: 79, and/or a LCDR1 comprising the sequence of SEQ ID NO: 102, a LCDR2 comprising the sequence of SEQ ID NO: 117, and a LCDR3 comprising the sequence of SEQ ID NO: 137.

In certain embodiments, the present disclosure provides anti-CLDN18 (in particular, anti-CLDN18.2) antibodies and antigen-binding fragments thereof comprising a HCDR1 comprising the sequence of SEQ ID NO: 16, the HCDR2 comprising the sequence of SEQ ID NO: 47, the HCDR3 comprising the sequence of SEQ ID NO: 80, and/or a LCDR1 comprising the sequence of SEQ ID NO: 103, a LCDR2 comprising the sequence of SEQ ID NO: 118, and a LCDR3 comprising the sequence of SEQ ID NO: 138.

In certain embodiments, the present disclosure provides anti-CLDN18 (in particular, anti-CLDN18.2) antibodies and antigen-binding fragments thereof comprising a HCDR1 comprising the sequence of SEQ ID NO: 17, the HCDR2 comprising the sequence of SEQ ID NO: 48, the HCDR3 comprising the sequence of SEQ ID NO: 81, and/or a LCDR1 comprising the sequence of SEQ ID NO: 96, a LCDR2 comprising the sequence of SEQ ID NO: 119, and a LCDR3 comprising the sequence of SEQ ID NO: 139.

In certain embodiments, the present disclosure provides anti-CLDN18 (in particular, anti-CLDN18.2) antibodies and antigen-binding fragments thereof comprising a HCDR1 comprising the sequence of SEQ ID NO: 18, the HCDR2 comprising the sequence of SEQ ID NO: 49, the HCDR3 comprising the sequence of SEQ ID NO: 82, and/or a LCDR1 comprising the sequence of SEQ ID NO: 104, a LCDR2 comprising the sequence of SEQ ID NO: 118, and a LCDR3 comprising the sequence of SEQ ID NO: 140.

In certain embodiments, the present disclosure provides anti-CLDN18 (in particular, anti-CLDN18.2) antibodies and antigen-binding fragments thereof comprising a HCDR1 comprising the sequence of SEQ ID NO: 19, the HCDR2 comprising the sequence of SEQ ID NO: 50, the HCDR3 comprising the sequence of SEQ ID NO: 83, and/or a LCDR1 comprising the sequence of SEQ ID NO: 96, a LCDR2 comprising the sequence of SEQ ID NO: 120, and a LCDR3 comprising the sequence of SEQ ID NO: 141.

In certain embodiments, the present disclosure provides anti-CLDN18 (in particular, anti-CLDN18.2) antibodies and antigen-binding fragments thereof comprising a HCDR1 comprising the sequence of SEQ ID NO: 17, the HCDR2 comprising the sequence of SEQ ID NO: 51, the HCDR3 comprising the sequence of SEQ ID NO: 84, and/or a LCDR1 comprising the sequence of SEQ ID NO: 96, a LCDR2 comprising the sequence of SEQ ID NO: 121, and a LCDR3 comprising the sequence of SEQ ID NO: 142.

In certain embodiments, the present disclosure provides anti-CLDN18 (in particular, anti-CLDN18.2) antibodies and antigen-binding fragments thereof comprising a HCDR1 comprising the sequence of SEQ ID NO: 2, the HCDR2 comprising the sequence of SEQ ID NO: 52, the HCDR3 comprising the sequence of SEQ ID NO: 69, and/or a LCDR1 comprising the sequence of SEQ ID NO: 105, a LCDR2 comprising the sequence of SEQ ID NO: 115, and a LCDR3 comprising the sequence of SEQ ID NO: 143.

In certain embodiments, the present disclosure provides anti-CLDN18 (in particular, anti-CLDN18.2) antibodies and antigen-binding fragments thereof comprising a HCDR1 comprising the sequence of SEQ ID NO: 20, the HCDR2 comprising the sequence of SEQ ID NO: 53, the HCDR3 comprising the sequence of SEQ ID NO: 85, and/or a LCDR1 comprising the sequence of SEQ ID NO: 106, a LCDR2 comprising the sequence of SEQ ID NO: 122, and a LCDR3 comprising the sequence of SEQ ID NO: 144.

In certain embodiments, the present disclosure provides anti-CLDN18 (in particular, anti-CLDN18.2) antibodies and antigen-binding fragments thereof comprising a HCDR1 comprising the sequence of SEQ ID NO: 21, the HCDR2 comprising the sequence of SEQ ID NO: 54, the HCDR3 comprising the sequence of SEQ ID NO: 86, and/or a LCDR1 comprising the sequence of SEQ ID NO: 95, a LCDR2 comprising the sequence of SEQ ID NO: 115, and a LCDR3 comprising the sequence of SEQ ID NO: 128.

In certain embodiments, the present disclosure provides anti-CLDN18 (in particular, anti-CLDN18.2) antibodies and antigen-binding fragments thereof comprising a HCDR1 comprising the sequence of SEQ ID NO: 22, the HCDR2 comprising the sequence of SEQ ID NO: 55, the HCDR3 comprising the sequence of SEQ ID NO: 87, and/or a LCDR1 comprising the sequence of SEQ ID NO: 98, a LCDR2 comprising the sequence of SEQ ID NO: 115, and a LCDR3 comprising the sequence of SEQ ID NO: 145.

In certain embodiments, the present disclosure provides anti-CLDN18 (in particular, anti-CLDN18.2) antibodies and antigen-binding fragments thereof comprising a HCDR1 comprising the sequence of SEQ ID NO: 2, the HCDR2 comprising the sequence of SEQ ID NO: 56, the HCDR3 comprising the sequence of SEQ ID NO: 69, and/or a LCDR1 comprising the sequence of SEQ ID NO: 98, a LCDR2 comprising the sequence of SEQ ID NO: 115, and a LCDR3 comprising the sequence of SEQ ID NO: 127.

In certain embodiments, the present disclosure provides anti-CLDN18 (in particular, anti-CLDN18.2) antibodies and antigen-binding fragments thereof comprising a HCDR1 comprising the sequence of SEQ ID NO: 1, the HCDR2 comprising the sequence of SEQ ID NO: 57, the HCDR3 comprising the sequence of SEQ ID NO: 69, and/or a LCDR1 comprising the sequence of SEQ ID NO: 98, a LCDR2 comprising the sequence of SEQ ID NO: 115, and a LCDR3 comprising the sequence of SEQ ID NO: 127.

In certain embodiments, the present disclosure provides anti-CLDN18 (in particular, anti-CLDN18.2) antibodies and antigen-binding fragments thereof comprising a HCDR1 comprising the sequence of SEQ ID NO: 11, the HCDR2 comprising the sequence of SEQ ID NO: 43, the HCDR3 comprising the sequence of SEQ ID NO: 88, and/or a LCDR1 comprising the sequence of SEQ ID NO: 96, a LCDR2 comprising the sequence of SEQ ID NO: 115, and a LCDR3 comprising the sequence of SEQ ID NO: 146.

In certain embodiments, the present disclosure provides anti-CLDN18 (in particular, anti-CLDN18.2) antibodies and antigen-binding fragments thereof comprising a HCDR1 comprising the sequence of SEQ ID NO: 23, the HCDR2 comprising the sequence of SEQ ID NO: 58, the HCDR3 comprising the sequence of SEQ ID NO: 89, and/or a LCDR1 comprising the sequence of SEQ ID NO: 96, a LCDR2 comprising the sequence of SEQ ID NO: 115, and a LCDR3 comprising the sequence of SEQ ID NO: 147.

In certain embodiments, the present disclosure provides anti-CLDN18 (in particular, anti-CLDN18.2) antibodies and antigen-binding fragments thereof comprising a HCDR1 comprising the sequence of SEQ ID NO: 22, the HCDR2 comprising the sequence of SEQ ID NO: 55, the HCDR3 comprising the sequence of SEQ ID NO: 87, and/or a LCDR1 comprising the sequence of SEQ ID NO: 96, a LCDR2 comprising the sequence of SEQ ID NO: 115, and a LCDR3 comprising the sequence of SEQ ID NO: 130.

In certain embodiments, the present disclosure provides anti-CLDN18 (in particular, anti-CLDN18.2) antibodies and antigen-binding fragments thereof comprising a HCDR1 comprising the sequence of SEQ ID NO: 24, the HCDR2 comprising the sequence of SEQ ID NO: 59, the HCDR3 comprising the sequence of SEQ ID NO: 90, and/or a LCDR1 comprising the sequence of SEQ ID NO: 96, a LCDR2 comprising the sequence of SEQ ID NO: 115, and a LCDR3 comprising the sequence of SEQ ID NO: 148.

In certain embodiments, the present disclosure provides anti-CLDN18 (in particular, anti-CLDN18.2) antibodies and antigen-binding fragments thereof comprising a HCDR1 comprising the sequence of SEQ ID NO: 25, the HCDR2 comprising the sequence of SEQ ID NO: 60, the HCDR3 comprising the sequence of SEQ ID NO: 90, and/or a LCDR1 comprising the sequence of SEQ ID NO: 96, a LCDR2 comprising the sequence of SEQ ID NO: 115, and a LCDR3 comprising the sequence of SEQ ID NO: 148.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 61, the HCDR3 comprising the sequence of SEQ ID NO: 91, and/or a LCDR1 comprising the sequence of SEQ ID NO: 107, a LCDR2 comprising the sequence of SEQ ID NO: 115, and a LCDR3 comprising the sequence of SEQ ID NO: 149.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 62, the HCDR3 comprising the sequence of SEQ ID NO: 92, and/or a LCDR1 comprising the sequence of SEQ ID NO: 108, a LCDR2 comprising the sequence of SEQ ID NO: 115, and a LCDR3 comprising the sequence of SEQ ID NO: 150.

In certain embodiments, the present disclosure provides anti-CLDN18 (in particular, anti-CLDN18.2) antibodies and antigen-binding fragments thereof comprising a HCDR1 comprising the sequence of SEQ ID NO: 24, the HCDR2 comprising the sequence of SEQ ID NO: 59, the HCDR3 comprising the sequence of SEQ ID NO: 90, and/or a LCDR1 comprising the sequence of SEQ ID NO: 96, a LCDR2 comprising the sequence of SEQ ID NO: 115, and a LCDR3 comprising the sequence of SEQ ID NO: 148.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 61, the HCDR3 comprising the sequence of SEQ ID NO: 91, and/or a LCDR1 comprising the sequence of SEQ ID NO: 107, a LCDR2 comprising the sequence of SEQ ID NO: 115, and a LCDR3 comprising the sequence of SEQ ID NO: 149.

In certain embodiments, the present disclosure provides anti-CLDN18 (in particular, anti-CLDN18.2) antibodies and antigen-binding fragments thereof comprising a HCDR1 comprising the sequence of SEQ ID NO: 25, the HCDR2 comprising the sequence of SEQ ID NO: 60, the HCDR3 comprising the sequence of SEQ ID NO: 90, and/or a LCDR1 comprising the sequence of SEQ ID NO: 109, a LCDR2 comprising the sequence of SEQ ID NO: 123, and a LCDR3 comprising the sequence of SEQ ID NO: 151.

In certain embodiments, the present disclosure provides anti-CLDN18 (in particular, anti-CLDN18.2) antibodies and antigen-binding fragments thereof comprising a HCDR1 comprising the sequence of SEQ ID NO: 27, the HCDR2 comprising the sequence of SEQ ID NO: 367, the HCDR3 comprising the sequence of SEQ ID NO: 93, and/or a LCDR1 comprising the sequence of SEQ ID NO: 109, a LCDR2 comprising the sequence of SEQ ID NO: 123, and a LCDR3 comprising the sequence of SEQ ID NO: 151.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 63, the HCDR3 comprising the sequence of SEQ ID NO: 92, and/or a LCDR1 comprising the sequence of SEQ ID NO: 110, a LCDR2 comprising the sequence of SEQ ID NO: 115, and a LCDR3 comprising the sequence of SEQ ID NO: 150.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 64, the HCDR3 comprising the sequence of SEQ ID NO: 92, and/or a LCDR1 comprising the sequence of SEQ ID NO: 111, a LCDR2 comprising the sequence of SEQ ID NO: 115, and a LCDR3 comprising the sequence of SEQ ID NO: 150.

In certain embodiments, the present disclosure provides anti-CLDN18 (in particular, anti-CLDN18.2) antibodies and antigen-binding fragments thereof comprising a HCDR1 comprising the sequence of SEQ ID NO: 28, the HCDR2 comprising the sequence of SEQ ID NO: 65, the HCDR3 comprising the sequence of SEQ ID NO: 85, and/or a LCDR1 comprising the sequence of SEQ ID NO: 101, a LCDR2 comprising the sequence of SEQ ID NO: 120, and a LCDR3 comprising the sequence of SEQ ID NO: 152.

In certain embodiments, the present disclosure provides anti-CLDN18 (in particular, anti-CLDN18.2) antibodies and antigen-binding fragments thereof comprising a HCDR1 comprising the sequence of SEQ ID NO: 28, the HCDR2 comprising the sequence of SEQ ID NO: 66, the HCDR3 comprising the sequence of SEQ ID NO: 94, and/or a LCDR1 comprising the sequence of SEQ ID NO: 101, a LCDR2 comprising the sequence of SEQ ID NO: 120, and a LCDR3 comprising the sequence of SEQ ID NO: 153.

In certain embodiments, the present disclosure provides anti-CLDN18 (in particular, anti-CLDN18.2) antibodies and antigen-binding fragments thereof comprising a HCDR1 comprising the sequence of SEQ ID NO: 28, the HCDR2 comprising the sequence of SEQ ID NO: 65, the HCDR3 comprising the sequence of SEQ ID NO: 85, and/or a LCDR1 comprising the sequence of SEQ ID NO: 112, a LCDR2 comprising the sequence of SEQ ID NO: 120, and a LCDR3 comprising the sequence of SEQ ID NO: 152.

In certain embodiments, the present disclosure provides anti-CLDN18 (in particular, anti-CLDN18.2) antibodies and antigen-binding fragments thereof comprising a HCDR1 comprising the sequence of SEQ ID NO: 28, the HCDR2 comprising the sequence of SEQ ID NO: 66, the HCDR3 comprising the sequence of SEQ ID NO: 85, and/or a LCDR1 comprising the sequence of SEQ ID NO: 101, a LCDR2 comprising the sequence of SEQ ID NO: 120, and a LCDR3 comprising the sequence of SEQ ID NO: 154.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 67, the HCDR3 comprising the sequence of SEQ ID NO: 92, and/or a LCDR1 comprising the sequence of SEQ ID NO: 96, a LCDR2 comprising the sequence of SEQ ID NO: 115, and a LCDR3 comprising the sequence of SEQ ID NO: 155.

the In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 63, the HCDR3 comprising the sequence of SEQ ID NO: 92, and/or a LCDR1 comprising the sequence of SEQ ID NO: 108, a LCDR2 comprising the sequence of SEQ ID NO: 115, and a LCDR3 comprising the sequence of SEQ ID NO: 150.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 61, the HCDR3 comprising the sequence of SEQ ID NO: 91, and/or a LCDR1 comprising the sequence of SEQ ID NO: 107, a LCDR2 comprising the sequence of SEQ ID NO: 115, and a LCDR3 comprising the sequence of SEQ ID NO: 149.

In certain embodiments, the present disclosure provides anti-CLDN18 (in particular, anti-CLDN18.2) antibodies and antigen-binding fragments thereof comprising a HCDR1 comprising the sequence of SEQ ID NO: 11, the HCDR2 comprising the sequence of SEQ ID NO: 61, the HCDR3 comprising the sequence of SEQ ID NO: 91, and/or a LCDR1 comprising the sequence of SEQ ID NO: 113, a LCDR2 comprising the sequence of SEQ ID NO: 115, and a LCDR3 comprising the sequence of SEQ ID NO: 149.

In certain embodiments, the present disclosure provides anti-CLDN18 (in particular, anti-CLDN18.2) antibodies and antigen-binding fragments thereof comprising a HCDR1 comprising the sequence of SEQ ID NO: 26, the HCDR2 comprising the sequence of SEQ ID NO: 61, the HCDR3 comprising the sequence of SEQ ID NO: 91, and/or a LCDR1 comprising the sequence of SEQ ID NO: 107, a LCDR2 comprising the sequence of SEQ ID NO: 115, and a LCDR3 comprising the sequence of SEQ ID NO: 149.

In certain embodiments, the present disclosure provides anti-CLDN18 (in particular, anti-CLDN18.2) antibodies and antigen-binding fragments thereof comprising a HCDR1 comprising the sequence of SEQ ID NO: 11, the HCDR2 comprising the sequence of SEQ ID NO: 61, the HCDR3 comprising the sequence of SEQ ID NO: 91, and/or a LCDR1 comprising the sequence of SEQ ID NO: 96, a LCDR2 comprising the sequence of SEQ ID NO: 115, and a LCDR3 comprising the sequence of SEQ ID NO: 148.

In certain embodiments, the present disclosure provides anti-CLDN18 (in particular, anti-CLDN18.2) antibodies and antigen-binding fragments thereof comprising a HCDR1 comprising the sequence of SEQ ID NO: 28, a HCDR2 comprising the sequence of SEQ ID NO: 66, a HCDR3 comprising the sequence of SEQ ID NO: 85, and/or a LCDR1 comprising the sequence of SEQ ID NO: 101, a LCDR2 comprising the sequence of SEQ ID NO: 120, and a LCDR3 comprising the sequence of SEQ ID NO: 153.

In certain embodiments, the present disclosure provides anti-CLDN18 (in particular, anti-CLDN18.2) antibodies and antigen-binding fragments thereof comprising a HCDR1 comprising the sequence of SEQ ID NO: 16, a HCDR2 comprising the sequence of SEQ ID NO: 47, a HCDR3 comprising the sequence of SEQ ID NO: 80, and/or a LCDR1 comprising the sequence of SEQ ID NO: 103, a LCDR2 comprising the sequence of SEQ ID NO: 118, and a LCDR3 comprising the sequence of SEQ ID NO: 204.

In certain embodiments, the present disclosure provides anti-CLDN18 (in particular, anti-CLDN18.2) antibodies and antigen-binding fragments thereof comprising a HCDR1 comprising the sequence of SEQ ID NO: 201, a HCDR2 comprising the sequence of SEQ ID NO: 47, a HCDR3 comprising the sequence of SEQ ID NO: 80, and/or a LCDR1 comprising the sequence of SEQ ID NO: 103, a LCDR2 comprising the sequence of SEQ ID NO: 118, and a LCDR3 comprising the sequence of SEQ ID NO: 138.

In certain embodiments, the present disclosure provides anti-CLDN18 (in particular, anti-CLDN18.2) antibodies and antigen-binding fragments thereof comprising a HCDR1 comprising the sequence of SEQ ID NO: 201, a HCDR2 comprising the sequence of SEQ ID NO: 47, a HCDR3 comprising the sequence of SEQ ID NO: 80, and/or a LCDR1 comprising the sequence of SEQ ID NO: 103, a LCDR2 comprising the sequence of SEQ ID NO: 118, and a LCDR3 comprising the sequence of SEQ ID NO: 204.

In certain embodiments, the present disclosure provides anti-CLDN18 (in particular, anti-CLDN18.2) antibodies and antigen-binding fragments thereof comprising a HCDR1 comprising the sequence of SEQ ID NO: 202, a HCDR2 comprising the sequence of SEQ ID NO: 203, a HCDR3 comprising the sequence of SEQ ID NO: 83, and/or a LCDR1 comprising the sequence of SEQ ID NO: 96, a LCDR2 comprising the sequence of SEQ ID NO: 120, and a LCDR3 comprising the sequence of SEQ ID NO: 141.

In certain embodiments, the present disclosure provides anti-CLDN18 (in particular, anti-CLDN18.2) antibodies and antigen-binding fragments thereof comprising a HCDR1 comprising the sequence of SEQ ID NO: 202, a HCDR2 comprising the sequence of SEQ ID NO: 203, a HCDR3 comprising the sequence of SEQ ID NO: 83, and/or a LCDR1 comprising the sequence of SEQ ID NO: 205, a LCDR2 comprising the sequence of SEQ ID NO: 120, and a LCDR3 comprising the sequence of SEQ ID NO: 141.

In certain embodiments, the present disclosure provides anti-CLDN18 (in particular, anti-CLDN18.2) antibodies and antigen-binding fragments thereof comprising a HCDR1 comprising the sequence of SEQ ID NO: 19, a HCDR2 comprising the sequence of SEQ ID NO: 50, a HCDR3 comprising the sequence of SEQ ID NO: 83, and/or a LCDR1 comprising the sequence of SEQ ID NO: 205, a LCDR2 comprising the sequence of SEQ ID NO: 120, and a LCDR3 comprising the sequence of SEQ ID NO: 141.

The SEQ TD NOs of the heavy chain (denoted as “H”) variable region, light chain (denoted as “L”) variable region, HCDRs and LCDRs of each of the 60 monoclonal antibodies described above are shown in Table 1 below. Unless otherwise indicated, the CDR boundaries were defined or identified by the convention of Kabat. The amino acid sequences of each CDR of the 60 exemplary monoclonal antibodies are shown in Table 2 below. The amino acid sequences of each VH and VL of the 60 exemplary monoclonal antibodies are shown in Table 3 below.

TABLE 1
SEQ ID NOs of VH, VL, HCDRs and LCDRs
of 60 exemplary monoclonal antibodies.

| | | Variable | | | |
|---|---|---|---|---|---|
| | | Region | CDR1 | CDR2 | CDR3 |
| | | (SEQ | (SEQ | (SEQ | (SEQ |
| Antibody | | ID NO) | ID NO) | ID NO) | ID NO) |
| 99H8 | H | 254 | 1 | 29 | 68 |
| | L | 302 | 95 | 114 | 124 |
| 99G8 | H | 229 | 2 | 30 | 69 |
| | L | 282 | 96 | 114 | 125 |
| 99A7 | H | 235 | 3 | 31 | 70 |
| | L | 288 | 96 | 115 | 126 |
| 97A9 | H | 250 | 4 | 32 | 69 |
| | L | 290 | 96 | 115 | 127 |
| 84E8 | H | 236 | 5 | 33 | 71 |
| | L | 293 | 96 | 116 | 128 |
| 83H3 | H | 256 | 4 | 34 | 69 |
| | L | 268 | 97 | 115 | 129 |
| 80F10 | H | 251 | 6 | 32 | 69 |
| | L | 291 | 96 | 115 | 127 |
| 79C3 | H | 220 | 7 | 35 | 72 |
| | L | 284 | 96 | 115 | 130 |
| 78H6 | H | 222 | 8 | 36 | 72 |
| | L | 260 | 96 | 115 | 130 |
| 73E4 | H | 253 | 6 | 37 | 69 |
| | L | 270 | 98 | 115 | 127 |
| 69B2 | H | 232 | 5 | 38 | 73 |
| | L | 287 | 96 | 115 | 128 |
| 68E9 | H | 223 | 8 | 35 | 74 |
| | L | 285 | 96 | 115 | 130 |
| 68D1 | H | 252 | 6 | 32 | 69 |
| | L | 272 | 96 | 115 | 127 |
| 66E6 | H | 255 | 2 | 39 | 69 |
| | L | 299 | 99 | 115 | 131 |
| 66E12 | H | 233 | 5 | 33 | 71 |
| | L | 292 | 96 | 115 | 128 |
| 64C1 | H | 217 | 9 | 40 | 75 |
| | L | 262 | 99 | 115 | 132 |
| 64C10 | H | 219 | 9 | 41 | 76 |
| | L | 264 | 99 | 115 | 133 |
| 61A5 | H | 211 | 10 | 42 | 77 |
| | L | 261 | 100 | 115 | 134 |
| 60F11 | H | 234 | 11 | 43 | 71 |
| | L | 274 | 101 | 115 | 135 |
| 59G12 | H | 216 | 12 | 44 | 75 |
| | I | 263 | 99 | 115 | 132 |
| 59F5 | H | 218 | 13 | 40 | 75 |
| | L | 262 | 99 | 115 | 132 |
| 59E7 | H | 215 | 12 | 44 | 75 |
| | L | 263 | 99 | 115 | 132 |
| 56B2 | H | 227 | 14 | 45 | 78 |
| | L | 286 | 96 | 115 | 136 |
| 54F5 | H | 221 | 8 | 35 | 72 |
| | L | 281 | 96 | 115 | 130 |
| 38B9 | H | 213 | 15 | 46 | 79 |
| | L | 308 | 102 | 117 | 137 |
| 35B4 | H | 210 | 16 | 47 | 80 |
| | L | 307 | 103 | 118 | 138 |
| 35A10 | H | 207 | 17 | 48 | 81 |
| | L | 280 | 96 | 119 | 139 |
| 33G12 | H | 212 | 18 | 49 | 82 |
| | L | 309 | 104 | 118 | 140 |
| 22E12 | H | 246 | 19 | 50 | 83 |
| | L | 273 | 96 | 120 | 141 |
| 15E10 | H | 208 | 17 | 51 | 84 |
| | L | 278 | 96 | 121 | 142 |
| 100F4 | H | 249 | 2 | 52 | 69 |
| | L | 294 | 105 | 115 | 143 |
| 40C1 | H | 214 | 20 | 53 | 85 |
| | L | 269 | 106 | 122 | 144 |
| 41B3 | H | 206 | 21 | 54 | 86 |
| | L | 305 | 95 | 115 | 128 |
| 66D7-1 | H | 230 | 22 | 55 | 87 |
| | L | 279 | 98 | 115 | 145 |
| 66D7-2 | H | 257 | 2 | 56 | 69 |
| | L | 271 | 98 | 115 | 127 |
| 51G10 | H | 259 | 1 | 57 | 69 |
| | L | 271 | 98 | 115 | 127 |
| 365F6 | H | 241 | 11 | 43 | 88 |
| | L | 283 | 96 | 115 | 146 |
| 360C2 | H | 231 | 23 | 58 | 89 |
| | L | 306 | 96 | 115 | 147 |
| 319F2 | H | 230 | 22 | 55 | 87 |
| | L | 303 | 96 | 115 | 130 |
| 317A7 | H | 248 | 24 | 59 | 90 |
| | L | 289 | 96 | 115 | 148 |
| 315F10 | H | 258 | 25 | 60 | 90 |
| | L | 289 | 96 | 115 | 148 |
| 314D7 | H | 245 | 26 | 61 | 91 |
| | L | 266 | 107 | 115 | 149 |
| 310H5 | H | 228 | 26 | 62 | 92 |
| | L | 300 | 108 | 115 | 150 |
| 308E8 | H | 247 | 24 | 59 | 90 |
| | L | 289 | 96 | 115 | 148 |
| 305G8 | H | 244 | 26 | 61 | 91 |
| | L | 266 | 107 | 115 | 149 |
| 256C10-1 | H | 258 | 25 | 60 | 90 |
| | L | 301 | 109 | 123 | 151 |
| 256C10-2 | H | 209 | 27 | 367 | 93 |
| | L | 301 | 109 | 123 | 151 |
| 248D9 | H | 238 | 26 | 63 | 92 |
| | L | 275 | 110 | 115 | 150 |
| 246B8 | H | 240 | 26 | 64 | 92 |
| | L | 276 | 111 | 115 | 150 |
| 243A8 | H | 226 | 28 | 65 | 85 |
| | L | 297 | 101 | 120 | 152 |
| 242G5 | H | 224 | 28 | 66 | 94 |
| | L | 296 | 101 | 120 | 153 |
| 237E3 | H | 226 | 28 | 65 | 85 |
| | L | 298 | 112 | 120 | 152 |
| 226E9 | H | 225 | 28 | 66 | 85 |
| | L | 310 | 101 | 120 | 154 |
| 226D5 | H | 239 | 26 | 67 | 92 |
| | L | 277 | 96 | 115 | 155 |
| 217B5 | H | 237 | 26 | 63 | 92 |
| | L | 304 | 108 | 115 | 150 |
| 214E4 | H | 243 | 26 | 61 | 91 |
| | L | 267 | 107 | 115 | 149 |
| 213A9 | H | 242 | 11 | 61 | 91 |
| | L | 295 | 113 | 115 | 149 |
| 206C7 | H | 244 | 26 | 61 | 91 |
| | L | 265 | 107 | 115 | 149 |
| 203D12 | H | 242 | 11 | 61 | 91 |
| | L | 289 | 96 | 115 | 148 |
| 203A5 | H | 225 | 28 | 66 | 85 |
| | L | 296 | 101 | 120 | 153 |

TABLE 2
Amino acid sequences of each CDR of
60 exemplary monoclonal antibodies.

| | SEQ | | SEQ | |
|---|---|---|---|---|
| Descrip- | ID | Amino Acid | ID | Amino Acid |
| tion | NO | Sequence | NO | Sequence |
| HCDR1 | 1 | NEDIN | 18 | RYAMS |
| | 2 | NYDIN | 19 | NWVH |
| | 3 | RYWIQ | 202 | GYTFTNWVH |
| | 4 | RNDIN | 20 | DYNMD |
| | 5 | SYWIP | 21 | SGYLWN |
| | 6 | SYDIN | 22 | SYWIH |
| | 7 | DYNIH | 23 | TYGIS |
| | 8 | DYNMH | 24 | NYWMN |
| | 9 | DYTIH | 25 | SYWMN |
| | 10 | DYYMA | 26 | TYWMH |
| | 11 | SYWMH | 27 | NFGMY |
| | 12 | DYSMH | 28 | DYYMN |
| | 13 | DYTMH | 332 | X₁X₂DIN |
| | | | | X₁ = N, S or R; |
| | | | | X₂ = F, Y, or N |
| | 14 | DYGMH | 333 | X₃YX₄MH |
| | | | | X₃ = D, S or T; |
| | | | | X₄ = N, W, S or T |
| | 15 | SYAMS | 334 | X₅YAX₆S |
| | | | | X₅ = S or R; |
| | | | | X₆ = M or L |
| | 16 | SYALS | 335 | X₇YX₈MN |
| | | | | X₇ = N, D or S; |
| | | | | X₈ = Y or W |
| | 201 | SGFTLSSYALS | 336 | X₉YX₁₀IH |
| | | | | X₉ = D or S; |
| | | | | X₁₀ = N, T or W |
| | 17 | SFGMH | 337 | X₁₁X₁₂GMH |
| | | | | X₁₁ = D or S; |
| | | | | X₁₂ = Y or F |
| HCDR2 | 29 | RLYPRDGTTTYNE | 51 | YISSGSSSIYYVDTVKG |
| | | KFKG | | |
| | 30 | RIYPRDDSTTYNE | 52 | GIHPRDGNTKYNEKFKD |
| | | KFKG | | |
| | 31 | MIHPNSGSTNYNE | 53 | DVNPNYSTTRYNQKFKD |
| | | KFKK | | |
| | 32 | LSYPRDGTTQYNG | 54 | HITYDGSNNYNPSLKN |
| | | KFKG | | |
| | 33 | MIHPNSGSTNYNE | 55 | RIRPSDSDSTYNONFKG |
| | | KFKR | | |
| | 34 | RIYPRDGGTNYNE | 56 | WIFPRDGSTKYNEKFRG |
| | | KFKG | | |
| | 35 | YINPKNGGTRYNQ | 57 | LSYPRDSTTQYNGKFRG |
| | | KFKG | | |
| | 36 | YINPNNGGTTYNQ | 58 | VIWGDGSTHYHSALIS |
| | | KFKG | | |
| | 37 | LSYPRDSSTQYNG | 59 | QIYPGNGNTNYNGGFKG |
| | | RFRG | | |
| | 38 | MIHPNSDSTNYNE | 60 | QIYPGDGDTNYNGGFRG |
| | | KFKS | | |
| | 39 | LIYPRDKNTNYNG | 61 | RIRPSDSDSNYNQKFKG |
| | | KFKG | | |
| | 40 | YINPYNSGTRYNQ | 62 | GIRPSDSNTNYNHKFKG |
| | | KFKG | | |
| | 41 | SINPYNPGTRYNQ | 367 | FITSDSTSIYYVDTVKG |
| | | KFEG | | |
| | 42 | NINYDGSSTFYLD | 63 | GIRPFDSNTNYNHKEKG |
| | | SLKS | | |
| | 43 | MIHPNSGSTNYNE | 64 | GIRPSDSNNNYNHKFKG |
| | | KFKS | | |
| | 44 | FINPYSGSTTYNQ | 65 | DINPKNGGSRYNQKFRG |
| | | KFKG | | |
| | 45 | YISSGSSNIYYAD | 66 | DINPKNGGSRYNQKFKG |
| | | TVKG | | |
| | 46 | YISNLGGSTYYPD | 67 | RIRPSDTATNYNQKFKG |
| | | TVKG | | |
| | 47 | YISNLGGSTFYPD | 338 | X₁₃X₁₄YPRDX₁₅X₁₆T |
| | | TVKG | | X₁₇YNX₁₈KFKG |
| | | | | X₁₃ = R or L; |
| | | | | X₁₄ = L, I, or S; |
| | | | | X₁₅ = G, D, or K; |
| | | | | X₁₆ = T, S, G, or |
| | | | | N; |
| | | | | X₁₇ = T, N, or Q; |
| | | | | X₁₈ = E or G |
| | 48 | YISSGSSSFYYAD | 339 | YINPX₁₉NX₂₀GTX₂₁Y |
| | | TVKG | | NQKFKG |
| | | | | X₁₉ = K, N or Y; |
| | | | | X₂₀ = G or S; |
| | | | | X₂₁ = R or T |
| | 49 | YISIGGTTYYPDT | 340 | MIHPNSX₂₂STNYNEKF |
| | | IKG | | KX₂₃ |
| | | | | X₂₂ = G or D; |
| | | | | X₂₃ = K, R or S |
| | 50 | EINPTNGRSNYNE | 341 | YISNLGGSTX₂₄YPDTV |
| | | KFKK | | KG |
| | | | | X₂₄ = Y or F |
| | 203 | EINPTNARSNYNE | 342 | YISX₂₅GX₂₆X₂₇X₂₈ |
| | | KFKK | | X₂₉YYX₃₀DTX₃₁KG |
| | | | | X₂₅ = S or I; |
| | | | | X₂₆ = S or G; |
| | | | | X₂₇ = S or T; |
| | | | | X₂₈ = S or T; |
| | | | | X₂₉ = F, I or |
| | | | | absent; |
| | | | | X₃₀ = A, P or V; |
| | | | | X₃₁ = V or I |
| | 343 | EINPTNX₃₂RSNY | | |
| | | NEKFKK | | |
| | | X₃₂ = G or A | | |
| HCDR3 | 68 | GNYGNSFAY | 81 | NAYYGNALDY |
| | 69 | GYYGNSFAY | 82 | HYYGHDVMDY |
| | 70 | MGLGNAMDE | 83 | IYYGNSFAH |
| | 71 | MGLGNAMDY | 84 | NAYYGNAFDY |
| | 72 | IYYGNSFDY | 85 | LYYGNSFAY |
| | 73 | MGLGNALDY | 86 | GRYGNNRDY |
| | 74 | LYYGNSFDY | 87 | GAYYSNSFGY |
| | 75 | IFYGNSFDY | 88 | NGYYGNAMDY |
| | 76 | VFYGNSFDY | 89 | PYYSNAMDY |
| | 77 | QVGYYDPMDY | 90 | WGTGNTMDY |
| | 78 | FYYGNSFAY | 91 | GAYFSNSFAY |
| | 79 | HLYHYDAFAY | 92 | GAYYSNSFAY |
| | 80 | HLYNYDAFAS | 93 | TGYGNAMDY |
| | 344 | X₃₃X₃₄X₃₅GNSF | 94 | LYFGNSFAY |
| | | AX₃₆ | | |
| | | X₃₃ = G, F, L | | |
| | | or I; | | |
| | | X₃₄ = N or Y; | | |
| | | X₃₅ = Y or F; | | |
| | | X₃₆ = Y or H | | |
| | 345 | HLYX₃₇YDAFA | 346 | NAYYGNAX₃₉DY |
| | | X₃₈ | | X₃₉ = L or F |
| | | X₃₇ = H or N; | | |
| | | X₃₈ = Y or S | | |
| LCDR1 | 95 | KSSQSLFNSGNQK | 205 | KSSQSLLNAGNQKNYLT |
| | | NYLT | | |
| | 96 | KSSQSLLNSGNOK | 105 | KSGQSLLNSGNORNYLT |
| | | NYLT | | |
| | 97 | KSSQSLLNDGNQK | 106 | RSSQILLNSGNQKNYLT |
| | | NYLT | | |
| | 98 | KSSQSLLNGGNQK | 107 | KSSQTLLNRGNQKNYLT |
| | | NYLT | | |
| | 99 | KSSQSLLNSGNLK | 108 | KSSQSLLNSGNQKNYVT |
| | | NYLT | | |
| | 100 | KSSQSLLYSSNOK | 109 | KSSQSLENSGNOKNYLS |
| | | NYLA | | |
| | 101 | KSSQSLLNSGNQR | 110 | KSNQSLLNSGNQKNYVT |
| | | NYLT | | |
| | 102 | RASSSLSYMH | 111 | KSSQSLLNRGNQKNYVT |
| | 103 | RASSSVNYIH | 112 | KSSQSLLNSGNRRNYLT |
| | 104 | RATSSVSYMH | 113 | KSSQTLLNRGNQKNYVT |
| | 347 | X₄₀SSQX₄₁LX₄₂ | 348 | RAX₄₅SSX₄₆X₄₇YX₄₈ |
| | | NX₄₃GNQX₄₄NYL | | H |
| | | T | | X₄₅ = S or T; |
| | | X₄₀ = K or R; | | X₄₆ = L or V; |
| | | X₄₁ = S or I; | | X₄₇ = S or N; |
| | | X₄₂ = L or F; | | X₄₈ = M or I |
| | | X₄₃ = S or A; | | |
| | | X₄₄ = K or R | | |
| LCDR2 | 114 | WASTRQS | 119 | WASTRRS |
| | 115 | WASTRES | 120 | WSSTRES |
| | 116 | WASTRAS | 121 | WASTRTS |
| | 117 | GTSNLAS | 122 | WASTRDY |
| | 118 | ATSNLAS | 123 | WASTRKS |
| | 349 | WX₄₉STRX₅₀X₅₁ | 350 | X₅₂TSNLAS |
| | | X₄₉ = A or S; | | X₅₂ = G or A |
| | | X₅₀ = Q, R, | | |
| | | T, K, D or E; | | |
| | | X₅₁ = S or Y | | |
| LCDR3 | 124 | QNGFSFPYT | 140 | QQWSRNPLT |
| | 125 | QNDFGFPYT | 141 | QNNYYYPLT |
| | 126 | QNNYVYPLT | 142 | ONGYTYPLT |
| | 127 | QNDYYFPYT | 143 | QNAYFYPYT |
| | 128 | QNDYYYPLT | 144 | QNAYFYPFT |
| | 129 | QNGYSFPYT | 145 | QNDYFFPYT |
| | 130 | QNDYSFPFT | 146 | QNNYNYPVT |
| | 131 | QNDYYYPYT | 147 | QNVYSYPLT |
| | 132 | QNDYFYPFT | 148 | QNVYSYPIT |
| | 133 | QNNYFYPFT | 149 | QNDYFFPFT |
| | 134 | QQYYTYPLT | 150 | QNDYVYPFT |
| | 135 | QNAYSYPLT | 151 | QNNYFYPLT |
| | 136 | QNAYSFPFT | 152 | QNDYTYPLT |
| | 137 | QQWSSNPLT | 153 | QNDYSYPLT |
| | 138 | QQWNSNPLT | 154 | QNDYNYPLT |
| | 204 | QQWNANPLT | 155 | QNDYSYPFM |
| | 139 | QNVYVYPLT | 351 | QNX₅₃X₅₄X₅₅FPYT |
| | | | | X₅₃ = G or D; |
| | | | | X₅₄ = F or Y; |
| | | | | X₅₅ = S or Y |
| | 352 | QNAYX₅₆YPX₅₇T | 353 | QNX₅₈YX₅₉YPLT |
| | | X₅₆ = S or F; | | X₅₈ = N, V or G; |
| | | X₅₇ = L or F | | X₅₉ = Y, V or T |
| | 354 | QQWX₆₀X₆₁NPLT | | |
| | | X₆₀ = S or N; | | |
| | | X₆₁ = S, A or | | |
| | | R | | |

TABLE 3
Amino acid sequences of each VH and VL of 60 exemplary
monoclonal antibodies.

| | | SEQ | | SEQ |
|---|---|---|---|---|
| Antibody | VH | ID NO | VL | ID NO |
| 100F4 | QVQLQQSGPDLVKPGASV | 249 | DIVMTQSPSSLTVTAGEK | 294 |
| | KLSCKASGYTFTNYDINW | | VTMTCKSGQSLLNSGNQR | |
| | VKQRPGQGLEWIGGIHPR | | NYLTWYQQKPGQSPKLLI | |
| | DGNTKYNEKFKDKATLTI | | YWASTRESGVPDRFTGSG | |
| | DTSANTAYMEFHSLTSED | | SGADFTLTISSVQAEDLA | |
| | SAVYFCARGYYGNSFAYW | | LYYCQNAYFYPYTFGGGT | |
| | GQGTLVTVSA | | KLEIK | |
| 15E10 | DVQLVESGGGLVQPGGSR | 208 | DIVMTQSPSSLTVTAGEK | 278 |
| | KLSCAASGFTFSSFGMHW | | VTMNCKSSQSLLNSGNQK | |
| | VRQAPEKGLEWVAYISSG | | NYLTWYQQKPGQPPKLLI | |
| | SSSIYYVDTVKGRFTISR | | YWASTRTSGVPDRFTGSG | |
| | DNPKNTLFLQMTSLRSED | | SGTDFTLTISSVQAEDLA | |
| | TAMYYCARNAYYGNAFDY | | VYCCQNGYTYPLTFGAGT | |
| | WGQGTTLTVSS | | KLELK | |
| 203A5 | EVQLQQSGPEVVKPGTSV | 225 | DIVMTQSPSSLTVTAGEM | 296 |
| | KISCKASGYTFTDYYMNW | | VTMNCKSSQSLLNSGNQR | |
| | VKQSHGKSLEWIGDINPK | | NYLTWYQQKPGQPPKLLI | |
| | NGGSRYNQKFKGKATLTV | | YWSSTRESGVPDRFTGSG | |
| | DKSSNTAYMELRSLTSED | | SGTDFTLTISSVQAEDLA | |
| | SAVYYCARLYYGNSFAYW | | VYYCQNDYSYPLTFGAGT | |
| | GQGTLVTVSA | | KLELK | |
| 203D12 | QVQLQQPGTELVKPGASV | 242 | DIVMTQSPSSLTVTAGEK | 289 |
| | KVSCKASGYTFTSYWMHW | | VTMSCKSSQSLLNSGNQK | |
| | VKQRPGQGLEWIGRIRPS | | NYLTWYQQKPGQPPKLLI | |
| | DSDSNYNQKFKGKATLTV | | YWASTRESGVPDRFTGSG | |
| | DKSSDTAYMQLNSLPSED | | SGTDFTLTISSVQAEDLA | |
| | SAVYYCAMGAYFSNSFAY | | VYYCQNVYSYPITFGSGT | |
| | WGQGTLVTVSA | | KLEIK | |
| 206C7 | QVQLQQPGTELVKPGASV | 244 | DIVMTQSPSPLTVIVGEK | 265 |
| | KVSCKASGYTFTTYWMHW | | VTMTCKSSQTLLNRGNQK | |
| | VKQRPGQGLEWIGRIRPS | | NYLTWYQQKPGQPPKLLI | |
| | DSDSNYNQKFKGKATLTV | | YWASTRESGVPDRFTGSG | |
| | DKSSDTAYMQLNSLTSED | | SGTDFTLTINSVQAEDLA | |
| | SAVYYCAMGAYFSNSFAY | | VYYCQNDYFFPFTFGSGT | |
| | WGQGTLVTVSA | | KLEIR | |
| 213A9 | QVQLQQPGTELVKPGASV | 242 | DIVMTQSPSSLTVTAGEK | 295 |
| | KVSCKASGYTFTSYWMHW | | VTMTCKSSQTLLNRGNQK | |
| | VKQRPGQGLEWIGRIRPS | | NYVTWYQQKPGQPPKLLI | |
| | DSDSNYNQKFKGKATLTV | | YWASTRESGVPDRFTGSG | |
| | DKSSDTAYMQLNSLPSED | | SGTDFTLTISSVQAEDLA | |
| | SAVYYCAMGAYFSNSFAY | | VYYCQNDYFFPFTFGSGT | |
| | WGQGTLVTVSA | | KLEIR | |
| 214E4 | QVQLQQPGTELVKPGASV | 243 | DIVMTQSPSPLTVTAGEK | 267 |
| | KVSCKASGYTFTTYWMHW | | VTMTCKSSQTLLNRGNQK | |
| | VKQRPGQGLEWIGRIRPS | | NYLTWYQQKPGQPPKLLI | |
| | DSDSNYNQKFKGKATLTV | | YWASTRESGVPDRFTGSG | |
| | DKSSDTAYMQLNGLTSED | | SGTDETLTINSVQAEDLA | |
| | SAVYYCAMGAYFSNSFAY | | VYYCQNDYFFPFTFGSGT | |
| | WGQGTLVTVSA | | KLETR | |
| 217B5 | QVQLQQPGAELVKPGASV | 237 | DIVMTQSPSSLTVTPGEK | 304 |
| | KVSCKASGSTFTTYWMHW | | VTMNCKSSQSLLNSGNQK | |
| | VKKRPGQGLEWIGGIRPF | | NYVTWYQQKPGQPPKLLM | |
| | DSNTNYNHKFKGKATLTV | | FWASTRESGVPDRFTGSG | |
| | DKASNTAYMQLSSLTSED | | SGTDFTLIISSVQAEDLA | |
| | SAVYYCAMGAYYSNSFAY | | VYHCQNDYVYPFTFGSGT | |
| | WGQGTVVTVSA | | KLEIK | |
| 226D5 | QVQLQQPGAELVKPGASV | 239 | DIVMTQSPSSLTVTAGEK | 277 |
| | KVSCKASGYTFTTYWMHW | | VTMNCKSSQSLLNSGNQK | |
| | VRQRPGQGLEWIGRIRPS | | NYLTWYQQKPGQPPKLLI | |
| | DTATNYNQKFKGKATLTV | | YWASTRESGVPDRFTGSG | |
| | NKSSSTAYMQFSSLTSED | | SGTDFTLTINSVQAEDLA | |
| | SAVFYCAMGAYYSNSFAY | | VYYCQNDYSYPFMFGSGT | |
| | WGQGTLVTVSA | | KLEIK | |
| 226E9 | EVQLQQSGPEVVKPGTSV | 225 | DIVMTQSPSSLTVTAGEM | 310 |
| | KISCKASGYTFTDYYMNW | | VTMNCKSSQSLLNSGNQR | |
| | VKQSHGKSLEWIGDINPK | | NYLTWYQQKPGQPPKLLI | |
| | NGGSRYNQKFKGKATLTV | | YWSSTRESGVPDRFTGSG | |
| | DKSSNTAYMELRSLTSED | | SGTDFTLTISSVQAEDLA | |
| | SAVYYCARLYYGNSFAYW | | VYYCQNDYNYPLTFGAGT | |
| | GQGTLVTVSA | | KLELK | |
| 22E12 | QVQLQQPGTELVKTGTSV | 246 | DIVMTQSPSSLTVTAGEK | 273 |
| | KLSCKASGYTFTNWVHWV | | VTLSCKSSQSLLNSGNQK | |
| | IQRPGQGLEWIGEINPTN | | NYLTWYQQKPGQPPKLLI | |
| | GRSNYNEKFKKKATLTLD | | YWSSTRESGVPDRFTGSG | |
| | RSSTTAYMQLSSLTSEDS | | SGTDFTLTISSVQAEDLA | |
| | AVYFCAGIYYGNSFAHWG | | VYHCQNNYYYPLTFGAGT | |
| | QGTLVTVSA | | KLELK | |
| 237E3 | EVQLQQSGPEVVKPGTSV | 226 | DIVMTQSPSSLTVTAGEM | 298 |
| | KISCKASGYTFTDYYMNW | | VTMNCKSSQSLLNSGNRR | |
| | VKQSHGKSLEWIGDINPK | | NYLTWYQQKPGQPPKLLI | |
| | NGGSRYNQKFRGKATLTV | | YWSSTRESGVPDRFAGSG | |
| | DKSSNTAFMELRSLTSED | | SGTDFTLTISSVQAEDLA | |
| | SAVYYCARLYYGNSFAYW | | VYYCQNDYTYPLTFGAGT | |
| | GQGTLVTVSA | | KLELK | |
| 242G5 | EVQLQQSGPEVVKPGTSV | 224 | DIVMTQSPSSLTVTAGEM | 296 |
| | KISCKASGYTFTDYYMNW | | VTMNCKSSQSLLNSGNQR | |
| | VKQSHGKSLEWIGDINPK | | NYLTWYQQKPGQPPKLLI | |
| | NGGSRYNQKFKGKATLTA | | YWSSTRESGVPDRFTGSG | |
| | DKSSNTAYMELRSLTSED | | SGTDFTLTISSVQAEDLA | |
| | SAVYYCTRLYFGNSFAYW | | VYYCQNDYSYPLTFGAGT | |
| | GQGTLVTVSA | | KLELK | |
| 243A8 | EVQLQQSGPEVVKPGTSV | 226 | DIVMTQSPSSLTVTAGEM | 297 |
| | KISCKASGYTFTDYYMNW | | VTMNCKSSQSLLNSGNQR | |
| | VKQSHGKSLEWIGDINPK | | NYLTWYQQKPGQPPKLLI | |
| | NGGSRYNQKFRGKATLTV | | YWSSTRESGVPDRFTGSG | |
| | DKSSNTAFMELRSLTSED | | SGTDFTLTISSVQAEDLA | |
| | SAVYYCARLYYGNSFAYW | | VYYCQNDYTYPLTFGAGT | |
| | GQGTLVTVSA | | KLELK | |
| 246B8 | QVQLQQPGAELVNPGASV | 240 | DIVMTQSPSSLTVTAGEK | 276 |
| | KVSCKASGSTFTTYWMHW | | VTMNCKSSQSLLNRGNQK | |
| | VKKRPGQGLEWIGGIRPS | | NYVTWYQQKPGQPPKLLI | |
| | DSNNNYNHKFKGKATLTV | | FWASTRESGVPDRFTGSG | |
| | DKASSTAYLQLSSLTSED | | SGTDETLIISSVQAEDLA | |
| | SAVYYCAMGAYYSNSFAY | | VYYCQNDYVYPFTFGSGT | |
| | WGQGTVVTVSA | | KLEIK | |
| 248D9 | QVQLQQPGAELVKPGASV | 238 | DIVMTQSPSSLTVTAGEK | 275 |
| | KVSCKASGSTFTTYWMHW | | VTMNCKSNQSLLNSGNQK | |
| | VKKRPGQGLEWIGGIRPF | | NYVTWYQQKPGQPPKLLI | |
| | DSNTNYNHKFKGKATLTV | | FWASTRESGVPDRFTGSG | |
| | DKASSTAYMQLSSLTSED | | SETDFTLIISSVQAEDLA | |
| | SAVYYCAMGAYYSNSFAY | | VYYCQNDYVYPFTFGSGT | |
| | WGQGTVVTVSA | | KLEIK | |
| 256C10-1 | QVQLQQSGTELVKPGASV | 258 | DIVMTQSPSSLTVTAREK | 301 |
| | KISCKASGYAFNSYWMNW | | VIMNCKSSQSLENSGNQK | |
| | LKQRPGKGLEWIGQIYPG | | NYLSWYQQKPGQPPKLLI | |
| | DGDTNYNGGFRGKATLTA | | YWASTRKSGVPDRFTGSG | |
| | DKSSRTAYMHLNSLTSED | | SGTGFTLTISSVQAEDLA | |
| | SAVYFCARWGTGNTMDYW | | VYYCQNNYFYPLTFGAGT | |
| | GQGTSVTVSS | | KLELN | |
| 256C10-2 | DVQLVESGGGLVQPGGSR | 209 | DIVMTQSPSSLTVTAREK | 301 |
| | RLSCAASGFSFSNFGMYW | | VIMNCKSSQSLFNSGNQK | |
| | VRQAPEKGLEWVAFITSD | | NYLSWYQQKPGQPPKLLI | |
| | STSIYYVDTVKGRFTVSR | | YWASTRKSGVPDRFTGSG | |
| | DNPKNTLFLQMTSLRSED | | SGTGFTLTISSVQAEDLA | |
| | TAMYYCGRTGYGNAMDYW | | VYYCQNNYFYPLTFGAGT | |
| | GQGTSVTVSS | | KLELN | |
| 305G8 | QVQLQQPGTELVKPGASV | 244 | DIVMTQSPSPLTVTAGEK | 266 |
| | KVSCKASGYTFTTYWMHW | | VTMTCKSSQTLLNRGNQK | |
| | VKQRPGQGLEWIGRIRPS | | NYLTWYQQKPGQPPKLLI | |
| | DSDSNYNQKFKGKATLTV | | YWASTRESGVPDRFTGSG | |
| | DKSSDTAYMQLNSLTSED | | SGTDFTLTINSVQAEDLA | |
| | SAVYYCAMGAYFSNSFAY | | VYYCQNDYFFPFTFGSGT | |
| | WGQGTLVTVSA | | KLEIR | |
| 308E8 | QVQLQQSGAELVKPGASV | 247 | DIVMTQSPSSLTVTAGEK | 289 |
| | KISCKASGYAFSNYWMNW | | VTMSCKSSQSLLNSGNQK | |
| | VKQRPGKGLEWIGQIYPG | | NYLTWYQQKPGQPPKLLI | |
| | NGNTNYNGGFKGKATLTA | | YWASTRESGVPDRFTGSG | |
| | DKSSSTAYMHLNSLTSED | | SGTDFTLTISSVQAEDLA | |
| | SAVYFCARWGTGNTMDYW | | VYYCQNVYSYPITFGSGT | |
| | GQGTSVTVSS | | KLEIK | |
| 310H5 | LVQLQQPGAELVKPGASV | 228 | DIVMTQSPSSLTVTAGKK | 300 |
| | KVSCKASGSTFTTYWMHW | | VTMNCKSSQSLLNSGNQK | |
| | VKKRPGQGLEWIGGIRPS | | NYVTWYQQKPGQPPKLLI | |
| | DSNTNYNHKFKGKATLTV | | FWASTRESGVPDRFTGSG | |
| | DKASSTAYMQLSSLTSED | | SGTDESLIISTVQAEDLA | |
| | SAVYYCAMGAYYSNSFAY | | VYYCQNDYVYPFTFGSGT | |
| | WAQGTVVTVSA | | KLEIK | |
| 314D7 | QVQLQQPGTELVKPGASV | 245 | DIVMTQSPSPLTVTAGEK | 266 |
| | KVSCKASGYTFTTYWMHW | | VTMTCKSSQTLLNRGNQK | |
| | VTQRPGQGLEWIGRIRPS | | NYLTWYQQKPGQPPKLLI | |
| | DSDSNYNQKFKGKATLTV | | YWASTRESGVPDRFTGSG | |
| | DKSSDTAYMQLNSLTSED | | SGTDFTLTINSVQAEDLA | |
| | SAVYYCAMGAYESNSFAY | | VYYCQNDYFFPFTFGSGT | |
| | WGQGTLVTVSA | | KLEIR | |
| 315F10 | QVQLQQSGTELVKPGASV | 258 | DIVMTQSPSSLTVTAGEK | 289 |
| | KISCKASGYAFNSYWMNW | | VTMSCKSSQSLLNSGNQK | |
| | LKQRPGKGLEWIGQIYPG | | NYLTWYQQKPGQPPKLLI | |
| | DGDTNYNGGFRGKATLTA | | YWASTRESGVPDRETGSG | |
| | DKSSRTAYMHLNSLTSED | | SGTDFTLTISSVQAEDLA | |
| | SAVYFCARWGTGNTMDYW | | VYYCQNVYSYPITFGSGT | |
| | GQGTSVTVSS | | KLEIK | |
| 317A7 | QVQLQQSGAELVKPGASV | 248 | DIVMTQSPSSLTVTAGEK | 289 |
| | KISCKASGYAFSNYWMNW | | VTMSCKSSQSLLNSGNQK | |
| | VNQRPGKGLEWIGQIYPG | | NYLTWYQQKPGQPPKLLI | |
| | NGNTNYNGGFKGKATLTA | | YWASTRESGVPDRFTGSG | |
| | DKSSSTAYMHLNSLTSED | | SGTDFTLTISSVQAEDLA | |
| | SAVYFCARWGTGNTMDYW | | VYYCQNVYSYPITFGSGT | |
| | GQGTSVTVSS | | KLEIK | |
| 319F2 | QVLLQQPGTELVKPGASV | 230 | DIVMTQSPSSLTVTAREK | 303 |
| | KVSCKASAYTFTSYWIHW | | VTMNCKSSQSLLNSGNQK | |
| | VKQRPGQGLEWIGRIRPS | | NYLTWYQQKPGQPPKMLI | |
| | DSDSTYNQNFKGKATLTV | | YWASTRESGVPDRFTGSG | |
| | DKSSDTAYMQLTSLTSED | | SGTYFTLTISSVQAEDLA | |
| | SAVYYCSMGAYYSNSFGY | | VYYCQNDYSFPFTFGSGT | |
| | WGQGSLVTVSA | | KLEIK | |
| 33G12 | EVKLVESGGGLVQPGGSL | 212 | QIVLSQSPTILSASPGEK | 309 |
| | KLSCAASGFTFSRYAMSW | | VTMTCRATSSVSYMHWFQ | |
| | VRQTPEKRLEWVAYISIG | | QKPGSSPKPWIYATSNLA | |
| | GTTYYPDTIKGRFTISRD | | SGVPARFSGSGSGTSYSL | |
| | NAKNTLYLQMSSLKSEDT | | TISRVEAEDAATYYCQQW | |
| | AMYYCTRHYYGHDVMDYW | | SRNPLTFGAGTKLELK | |
| | GQGTSVTVSS | | | |
| 35A10 | DVQLVESGGGLVQPGGSR | 207 | DIVMTQSPSSLTVTAGEK | 280 |
| | KLSCAASGFTFSSFGMHW | | VTMSCKSSQSLLNSGNQK | |
| | VRQAPEKGLEWVAYISSG | | NYLTWYQHKPGQPPKLLI | |
| | SSSFYYADTVKGRFTISR | | YWASTRRSGVPDRETGSG | |
| | DNPKNTLFLQMTSLRSED | | SGTDFTLTITSVQAEDLA | |
| | TAMYYCARNAYYGNALDY | | VYCCQNVYVYPLTFGAGT | |
| | WGQGTTLTVSS | | KLELK | |
| 35B4 | EAKLVESGGDFMQPGGSL | 210 | QIVLSQSPAILSASPGEK | 307 |
| | KLSCAASGFTLSSYALSW | | VTMTCRASSSVNYIHWYQ | |
| | VRQTPEKRLEWVAYISNL | | QKPGSSPKAWIYATSNLA | |
| | GGSTFYPDTVKGRFTISR | | SGVPTRESGSGSGTSYSL | |
| | DNARNTLFLQMSSLQSED | | TIDRVEAEDAATYYCQQW | |
| | TAIYYCATHLYNYDAFAS | | NSNPLTFGAGTKLELK | |
| | WGQGTLVTVSA | | | |
| 360C2 | QVQLKESGPGLVAPSQSL | 231 | DIVMTQSPSSLTVTVGEK | 306 |
| | SITCIVSGFSLTTYGISW | | VTMSCKSSQSLLNSGNQK | |
| | VRQPPGKGLEWLGVIWGD | | NYLTWYRQKPGQPPELLI | |
| | GSTHYHSALISRLSISKD | | YWASTRESGVPDRFTGSG | |
| | NSKSQVFLKLNSLQTDDT | | SGTDFTLTISSVQAEDLA | |
| | ATYYCAKPYYSNAMDYWG | | VYYCQNVYSYPLTFGAGT | |
| | QGTSVTVSS | | KLELK | |
| 365F6 | QVQLQQPGPELVKPGASV | 241 | DIVMTQSPSSLTVTAGEK | 283 |
| | KLSCKASGYTFTSYWMHW | | VTMSCKSSQSLLNSGNQK | |
| | VRQRPGQGLEWIGMIHPN | | NYLTWYQQKPGQPPKLLI | |
| | SGSTNYNEKFKSKATLTV | | YWASTRESGVPDRFTGRG | |
| | DKSSSTAYMQLSSLTSED | | SGTDFTLTISSVQAEDLA | |
| | SAVYFCARNGYYGNAMDY | | VYYCQNNYNYPVTFGAGT | |
| | WGQGTSVTVSS | | KLELK | |
| 38B9 | EVNLVESGGGFVQPGGSL | 213 | QIVLSQSPAILSASPGEK | 308 |
| | KLSCVASGFTFSSYAMSW | | VTVTCRASSSLSYMHWYQ | |
| | VRQTPDTRLEWVAYISNL | | QRPGSSPKPWIYGTSNLA | |
| | GGSTYYPDTVKGRFTISR | | SGVPARFSGSGSGTSYSL | |
| | DNARNTLFLQMSSLQSED | | TISRVEAEDAATYYCQQW | |
| | TAMYYCTGHLYHYDAFAY | | SSNPLTFGAGTKLELK | |
| | WGQGTLVTVSA | | | |
| 40C1 | EVQLQQFGAELVKPGTSV | 214 | DIVMTQSPSSLPVTTGER | 269 |
| | KISCKASGYTFTDYNMDW | | VTMSCRSSQILLNSGNQK | |
| | VKQSHGKSLEWIGDVNPN | | NYLTWYQQKPGQPPKLLI | |
| | YSTTRYNQKFKDKATLTV | | YWASTRDYGVPDRFTGSG | |
| | DKSSSTAYMELRSLTSED | | SGTDFTLTISSVQAEDLA | |
| | TAVYYCARLYYGNSFAYW | | VYYCQNAYFYPFTFGAGT | |
| | GQGTLVTVSA | | KLELK | |
| 41B3 | DVQLQESGPGLVKPSQSL | 206 | DIVMTQSPSSLTVTSGEK | 305 |
| | SLTCSVTGYSITSGYLWN | | VTMSCKSSQSLENSGNQK | |
| | WIRQSPGNKLEWMGHITY | | NYLTWYQQKLGQPPKLLI | |
| | DGSNNYNPSLKNRISITR | | FWASTRESGVPDRFTGSG | |
| | DTSKNQFFLKLNSVTTED | | SGTDFTLTISSVQTEDLA | |
| | TATYFCSRGRYGNNRDYW | | VYYCQNDYYYPLTFGAGT | |
| | GQGTTLTVSS | | KLELK | |
| 51G10 | RVQLQQSGPELVKPGASM | 259 | DIVMTQSPSSLTVTAGEK | 271 |
| | KLSCKTSGYTFTNFDINW | | ATMSCKSSQSLLNGGNQK | |
| | VKQRPGQGLEWIGLSYPR | | NYLTWYQQKPGQPPTLLI | |
| | DSTTQYNGKFRGKATLTV | | YWASTRESGVPDRFTGSG | |
| | DTSSTTAYMELRSLTSED | | SGTYFTLTISSVQAEDLA | |
| | SAVYFCARGYYGNSFAYW | | VYYCQNDYYFPYTFGGGT | |
| | GQGTLVTVSA | | KLEIK | |
| 54F5 | EVQLQQSGPELVKPGASV | 221 | DIVMTQSPSSLTVTAGEK | 281 |
| | KMSCKASGYTFTDYNMHW | | VTMSCKSSQSLLNSGNQK | |
| | LKQSHGKSLEWIGYINPK | | NYLTWYQKKPGQPPKLLI | |
| | NGGTRYNQKFKGKATLTV | | YWASTRESGVPDRFTGSG | |
| | NKSSSTAYMELRSLTSED | | SGTDFTLTISSVQAEDLA | |
| | SAVYYCARIYYGNSFDYW | | VYFCQNDYSFPFTFGSGT | |
| | GQGTTLTVSS | | KLEIK | |
| 56B2 | EVQLVESGGGLVKPGGSL | 227 | DIVMTQSPSSLTVTAGEK | 286 |
| | KLSCAASGFTFSDYGMHW | | VTMSCKSSQSLLNSGNQK | |
| | VRQAPEKGLEWVAYISSG | | NYLTWYQQKPGQPPKLLI | |
| | SSNIYYADTVKGRFTISR | | YWASTRESGVPDRFTGSG | |
| | DNAKNTLFLQMTSLRSED | | SGTDFTLTISSVQAEDLA | |
| | TAMYYCARFYYGNSFAYW | | VYYCQNAYSFPFTFGSGT | |
| | GQGTLVTVSA | | QLEIR | |
| 59E7 | EVQLQQSGPDLLKPGASV | 215 | DIVMTQSPSFLTVTAGEK | 263 |
| | KMSCKASGYTFTDYSMHW | | VTMSCKSSQSLLNSGNLK | |
| | VRQSHGKRLEWIGFINPY | | NYLTWYQQKPGQPPKLLI | |
| | SGSTTYNQKFKGKATLTV | | YWASTRESGVPDRFTGSG | |
| | NKSSSTVYMEVRSLTSDD | | SGSDFTLTISSVQAEDLA | |
| | SAVYYCTRIFYGNSFDYW | | VYYCQNDYFYPFTFGSGT | |
| | GQGTTLTVSS | | RLEMK | |
| 59F5 | EVQLQQSGPELLKPGASV | 218 | DIVMTQSPSFLTVTAGEK | 262 |
| | KMSCKASGYTFTDYTMHW | | VTMSCKSSQSLLNSGNLK | |
| | VKQSHGKSLEWIGYINPY | | NYLTWYQQKPGQPPKLLI | |
| | NSGTRYNQKFKGKATLTV | | YWASTRESGVPDRFTGSG | |
| | NKSSNTAYMEVRSLTSED | | SGSDFTLTISSVQAEDLA | |
| | SAVYYCTRIFYGNSFDYW | | VYYCQNDYFYPFTFGSGT | |
| | GQGTTLTVSS | | RLEIK | |
| 59G12 | EVQLQQSGPELLKPGASV | 216 | DIVMTQSPSFLTVTAGEK | 263 |
| | KMSCKASGYTFTDYSMHW | | VTMSCKSSQSLLNSGNLK | |
| | VRQSHGKRLEWIGFINPY | | NYLTWYQQKPGQPPKLLI | |
| | SGSTTYNQKFKGKATLTV | | YWASTRESGVPDRFTGSG | |
| | NKSSSTVYMEVRSLTSDD | | SGSDFTLTISSVQAEDLA | |
| | SAVYYCTRIFYGNSFDYW | | VYYCQNDYFYPFTFGSGT | |
| | GQGTTLTVSS | | RLEMK | |
| 60F11 | QVQLQQPGAELVKPGASV | 234 | DIVMTQSPSSLTVTAGEK | 274 |
| | KLSCKASGYTFTSYWMHW | | VTLSCKSSQSLLNSGNQR | |
| | VKQRPGQGLEWIGMIHPN | | NYLTWYQQKPGQPPKLLI | |
| | SGSTNYNEKFKSKATLTV | | YWASTRESGVPDRFTGSG | |
| | DKSSSTAYMQLSSLTSED | | SGTDFTLTISSVQAEDLA | |
| | SAVYYCARMGLGNAMDYW | | VYYCQNAYSYPLTFGAGT | |
| | GQGTSVTVSS | | KLELK | |
| 61A5 | EVKLVESEGGLVQPGSSM | 211 | DIVMSQSPSSLAVSVGEK | 261 |
| | KLSCTASGFTFSDYYMAW | | VTMSCKSSQSLLYSSNQK | |
| | VRQVPEKGLEWVANINYD | | NYLAWYQQKPGQSPKLLI | |
| | GSSTFYLDSLKSRFIISR | | YWASTRESGVPDRFTGSG | |
| | DNARNILYLQMTSLKSED | | SGTDFTLTISSVKAEDLA | |
| | TATYFCGRQVGYYDPMDY | | VYYCQQYYTYPLTFGAGT | |
| | WGQGTSVTVAS | | KLELK | |
| 64C1 | EVQLQQSGPELLKPGASV | 217 | DIVMTQSPSFLTVTAGEK | 262 |
| | KMSCKASGYTFTDYTIHW | | VTMSCKSSQSLLNSGNLK | |
| | VKQSHGESLEWIGYINPY | | NYLTWYQQKPGQPPKLLI | |
| | NSGTRYNQKFKGKATLTV | | YWASTRESGVPDRFTGSG | |
| | NKSSSTAYMEVRSLTSED | | SGSDFTLTISSVQAEDLA | |
| | SAVYFCTRIFYGNSFDYW | | VYYCQNDYFYPFTFGSGT | |
| | GQGTTLTVSS | | RLEIK | |
| 64C10 | EVQLQQSGPELLKPGASV | 219 | DIVMTQSPSFLTVTAGEK | 264 |
| | TMSCKASGYTFTDYTIHW | | VTMSCKSSQSLLNSGNLK | |
| | VKQSHGKSLEWIGSINPY | | NYLTWYQQKPGQPPKLLI | |
| | NPGTRYNQKFEGKATLTV | | YWASTRESGVPDRFTGSG | |
| | NKSSNTAYMEFRSLTSED | | SGSDFTLTISSVQAEDLA | |
| | SAVYYCTRVFYGNSFDYW | | VYYCQNNYFYPFTFGSGT | |
| | GQGTTLTVSS | | RLEIK | |
| 66D7-1 | QVLLQQPGTELVKPGASV | 230 | DIVMTQSPSSLTVTAGEK | 279 |
| | KVSCKASAYTFTSYWIHW | | VTMSCKSSQSLLNGGNQK | |
| | VKQRPGQGLEWIGRIRPS | | NYLTWYQQKPGQPPKLLI | |
| | DSDSTYNQNFKGKATLTV | | YWASTRESGVPDRFTGSG | |
| | DKSSDTAYMQLTSLTSED | | SGTDFTLTISTMQAEDLA | |
| | SAVYYCSMGAYYSNSFGY | | VYYCQNDYFFPYTFGGGT | |
| | WGQGSLVTVSA | | KLEIK | |
| 66D7-2 | QVQLQQSGPELVKPGTSV | 257 | DIVMTQSPSSLTVTAGEK | 271 |
| | KLSCKASGYTFINYDINW | | ATMSCKSSQSLLNGGNQK | |
| | VKQRPGQGLEWIAWIFPR | | NYLTWYQQKPGQPPTLLI | |
| | DGSTKYNEKFRGEATLTV | | YWASTRESGVPDRFTGSG | |
| | DTSSSTAYLGLHSLTSED | | SGTYFTLTISSVQAEDLA | |
| | SAVYFCARGYYGNSFAYW | | VYYCQNDYYFPYTFGGGT | |
| | GQGTLVTVSA | | KLEIK | |
| 66E12 | QVQLQQPGAELVKPGASV | 233 | DIVMTQSPSSLTVTAGEK | 292 |
| | KLSCKASGYTFSSYWIPW | | VTMSCKSSQSLLNSGNQK | |
| | VKQRPGQGLEWIGMIHPN | | NYLTWYQQKPGQPPKMLI | |
| | SGSTNYNEKFKRKAILIV | | YWASTRESGVPDRFTGSG | |
| | DKSSNTAYMQLSSLTSDD | | SGTDFTLTLSSVKAEDLA | |
| | SAVYYCGRMGLGNAMDYW | | VYYCQNDYYYPLTFGAGT | |
| | GQGTSVTVSS | | KLELR | |
| 66E6 | QVQLQQSGPELVKPGASV | 255 | DIVMTQSPSSLTVTAGER | 299 |
| | KLSCKASGYTFTNYDINW | | VTMSCKSSQSLLNSGNLK | |
| | VKQRPGQGLEWIGLIYPR | | NYLTWYQQKPGQPPKLLI | |
| | DKNTNYNGKFKGKATLTV | | YWASTRESGVPDRFTGSG | |
| | DTSSSTAYMELHSLTSED | | SGTYFTLTISSVQAEDLA | |
| | SAVYFCARGYYGNSFAYW | | VYYCQNDYYYPYTFGGGT | |
| | GQGTLVTVFA | | KLEIK | |
| 68D1 | QVQLQQSGPELVKPGASM | 252 | DIVMTQSPSSLTVTAGEK | 272 |
| | KLSCKASGYTFTSYDINW | | VTLSCKSSQSLLNSGNQK | |
| | VKQRPGQGLEWIGLSYPR | | NYLTWYQQKPGQPPKLLI | |
| | DGTTQYNGKFKGKATLTV | | YWASTRESGVPDRFTGSG | |
| | DTSSSTAYMELRSLTSED | | SGTYFTLTISSVQAEDLA | |
| | SAVYFCARGYYGNSFAYW | | VYYCQNDYYFPYTFGGGT | |
| | GQGTLVTVSA | | KLEIK | |
| 68E9 | EVQLQQSGPELVKPGSSV | 223 | DIVMTQSPSSLTVTAGEK | 285 |
| | KMSCKASGYTFTDYNMHW | | VTMSCKSSQSLLNSGNQK | |
| | LKQSHGKSLEWIGYINPK | | NYLTWYQQKPGQPPKLLI | |
| | NGGTRYNQKFKGKATLTV | | YWASTRESGVPDRFTGSG | |
| | NKSSSTAYMELRSLTSED | | SGTDFTLTISSVQAEDLA | |
| | SAVYYCARLYYGNSFDYW | | VYFCQNDYSFPFTFGSGT | |
| | GQGTTLTVSS | | KLEIK | |
| 69B2 | QVQLQQPGAELIKPGASV | 232 | DIVMTQSPSSLTVTAGEK | 287 |
| | KLSCKASGYTFTSYWIPW | | VTMSCKSSQSLLNSGNQK | |
| | VKQRPGQGLEWIGMIHPN | | NYLTWYQQKPGQPPKLLI | |
| | SDSTNYNEKFKSKATLTV | | YWASTRESGVPDRFTGSG | |
| | DKSSSTAYIQLSSLTSDD | | SGTDFTLTISSVQAEDLA | |
| | SAVYYCARMGLGNALDYW | | VYYCQNDYYYPLTFGAGT | |
| | GQGTSVTVSS | | KLELK | |
| 73E4 | QVQLQQSGPELVKPGASM | 253 | DIVMTQSPSSLTVTAGEK | 270 |
| | KLSCKASGYTFTSYDINW | | ATMSCKSSQSLLNGGNQK | |
| | VKQRPGQGPEWIGLSYPR | | NYLTWYQQKPGQPPKLLI | |
| | DSSTQYNGRERGKATLTV | | YWASTRESGVPDRFTGSG | |
| | DTSSTTAYMELRSLTSED | | SGTYFTLTISSVQAEDLA | |
| | SAVYFCARGYYGNSFAYW | | VYYCQNDYYFPYTFGGGT | |
| | GQGTLVTVSA | | KLEIK | |
| 78H6 | EVQLQQSGPELVKPGASV | 222 | DIMMTQSPSSLTVTAGEK | 260 |
| | KMSCKASGYTFTDYNMHW | | VTMSCKSSQSLLNSGNQK | |
| | VKQSHGKSLEWIGYINPN | | NYLTWYQQKPGQPPKLLI | |
| | NGGTTYNQKFKGKATLTV | | YWASTRESGVPDRFTGSG | |
| | NKSSSTAYMELRGLTSED | | SGTDFTLTISSVQAEDLA | |
| | SAIYYCARIYYGNSFDYW | | VYYCQNDYSFPFTFGSGT | |
| | GQGTTLTVSS | | KLEIK | |
| 79C3 | EVQLQQSGPELVKPGASV | 220 | DIVMTQSPSSLTVTAGEK | 284 |
| | KMSCKASGYTFTDYNIHW | | VTMSCKSSQSLLNSGNQK | |
| | LKQSPGKSLEWIGYINPK | | NYLTWYQQKPGQPPKLLI | |
| | NGGTRYNQKFKGKATLTV | | YWASTRESGVPDRFTGSG | |
| | NKSSSTAYMELRSLTSED | | SGTDFTLTISSVQAADLA | |
| | SAVYYCSRIYYGNSFDYW | | VYFCQNDYSFPFTFGSGT | |
| | GQGTTLTVSS | | KLEIK | |
| 80F10 | QVQLQQSGPELVKPGASM | 251 | DIVMTQSPSSLTVTAGEK | 291 |
| | KLSCKASGYTFTSYDINW | | VTMSCKSSQSLLNSGNQK | |
| | VKQRPGQGLEWIGLSYPR | | NYLTWYQQKPGQPPKLLM | |
| | DGTTQYNGKFKGEATLTV | | YWASTRESGVPDRFTGSG | |
| | DRSSSTAYMELRSLTSED | | SGTYFTLTISSVQAEDLA | |
| | SAVYFCARGYYGNSFAYW | | VYYCQNDYYFPYTFGGGT | |
| | GQGTLVTVSA | | KLEIK | |
| 83H3 | QVQLQQSGPELVKPGSSV | 256 | DIVMTQSPSSLAVTPGEK | 268 |
| | KLSCKASGYTFTRNDINW | | VTMNCKSSQSLLNDGNQK | |
| | VKQRPGQGLEWIGRIYPR | | NYLTWYQQKPGQPPKLLI | |
| | DGGTNYNEKFKGKATLTV | | YWASTRESGVPDRFAGSG | |
| | DTLSSTAYMELHSLTSED | | SGTSFTLTINSVQAEDLA | |
| | SAVHFCARGYYGNSFAYW | | VYYCQNGYSFPYTFGGGT | |
| | GQGTLVTVSA | | NLEIK | |
| 84E8 | QVQLQQPGAELVKPGASV | 236 | DIVMTQSPSSLTVTAGEK | 293 |
| | KLSCKPSGYTFSSYWIPW | | VTMSCKSSQSLLNSGNQK | |
| | VKQRPGQGLEWIGMIHPN | | NYLTWYQQKPGQSPKMLI | |
| | SGSTNYNEKFKRKAILTV | | YWASTRASGVPDRFTGSG | |
| | DKSSSTAYMQLSSLTSDD | | SGTDFTLTLSSVKAEDLA | |
| | SAVYYCGRMGLGNAMDYW | | VYYCQNDYYYPLTFGAGT | |
| | GQGTSVTVSS | | KLELR | |
| 97A9 | QVQLQQSGPELVKPGASM | 250 | DIVMTQSPSSLTVTAGEK | 290 |
| | KLSCKASGYSFTRNDINW | | VTMSCKSSQSLLNSGNQK | |
| | VKQRPGQGLEWIGLSYPR | | NYLTWYQQKPGQPPKLLI | |
| | DGTTQYNGKFKGKATLTV | | YWASTRESGVPDRFTGSG | |
| | DTSSSTAYMELRSLTSED | | SGTYFTLTISSVQAEDLA | |
| | SAVYFCARGYYGNSFAYW | | VYFCQNDYYFPYTFGGGT | |
| | GQGTLVTVSA | | KLEIK | |
| 99A7 | QVQLQQPGAELVKPGASV | 235 | DIVMTQSPSSLTVTAGEK | 288 |
| | KLSCKASGYTVTRYWIQW | | VTMSCKSSQSLLNSGNQK | |
| | VKQRPGQGLEWIGMIHPN | | NYLTWYQQKPGQPPKLLI | |
| | SGSTNYNEKFKKKAALTL | | YWASTRESGVPDRFTGSG | |
| | DKSSSTAYMQLSSPTSED | | SGTDFTLTISSVQAEDLA | |
| | SAVYYCVRMGLGNAMDEW | | VYYCQNNYVYPLTFGAGT | |
| | GQGTSVTVSS | | KLELR | |
| 99G8 | QVHLQQSGPELVKPGASV | 229 | DIVMTQSPSSLTVTAGEK | 282 |
| | KVSCKASGYSFRNYDINW | | VTMSCKSSQSLLNSGNQK | |
| | VKQRPGQGLEWIGRIYPR | | NYLTWYQQKPGQAPKLLI | |
| | DDSTTYNEKFKGKASLTV | | YWASTRQSGVPDRFTGSG | |
| | DTSSSTAYMEFHSLTSED | | FGTDFTLIITTVQTEDLA | |
| | SAVYFCARGYYGNSFAYW | | VYFCQNDFGFPYTFGGGT | |
| | GQGTLVTVSA | | KLEMN | |
| 99H8 | QVQLQQSGPELVKPGASV | 254 | DIVMTQSPSSLTVTAREK | 302 |
| | KLSCKASGYSFTNFDINW | | VIMNCKSSQSLENSGNQK | |
| | VKQRPGQGLQWIGRLYPR | | NYLTWYQQKPGQSPKLLI | |
| | DGTTTYNEKFKGKASLTV | | YWASTRQSGVPDRFTGSG | |
| | DTSSTTSYMDLHSLTSED | | SGTDFTLTISTVQAEDLA | |
| | SAVYFCVRGNYGNSFAYW | | VYFCQNGFSFPYTFGGGT | |
| | GQGTLVTVSA | | KLEMN | |

Given that each of the 60 exemplary monoclonal antibodies can bind to CLDN18 (in particular, CLDN18.2) and that antigen-binding specificity is provided primarily by the CDR1, CDR2 and CDR3 regions, the HCDR1, HCDR2 and HCDR3 sequences and LCDR1, LCDR2 and LCDR3 sequences of each of the 60 exemplary monoclonal antibodies can be “mixed and matched” (i.e., CDRs from different antibodies can be mixed and matched, but each antibody must contain a HCDR1, HCDR2 and HCDR3 and a LCDR1, LCDR2 and LCDR3) to create anti-CLDN18 (in particular, anti-CLDN18.2) binding molecules of the present disclosure. CLDN18 (in particular, CLDN18.2) binding of such “mixed and matched” antibodies can be tested using the binding assays described above and in the Examples. Preferably, when V_{H} CDR sequences are mixed and matched, the HCDR1, HCDR2 and/or HCDR3 sequence from a particular V_{H} sequence is replaced with a structurally similar CDR sequence (s). Likewise, when VL CDR sequences are mixed and matched, the LCDR1, LCDR2 and/or LCDR3 sequence from a particular VL sequence preferably is replaced with a structurally similar CDR sequence (s). For example, the HCDR1s of antibodies 99H8 and 99G8 share some structural similarity and therefore are amenable to mixing and matching. It will be readily apparent to a person skilled in the art that novel V_{H} and VL sequences can be created by substituting one or more V_{H} and/or VL CDR sequences with structurally similar sequences from the CDR sequences disclosed herein for the 60 exemplary monoclonal antibodies.

CDRs are known to be responsible for antigen binding. However, it has been found that not all of the 6 CDRs are indispensable or unchangeable. In other words, it is possible to replace or change or modify one or more CDRs in each of the 60 exemplary monoclonal antibodies, yet substantially retain the specific binding affinity to CLDN18 (in particular, CLDN18.2).

In certain embodiments, the antibodies and antigen-binding fragments thereof provided herein comprise suitable framework region (FR) sequences, as long as the antibodies and antigen-binding fragments thereof can specifically bind to CLDN18 (in particular, CLDN18.2). The CDR sequences provided in Table 2 above are obtained from mouse antibodies, but they can be grafted to any suitable FR sequences of any suitable species such as mouse, human, rat, rabbit, among others, using suitable methods known in the art such as recombinant techniques. In some embodiments, the antibodies or antigen-binding fragments thereof provided herein comprise a HFR1 comprising the sequence of SEQ ID NO: 156, a HFR2 comprising the sequence of SEQ ID NO: 162, a HFR3 comprising the sequence of SEQ ID NO: 169, a HFR4 comprising the sequence of SEQ ID NO: 177, a LFR1 comprising the sequence of SEQ ID NO: 179, a LFR2 comprising the sequence of SEQ ID NO: 185, a LFR3 comprising the sequence of SEQ ID NO: 190, a LFR4 comprising the sequence of SEQ ID NO: 198. In some embodiments, the antibodies or antigen-binding fragments thereof provided herein comprise a HFR1 comprising the sequence of SEQ ID NO: 158, a HFR2 comprising the sequence of SEQ ID NO: 165, a HFR3 comprising the sequence of SEQ ID NO: 172, a HFR4 comprising the sequence of SEQ ID NO: 177, a LFR1 comprising the sequence of SEQ ID NO: 182, a LFR2 comprising the sequence of SEQ ID NO: 189, a LFR3 comprising the sequence of SEQ ID NO: 194, a LFR4 comprising the sequence of SEQ ID NO: 198. The amino acid sequences of the FRs above are shown in Table 4 below.

TABLE 4
Amino acid sequences of murine FRs

| Amino Acid Sequence | SEQ ID NO | Amino Acid Sequence | SEQ ID NO |
|---|---|---|---|
| EAKLVESGGDFMQPGGSLK | 156 | WVRQTPEKRLEWVA | 162 |
| LSCAA | | | |
| RFTISRDNARNTLFLQMSS | 169 | WGQGTLVTVSA | 177 |
| LQSEDTAIYYCAT | | | |
| QIVLSQSPAILSASPGEKV | 179 | WYQQKPGSSPKAWIY | 185 |
| TMTC | | | |
| GVPTRFSGSGSGTSYSLTI | 190 | FGAGTKLELK | 198 |
| DRVEAEDAATYYC | | | |
| QVQLQQPGTELVKTGTSVK | 158 | WVIQRPGQGLEWIG | 165 |
| LSCKAS | | | |
| KATLTLDRSSTTAYMQLSS | 172 | DIVMTQSPSSLTVTAGEK | 182 |
| LTSEDSAVYFCAG | | VTLSC | |
| WYQQKPGQPPKLLIY | 189 | GVPDRFTGSGSGTDETLT | 194 |
| | | ISSVQAEDLAVYHC | |

In certain embodiments, the antibodies and antigen-binding fragments thereof provided herein are humanized. A humanized antibody or antigen-binding fragment thereof is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536).

Suitable human heavy chain and light chain variable domains can be selected to achieve this purpose using methods known in the art. In an illustrative example, “best-fit” approach can be used, where a non-human (e.g. rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) 1. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In some embodiments, the present disclosure provides 15 humanized antibodies of 35B4, which are designated as hu35B4.H1L1, hu35B4.H1L2, hu35B4.H1L3, hu35B4.H1L4, hu35B4.H1L1S92A, hu35B4.H2L1, hu35B4.H2L2, hu35B4.H2L3, hu35B4.H2L4, hu35B4.H2L1S92A, hu35B4.H3L1, hu35B4.H3L2, hu35B4.H3L3, hu35B4.H3L4, hu35B4.H3L1S92A, respectively. The SEQ ID NOs and specific amino acid sequences of the heavy and light chain variable regions of each humanized antibody of 35B4 are shown in Table 5 and Table 6 below. The SEQ ID NOs and specific amino acid sequences of the FRs of each humanized antibody of 35B4 are shown in Table 7 and Table 8 below. Each of the humanized antibodies hu35B4.H1L1, hu35B4.H1L2, hu35B4.H1L3, hu35B4.H1L4, hu35B4.H2L1, hu35B4.H2L2, hu35B4.H2L3, hu35B4.H2L4, hu35B4.H3L1, hu35B4.H3L2, hu35B4.H3L3, hu35B4.H3L4 comprises a HCDR1 comprising the sequence of SEQ ID NO: 201, a HCDR2 comprising the sequence of SEQ ID NO: 47, a HCDR3 comprising the sequence of SEQ ID NO: 80, a LCDR1 comprising the sequence of SEQ ID NO: 103, a LCDR2 comprising the sequence of SEQ ID NO: 118, and a LCDR3 comprising the sequence of SEQ ID NO: 138; each of the humanized antibodies hu35B4.H1L1S92A, hu35B4.H2L1S92A, hu35B4.H3L1S92A comprises a HCDR1 comprising the sequence of SEQ ID NO: 201, a HCDR2 comprising the sequence of SEQ ID NO: 47, a HCDR3 comprising the sequence of SEQ ID NO: 80, a LCDR1 comprising the sequence of SEQ ID NO: 103, a LCDR2 comprising the sequence of SEQ ID NO: 118, and a LCDR3 comprising the sequence of SEQ ID NO: 204. The CDR boundaries of the 15 humanized antibodies of 35B4 described above were defined or identified by the convention of IMGT.

TABLE 5
SEQ ID NOs of the humanized variable regions
for each of the humanized antibody of 35B4.

| Antibody | VH (SEQ ID NO) | VL (SEQ ID NO) |
|---|---|---|
| hu35B4.H1L1 | 311 | 314 |
| hu35B4.H1L2 | 311 | 315 |
| hu35B4.H1L3 | 311 | 316 |
| hu35B4.H1L4 | 311 | 317 |
| hu35B4.H1L1S92A | 311 | 402 |
| hu35B4.H2L1 | 312 | 314 |
| hu35B4.H2L2 | 312 | 315 |
| hu35B4.H2L3 | 312 | 316 |
| hu35B4.H2L4 | 312 | 317 |
| hu35B4.H2L1S92A | 312 | 402 |
| hu35B4.H3L1 | 313 | 314 |
| hu35B4.H3L2 | 313 | 315 |
| hu35B4.H3L3 | 313 | 316 |
| hu35B4.H3L4 | 313 | 317 |
| hu35B4.H3L1S92A | 313 | 402 |

TABLE 6
Amino acid sequence of the humanized variable regions for each
humanized antibody of 35B4.

| SEQ ID NO | Amino Acid Sequence |
|---|---|
| 311 | EVQLLESGGGLVQPGGSLRLSCAASGFTLSSYALSWVRQAPGK |
| | GLEWVSYISNLGGSTFYPDTVKGRFTISRDNSKNTLYLQMNSL |
| | RAEDTAVYYCAKHLYNYDAFASWGQGTLVTVSS |
| 312 | EVQLLESGGGLVQPGGSLRLSCAASGFTLSSYALSWVRQAPGK |
| | GLEWVSYISNLGGSTFYPDTVKGRFTISRDNSKNTLYLQMNSL |
| | RAEDTAVYYCATHLYNYDAFASWGQGTLVTVSS |
| 313 | EVQLLESGGGLVQPGGSLRLSCAASGFTLSSYALSWVRQAPGK |
| | GLEWVAYISNLGGSTFYPDTVKGRFTISRDNSKNTLYLQMNSL |
| | RAEDTAVYYCATHLYNYDAFASWGQGTLVTVSS |
| 314 | DIQLTQSPSFLSASVGDRVTITCRASSSVNYIHWYQQKPGKAP |
| | KLLIYATSNLASGVPSRFSGSGSGTEFTLTISSLQPEDFATYY |
| | CQQWNSNPLTFGQGTKLEIK |
| 315 | DIQLTQSPSFLSASVGDRVTITCRASSSVNYIHWYQQKPGKAP |
| | KALIYATSNLASGVPSRFSGSGSGTEYTLTISSLQPEDFATYY |
| | CQQWNSNPLTFGQGTKLEIK |
| 316 | DIQLTQSPSFLSASVGDRVTITCRASSSVNYIHWYQQKPGKSP |
| | KALIYATSNLASGVPSRFSGSGSGTEYTLTISSLQPEDFATYY |
| | CQQWNSNPLTFGQGTKLEIK |
| 317 | DIQLTQSPSFLSASVGDRVTMTCRASSSVNYIHWYQQKPGKSP |
| | KALIYATSNLASGVPSRFSGSGSGTEYTLTISSVQPEDFATYY |
| | CQQWNSNPLTFGQGTKLEIK |
| 402 | DIQLTQSPSFLSASVGDRVTITCRASSSVNYIHWYQQKPGKAP |
| | KALIYATSNLASGVPSRFSGSGSGTEYTLTISSLQPEDFATYY |
| | CQQWNANPLTFGQGTKLEIK |

TABLE 7
The SEQ ID NOs of FRs for each humanized heavy and light
chain variable regions for humanized antibody of 35B4.

| | FR1 | FR2 | FR3 | FR4 |
|---|---|---|---|---|
| VH or VL | (SEQ | (SEQ | (SEQ | (SEQ |
| Name | ID NO) | ID NO) | ID NO) | ID NO) |
| hu35B4.H1 | 157 | 163 | 170 | 178 |
| hu35B4.H2 | 157 | 163 | 171 | 178 |
| hu35B4.H3 | 157 | 164 | 171 | 178 |
| hu35B4.L1 | 180 | 186 | 191 | 199 |
| hu35B4.L2 | 180 | 187 | 192 | 199 |
| hu35B4.L3 | 180 | 188 | 192 | 199 |
| hu35B4.L4 | 181 | 188 | 193 | 199 |
| hu35B4.L1S92A | 180 | 187 | 192 | 199 |

TABLE 8
Amino acid sequences of the humanized FR for
humanized antibody of 35B4.

| SEQ ID NO | Amino Acid Sequence |
|---|---|
| 157 | EVQLLESGGGLVQPGGSLRLSCAA |
| 163 | WVRQAPGKGLEWVS |
| 164 | WVRQAPGKGLEWVA |
| 170 | RFTISRDNSKNTLYLQMNSLRAEDTAVYYCAK |
| 171 | RFTISRDNSKNTLYLQMNSLRAEDTAVYYCAT |
| 178 | WGQGTLVTVSS |
| 180 | DIQLTQSPSFLSASVGDRVTITC |
| 181 | DIQLTQSPSFLSASVGDRVTMTC |
| 186 | WYQQKPGKAPKLLIY |
| 187 | WYQQKPGKAPKALIY |
| 188 | WYQQKPGKSPKALIY |
| 191 | GVPSRFSGSGSGTEFTLTISSLQPEDFATYYC |
| 192 | GVPSRFSGSGSGTEYTLTISSLQPEDFATYYC |
| 193 | GVPSRFSGSGSGTEYTLTISSVQPEDFATYYC |
| 199 | FGQGTKLEIK |

In some embodiments, the present disclosure provides 12 humanized antibodies of 22E12, which are designated as hu22E12.H1L1, hu22E12.H1L2, hu22E12.H1L3, hu22E12.H2L1, hu22E12.H2L2, hu22E12.H2L3, hu22E12.H3L1, hu22E12.H3L2, hu22E12.H3L3, hu22E12.H4L1, hu22E12.H4L2, hu22E12.H4L3, respectively. The SEQ ID NOs and specific amino acid sequences of the heavy and light chain variable regions of each humanized antibody of 22E12 are shown in Table 9 and Table 10 below. The SEQ ID NOs and specific amino acid sequences of the FRs of each humanized antibody of 22E12 are shown in Table 11 and Table 12 below. Each of the 12 humanized antibodies for 22E12 above comprises a HCDR1 comprising the sequence of SEQ ID NO: 202, a HCDR2 comprising the sequence of SEQ ID NO: 203, a HCDR3 comprising the sequence of SEQ ID NO: 83; a LCDR1 comprising the sequence of SEQ ID NO: 205, a LCDR2 comprising the sequence of SEQ ID NO: 120, and a LCDR3 comprising the sequence of SEQ ID NO: 141. The CDR boundaries of the 12 humanized antibodies of 22E12 described above were defined or identified by the convention of IMGT.

TABLE 9
SEQ ID NOs of the humanized variable regions
for each of the humanized antibody of 22E12.

| Antibody | VH (SEQ ID NO) | VL (SEQ ID NO) |
|---|---|---|
| hu22E12.H1L1 | 318 | 322 |
| hu22E12.H1L2 | 318 | 323 |
| hu22E12.H1L3 | 318 | 324 |
| hu22E12.H2L1 | 319 | 322 |
| hu22E12.H2L2 | 319 | 323 |
| hu22E12.H2L3 | 319 | 324 |
| hu22E12.H3L1 | 320 | 322 |
| hu22E12.H3L2 | 320 | 323 |
| hu22E12.H3L3 | 320 | 324 |
| hu22E12.H4L1 | 321 | 322 |
| hu22E12.H4L2 | 321 | 323 |
| hu22E12.H4L3 | 321 | 324 |

TABLE 10
Amino acid sequence of the humanized variable regions for each of
the humanized antibody of 22E12.

| SEQ ID NO | Amino Acid Sequence |
|---|---|
| 318 | QVQLVQSGAEVKKPGASVKVSCKASGYTFTNWVHWVRQAPGQGLEWMGE |
| | INPTNARSNYNEKFKKRVTMTRDTSTSTVYMELSSLRSEDTAVYYCARI |
| | YYGNSFAHWGQGTLVTVSS |
| 319 | QVQLVQSGAEVKKPGASVKVSCKASGYTFTNWVHWVRQAPGQGLEWMGE |
| | INPTNARSNYNEKFKKRVTMTRDRSTSTVYMELSSLRSEDTAVYFCAGI |
| | YYGNSFAHWGQGTLVTVSS |
| 320 | QVQLVQSGAEVVKPGASVKVSCKASGYTFTNWVHWVIQAPGQGLEWMGE |
| | INPTNARSNYNEKFKKRVTMTLDRSTSTVYMELSSLRSEDTAVYFCAGI |
| | YYGNSFAHWGQGTLVTVSS |
| 321 | QVQLVQSGAEVVKPGASVKLSCKASGYTFTNWVHWVIQAPGQGLEWIGE |
| | INPTNARSNYNEKFKKRVTLTLDRSTSTVYMELSSLRSEDTAVYFCAGI |
| | YYGNSFAHWGQGTLVTVSS |
| 322 | DIVMTQSPDSLAVSLGERATINCKSSQSLLNAGNQKNYLTWYQQKPGQP |
| | PKLLIYWSSTRESGVPDRESGSGSGTDFTLTISSLQAEDVAVYYCQNNY |
| | YYPLTFGGGTKLEIK |
| 323 | DIVMTQSPDSLAVSLGERATINCKSSQSLLNAGNQKNYLTWYQQKPGQP |
| | PKLLIYWSSTRESGVPDRESGSGSGTDFTLTISSLQAEDVAVYHCQNNY |
| | YYPLTFGGGTKLEIK |
| 324 | DIVMTQSPDSLAVSLGERVTLNCKSSQSLLNAGNQKNYLTWYQQKPGQP |
| | PKLLIYWSSTRESGVPDRFSGSGSGTDFTLTISSVQAEDVAVYHCQNNY |
| | YYPLTFGGGTKLEIK |

TABLE 11
The SEQ ID NOs of FRs for each humanized heavy and light
chain variable regions for humanized antibody of 22E12.

| | FR1 | FR2 | FR3 | FR4 |
|---|---|---|---|---|
| VH or VL | (SEQ | (SEQ | (SEQ | (SEQ |
| Name | ID NO) | ID NO) | ID NO) | ID NO) |
| hu22E12.H1 | 159 | 166 | 173 | 178 |
| hu22E12.H2 | 159 | 166 | 174 | 178 |
| hu22E12.H3 | 160 | 167 | 175 | 178 |
| hu22E12.H4 | 161 | 168 | 176 | 178 |
| hu22E12.L1 | 183 | 189 | 195 | 200 |
| hu22E12.L2 | 183 | 189 | 196 | 200 |
| hu22E12.L3 | 184 | 189 | 197 | 200 |

TABLE 12
Amino acid sequences of the humanized FR for
humanized antibody of 22E12.

| SEQ ID NO | Amino Acid Sequence |
|---|---|
| 159 | QVQLVQSGAEVKKPGASVKVSCKAS |
| 160 | QVQLVQSGAEVVKPGASVKVSCKAS |
| 161 | QVQLVQSGAEVVKPGASVKLSCKAS |
| 166 | WVRQAPGQGLEWMG |
| 167 | WVIQAPGQGLEWMG |
| 168 | WVIQAPGQGLEWIG |
| 173 | RVTMTRDTSTSTVYMELSSLRSEDTAVYYCAR |
| 174 | RVTMTRDRSTSTVYMELSSLRSEDTAVYFCAG |
| 175 | RVTMTLDRSTSTVYMELSSLRSEDTAVYFCAG |
| 176 | RVTLTLDRSTSTVYMELSSLRSEDTAVYFCAG |
| 178 | WGQGTLVTVSS |
| 183 | DIVMTQSPDSLAVSLGERATINC |
| 184 | DIVMTQSPDSLAVSLGERVTLNC |
| 189 | WYQQKPGQPPKLLIY |
| 195 | GVPDRFSGSGSGTDFTLTISSLQAEDVAVYYC |
| 196 | GVPDRFSGSGSGTDFTLTISSLQAEDVAVYHC |
| 197 | GVPDRFSGSGSGTDFTLTISSVQAEDVAVYHC |
| 200 | FGGGTKLEIK |

In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1-4, and/or light chain LFR1-4.

In some embodiments, the FR regions derived from human may comprise the same amino acid sequence as the human immunoglobulin from which it is derived.

In some embodiments, one or more amino acid residues of the human FR are substituted with the corresponding residues from the parent non-human antibody.

This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In certain embodiments, the present disclosure also provides humanized anti-CLDN18 (in particular, anti-CLDN18.2) antibodies and antigen-binding fragments thereof comprising a heavy chain HFR1 comprising the sequence of EX₆₂X₆₃LX₆₄ESGGX₆₅X₆₆X₆₇QPGGSLX₆₈LSCAA (SEQ ID NO: 355) or QVQLX₆₉QX₇₀GX₇₁EX₇₂X₇₃KX₇₄GX₇₅SVKX₇₆SCKAS (SEQ ID NO: 356), or a homologous sequence of at least 80% sequence identity thereof, a heavy chain HFR2 comprising the sequence of WVRQX₇₇PX₇₈KX₇₉LEWVX₈₀ (SEQ ID NO: 357) or WVX₈₁QX₈₂PGQGLEWX₈₃G (SEQ ID NO: 358) or a homologous sequence of at least 80% sequence identity thereof, a heavy chain HFR3 comprising the sequence of RFTISRDNX₈₄X₈₅NTLX₈₆LQMX₈₇SLX₈₈X₈₉EDTAX₉₀YYCAX₉₁ (SEQ ID NO: 359) or X₉₃X₉₄TX₉₅TX₉₆DX₉₇SX₉₈X₉₉TX₁₀₀YMX₁₀₁LSSLX₁₀₂SEDX₁₀₃AVYX₁₀₄CAX₁₀₅ (SEQ ID NO: 360) or a homologous sequence of at least 80% sequence identity thereof, and a heavy chain HFR4 comprising the sequence of WGQGTLVTVSX₉₂ (SEQ ID NO: 361) or WGQGTLVTVSX₁₀₆ (SEQ ID NO: 362) or a homologous sequence of at least 80% sequence identity thereof, wherein X₆₂=A or V; X₆₃=K or Q; X₆₄=V or L; X₆₅=D or G; X₆₆=F or L; X₆₇=M or V; X₆₈=K or R; X₆₉=Q or V; X₇₀=P or S; X₇₁=T or A; X₇₂=L or V; X₇₃=V or K; X₇₄=T or P; X₇₅=T or A; X₇₆=L or V; X₇₇=T or A; X₇₈=E or G; X₇₉=R or G; X₈₀=A or S; X₈₁=I or R; X₈₂=R or A; X₈₃=I or M; X₈₄=A or S; X₈₅=R or K; X₈₆=F or Y; X₈₇=S or N; X₈₈=Q or R; X₈₉=S or A; X₉₀=I or V; X₉₁=T or K; X₉₃=K or R; X₉₄=A or V; X₉₅=L or M; X₉₆=L or R; X₉₇=R or T; X₉₈=S or T; X₉₉=T or S; X₁₀₀=A or V; X₁₀₁=Q or E; X₁₀₂=T or R; X₁₀₃=S or T; X₁₀₄=F or Y; X₁₀₅=G or R; X₉₂=A or S; X₁₀₆=A or S.

In certain embodiments, the present disclosure also provides humanized anti-CLDN18 (in particular, anti-CLDN18.2) antibodies and antigen-binding fragments thereof comprising a light chain LFR1 comprising the sequence of X₁₀₇IX₁₀₈X₁₀₉X₁₁₀QSPX₁₁₁X₁₁₂LX₁₁₃X₁₁₄X₁₁₅X₁₁₆GX₁₁₇X₁₁₈X₁₁₉TX₁₂₀X₁₂₁C (SEQ ID NO: 363) or a homologous sequence of at least 80% sequence identity thereof, a light chain LFR2 comprising the sequence of WYQQKPGX₁₂₂X₁₂₃PKX₁₂₄X₁₂₅IY (SEQ ID NO: 364) or a homologous sequence of at least 80% sequence identity thereof, a light chain LFR3 comprising the sequence of GVPX₁₂₆RFX₁₂₇GSGSGTX₁₂₈X₁₂₉X₁₃₀LTIX₁₃₁X₁₃₂X₁₃₃X₁₃₄X₁₃₅EDX₁₃₆AX₁₃₇YX₁₃₈C (SEQ ID NO: 365) or a homologous sequence of at least 80% sequence identity thereof, and a light chain LFR4 comprising the sequence of FGX₁₃₉GTKLEX₁₄₀K (SEQ ID NO: 366) or a homologous sequence of at least 80% sequence identity thereof, wherein X₁₀₇=Q or D; X₁₀₈=V or Q; X₁₀₉=L or M; X₁₁₀=S or T; X₁₁₁=A or S or D; X₁₁₂=I or F or S; X₁₁₃=S or T or A; X₁₁₄=A or V; X₁₁₅=S or T; X₁₁₆=P or V or A or L; X₁₁₇=E or D; X₁₁₈=K or R; X₁₁₉=V or A; X₁₂₀=M or I or L; X₁₂₁=T or S or N; X₁₂₂=S or K or Q; X₁₂₃=S or A or P; X₁₂₄=A or L; X₁₂₅=W or L; X₁₂₆=T or S or D; X₁₂₇=S or T; X₁₂₈=S or E or D; X₁₂₉=Y or F; X₁₃₀=S or T; X₁₃₁=D or S; X₁₃₂=R or S; X₁₃₃=V or L; X₁₃₄=E or Q; X₁₃₅=A or P; X₁₃₆=A or F or L or V; X₁₃₇=T or V; X₁₃₈=Y or H; X₁₃₉=A or Q or A or G; X₁₄₀=L or I.

In certain embodiments, the present disclosure also provides humanized anti-CLDN18 (in particular, anti-CLDN18.2) antibodies and antigen-binding fragments thereof comprising a heavy chain HFR1 comprising a sequence selected from the group consisting of SEQ ID NOs: 157-161, a heavy chain HFR2 comprising the sequence of SEQ ID NOs: 163-168, a heavy chain HFR3 comprising a sequence selected from the group consisting of SEQ ID NOs: 170-176, and a heavy chain HFR4 comprising a sequence of SEQ ID NO: 178; and/or a light chain LFR1 comprising a sequence from the group consisting of SEQ ID NOs: 180-184, a light chain LFR2 comprising a sequence selected from the group consisting of SEQ ID NOs: 186-189, a light chain LFR3 comprising a sequence selected from the group consisting of SEQ ID NOs: 191-197, and a light chain LFR4 comprising a sequence selected from the group consisting of SEQ ID NOs: 199-200.

In certain embodiments, the humanized anti-CLDN18 (in particular, anti-CLDN18.2) antibodies and antigen-binding fragments thereof provided herein comprise a heavy chain variable domain sequence selected from the group consisting of SEQ ID NOs: 311-313, 318-321, and a homologous sequence thereof having at least 80% sequence identity yet retaining specific binding affinity to CLDN18; and/or a light chain variable domain sequence selected from the group consisting of SEQ ID NOs: 314-317, 322-324, and a homologous sequence thereof having at least 80% sequence identity yet retaining specific binding affinity to CLDN18.

The present disclosure also provides 15 exemplary humanized antibodies of 35B4, including:
1) “hu35B4.H1L1” comprising the heavy chain variable region of hu35B4.H1 (SEQ ID NO: 311) and the light chain variable region of hu35B4.L1 (SEQ ID NO: 314);
2) “hu35B4.H1L2” comprising the heavy chain variable region of hu35B4.H1 (SEQ ID NO: 311) and the light chain variable region of hu35B4.L2 (SEQ ID NO: 315);
3) “hu35B4.H1L3” comprising the heavy chain variable region of hu35B4.H1 (SEQ ID NO: 311) and the light chain variable region of hu35B4.L3 (SEQ ID NO: 316);
4) “hu35B4.H1L4” comprising the heavy chain variable region of hu35B4.H1 (SEQ ID NO: 311) and the light chain variable region of hu35B4.L4 (SEQ ID NO: 317);
5) “hu35B4.H1L1S92A” comprising the heavy chain variable region of hu35B4.H1 (SEQ ID NO: 311) and the light chain variable region of hu35B4.L1S92A (SEQ ID NO: 402);
6) “hu35B4.H2L1” comprising the heavy chain variable region of hu35B4.H2 (SEQ ID NO: 312) and the light chain variable region of hu35B4.L1 (SEQ ID NO: 314);
7) “hu35B4.H2L2” comprising the heavy chain variable region of hu35B4.H2 (SEQ ID NO: 312) and the light chain variable region of hu35B4.L2 (SEQ ID NO: 315);
8) “hu35B4.H2L3” comprising the heavy chain variable region of hu35B4.H2 (SEQ ID NO: 312) and the light chain variable region of hu35B4.L3 (SEQ ID NO: 316);
9) “hu35B4.H2L4” comprising the heavy chain variable region of hu35B4.H2 (SEQ ID NO: 312) and the light chain variable region of hu35B4.L4 (SEQ ID NO: 317);
10) “hu35B4.H2L1S92A” comprising the heavy chain variable region of hu35B4.H2 (SEQ ID NO: 312) and the light chain variable region of hu35B4.L1S92A (SEQ ID NO: 402);
11) “hu35B4.H3L1” comprising the heavy chain variable region of hu35B4.H3 (SEQ ID NO: 313) and the light chain variable region of hu35B4.L1 (SEQ ID NO: 314);
12) “hu35B4.H3L2” comprising the heavy chain variable region of hu35B4.H3 (SEQ ID NO: 313) and the light chain variable region of hu35B4.L2 (SEQ ID NO: 315);
13) “hu35B4.H3L3” comprising the heavy chain variable region of hu35B4.H3 (SEQ ID NO: 313) and the light chain variable region of hu35B4.L3 (SEQ ID NO: 316);
14) “hu35B4.H3L4” comprising the heavy chain variable region of hu35B4.H3 (SEQ ID NO: 313) and the light chain variable region of hu35B4.L4 (SEQ ID NO: 317);
15) “hu35B4.H3L1S92A” comprising the heavy chain variable region of hu35B4.H3 (SEQ ID NO: 313) and the light chain variable region of hu35B4.L1S92A (SEQ ID NO: 402).

The present disclosure also provides 12 exemplary humanized antibodies of 22E12, including:
1) “hu22E12.H1L1” comprising the heavy chain variable region of hu22E12.H1 (SEQ ID NO: 318) and the light chain variable region of hu22E12.L1 (SEQ ID NO: 322);
2) “hu22E12.H1L2” comprising the heavy chain variable region of hu22E12.H1 (SEQ ID NO: 318) and the light chain variable region of hu22E12.L2 (SEQ ID NO: 323);
3) “hu22E12.H1L3” comprising the heavy chain variable region of hu22E12.H1 (SEQ ID NO: 318) and the light chain variable region of hu22E12.L3 (SEQ ID NO: 324);
4) “hu22E12.H2L1” comprising the heavy chain variable region of hu22E12.H2 (SEQ ID NO: 319) and the light chain variable region of hu22E12.L1 (SEQ ID NO: 322);
5) “hu22E12.H2L2” comprising the heavy chain variable region of hu22E12.H2 (SEQ ID NO: 319) and the light chain variable region of hu22E12.L2 (SEQ ID NO: 323);
6) “hu22E12.H2L3” comprising the heavy chain variable region of hu22E12.H2 (SEQ ID NO: 319) and the light chain variable region of hu22E12.L3 (SEQ ID NO: 324);
7) “hu22E12.H3L1” comprising the heavy chain variable region of hu22E12.H3 (SEQ ID NO: 320) and the light chain variable region of hu22E12.L1 (SEQ ID NO: 322);
8) “hu22E12.H3L2” comprising the heavy chain variable region of hu22E12.H3 (SEQ ID NO: 320) and the light chain variable region of hu22E12.L2 (SEQ ID NO: 323);
9) “hu22E12.H3L3” comprising the heavy chain variable region of hu22E12.H3 (SEQ ID NO: 320) and the light chain variable region of hu22E12.L3 (SEQ ID NO: 324);
10) “hu22E12.H4L1” comprising the heavy chain variable region of hu22E12.H4 (SEQ ID NO: 321) and the light chain variable region of hu22E12.L1 (SEQ ID NO: 322);
11) “hu22E12.H4L2” comprising the heavy chain variable region of hu22E12.H4 (SEQ ID NO: 321) and the light chain variable region of hu22E12.L2 (SEQ ID NO: 323);
12) “hu22E12.H4L3” comprising the heavy chain variable region of hu22E12.H4 (SEQ ID NO: 321) and the light chain variable region of hu22E12.L3 (SEQ ID NO: 324).

These exemplary humanized anti-CLDN18 (in particular, anti-CLDN18.2) antibodies retained the specific binding capacity or affinity to CLDN18 (in particular, CLDN18.2), and are at least comparable to, or even better than, the parent mouse antibody 35B4 or 22E12 in that aspect.

In some embodiments, the anti-CLDN18 (in particular, anti-CLDN18.2) antibodies and antigen-binding fragments provided herein comprise all or a portion of the heavy chain variable domain and/or all or a portion of the light chain variable domain. In one embodiment, the anti-CLDN18 (in particular, anti-CLDN18.2) antibody or an antigen-binding fragment thereof provided herein is a single domain antibody which consists of all or a portion of the heavy chain variable domain provided herein. More information of such a single domain antibody is available in the art (see, e.g. U.S. Pat. No. 6,248,516).

In certain embodiments, the anti-CLDN18 (in particular, anti-CLDN18.2) antibodies or the antigen-binding fragments thereof provided herein further comprise an immunoglobulin (Ig) constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-CLDN18 (in particular, anti-CLDN18.2) antibodies or the antigen-binding fragments thereof provided herein comprises heavy chain constant regions of human IgG1, IgG2, IgG3, IgG4, IgA1, IgA2 or IgM. In certain embodiments, the light chain constant region comprises Cκ or Cλ. The constant region of the anti-CLDN18 (in particular, anti-CLDN18.2) antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as antibody-dependent cellular cytotoxicity (ADCC) and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-CLDN18 (in particular, anti-CLDN18.2) antibodies and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fc region derived from human IgG1 with enhanced effector functions. In some embodiments, the Fc region derived from human IgG1 comprises one or more mutations selected from the group consisting of L235V, G236A, S239D, F243L, H268F, R292P, Y300L, V305I, S324T, A330L, I332E, and P396L. In certain embodiments, the Fc region derived from human IgG1 comprises a mutation selected from the group consisting of: (1) G236A, S239D and I332E; (2) S239D, A330L and I332E; (3) S239D and I332E; (4) S239D, H268F, S324T and I332E; (5) F243L, R292P, Y300L, V305I and P396L; (6) L235V, F243L, R292P, Y300L and P396L. In certain embodiments, the amino acid sequence of wild type human IgG1 is set forth in SEQ TD NO: 325. In certain embodiments, the Fc region comprises an amino acid sequence selected from the group consisting of SEQ ID NOs: 326-331. The amino acid sequences of SEQ ID NOs: 325-331 are shown in Table 13 below, and the mutation sites of each Fc region are underlined.

TABLE 13
Amino acid sequences of wild type human IgG1 and several Fc
regions

| | Mutation | | SEQ ID |
|---|---|---|---|
| Name | Site | Amino Acid Sequence | NO |
| Wild type | | ASTKGPSVFPLAPSSKSTSGGTAALGCLVKDYFPEP | 325 |
| hIgG1 | | VTVSWNSGALTSGVHTFPAVLQSSGLYSLSSVVTVP | |
| | | SSSLGTQTYICNVNHKPSNTKVDKKVEPKSCDKTHT | |
| | | CPPCPAPELLGGPSVFLFPPKPKDTLMISRTPEVTC | |
| | | VVVDVSHEDPEVKFNWYVDGVEVHNAKTKPREEQYN | |
| | | STYRVVSVLTVLHQDWLNGKEYKCKVSNKALPAPIE | |
| | | KTISKAKGQPREPQVYTLPPSREEMTKNQVSLTCLV | |
| | | KGFYPSDIAVEWESNGQPENNYKTTPPVLDSDGSFF | |
| | | LYSKLTVDKSRWQQGNVFSCSVMHEALHNHYTQKSL | |
| | | SLSPGK | |
| ADE | G236A, | ASTKGPSVFPLAPSSKSTSGGTAALGCLVKDYFPEP | 326 |
| | S239D, | VTVSWNSGALTSGVHTFPAVLQSSGLYSLSSVVTVP | |
| | I332E | SSSLGTQTYICNVNHKPSNTKVDKKVEPKSCDKTHT | |
| | | CPPCPAPELLAGPDVFLFPPKPKDTLMISRTPEVTC | |
| | | VVVDVSHEDPEVKFNWYVDGVEVHNAKTKPREEQYN | |
| | | STYRVVSVLTVLHQDWLNGKEYKCKVSNKALPAPEE | |
| | | KTISKAKGQPREPQVYTLPPSREEMTKNQVSLTCLV | |
| | | KGFYPSDIAVEWESNGQPENNYKTTPPVLDSDGSFF | |
| | | LYSKLTVDKSRWQQGNVFSCSVMHEALHNHYTQKSL | |
| | | SLSPGK | |
| DLE | S239D, | ASTKGPSVFPLAPSSKSTSGGTAALGCLVKDYFPEP | 327 |
| | A330L, | VTVSWNSGALTSGVHTFPAVLQSSGLYSLSSVVTVP | |
| | I332E | SSSLGTQTYICNVNHKPSNTKVDKKVEPKSCDKTHT | |
| | | CPPCPAPELLGGPDVFLFPPKPKDTLMISRTPEVTC | |
| | | VVVDVSHEDPEVKFNWYVDGVEVHNAKTKPREEQYN | |
| | | STYRVVSVLTVLHQDWLNGKEYKCKVSNKALPLPEE | |
| | | KTISKAKGQPREPQVYTLPPSREEMTKNQVSLTCLV | |
| | | KGFYPSDIAVEWESNGQPENNYKTTPPVLDSDGSFF | |
| | | LYSKLTVDKSRWQQGNVFSCSVMHEALHNHYTQKSL | |
| | | SLSPGK | |
| DE | S239D, | ASTKGPSVFPLAPSSKSTSGGTAALGCLVKDYFPEP | 328 |
| | I332E | VTVSWNSGALTSGVHTFPAVLQSSGLYSLSSVVTVP | |
| | | SSSLGTQTYICNVNHKPSNTKVDKKVEPKSCDKTHT | |
| | | CPPCPAPELLGGPDVFLFPPKPKDTLMISRTPEVTC | |
| | | VVVDVSHEDPEVKFNWYVDGVEVHNAKTKPREEQYN | |
| | | STYRVVSVLTVLHQDWLNGKEYKCKVSNKALPAPEE | |
| | | KTISKAKGQPREPQVYTLPPSREEMTKNQVSLTCLV | |
| | | KGFYPSDIAVEWESNGQPENNYKTTPPVLDSDGSFF | |
| | | LYSKLTVDKSRWQQGNVFSCSVMHEALHNHYTQKSL | |
| | | SLSPGK | |
| DFTE | S239D, | ASTKGPSVFPLAPSSKSTSGGTAALGCLVKDYFPEP | 329 |
| | H268F, | VTVSWNSGALTSGVHTFPAVLQSSGLYSLSSVVTVP | |
| | S324T, | SSSLGTQTYICNVNHKPSNTKVDKKVEPKSCDKTHT | |
| | I332E | CPPCPAPELLGGPDVFLFPPKPKDTLMISRTPEVTC | |
| | | VVVDVSFEDPEVKFNWYVDGVEVHNAKTKPREEQYN | |
| | | STYRVVSVLTVLHQDWLNGKEYKCKVTNKALPAPEE | |
| | | KTISKAKGQPREPQVYTLPPSREEMTKNQVSLTCLV | |
| | | KGFYPSDIAVEWESNGQPENNYKTTPPVLDSDGSFF | |
| | | LYSKLTVDKSRWQQGNVFSCSVMHEALHNHYTQKSL | |
| | | SLSPGK | |
| LPLIL | F243L, | ASTKGPSVFPLAPSSKSTSGGTAALGCLVKDYFPEP | 330 |
| | R292P, | VTVSWNSGALTSGVHTFPAVLQSSGLYSLSSVVTVP | |
| | Y300L, | SSSLGTQTYICNVNHKPSNTKVDKKVEPKSCDKTHT | |
| | V305I, | CPPCPAPELLGGPSVFLLPPKPKDTLMISRTPEVTC | |
| | P396L | VVVDVSHEDPEVKFNWYVDGVEVHNAKTKPPEEQYN | |
| | | STLRVVSILTVLHQDWLNGKEYKCKVSNKALPAPIE | |
| | | KTISKAKGQPREPQVYTLPPSREEMTKNQVSLTCLV | |
| | | KGFYPSDIAVEWESNGQPENNYKTTPLVLDSDGSFF | |
| | | LYSKLTVDKSRWQQGNVFSCSVMHEALHNHYTQKSL | |
| | | SLSPGK | |
| VLPLL | L235V, | ASTKGPSVFPLAPSSKSTSGGTAALGCLVKDYFPEP | 331 |
| | F243L, | VTVSWNSGALTSGVHTFPAVLQSSGLYSLSSVVTVP | |
| | R292P, | SSSLGTQTYICNVNHKPSNTKVDKKVEPKSCDKTHT | |
| | Y300L, | CPPCPAPELVGGPSVFLLPPKPKDTLMISRTPEVTC | |
| | P396L | VVVDVSHEDPEVKFNWYVDGVEVHNAKTKPPEEQYN | |
| | | STLRVVSVLTVLHQDWLNGKEYKCKVSNKALPAPIE | |
| | | KTISKAKGQPREPQVYTLPPSREEMTKNQVSLTCLV | |
| | | KGFYPSDIAVEWESNGQPENNYKTTPLVLDSDGSFF | |
| | | LYSKLTVDKSRWQQGNVFSCSVMHEALHNHYTQKSL | |
| | | SLSPGK | |

In certain embodiments, the antibodies or the antigen-binding fragments thereof provided herein have a specific binding affinity to human CLDN18.2 which is sufficient to provide for diagnostic and/or therapeutic use.

The antibodies or antigen-binding fragments thereof provided herein can be a monoclonal antibody, a polyclonal antibody, a humanized antibody, a chimeric antibody, a recombinant antibody, a bispecific antibody, a multi-specific antibody, a labeled antibody, a bivalent antibody, an anti-idiotypic antibody, or a fusion protein.

A recombinant antibody is an antibody prepared in vitro using recombinant methods rather than in animals.

In certain embodiments, the present disclosure provides an anti-CLDN18 (in particular, anti-CLDN18.2) antibody or antigen-binding fragment thereof, which competes for binding to CLDN18 (in particular CLDN18.2) with the antibody or antigen-binding fragment thereof provided herein. In certain embodiments, the present disclosure provides an anti-CLDN18 (in particular, anti-CLDN18.2) antibody or antigen-binding fragment thereof, which competes for binding to human CLDN18 (in particular, CLDN18.2) with any one of antibodies 99H8, 99G8, 99A7, 97A9, 84E8, 83H3, 80F10, 79C3, 78H6, 73E4, 69B2, 68E9, 68D1, 66E6, 66E12, 64C1, 64C10, 61A5, 60F11, 59G12, 59F5, 59E7, 56B2, 54F5, 38B9, 35B4, 35A10, 33G12, 22E12, 15E10, 100F4, 40C1, 41B3, 66D7-1, 66D7-2, 51G10, 365F6, 360C2, 319F2, 317A7, 315F10, 314D7, 310H5, 308E8, 305G8, 256C10-1, 256C10-2, 248D9, 246B8, 243A8, 242G5, 237E3, 226E9, 226D5, 217B5, 214E4, 213A9, 206C7, 203D12 or 203A5.

In some embodiments, the CLDN18 provided herein is a human CLDN18.2.

In some embodiments, the CLDN18 is a human CLDN18.2 comprising an amino acid sequence of SEQ ID NO: 401, which is shown in Table 14 below.

In certain embodiments, the anti-CLDN18 (in particular, anti-CLDN18.2) antibody or antigen-binding fragment thereof provided herein is not IMAB362.

“IMAB362” as used herein refers to an antibody or antigen binding fragment thereof comprising a heavy chain variable region having an amino acid sequence of SEQ ID NO: 397, and a light chain variable region having an amino acid sequence of SEQ ID NO: 398. The amino acid sequences of full-length heavy chain and full-length light chain of IMAB362 are set forth in SEQ ID NO: 399 and 400, respectively. The amino acid sequences of SEQ ID NOs: 397-400 are shown in Table 14 below.

TABLE 14
Amino acid sequences of SEQ ID NOs: 397-401

| | SEQ ID | |
|---|---|---|
| Description | NO | Amino Acid Sequence |
| VH of IMAB362 | 397 | QVQLQQPGAE LVRPGASVKL SCKASGYTFT |
| | | SYWINWVKQR PGQGLEWIGN IYPSDSYTNY |
| | | NQKFKDKATL TVDKSSSTAY MQLSSPTSED |
| | | SAVYYCTRSW RGNSFDYWGQ GTTLTVSS |
| VL of IMAB362 | 398 | DIVMTQSPSS LTVTAGEKVT MSCKSSQSLL |
| | | NSGNQKNYLT WYQQKPGQPP KLLIYWASTR |
| | | ESGVPDRFTG SGSGTDFTLT ISSVQAEDLA |
| | | VYYCQNDYSY PFTFGSGTKL EIK |
| Heavy chain of | 399 | QVQLQQPGAE LVRPGASVKL SCKASGYTFT |
| IMAB362 | | SYWINWVKQR PGQGLEWIGN IYPSDSYTNY |
| | | NQKFKDKATL TVDKSSSTAY MQLSSPTSED |
| | | SAVYYCTRSW RGNSFDYWGQ GTTLTVSSAS |
| | | TKGPSVFPLA PSSKSTSGGT AALGCLVKDY |
| | | FPEPVTVSWN SGALTSGVHT FPAVLQSSGL |
| | | YSLSSVVTVP SSSLGTQTYI CNVNHKPSNT |
| | | KVDKRVEPKS CDKTHTCPPC PAPELLGGPS |
| | | VFLFPPKPKD TLMISRTPEV TCVVVDVSHE |
| | | DPEVKFNWYV DGVEVHNAKT KPREEQYNST |
| | | YRVVSVLTVL HQDWLNGKEY KCKVSNKALP |
| | | APIEKTISKA KGQPREPQVY TLPPSREEMT |
| | | KNQVSLTCLV KGFYPSDIAV EWESNGQPEN |
| | | NYKTTPPVLD SDGSFFLYSK LTVDKSRWQQ |
| | | GNVFSCSVMH EALHNHYTQK SLSLSPGK |
| Light chain of | 400 | DIVMTQSPSS LTVTAGEKVT MSCKSSQSLL |
| IMAB362 | | NSGNQKNYLT WYQQKPGQPP KLLIYWASTR |
| | | ESGVPDRFTG SGSGTDFTLT ISSVQAEDLA |
| | | VYYCQNDYSY PFTFGSGTKL EIKRTVAAPS |
| | | VFIFPPSDEQ LKSGTASVVC LLNNFYPREA |
| | | KVQWKVDNAL QSGNSQESVT EQDSKDSTYS |
| | | LSSTLTLSKA DYEKHKVYAC EVTHQGLSSP |
| | | VTKSFNRGEC |
| Human CLDN18.2 | 401 | MAVTACQGLG FVVSLIGIAG IIAATCMDQW |
| | | STQDLYNNPV TAVENYQGLW RSCVRESSGF |
| | | TECRGYFTLL GLPAMLQAVR ALMIVGIVLG |
| | | AIGLLVSIFA LKCIRIGSME DSAKANMTLT |
| | | SGIMFIVSGL CAIAGVSVFA NMLVTNEWMS |
| | | TANMYTGMGG MVQTVQTRYT FGAALFVGWV |
| | | AGGLTLIGGV MMCIACRGLA PEETNYKAVS |
| | | YHASGHSVAY KPGGFKASTG FGSNTKNKKI |
| | | YDGGARTEDE VQSYPSKHDY V |

### Antibody Variants

The antibodies and antigen-binding fragments thereof provided herein also encompass various variants of the antibody sequences provided herein.

In certain embodiments, the antibody variants comprise one or more modifications or substitutions in one or more of the CDR sequences provided in Table 2 above, one or more of the non-CDR sequences of the heavy chain variable region or light chain variable region provided in Table 3 above, and/or the constant region (e.g. Fc region). Such variants retain binding specificity to CLDN18 (in particular, CLDN18.2) of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g. one or more introduced cysteine residues).

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g. charged residues such as Arg, Asp, His, Lys, and Glu) can be identified and replaced by a neutral or negatively charged amino acid (e.g. alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g. cysteine residue, positively charged residue, etc.).

### Affinity Variants

Affinity variants of antibodies may contain modifications or substitutions in one or more CDR sequences provided in Table 2 above, one or more FR sequences provided in Tables 4, 8, and 12 above, or the heavy or light chain variable region sequences provided in Tables 3, 6 and 10 above. FR sequences can be readily identified by a person skilled in the art based on the CDR sequences in Table 2 above and variable region sequences in Tables 3, 6 and 10 above, as it is well-known in the art that a CDR region is flanked by two FR regions in the variable region. The affinity variants retain specific binding affinity to CLDN18 (in particular, CLDN18.2) of the parent antibody, or even have improved CLDN18 specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Table 2 above, and variable region sequences provided in Tables 3, 6 and 10 above, one or more amino acid residues may be substituted yet the resulting antibody or antigen-binding fragment still retain the binding affinity or binding capacity to CLDN18 (in particular, CLDN18.2), or even have an improved binding affinity or capacity. Various methods known in the art can be used to achieve this purpose. For example, a library of antibody variants (such as Fab or scFv variants) can be generated and expressed with phage display technology, and then screened for the binding affinity to human CLDN18 (in particular, CLDN18.2). For another example, computer software can be used to virtually simulate the binding of the antibodies to human CLDN18 (in particular, CLDN18.2), and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-CLDN18 (in particular, anti-CLDN18.2) antibodies or antigen-binding fragments thereof comprise 1, 2, or 3 CDR sequences having at least 80% (e.g. at least 85%, 88%, 90%, 91%, 92%, 93%, 94%, 95%, 96%, 97%, 98%, 99%) sequence identity to that (or those) listed in Table 2 above yet retaining the specific binding affinity to CLDN18 (in particular, CLDN18.2) at a level similar to or even higher than its parent antibody.

In certain embodiments, the anti-CLDN18 (in particular, anti-CLDN18.2) antibodies or antigen-binding fragments thereof comprise one or more variable region sequences having at least 80% (e.g. at least 85%, 88%, 90%, 91%, 92%, 93%, 94%, 95%, 96%, 97%, 98%, 99%) sequence identity to that (or those) listed in Tables 3, 6 and 10 above yet retaining the specific binding affinity to CLDN18 (in particular, CLDN18.2) at a level similar to or even higher than its parent antibody. In some embodiments, a total of 1 to 10 amino acids have been substituted, inserted, or deleted in a variable region sequence listed in Tables 3, 6 and 10 above. In some embodiments, the substitutions, insertions, or deletions occur in regions outside the CDRs (e.g. in the FRs).

### Glycosylation Variants

The anti-CLDN18 (in particular, anti-CLDN18.2) antibodies or antigen-binding fragments thereof provided herein also encompass glycosylation variants, which can be obtained to either increase or decrease the extent of glycosylation of the antibodies or antigen binding fragments thereof.

The antibodies or antigen binding fragments thereof may comprise one or more modifications that introduce or remove a glycosylation site. A glycosylation site is an amino acid residue with a side chain to which a carbohydrate moiety (e.g. an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

In certain embodiments, the anti-CLDN18 (in particular, anti-CLDN18.2) antibodies and antigen-binding fragments provided herein comprise a mutation at position 92 and/or position 32 of the light chain and/or a mutation at position 55 of the heavy chain to remove one or more deamidation sites. In certain embodiments, the anti-CLDN18 (in particular, anti-CLDN18.2) antibodies and antigen-binding fragments provided herein comprise a mutation at S92 (for example, S92A), and/or a mutation at S32 (for example, S32A), and/or a mutation at G55 (for example, G55A) to remove one or more deamidation sites. These mutations are tested and are believed not to negatively affect the binding affinity of the antibodies provided herein.

### Cysteine-Engineered Variants

The anti-CLDN18 antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-CLDN18 antibodies or antigen-binding fragments thereof provided herein also encompass Fc variants, which comprise one or more amino acid residue modifications or substitutions at the Fc region and/or hinge region, for example, to provide for altered effector functions such as ADCC and CDC. Methods of altering ADCC activity by antibody engineering have been described in the art, see for example, Shields R L. et al., J Biol Chem. 2001. 276(9): 6591-604; Idusogie E E. et al., J. Immunol. 2000.164(8):4178-84; Steurer W. et al., J. Immunol. 1995, 155(3): 1165-74; Idusogie E E. et al., J. Immunol. 2001, 166(4): 2571-5; Lazar G A. et al., PNAS, 2006, 103(11): 4005-4010; Ryan M C. et al., Mol. Cancer Ther., 2007, 6: 3009-3018; Richards J O, et al., Mol Cancer Ther. 2008, 7(8): 2517-27; Shields R. L. et al., J. Biol. Chem, 2002, 277: 26733-26740; Shinkawa T. et al., J. Biol. Chem, 2003, 278: 3466-3473.

CDC activity of the antibodies or antigen-binding fragments provided herein can also be altered, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or enhance complement dependent cytotoxicity (CDC) (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-CLDN18 (in particular, anti-CLDN18.2) antibodies or antigen-binding fragments thereof provided herein have enhanced effector functions (for example, enhanced ADCC activity), and comprise one or more amino acid substitution(s) in human IgG1 at a position selected from the group consisting of: 235, 236, 239, 243, 268, 292, 300, 305, 324, 330, 332 and 396 (according to IMGT numbering). In certain embodiments, the anti-CLDN18 (in particular, anti-CLDN18.2) antibodies or antigen-binding fragments thereof provided herein are of IgG1 isotype and comprise one or more amino acid substitution(s) selected from the group consisting of: L235V, G236A, S239D, F243L, H268F, R292P, Y300L, V305I, S324T, A330L, I332E, and P396L (according to IMGT numbering), and any combination thereof. In certain embodiments, the anti-CLDN18 (in particular, anti-CLDN18.2) antibodies or antigen-binding fragments thereof provided herein are of IgG1 isotype and comprise a mutation (according to IMGT numbering) selected from the group consisting of: (1) G236A, S239D and I332E; (2) S239D, A330L and I332E; (3) S239D and I332E; (4) S239D, H268F, S324T and I332E; (5) F243L, R292P, Y300L, V305I and P396L; (6) L235V, F243L, R292P, Y300L and P396L.

In certain embodiments, the anti-CLDN18 (in particular, anti-CLDN18.2) antibodies or antigen-binding fragments thereof comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-III known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70: 3269-3277 (2010); and Hinton, P. et al., J. Immunology, 176:346-356 (2006).

In certain embodiments, anti-CLDN18 (in particular, anti-CLDN18.2) antibodies or antigen-binding fragments thereof comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g. as described in U.S. Pat. No. 5,731,168.

### Antigen-Binding Fragments

Provided herein are also anti-CLDN18 (in particular, anti-CLDN18.2) antigen-binding fragments. Various types of antigen-binding fragments are known in the art and can be developed based on the anti-CLDN18 (in particular, anti-CLDN18.2) antibodies provided herein, including for example, the exemplary antibodies whose CDRs are shown in Table 2 above, and variable sequences are shown in Tables 3, 6 and 10, and their different variants (such as affinity variants, glycosylation variants, Fc variants, cysteine-engineered variants and so on).

In certain embodiments, an anti-CLDN18 (in particular, anti-CLDN18.2) antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

Various techniques can be used for the production of such antigen-binding fragments. Illustrative methods include, enzymatic digestion of intact antibodies (see, e.g. Morimoto et al., Journal of Biochemical and Biophysical Methods 24:107-117 (1992); and Brennan et al., Science, 229:81 (1985)), recombinant expression by host cells such as E. coli (e.g. for Fab, Fv and ScFv antibody fragments), screening from a phage display library as discussed above (e.g. for ScFv), and chemical coupling of two Fab′-SH fragments to form F(ab′)₂ fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-CLDN18 (in particular, anti-CLDN18.2) antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific Antibodies

In certain embodiments, the anti-CLDN18 (in particular, anti-CLDN18.2) antibody or an antigen-binding fragment thereof is bispecific. In certain embodiments, the antibody or antigen-binding fragment thereof is further linked to a second functional moiety having a different binding specificity from said anti-CLDN18 (in particular, anti-CLDN18.2) antibody, or antigen binding fragment thereof.

In certain embodiments, the bispecific antibodies or antigen-binding fragments thereof provided herein are capable of specifically binding to a second antigen other than CLDN18 (in particular, CLDN18.2), or a second epitope on CLDN18 (in particular, CLDN18.2). In certain embodiments, the second antigen is selected from the group consisting of EGFR, FGFR, VEGF, OX40, CD3, CD37, c-MET, Her2, CD19, CD20, CD39, SIRPα, TGFbeta, CD73, PD1, PDL1, 4-1BB, CTLA4, TIGIT, GITA, VISTA, TIGIT, B7-H3, B7-H4, B7-H5, CD112R, Siglec-15, LAG3 and TIM-3. In certain embodiments, the bispecific antibodies or antigen-binding fragments thereof provided herein are capable of specifically binding to CLDN18 (in particular, CLDN18.2) and SIRPα. In certain embodiments, the bispecific antibodies or antigen-binding fragments thereof provided herein are capable of specifically binding to CLDN18 (in particular, CLDN18.2) and CD39.

### Conjugates

In some embodiments, the anti-CLDN18 (in particular, anti-CLDN18.2) antibodies or antigen-binding fragments thereof further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding (e.g. disulfide bond), affinity binding, intercalation, coordinate binding, complexation, association, blending, or addition, among other methods. In some embodiments, the antibodies or antigen-binding fragments thereof can be linked to one or more conjugates via a linker or a crosslinking agent. The linker or crosslinking agent comprises a reactive chemical group that can react with the anti-CLDN18 antibody or fragment thereof. The reactive chemical groups can be N-succinimidyl esters and N-sulfosuccinimidyl esters. Additionally the linker comprises a reactive chemical group, which can be a dithiopyridyl group that can react with the drug to form a disulfide bond. Linker molecules include, for example, N-succinimidyl 4-(maleimidomethyl) cyclohexanecarboxylate (SMCC), N-succinimidyl 3-(2-pyridyldithio) propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof provided herein may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g. a polymer such as PEG which extends half-life), a chemotherapeutic agent, a toxin, a radioactive isotope, a lanthanide, a detectable label (e.g. a luminescent label, a fluorescent label, an enzyme-substrate label), a DNA-alkylator, a topoisomerase inhibitor, a tubulin-binder, a purification moiety or other anticancer drugs (e.g. agonist of toll-like receptor 7 (TLR-7), TLR-8 and/or TLR-9, siRNA, antibody or antigen-binding fragments thereof, a peptide (such as a short peptide), etc.).

A “toxin” can be any agent that is detrimental to cells or that can damage or kill cells. Examples of toxin include, without limitation, taxol, taxoids, CC-1065 and CC-1065 analogs, duocarmycins and duocarmycin analogs, enediynes such as calicheamicins, dolastatin and dolastatin analogs including auristatins, tomaymycin derivatives, leptomycin derivatives, cisplatin, carboplatin, daunorubicin, doxorubicin, vincristine, vinblastine, melphalan, mitomycin C, chlorambucil and morpholino doxorubicin, cytochalasin B, gramicidin D, ethidium bromide, emetine, mitomycin, etoposide, tenoposide, vincristine, MMAE, MMAF, DM1, DM4, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, and anthramycin (AMC)), anti-mitotic agents (e.g. vincristine and vinblastine), a topoisomerase inhibitor, and a tubulin-binders.

Examples of detectable label may include a fluorescent label (e.g. fluorescein, rhodamine, dansyl, phycoerythrin, or Texas Red), an enzyme-substrate label (e.g. horseradish peroxidase, alkaline phosphatase, luceriferases, glucoamylase, lysozyme, saccharide oxidases or β-D-galactosidase), a radioisotope (e.g. ¹²³I, ¹²⁴I, ¹²⁵I, ¹³¹J ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), a luminescent label, a chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody or an antigen-binding fragment thereof provided herein is used as a base for a conjugate.

In certain embodiments, the antibody or an antigen-binding fragment thereof provided herein is conjugated to a signal peptide. A signal peptide (sometimes referred to as signal sequence, leader sequence or leader peptide) can be used to facilitate secretion and isolation of the antibodies or antigen-binding fragments thereof provided herein. Signal peptides are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain. In some embodiments, the signal peptides used in the present disclosure have an amino acid sequence selected from the group consisting of SEQ ID NOs: 368-396, and their sequences are shown in Table 15 below.

TABLE 15
Amino acid sequences of signal peptides

| SEQ | | SEQ | |
|---|---|---|---|
| ID NO | Amino Acid Sequence | ID NO | Amino Acid Sequence |
| 368 | MGWNWIFLFLLSGTAGVHS | 383 | MGWSWIFLFLLSETAGVLS |
| 369 | MDSRLNLVFLVLILKGVQC | 384 | MYFRLSSVFLLLILKGVQC |
| 370 | MGWSWIFLFLLSGTAGVLS | 385 | MGWSWIFLFLLSEAAGVLS |
| 371 | MRWSCIILFLVATATGVHS | 386 | MGWSWIFLFLLSGTAGVHS |
| 372 | MRWSCIIFLFIATATGVHS | 387 | MGWYWIFLFLLSGTAGVHS |
| 373 | MEWPLIFLFLLSGTAGVQS | 388 | MGWSWIFLFLLSGTAGVRS |
| 374 | MRWSCIILFLVASATGVHS | 389 | MESQTQVLMSLLFWVSGTYG |
| 375 | MGWSYIILFLVATATDVHS | 390 | MESQTQVLMSLLFWVSGTCG |
| 376 | MRWSCIIFLFIATATGIHS | 391 | MESQTQVLMSLLFWVSGSCG |
| 377 | MNFGLSLIFLVLVLKGVLC | 392 | MDFQVQIFSFLLISASVIMSRG |
| 378 | MAVLALLLCLVTFPSCVLS | 393 | MDFQVQIFSLLLISASVILSRG |
| 379 | MGWSYIILFLVATATGVHS | 394 | MDFQVQIFSFLLISASVMMSRG |
| 380 | MEWSWIFLFLLSGTASVLS | 395 | MESQTQVLMSLLFWVSGICG |
| 381 | MKVLSLLYLLTAIPGILS | 396 | MDSQAQVLMLLLLWVSGTCG |
| 382 | MGWSWIFLFLLSRTAGVHS | | |

In some embodiments, the antibody or an antigen-binding fragment thereof provided herein conjugated to a signal peptide has a heavy chain variable region comprising a sequence selected from the group consisting of SEQ ID NOs: 403-457, and a light chain variable region comprising a sequence selected from the group consisting of SEQ ID NOs: 458-508. The resulting antibodies are referred to herein as sg100F4, sg15E10, etc., where the prefix “sg” indicates “signal peptide”, and the suffix indicates the monoclonal antibody, for example, “100F4” indicates that it is from the monoclonal antibody 100F4, wherein the N-terminals of VH and VL of antibody 100F4 are conjugated to a signal peptide, respectively. The amino acid sequences of SEQ ID NOs: 403-508 are shown in Table 16 below, and the signal peptides are underlined.

TABLE 16
Amino acid sequences of exemplary antibodies (including signal
peptide)

| | Heavy chain AA | | Light chain AA | |
|---|---|---|---|---|
| | (signal | | (Signal | |
| | peptide + variable | SEQ ID | peptide + variable | SEQ ID |
| Clone ID | domain) | NO | domain) | NO |
| sg100F4 | MGWNWIFLFLLSGTA | 412 | MESQTQVLMSLLFWVS | 508 |
| | GVHSQVQLQQSGPDL | | GTYGDIVMTQSPSSLT | |
| | VKPGASVKLSCKASG | | VTAGEKVTMTCKSGQS | |
| | YTFTNYDINWVKQRP | | LLNSGNQRNYLTWYQQ | |
| | GQGLEWIGGIHPRDG | | KPGQSPKLLIYWASTR | |
| | NTKYNEKFKDKATLT | | ESGVPDRFTGSGSGAD | |
| | IDTSANTAYMEFHSL | | FTLTISSVQAEDLALY | |
| | TSEDSAVYFCARGYY | | YCQNAYFYPYTFGGGT | |
| | GNSFAYWGQGTLVTV | | KLEIK | |
| | SA | | | |
| sg15E10 | MDSRLNLVFLVLILK | 405 | MESQTQVLMSLLFWVS | 483 |
| | GVQCDVQLVESGGGL | | GTCGDIVMTQSPSSLT | |
| | VQPGGSRKLSCAASG | | VTAGEKVTMNCKSSQS | |
| | FTFSSFGMHWVRQAP | | LLNSGNQKNYLTWYQQ | |
| | EKGLEWVAYISSGSS | | KPGQPPKLLIYWASTR | |
| | SIYYVDTVKGRFTIS | | TSGVPDRFTGSGSGTD | |
| | RDNPKNTLFLQMTSL | | FTLTISSVQAEDLAVY | |
| | RSEDTAMYYCARNAY | | CCQNGYTYPLTFGAGT | |
| | YGNAFDYWGQGTTLT | | KLELK | |
| | VSS | | | |
| sg203A5 | MGWSWIFLFLLSGTA | 429 | MESQTQVLMSLLFWVS | 498 |
| | GVLSEVQLQQSGPEV | | GTCGDIVMTQSPSSLT | |
| | VKPGTSVKISCKASG | | VTAGEMVTMNCKSSQS | |
| | YTFTDYYMNWVKQSH | | LLNSGNQRNYLTWYQQ | |
| | GKSLEWIGDINPKNG | | KPGQPPKLLIYWSSTR | |
| | GSRYNQKFKGKATLT | | ESGVPDRFTGSGSGTD | |
| | VDKSSNTAYMELRSL | | FTLTISSVQAEDLAVY | |
| | TSEDSAVYYCARLYY | | YCQNDYSYPLTFGAGT | |
| | GNSFAYWGQGTLVTV | | KLELK | |
| | SA | | | |
| sg203D12 | MRWSCIILFLVATAT | 454 | MESQTQVLMSLLFWVS | 494 |
| | GVHSQVQLQQPGTEL | | GTCGDIVMTQSPSSLT | |
| | VKPGASVKVSCKASG | | VTAGEKVTMSCKSSQS | |
| | YTFTSYWMHWVKQRP | | LLNSGNQKNYLTWYQQ | |
| | GQGLEWIGRIRPSDS | | KPGQPPKLLIYWASTR | |
| | DSNYNQKFKGKATLT | | ESGVPDRFTGSGSGTD | |
| | VDKSSDTAYMQLNSL | | FTLTISSVQAEDLAVY | |
| | PSEDSAVYYCAMGAY | | YCQNVYSYPITFGSGT | |
| | FSNSFAYWGQGTLVT | | KLEIK | |
| | VSA | | | |
| sg206C7 | MRWSCIILFLVATAT | 455 | MESQTQVLMSLLFWVS | 473 |
| | GVHSQVQLQQPGTEL | | GTCGDIVMTQSPSPLT | |
| | VKPGASVKVSCKASG | | VIVGEKVTMTCKSSQT | |
| | YTFTTYWMHWVKQRP | | LLNRGNQKNYLTWYQQ | |
| | GQGLEWIGRIRPSDS | | KPGQPPKLLIYWASTR | |
| | DSNYNQKFKGKATLT | | ESGVPDRFTGSGSGTD | |
| | VDKSSDTAYMQLNSL | | FTLTINSVQAEDLAVY | |
| | TSEDSAVYYCAMGAY | | YCQNDYFFPFTFGSGT | |
| | FSNSFAYWGQGTLVT | | KLEIR | |
| | VSA | | | |
| sg213A9 | MRWSCIILFLVATAT | 454 | MESQTQVLMSLLFWVS | 468 |
| | GVHSQVQLQQPGTEL | | GSCGDIVMTQSPSSLT | |
| | VKPGASVKVSCKASG | | VTAGEKVTMTCKSSQT | |
| | YTFTSYWMHWVKQRP | | LLNRGNQKNYVTWYQQ | |
| | GQGLEWIGRIRPSDS | | KPGQPPKLLIYWASTR | |
| | DSNYNQKFKGKATLT | | ESGVPDRFTGSGSGTD | |
| | VDKSSDTAYMQLNSL | | FTLTISSVQAEDLAVY | |
| | PSEDSAVYYCAMGAY | | YCQNDYFFPFTFGSGT | |
| | FSNSFAYWGQGTLVT | | KLEIR | |
| | VSA | | | |
| sg214E4 | MRWSCIILFLVASAT | 450 | MESQTQVLMSLLFWVS | 475 |
| | GVHSQVQLQQPGTEL | | GTCGDIVMTQSPSPLT | |
| | VKPGASVKVSCKASG | | VTAGEKVTMTCKSSQT | |
| | YTFTTYWMHWVKQRP | | LLNRGNQKNYLTWYQQ | |
| | GQGLEWIGRIRPSDS | | KPGQPPKLLIYWASTR | |
| | DSNYNQKFKGKATLT | | ESGVPDRFTGSGSGTD | |
| | VDKSSDTAYMQLNGL | | FTLTINSVQAEDLAVY | |
| | TSEDSAVYYCAMGAY | | YCQNDYFFPFTFGSGT | |
| | FSNSFAYWGQGTLVT | | KLETR | |
| | VSA | | | |
| sg217B5 | MRWSCIIFLFIATAT | 447 | MESQTQVLMSLLFWVS | 505 |
| | GVHSQVQLQQPGAEL | | GTCGDIVMTQSPSSLT | |
| | VKPGASVKVSCKASG | | VTPGEKVTMNCKSSQS | |
| | STFTTYWMHWVKKRP | | LLNSGNQKNYVTWYQQ | |
| | GQGLEWIGGIRPFDS | | KPGQPPKLLMFWASTR | |
| | NTNYNHKFKGKATLT | | ESGVPDRFTGSGSGTD | |
| | VDKASNTAYMQLSSL | | FTLIISSVQAEDLAVY | |
| | TSEDSAVYYCAMGAY | | HCQNDYVYPFTFGSGT | |
| | YSNSFAYWGQGTVVT | | KLEIK | |
| | VSA | | | |
| sg226D5 | MRWSCIILFLVATAT | 453 | MESQTQVLMSLLFWVS | 482 |
| | GVHSQVQLQQPGAEL | | GTCGDIVMTQSPSSLT | |
| | VKPGASVKVSCKASG | | VTAGEKVTMNCKSSQS | |
| | YTFTTYWMHWVRQRP | | LLNSGNQKNYLTWYQQ | |
| | GQGLEWIGRIRPSDT | | KPGQPPKLLIYWASTR | |
| | ATNYNQKFKGKATLT | | ESGVPDRFTGSGSGTD | |
| | VNKSSSTAYMQFSSL | | FTLTINSVQAEDLAVY | |
| | TSEDSAVFYCAMGAY | | YCQNDYSYPFMFGSGT | |
| | YSNSFAYWGQGTLVT | | KLEIK | |
| | VSA | | | |
| sg226E9 | MGWSWIFLFLLSGTA | 429 | MESQTQVLMSLLFWVS | 497 |
| | GVLSEVQLQQSGPEV | | GTCGDIVMTQSPSSLT | |
| | VKPGTSVKISCKASG | | VTAGEMVTMNCKSSQS | |
| | YTFTDYYMNWVKQSH | | LLNSGNQRNYLTWYQQ | |
| | GKSLEWIGDINPKNG | | KPGQPPKLLIYWSSTR | |
| | GSRYNQKFKGKATLT | | ESGVPDRFTGSGSGTD | |
| | VDKSSNTAYMELRSL | | FTLTISSVQAEDLAVY | |
| | TSEDSAVYYCARLYY | | YCQNDYNYPLTFGAGT | |
| | GNSFAYWGQGTLVTV | | KLELK | |
| | SA | | | |
| sg22E12 | MGWSYIILFLVATAT | 434 | MESQTQVLMSLLFWVS | 478 |
| | DVHSQVQLQQPGTEL | | GTCGDIVMTQSPSSLT | |
| | VKTGTSVKLSCKASG | | VTAGEKVTLSCKSSQS | |
| | YTFTNWVHWVIQRPG | | LLNSGNQKNYLTWYQQ | |
| | QGLEWIGEINPTNGR | | KPGQPPKLLIYWSSTR | |
| | SNYNEKFKKKATLTL | | ESGVPDRFTGSGSGTD | |
| | DRSSTTAYMQLSSLT | | FTLTISSVQAEDLAVY | |
| | SEDSAVYFCAGIYYG | | HCQNNYYYPLTFGAGT | |
| | NSFAHWGQGTLVTVS | | KLELK | |
| | A | | | |
| sg237E3 | MGWSWIFLFLLSGTA | 430 | MESQTQVLMSLLFWVS | 500 |
| | GVLSEVQLQQSGPEV | | GTCGDIVMTQSPSSLT | |
| | VKPGTSVKISCKASG | | VTAGEMVTMNCKSSQS | |
| | YTFTDYYMNWVKQSH | | LLNSGNRRNYLTWYQQ | |
| | GKSLEWIGDINPKNG | | KPGQPPKLLIYWSSTR | |
| | GSRYNQKFRGKATLT | | ESGVPDRFAGSGSGTD | |
| | VDKSSNTAFMELRSL | | FTLTISSVQAEDLAVY | |
| | TSEDSAVYYCARLYY | | YCQNDYTYPLTFGAGT | |
| | GNSFAYWGQGTLVTV | | KLELK | |
| | SA | | | |
| sg242G5 | MGWSWIFLFLLSGTA | 428 | MESQTQVLMSLLFWVS | 498 |
| | GVLSEVQLQQSGPEV | | GTCGDIVMTQSPSSLT | |
| | VKPGTSVKISCKASG | | VTAGEMVTMNCKSSQS | |
| | YTFTDYYMNWVKQSH | | LLNSGNQRNYLTWYQQ | |
| | GKSLEWIGDINPKNG | | KPGQPPKLLIYWSSTR | |
| | GSRYNQKFKGKATLT | | ESGVPDRFTGSGSGTD | |
| | ADKSSNTAYMELRSL | | FTLTISSVQAEDLAVY | |
| | TSEDSAVYYCTRLYF | | YCQNDYSYPLTFGAGT | |
| | GNSFAYWGQGTLVTV | | KLELK | |
| | SA | | | |
| sg243A8 | MGWSWIFLFLLSGTA | 430 | MESQTQVLMSLLFWVS | 499 |
| | GVLSEVQLQQSGPEV | | GTCGDIVMTQSPSSLT | |
| | VKPGTSVKISCKASG | | VTAGEMVTMNCKSSQS | |
| | YTFTDYYMNWVKQSH | | LLNSGNQRNYLTWYQQ | |
| | GKSLEWIGDINPKNG | | KPGQPPKLLIYWSSTR | |
| | GSRYNQKFRGKATLT | | ESGVPDRFTGSGSGTD | |
| | VDKSSNTAFMELRSL | | FTLTISSVQAEDLAVY | |
| | TSEDSAVYYCARLYY | | YCQNDYTYPLTFGAGT | |
| | GNSFAYWGQGTLVTV | | KLELK | |
| | SA | | | |
| sg246B8 | MRWSCIIFLFIATAT | 449 | MESQTQVLMSLLFWVS | 481 |
| | GVHSQVQLQQPGAEL | | GTCGDIVMTQSPSSLT | |
| | VNPGASVKVSCKASG | | VTAGEKVTMNCKSSQS | |
| | STFTTYWMHWVKKRP | | LLNRGNQKNYVTWYQQ | |
| | GQGLEWIGGIRPSDS | | KPGQPPKLLIFWASTR | |
| | NNNYNHKFKGKATLT | | ESGVPDRFTGSGSGTD | |
| | VDKASSTAYLQLSSL | | FTLIISSVQAEDLAVY | |
| | TSEDSAVYYCAMGAY | | YCQNDYVYPFTFGSGT | |
| | YSNSFAYWGQGTVVT | | KLEIK | |
| | VSA | | | |
| sg248D9 | MRWSCIIFLFIATAT | 448 | MESQTQVLMSLLFWVS | 480 |
| | GVHSQVQLQQPGAEL | | GTCGDIVMTQSPSSLT | |
| | VKPGASVKVSCKASG | | VTAGEKVTMNCKSNQS | |
| | STFTTYWMHWVKKRP | | LLNSGNQKNYVTWYQQ | |
| | GQGLEWIGGIRPFDS | | KPGQPPKLLIFWASTR | |
| | NTNYNHKFKGKATLT | | ESGVPDRFTGSGSETD | |
| | VDKASSTAYMQLSSL | | FTLIISSVQAEDLAVY | |
| | TSEDSAVYYCAMGAY | | YCQNDYVYPFTFGSGT | |
| | YSNSFAYWGQGTVVT | | KLEIK | |
| | VSA | | | |
| sg256C10- | MEWPLIFLFLLSGTA | 410 | MESQTQVLMSLLFWVS | 502 |
| 1 | GVQSQVQLQQSGTEL | | GTCGDIVMTQSPSSLT | |
| | VKPGASVKISCKASG | | VTAREKVIMNCKSSQS | |
| | YAFNSYWMNWLKQRP | | LENSGNQKNYLSWYQQ | |
| | GKGLEWIGQIYPGDG | | KPGQPPKLLIYWASTR | |
| | DTNYNGGFRGKATLT | | KSGVPDRFTGSGSGTG | |
| | ADKSSRTAYMHLNSL | | FTLTISSVQAEDLAVY | |
| | TSEDSAVYFCARWGT | | YCQNNYFYPLTFGAGT | |
| | GNTMDYWGQGTSVTV | | KLELN | |
| | SS | | | |
| sg256C10- | MDSRLNLVFLVLILK | 406 | MESQTQVLMSLLFWVS | 502 |
| 2 | GVQCDVQLVESGGGL | | GTCGDIVMTQSPSSLT | |
| | VQPGGSRRLSCAASG | | VTAREKVIMNCKSSQS | |
| | FSFSNFGMYWVRQAP | | LENSGNQKNYLSWYQQ | |
| | EKGLEWVAFITSDST | | KPGQPPKLLIYWASTR | |
| | SIYYVDTVKGRFTVS | | KSGVPDRFTGSGSGTG | |
| | RDNPKNTLFLQMTSL | | FTLTISSVQAEDLAVY | |
| | RSEDTAMYYCGRTGY | | YCQNNYFYPLTFGAGT | |
| | GNAMDYWGQGTSVTV | | KLELN | |
| | SS | | | |
| sg305G8 | MRWSCIILELVASAT | 451 | MESQTQVLMSLLFWVS | 474 |
| | GVHSQVQLQQPGTEL | | GTCGDIVMTQSPSPLT | |
| | VKPGASVKVSCKASG | | VTAGEKVTMTCKSSQT | |
| | YTFTTYWMHWVKQRP | | LLNRGNQKNYLTWYQQ | |
| | GQGLEWIGRIRPSDS | | KPGQPPKLLIYWASTR | |
| | DSNYNQKFKGKATLT | | ESGVPDRFTGSGSGTD | |
| | VDKSSDTAYMQLNSL | | FTLTINSVQAEDLAVY | |
| | TSEDSAVYYCAMGAY | | YCQNDYFFPFTFGSGT | |
| | FSNSFAYWGQGTLVT | | KLEIR | |
| | VSA | | | |
| sg308E8 | MEWPLIFLFLLSGTA | 408 | MESQTQVLMSLLFWVS | 494 |
| | GVQSQVQLQQSGAEL | | GTCGDIVMTQSPSSLT | |
| | VKPGASVKISCKASG | | VTAGEKVTMSCKSSQS | |
| | YAFSNYWMNWVKQRP | | LLNSGNQKNYLTWYQQ | |
| | GKGLEWIGQIYPGNG | | KPGQPPKLLIYWASTR | |
| | NTNYNGGFKGKATLT | | ESGVPDRFTGSGSGTD | |
| | ADKSSSTAYMHLNSL | | FTLTISSVQAEDLAVY | |
| | TSEDSAVYFCARWGT | | YCQNVYSYPITFGSGT | |
| | GNTMDYWGQGTSVTV | | KLEIK | |
| | SS | | | |
| sg310H5 | MRWSCIIFLFIATAT | 446 | MESQTQVLMSLLFWVS | 501 |
| | GIHSLVQLQQPGAEL | | GTCGDIVMTQSPSSLT | |
| | VKPGASVKVSCKASG | | VTAGKKVTMNCKSSQS | |
| | STFTTYWMHWVKKRP | | LLNSGNQKNYVTWYQQ | |
| | GQGLEWIGGIRPSDS | | KPGQPPKLLIFWASTR | |
| | NTNYNHKFKGKATLT | | ESGVPDRFTGSGSGTD | |
| | VDKASSTAYMQLSSL | | FSLIISTVQAEDLAVY | |
| | TSEDSAVYYCAMGAY | | YCQNDYVYPFTFGSGT | |
| | YSNSFAYWAQGTVVT | | KLEIK | |
| | VSA | | | |
| sg314D7 | MRWSCIILFLVATAT | 456 | MESQTQVLMSLLFWVS | 474 |
| | GVHSQVQLQQPGTEL | | GTCGDIVMTQSPSPLT | |
| | VKPGASVKVSCKASG | | VTAGEKVTMTCKSSQT | |
| | YTFTTYWMHWVTQRP | | LLNRGNQKNYLTWYQQ | |
| | GQGLEWIGRIRPSDS | | KPGQPPKLLIYWASTR | |
| | DSNYNQKFKGKATLT | | ESGVPDRFTGSGSGTD | |
| | VDKSSDTAYMQLNSL | | FTLTINSVQAEDLAVY | |
| | TSEDSAVYYCAMGAY | | YCQNDYFFPFTFGSGT | |
| | FSNSFAYWGQGTLVT | | KLEIR | |
| | VSA | | | |
| sg315F10 | MEWPLIFLFLLSGTA | 410 | MESQTQVLMSLLFWVS | 494 |
| | GVQSQVQLQQSGTEL | | GTCGDIVMTQSPSSLT | |
| | VKPGASVKISCKASG | | VTAGEKVTMSCKSSQS | |
| | YAFNSYWMNWLKQRP | | LLNSGNQKNYLTWYQQ | |
| | GKGLEWIGQIYPGDG | | KPGQPPKLLIYWASTR | |
| | DTNYNGGFRGKATLT | | ESGVPDRFTGSGSGTD | |
| | ADKSSRTAYMHLNSL | | FTLTISSVQAEDLAVY | |
| | TSEDSAVYFCARWGT | | YCQNVYSYPITFGSGT | |
| | GNTMDYWGQGTSVTV | | KLEIK | |
| | SS | | | |
| sg317A7 | MEWPLIFLFLLSGTA | 409 | MESQTQVLMSLLFWVS | 494 |
| | GVQSQVQLQQSGAEL | | GTCGDIVMTQSPSSLT | |
| | VKPGASVKISCKASG | | VTAGEKVTMSCKSSQS | |
| | YAFSNYWMNWVNQRP | | LLNSGNQKNYLTWYQQ | |
| | GKGLEWIGQIYPGNG | | KPGQPPKLLIYWASTR | |
| | NTNYNGGFKGKATLT | | ESGVPDRFTGSGSGTD | |
| | ADKSSSTAYMHLNSL | | FTLTISSVQAEDLAVY | |
| | TSEDSAVYFCARWGT | | YCQNVYSYPITFGSGT | |
| | GNTMDYWGQGTSVTV | | KLEIK | |
| | SS | | | |
| sg319F2 | MRWSCIILFLVATAT | 452 | MESQTQVLMSLLFWVS | 504 |
| | GVHSQVLLQQPGTEL | | GTCGDIVMTQSPSSLT | |
| | VKPGASVKVSCKASA | | VTAREKVTMNCKSSQS | |
| | YTFTSYWIHWVKQRP | | LLNSGNQKNYLTWYQQ | |
| | GQGLEWIGRIRPSDS | | KPGQPPKMLIYWASTR | |
| | DSTYNQNFKGKATLT | | ESGVPDRFTGSGSGTY | |
| | VDKSSDTAYMQLTSL | | FTLTISSVQAEDLAVY | |
| | TSEDSAVYYCSMGAY | | YCQNDYSFPFTFGSGT | |
| | YSNSFGYWGQGSLVT | | KLEIK | |
| | VSA | | | |
| sg33G12 | MNFGLSLIFLVLVLK | 444 | MDFQVQIFSFLLISAS | 458 |
| | GVLCEVKLVESGGGL | | VIMSRGQIVLSQSPTI | |
| | VQPGGSLKLSCAASG | | LSASPGEKVTMTCRAT | |
| | FTFSRYAMSWVRQTP | | SSVSYMHWFQQKPGSS | |
| | EKRLEWVAYISIGGT | | PKPWIYATSNLASGVP | |
| | TYYPDTIKGRFTISR | | ARFSGSGSGTSYSLTI | |
| | DNAKNTLYLQMSSLK | | SRVEAEDAATYYCQQW | |
| | SEDTAMYYCTRHYYG | | SRNPLTFGAGTKLELK | |
| | HDVMDYWGQGTSVTV | | | |
| | SS | | | |
| sg35A10 | MDSRLNLVFLVLILK | 404 | MESQTQVLMSLLFWVS | 485 |
| | GVQCDVQLVESGGGL | | GTCGDIVMTQSPSSLT | |
| | VQPGGSRKLSCAASG | | VTAGEKVTMSCKSSQS | |
| | FTFSSFGMHWVRQAP | | LLNSGNQKNYLTWYQH | |
| | EKGLEWVAYISSGSS | | KPGQPPKLLIYWASTR | |
| | SFYYADTVKGRFTIS | | RSGVPDRFTGSGSGTD | |
| | RDNPKNTLFLQMTSL | | FTLTITSVQAEDLAVY | |
| | RSEDTAMYYCARNAY | | CCQNVYVYPLTFGAGT | |
| | YGNALDYWGQGTTLT | | KLELK | |
| | VSS | | | |
| sg35B4 | MNFGLSLIFLVLVLK | 443 | MDFQVQIFSLLLISAS | 460 |
| | GVLCEAKLVESGGDF | | VILSRGQIVLSQSPAI | |
| | MQPGGSLKLSCAASG | | LSASPGEKVTMTCRAS | |
| | FTLSSYALSWVRQTP | | SSVNYIHWYQQKPGSS | |
| | EKRLEWVAYISNLGG | | PKAWIYATSNLASGVP | |
| | STFYPDTVKGRFTIS | | TRESGSGSGTSYSLTI | |
| | RDNARNTLFLQMSSL | | DRVEAEDAATYYCQQW | |
| | QSEDTAIYYCATHLY | | NSNPLTFGAGTKLELK | |
| | NYDAFASWGQGTLVT | | | |
| | VSA | | | |
| sg360C2 | MAVLALLLCLVTFPS | 403 | MESQTQVLMSLLFWVS | 507 |
| | CVLSQVQLKESGPGL | | GTCGDIVMTQSPSSLT | |
| | VAPSQSLSITCIVSG | | VTVGEKVTMSCKSSQS | |
| | FSLTTYGISWVRQPP | | LLNSGNQKNYLTWYRQ | |
| | GKGLEWLGVIWGDGS | | KPGQPPELLIYWASTR | |
| | THYHSALISRLSISK | | ESGVPDRFTGSGSGTD | |
| | DNSKSQVFLKLNSLQ | | FTLTISSVQAEDLAVY | |
| | TDDTATYYCAKPYYS | | YCQNVYSYPLTFGAGT | |
| | NAMDYWGQGTSVTVS | | KLELK | |
| | S | | | |
| sg365F6 | MGWSYIILFLVATAT | 440 | MESQTQVLMSLLFWVS | 488 |
| | GVHSQVQLQQPGPEL | | GTCGDIVMTQSPSSLT | |
| | VKPGASVKLSCKASG | | VTAGEKVTMSCKSSQS | |
| | YTFTSYWMHWVRQRP | | LLNSGNQKNYLTWYQQ | |
| | GQGLEWIGMIHPNSG | | KPGQPPKLLIYWASTR | |
| | STNYNEKFKSKATLT | | ESGVPDRFTGRGSGTD | |
| | VDKSSSTAYMQLSSL | | FTLTISSVQAEDLAVY | |
| | TSEDSAVYFCARNGY | | YCQNNYNYPVTFGAGT | |
| | YGNAMDYWGQGTSVT | | KLELK | |
| | VSS | | | |
| sg38B9 | MNFGLSLIFLVLVLK | 445 | MDFQVQIFSFLLISAS | 459 |
| | GVLCEVNLVESGGGF | | VMMSRGQIVLSQSPAI | |
| | VQPGGSLKLSCVASG | | LSASPGEKVTVTCRAS | |
| | FTFSSYAMSWVRQTP | | SSLSYMHWYQQRPGSS | |
| | DTRLEWVAYISNLGG | | PKPWIYGTSNLASGVP | |
| | STYYPDTVKGRFTIS | | ARFSGSGSGTSYSLTI | |
| | RDNARNTLFLQMSSL | | SRVEAEDAATYYCQQW | |
| | QSEDTAMYYCTGHLY | | SSNPLTFGAGTKLELK | |
| | HYDAFAYWGQGTLVT | | | |
| | VSA | | | |
| sg40C1 | MEWSWIFLFLLSGTA | 411 | MESQTQVLMSLLFWVS | 477 |
| | SVLSEVQLQQFGAEL | | GTCGDIVMTQSPSSLP | |
| | VKPGTSVKISCKASG | | VTTGERVTMSCRSSQI | |
| | YTFTDYNMDWVKQSH | | LLNSGNQKNYLTWYQQ | |
| | GKSLEWIGDVNPNYS | | KPGQPPKLLIYWASTR | |
| | TTRYNQKFKDKATLT | | DYGVPDRFTGSGSGTD | |
| | VDKSSSTAYMELRSL | | FTLTISSVQAEDLAVY | |
| | TSEDTAVYYCARLYY | | YCQNAYFYPFTFGAGT | |
| | GNSFAYWGQGTLVTV | | KLELK | |
| | SA | | | |
| sg41B3 | MKVLSLLYLLTAIPG | 442 | MESQTQVLMSLLFWVS | 506 |
| | ILSDVQLQESGPGLV | | GTCGDIVMTQSPSSLT | |
| | KPSQSLSLTCSVTGY | | VTSGEKVTMSCKSSQS | |
| | SITSGYLWNWIRQSP | | LENSGNQKNYLTWYQQ | |
| | GNKLEWMGHITYDGS | | KLGQPPKLLIFWASTR | |
| | NNYNPSLKNRISITR | | ESGVPDRFTGSGSGTD | |
| | DTSKNQFFLKLNSVT | | FTLTISSVQTEDLAVY | |
| | TEDTATYFCSRGRYG | | YCQNDYYYPLTFGAGT | |
| | NNRDYWGQGTTLTVS | | KLELK | |
| | S | | | |
| sg51G10 | MGWSWIFLFLLSRTA | 433 | MESQTQVLMSLLFWVS | 463 |
| | GVHSRVQLQQSGPEL | | GICGDIVMTQSPSSLT | |
| | VKPGASMKLSCKTSG | | VTAGEKATMSCKSSQS | |
| | YTFTNFDINWVKQRP | | LLNGGNQKNYLTWYQQ | |
| | GQGLEWIGLSYPRDS | | KPGQPPTLLIYWASTR | |
| | TTQYNGKFRGKATLT | | ESGVPDRFTGSGSGTY | |
| | VDTSSTTAYMELRSL | | FTLTISSVQAEDLAVY | |
| | TSEDSAVYFCARGYY | | YCQNDYYFPYTFGGGT | |
| | GNSFAYWGQGTLVTV | | KLEIK | |
| | SA | | | |
| sg54F5 | MGWSWIFLFLLSETA | 419 | MESQTQVLMSLLFWVS | 486 |
| | GVLSEVQLQQSGPEL | | GTCGDIVMTQSPSSLT | |
| | VKPGASVKMSCKASG | | VTAGEKVTMSCKSSQS | |
| | YTFTDYNMHWLKQSH | | LLNSGNQKNYLTWYQK | |
| | GKSLEWIGYINPKNG | | KPGQPPKLLIYWASTR | |
| | GTRYNQKFKGKATLT | | ESGVPDRFTGSGSGTD | |
| | VNKSSSTAYMELRSL | | FTLTISSVQAEDLAVY | |
| | TSEDSAVYYCARIYY | | FCQNDYSFPFTFGSGT | |
| | GNSFDYWGQGTTLTV | | KLEIK | |
| | SS | | | |
| sg56B2 | MDSRLNLVFLVLILK | 407 | MESQTQVLMSLLFWVS | 491 |
| | GVQCEVQLVESGGGL | | GTCGDIVMTQSPSSLT | |
| | VKPGGSLKLSCAASG | | VTAGEKVTMSCKSSQS | |
| | FTFSDYGMHWVRQAP | | LLNSGNQKNYLTWYQQ | |
| | EKGLEWVAYISSGSS | | KPGQPPKLLIYWASTR | |
| | NIYYADTVKGRFTIS | | ESGVPDRFTGSGSGTD | |
| | RDNAKNTLFLQMTSL | | FTLTISSVQAEDLAVY | |
| | RSEDTAMYYCARFYY | | YCQNAYSFPFTFGSGT | |
| | GNSFAYWGQGTLVTV | | QLEIR | |
| | SA | | | |
| sg59E7 | MGWSWIFLFLLSETA | 414 | MESQTQVLMSLLFWVS | 471 |
| | GVLSEVQLQQSGPDL | | GTCGDIVMTQSPSFLT | |
| | LKPGASVKMSCKASG | | VTAGEKVTMSCKSSQS | |
| | YTFTDYSMHWVRQSH | | LLNSGNLKNYLTWYQQ | |
| | GKRLEWIGFINPYSG | | KPGQPPKLLIYWASTR | |
| | STTYNQKFKGKATLT | | ESGVPDRFTGSGSGSD | |
| | VNKSSSTVYMEVRSL | | FTLTISSVQAEDLAVY | |
| | TSDDSAVYYCTRIFY | | YCQNDYFYPFTFGSGT | |
| | GNSFDYWGQGTTLTV | | RLEMK | |
| | SS | | | |
| sg59F5 | MGWSWIFLFLLSETA | 416 | MESQTQVLMSLLFWVS | 470 |
| | GVLSEVQLQQSGPEL | | GTCGDIVMTQSPSELT | |
| | LKPGASVKMSCKASG | | VTAGEKVTMSCKSSQS | |
| | YTFTDYTMHWVKQSH | | LLNSGNLKNYLTWYQQ | |
| | GKSLEWIGYINPYNS | | KPGQPPKLLIYWASTR | |
| | GTRYNQKFKGKATLT | | ESGVPDRFTGSGSGSD | |
| | VNKSSNTAYMEVRSL | | FTLTISSVQAEDLAVY | |
| | TSEDSAVYYCTRIFY | | YCQNDYFYPFTFGSGT | |
| | GNSFDYWGQGTTLTV | | RLEIK | |
| | SS | | | |
| sg59G12 | MGWSWIFLFLLSETA | 415 | MESQTQVLMSLLFWVS | 471 |
| | GVLSEVQLQQSGPEL | | GTCGDIVMTQSPSELT | |
| | LKPGASVKMSCKASG | | VTAGEKVTMSCKSSQS | |
| | YTFTDYSMHWVRQSH | | LLNSGNLKNYLTWYQQ | |
| | GKRLEWIGFINPYSG | | KPGQPPKLLIYWASTR | |
| | STTYNQKFKGKATLT | | ESGVPDRFTGSGSGSD | |
| | VNKSSSTVYMEVRSL | | FTLTISSVQAEDLAVY | |
| | TSDDSAVYYCTRIFY | | YCQNDYFYPFTFGSGT | |
| | GNSFDYWGQGTTLTV | | RLEMK | |
| | SS | | | |
| sg60F11 | MGWSYIILFLVATAT | 437 | MESQTQVLMSLLFWVS | 479 |
| | GVHSQVQLQQPGAEL | | GTCGDIVMTQSPSSLT | |
| | VKPGASVKLSCKASG | | VTAGEKVTLSCKSSQS | |
| | YTFTSYWMHWVKQRP | | LLNSGNQRNYLTWYQQ | |
| | GQGLEWIGMIHPNSG | | KPGQPPKLLIYWASTR | |
| | STNYNEKFKSKATLT | | ESGVPDRFTGSGSGTD | |
| | VDKSSSTAYMQLSSL | | FTLTISSVQAEDLAVY | |
| | TSEDSAVYYCARMGL | | YCQNAYSYPLTFGAGT | |
| | GNAMDYWGQGTSVTV | | KLELK | |
| | SS | | | |
| sg61A5 | MYFRLSSVFLLLILK | 457 | MDSQAQVLMLLLLWVS | 461 |
| | GVQCEVKLVESEGGL | | GTCGDIVMSQSPSSLA | |
| | VQPGSSMKLSCTASG | | VSVGEKVTMSCKSSQS | |
| | FTFSDYYMAWVRQVP | | LLYSSNQKNYLAWYQQ | |
| | EKGLEWVANINYDGS | | KPGQSPKLLIYWASTR | |
| | STFYLDSLKSRFIIS | | ESGVPDRFTGSGSGTD | |
| | RDNARNILYLQMTSL | | FTLTISSVKAEDLAVY | |
| | KSEDTATYFCGRQVG | | YCQQYYTYPLTFGAGT | |
| | YYDPMDYWGQGTSVT | | KLELK | |
| | VAS | | | |
| sg64C1 | MGWSWIFLFLLSEAA | 413 | MESQTQVLMSLLFWVS | 470 |
| | GVLSEVQLQQSGPEL | | GTCGDIVMTQSPSFLT | |
| | LKPGASVKMSCKASG | | VTAGEKVTMSCKSSQS | |
| | YTFTDYTIHWVKQSH | | LLNSGNLKNYLTWYQQ | |
| | GESLEWIGYINPYNS | | KPGQPPKLLIYWASTR | |
| | GTRYNQKFKGKATLT | | ESGVPDRFTGSGSGSD | |
| | VNKSSSTAYMEVRSL | | FTLTISSVQAEDLAVY | |
| | TSEDSAVYFCTRIFY | | YCQNDYFYPFTFGSGT | |
| | GNSFDYWGQGTTLTV | | RLEIK | |
| | SS | | | |
| sg64C10 | MGWSWIFLFLLSETA | 417 | MESQTQVLMSLLFWVS | 472 |
| | GVLSEVQLQQSGPEL | | GTCGDIVMTQSPSELT | |
| | LKPGASVTMSCKASG | | VTAGEKVTMSCKSSQS | |
| | YTFTDYTIHWVKQSH | | LLNSGNLKNYLTWYQQ | |
| | GKSLEWIGSINPYNP | | KPGQPPKLLIYWASTR | |
| | GTRYNQKFEGKATLT | | ESGVPDRFTGSGSGSD | |
| | VNKSSNTAYMEFRSL | | FTLTISSVQAEDLAVY | |
| | TSEDSAVYYCTRVFY | | YCQNNYFYPFTFGSGT | |
| | GNSFDYWGQGTTLTV | | RLEIK | |
| | SS | | | |
| sg66D7-1 | MRWSCIILFLVATAT | 452 | MESQTQVLMSLLFWVS | 484 |
| | GVHSQVLLQQPGTEL | | GTCGDIVMTQSPSSLT | |
| | VKPGASVKVSCKASA | | VTAGEKVTMSCKSSQS | |
| | YTFTSYWIHWVKQRP | | LLNGGNQKNYLTWYQQ | |
| | GQGLEWIGRIRPSDS | | KPGQPPKLLIYWASTR | |
| | DSTYNQNFKGKATLT | | ESGVPDRFTGSGSGTD | |
| | VDKSSDTAYMQLTSL | | FTLTISTMQAEDLAVY | |
| | TSEDSAVYYCSMGAY | | YCQNDYFFPYTFGGGT | |
| | YSNSFGYWGQGSLVT | | KLEIK | |
| | VSA | | | |
| sg66D7-2 | MGWSWIFLFLLSGTA | 427 | MESQTQVLMSLLFWVS | 463 |
| | GVHSQVQLQQSGPEL | | GICGDIVMTQSPSSLT | |
| | VKPGTSVKLSCKASG | | VTAGEKATMSCKSSQS | |
| | YTFINYDINWVKQRP | | LLNGGNQKNYLTWYQQ | |
| | GQGLEWIAWIFPRDG | | KPGQPPTLLIYWASTR | |
| | STKYNEKFRGEATLT | | ESGVPDRFTGSGSGTY | |
| | VDTSSSTAYLGLHSL | | FTLTISSVQAEDLAVY | |
| | TSEDSAVYFCARGYY | | YCQNDYYFPYTFGGGT | |
| | GNSFAYWGQGTLVTV | | KLEIK | |
| | SA | | | |
| sg66E12 | MGWSYIILELVATAT | 436 | MESQTQVLMSLLFWVS | 495 |
| | GVHSQVQLQQPGAEL | | GTCGDIVMTQSPSSLT | |
| | VKPGASVKLSCKASG | | VTAGEKVTMSCKSSQS | |
| | YTFSSYWIPWVKQRP | | LLNSGNQKNYLTWYQQ | |
| | GQGLEWIGMIHPNSG | | KPGQPPKMLIYWASTR | |
| | STNYNEKFKRKAILI | | ESGVPDRFTGSGSGTD | |
| | VDKSSNTAYMQLSSL | | FTLTLSSVKAEDLAVY | |
| | TSDDSAVYYCGRMGL | | YCQNDYYYPLTFGAGT | |
| | GNAMDYWGQGTSVTV | | KLELR | |
| | SS | | | |
| sg66E6 | MGWSWIFLFLLSGTA | 425 | MESQTQVLMSLLFWVS | 467 |
| | GVHSQVQLQQSGPEL | | GICGDIVMTQSPSSLT | |
| | VKPGASVKLSCKASG | | VTAGERVTMSCKSSQS | |
| | YTFTNYDINWVKQRP | | LLNSGNLKNYLTWYQQ | |
| | GQGLEWIGLIYPRDK | | KPGQPPKLLIYWASTR | |
| | NTNYNGKFKGKATLT | | ESGVPDRFTGSGSGTY | |
| | VDTSSSTAYMELHSL | | FTLTISSVQAEDLAVY | |
| | TSEDSAVYFCARGYY | | YCQNDYYYPYTFGGGT | |
| | GNSFAYWGQGTLVTV | | KLEIK | |
| | FA | | | |
| sg68D1 | MGWSWIFLFLLSGTA | 424 | MESQTQVLMSLLFWVS | 464 |
| | GVHSQVQLQQSGPEL | | GICGDIVMTQSPSSLT | |
| | VKPGASMKLSCKASG | | VTAGEKVTLSCKSSQS | |
| | YTFTSYDINWVKQRP | | LLNSGNQKNYLTWYQQ | |
| | GQGLEWIGLSYPRDG | | KPGQPPKLLIYWASTR | |
| | TTQYNGKFKGKATLT | | ESGVPDRFTGSGSGTY | |
| | VDTSSSTAYMELRSL | | FTLTISSVQAEDLAVY | |
| | TSEDSAVYFCARGYY | | YCQNDYYFPYTFGGGT | |
| | GNSFAYWGQGTLVTV | | KLEIK | |
| | SA | | | |
| sg68E9 | MGWSWIFLFLLSETA | 421 | MESQTQVLMSLLFWVS | 490 |
| | GVLSEVQLQQSGPEL | | GTCGDIVMTQSPSSLT | |
| | VKPGSSVKMSCKASG | | VTAGEKVTMSCKSSQS | |
| | YTFTDYNMHWLKQSH | | LLNSGNQKNYLTWYQQ | |
| | GKSLEWIGYINPKNG | | KPGQPPKLLIYWASTR | |
| | GTRYNQKFKGKATLT | | ESGVPDRFTGSGSGTD | |
| | VNKSSSTAYMELRSL | | FTLTISSVQAEDLAVY | |
| | TSEDSAVYYCARLYY | | FCQNDYSFPFTFGSGT | |
| | GNSFDYWGQGTTLTV | | KLEIK | |
| | SS | | | |
| sg69B2 | MGWSYIILFLVATAT | 435 | MESQTQVLMSLLFWVS | 492 |
| | GVHSQVQLQQPGAEL | | GTCGDIVMTQSPSSLT | |
| | IKPGASVKLSCKASG | | VTAGEKVTMSCKSSQS | |
| | YTFTSYWIPWVKQRP | | LLNSGNQKNYLTWYQQ | |
| | GQGLEWIGMIHPNSD | | KPGQPPKLLIYWASTR | |
| | STNYNEKFKSKATLT | | ESGVPDRFTGSGSGTD | |
| | VDKSSSTAYIQLSSL | | FTLTISSVQAEDLAVY | |
| | TSDDSAVYYCARMGL | | YCQNDYYYPLTFGAGT | |
| | GNALDYWGQGTSVTV | | KLELK | |
| | SS | | | |
| sg73E4 | MGWSWIFLFLLSRTA | 432 | MESQTQVLMSLLFWVS | 462 |
| | GVHSQVQLQQSGPEL | | GICGDIVMTQSPSSLT | |
| | VKPGASMKLSCKASG | | VTAGEKATMSCKSSQS | |
| | YTFTSYDINWVKQRP | | LLNGGNQKNYLTWYQQ | |
| | GQGPEWIGLSYPRDS | | KPGQPPKLLIYWASTR | |
| | STQYNGRERGKATLT | | ESGVPDRFTGSGSGTY | |
| | VDTSSTTAYMELRSL | | FTLTISSVQAEDLAVY | |
| | TSEDSAVYFCARGYY | | YCQNDYYFPYTFGGGT | |
| | GNSFAYWGQGTLVTV | | KLEIK | |
| | SA | | | |
| sg78H6 | MGWSWIFLFLLSETA | 420 | MESQTQVLMSLLFWVS | 469 |
| | GVLSEVQLQQSGPEL | | GTCGDIMMTQSPSSLT | |
| | VKPGASVKMSCKASG | | VTAGEKVTMSCKSSQS | |
| | YTFTDYNMHWVKQSH | | LLNSGNQKNYLTWYQQ | |
| | GKSLEWIGYINPNNG | | KPGQPPKLLIYWASTR | |
| | GTTYNQKFKGKATLT | | ESGVPDRFTGSGSGTD | |
| | VNKSSSTAYMELRGL | | FTLTISSVQAEDLAVY | |
| | TSEDSAIYYCARIYY | | YCQNDYSFPFTFGSGT | |
| | GNSFDYWGQGTTLTV | | KLEIK | |
| | SS | | | |
| sg79C3 | MGWSWIFLFLLSETA | 418 | MESQTQVLMSLLFWVS | 489 |
| | GVLSEVQLQQSGPEL | | GTCGDIVMTQSPSSLT | |
| | VKPGASVKMSCKASG | | VTAGEKVTMSCKSSQS | |
| | YTFTDYNIHWLKQSP | | LLNSGNQKNYLTWYQQ | |
| | GKSLEWIGYINPKNG | | KPGQPPKLLIYWASTR | |
| | GTRYNQKFKGKATLT | | ESGVPDRFTGSGSGTD | |
| | VNKSSSTAYMELRSL | | FTLTISSVQAADLAVY | |
| | TSEDSAVYYCSRIYY | | FCQNDYSFPFTFGSGT | |
| | GNSFDYWGQGTTLTV | | KLEIK | |
| | SS | | | |
| sg80F10 | MGWSWIFLFLLSGTA | 423 | MESQTQVLMSLLFWVS | 466 |
| | GVHSQVQLQQSGPEL | | GICGDIVMTQSPSSLT | |
| | VKPGASMKLSCKASG | | VTAGEKVTMSCKSSQS | |
| | YTFTSYDINWVKQRP | | LLNSGNQKNYLTWYQQ | |
| | GQGLEWIGLSYPRDG | | KPGQPPKLLMYWASTR | |
| | TTQYNGKFKGEATLT | | ESGVPDRFTGSGSGTY | |
| | VDRSSSTAYMELRSL | | FTLTISSVQAEDLAVY | |
| | TSEDSAVYFCARGYY | | YCQNDYYFPYTFGGGT | |
| | GNSFAYWGQGTLVTV | | KLEIK | |
| | SA | | | |
| sg83H3 | MGWSWIFLFLLSGTA | 426 | MESQTQVLMSLLFWVS | 476 |
| | GVHSQVQLQQSGPEL | | GTCGDIVMTQSPSSLA | |
| | VKPGSSVKLSCKASG | | VTPGEKVTMNCKSSQS | |
| | YTFTRNDINWVKQRP | | LLNDGNQKNYLTWYQQ | |
| | GQGLEWIGRIYPRDG | | KPGQPPKLLIYWASTR | |
| | GTNYNEKFKGKATLT | | ESGVPDRFAGSGSGTS | |
| | VDTLSSTAYMELHSL | | FTLTINSVQAEDLAVY | |
| | TSEDSAVHFCARGYY | | YCQNGYSFPYTFGGGT | |
| | GNSFAYWGQGTLVTV | | NLEIK | |
| | SA | | | |
| sg84E8 | MGWSYIILFLVATAT | 439 | MESQTQVLMSLLFWVS | 496 |
| | GVHSQVQLQQPGAEL | | GTCGDIVMTQSPSSLT | |
| | VKPGASVKLSCKPSG | | VTAGEKVTMSCKSSQS | |
| | YTFSSYWIPWVKQRP | | LLNSGNQKNYLTWYQQ | |
| | GQGLEWIGMIHPNSG | | KPGQSPKMLIYWASTR | |
| | STNYNEKFKRKAILT | | ASGVPDRFTGSGSGTD | |
| | VDKSSSTAYMQLSSL | | FTLTLSSVKAEDLAVY | |
| | TSDDSAVYYCGRMGL | | YCQNDYYYPLTFGAGT | |
| | GNAMDYWGQGTSVTV | | KLELR | |
| | SS | | | |
| sg97A9 | MGWSWIFLFLLSGTA | 422 | MESQTQVLMSLLFWVS | 465 |
| | GVHSQVQLQQSGPEL | | GICGDIVMTQSPSSLT | |
| | VKPGASMKLSCKASG | | VTAGEKVTMSCKSSQS | |
| | YSFTRNDINWVKQRP | | LLNSGNQKNYLTWYQQ | |
| | GQGLEWIGLSYPRDG | | KPGQPPKLLIYWASTR | |
| | TTQYNGKFKGKATLT | | ESGVPDRFTGSGSGTY | |
| | VDTSSSTAYMELRSL | | FTLTISSVQAEDLAVY | |
| | TSEDSAVYFCARGYY | | FCQNDYYFPYTFGGGT | |
| | GNSFAYWGQGTLVTV | | KLEIK | |
| | SA | | | |
| sg99A7 | MGWSYIILELVATAT | 438 | MESQTQVLMSLLFWVS | 493 |
| | GVHSQVQLQQPGAEL | | GTCGDIVMTQSPSSLT | |
| | VKPGASVKLSCKASG | | VTAGEKVTMSCKSSQS | |
| | YTVTRYWIQWVKQRP | | LLNSGNQKNYLTWYQQ | |
| | GQGLEWIGMIHPNSG | | KPGQPPKLLIYWASTR | |
| | STNYNEKFKKKAALT | | ESGVPDRFTGSGSGTD | |
| | LDKSSSTAYMQLSSP | | FTLTISSVQAEDLAVY | |
| | TSEDSAVYYCVRMGL | | YCQNNYVYPLTFGAGT | |
| | GNAMDFWGQGTSVTV | | KLELR | |
| | SS | | | |
| sg99G8 | MGWYWIFLFLLSGTA | 441 | MESQTQVLMSLLFWVS | 487 |
| | GVHSQVHLQQSGPEL | | GTCGDIVMTQSPSSLT | |
| | VKPGASVKVSCKASG | | VTAGEKVTMSCKSSQS | |
| | YSFRNYDINWVKQRP | | LLNSGNQKNYLTWYQQ | |
| | GQGLEWIGRIYPRDD | | KPGQAPKLLIYWASTR | |
| | STTYNEKFKGKASLT | | QSGVPDRFTGSGFGTD | |
| | VDTSSSTAYMEFHSL | | FTLIITTVQTEDLAVY | |
| | TSEDSAVYFCARGYY | | FCQNDFGFPYTFGGGT | |
| | GNSFAYWGQGTLVTV | | KLEMN | |
| | SA | | | |
| sg99H8 | MGWSWIFLFLLSGTA | 431 | MESQTQVLMSLLFWVS | 503 |
| | GVRSQVQLQQSGPEL | | GTCGDIVMTQSPSSLT | |
| | VKPGASVKLSCKASG | | VTAREKVIMNCKSSQS | |
| | YSFTNFDINWVKQRP | | LENSGNQKNYLTWYQQ | |
| | GQGLQWIGRLYPRDG | | KPGQSPKLLIYWASTR | |
| | TTTYNEKFKGKASLT | | QSGVPDRFTGSGSGTD | |
| | VDTSSTTSYMDLHSL | | FTLTISTVQAEDLAVY | |
| | TSEDSAVYFCVRGNY | | FCQNGFSFPYTFGGGT | |
| | GNSFAYWGQGTLVTV | | KLEMN | |
| | SA | | | |

### Polynucleotides and Recombinant Methods

The present disclosure provides isolated polynucleotides that encode the anti-CLDN18 (in particular, anti-CLDN18.2) antibodies or antigen-binding fragments thereof provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The isolated polynucleotide that encodes the anti-CLDN18 (in particular, anti-CLDN18.2) antibodies or antigen-binding fragments thereof provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g. SV40, CMV, EF-la), and a transcription termination sequence.

The present disclosure provides vectors comprising the isolated polynucleotides provided herein. In certain embodiments, the polynucleotides provided herein encodes the antibodies or antigen-binding fragments thereof, at least one promoter (e.g. SV40, CMV, EF-la) operably linked to the nucleic acid sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. lichenformis, Pseudomonas such as P. aeruginosa, and Streptomyces.

In addition to prokaryotes, eukaryotic microbes such as filamentous fungi or yeast are suitable cloning or expression hosts for anti-CLDN18 (in particular, anti-CLDN18.2) antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; mouse forestomach carcinoma cells (MFC), SNU620 cells, and a human hepatoma line (Hep G2). In some embodiments, the host cell is a mammalian cultured cell line, such as CHO, BHK, NS0, 293, MFC, SNU620 and their derivatives.

Host cells are transformed with the above-described expression or cloning vectors for anti-CLDN18 (in particular, anti-CLDN18.2) antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CLDN18 (in particular, anti-CLDN18.2) antibodies or antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g. from about 0-0.25M salt).

### Pharmaceutical Composition

The present disclosure further provides pharmaceutical compositions comprising the anti-CLDN18 (in particular anti-CLDN18.2) antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gels, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate.

As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-CLDN18 (in particular, anti-CLDN18.2) antibody or antigen-binding fragment thereof or composition thereof. Overfilling vials with a small amount above that needed for a dose or set of doses (e.g. about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Chimeric Antigen Receptor

In certain embodiments, the present disclosure provides a chimeric antigen receptor comprising the antibody or an antigen-binding fragment thereof provided herein, a transmembrane region and an intracellular signal region.

The term “chimeric antigen receptor” or “CAR” or “CARs” as used herein refers to engineered receptors, which graft an antigen specificity onto cells (for example, T cells such as naive T cells, central memory T cells, effector memory T cells, regulatory T cells or combination thereof). CARs are also known as artificial T-cell receptors, chimeric T-cell receptors or chimeric immunoreceptors. In some embodiments, CARs comprise an antigen-specific targeting region (for example, the antigen-binding fragments of the anti-CLDN18 antibody as provided herein), an extracellular region, a transmembrane region, one or more co-stimulatory regions, and an intracellular signal region.

In some embodiments, the antigen-specific targeting region is an scFv. In some embodiments, the transmembrane region comprises a transmembrane region of CD3, CD4, CD8 or CD28. In some embodiments, the co-stimulatory region comprises a co-stimulatory domain of CD28, ICOS, CD27, 4-1BB, OX40 and CD40L. In some embodiments, the intracellular signal region is selected from the group consisting of: an intracellular signal region sequence of CD3, CD27, CD28, CD137, CD134, MyD88, CD40, CD278, TLRs, or a combination thereof.

### Kits

In certain embodiments, the present disclosure provides a kit comprising the antibody or an antigen-binding fragment thereof provided herein and/or the pharmaceutical composition provided herein and/or the chimeric antigen receptor provided herein. In certain embodiments, the present disclosure provides a kit comprising the antibody or an antigen-binding fragment thereof provided herein and/or the pharmaceutical composition provided herein and/or the chimeric antigen receptor provided herein, and a second therapeutic agent. In certain embodiments, the second therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating, preventing or alleviating a CLDN18 related disease, disorder or condition in a subject, comprising administering to the subject a therapeutically effective amount of the antibody or antigen-binding fragment thereof provided herein, and/or the pharmaceutical composition provided herein, and/or the chimeric antigen receptor provided herein. In certain embodiments, the CLDN18 related disease, disorder or condition is a CLDN18.2 related disease, disorder or condition. In certain embodiments, the subject is human.

In some embodiments, the CLDN18 related disease, disorder or condition is characterized in expressing or over-expressing of CLDN18 (in particular, CLDN18.2).

In certain embodiments, the CLDN18 related disease, disorder or condition is cancer. In certain embodiments, the cancer is a CLDN18-expressing cancer. “CLDN18-expressing” cancer as used herein refers to a cancer characterized in expressing CLDN18 (in particular, CLDN18.2) protein in a cancer cell, a tumor infiltrating immune cell, or expressing CLDN18 (in particular, CLDN18.2) in a cancer cell, a tumor infiltrating immune cell at a level significantly higher than that would have been expected of a normal cell. Various methods can be used to determine the presence and/or amount of CLDN18 in a test biological sample from the subject. For example, the test biological sample can be exposed to an anti-CLDN18 (in particular, anti-CLDN18.2) antibody or antigen-binding fragment thereof, which binds to and detects the expressed CLDN18 (in particular, CLDN18.2) protein. Alternatively, CLDN18 (in particular, CLDN18.2) can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g. tumor).

In certain embodiments, the cancer is an epithelial cell-derived cancer. “Epithelial cell-derived cancer” refers to a cancer that is originated from epithelial cells, for example, alveolar epithelial cells, epithelial cells of gastric mucosa, epithelial cells of skin, blood vessels, urinary tract, etc.

In certain embodiments, the cancer is selected from the group consisting of anal cancer, appendix cancer, astrocytoma, basal cell carcinoma, gallbladder cancer, gastric cancer, lung cancer, bronchial cancer, bone cancer, liver and bile duct cancer, pancreatic cancer, breast cancer, liver cancer, ovarian cancer, testicle cancer, kidney cancer, renal pelvis and ureter cancer, salivary gland cancer, small intestine cancer, urethral cancer, bladder cancer, head and neck cancer, spine cancer, brain cancer, cervix cancer, uterine cancer, endometrial cancer, colon cancer, colorectal cancer, rectal cancer, esophageal cancer, gastrointestinal cancer, skin cancer, prostate cancer, pituitary cancer, vagina cancer, thyroid cancer, throat cancer, glioblastoma, melanoma, myelodysplastic syndrome, sarcoma, teratoma, chronic lymphocytic leukemia (CLL), chronic myeloid leukemia (CML), acute lymphocytic leukemia (ALL), acute myeloid leukemia (AML), Hodgkin lymphoma, non-Hodgkin lymphoma, multiple myeloma, T or B cell lymphoma, GI organ interstitialoma, soft tissue tumor, hepatocellular carcinoma, or adenocarcinoma, or the metastases thereof.

In certain embodiments, the cancer is gastric cancer, pancreatic cancer, esophagus cancer, ovarian cancer, or the metastases thereof.

In some embodiments, the subject has been identified as having a cancer cell or tumor infiltrating immune cells expressing CLDN18 (in particular, CLDN18.2), optionally at a level significantly higher from the level normally found on non-cancer cells.

In another aspect, methods are provided to treat, prevent or alleviate a disease, disorder or condition in a subject that would benefit from modulation of CLDN18 (in particular, CLDN18.2) activity, comprising administering to the subject a therapeutically effective amount of the antibody or antigen-binding fragment thereof provided herein, and/or the pharmaceutical composition provided herein, and/or the chimeric antigen receptor provided herein. In certain embodiments, the disease, disorder or condition is a CLDN18 (in particular, CLDN18.2) related disease, disorder or condition, which is defined above.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g. physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g. a therapeutic response). For example, a single dose may be administered, or several divided doses may be administered over time.

The antibodies or antigen-binding fragments thereof provided herein may be administered by any route known in the art, such as for example parenteral (e.g. subcutaneous, intraperitoneal, intravenous, including intravenous infusion, intramuscular, or intradermal injection) or non-parenteral (e.g. oral, nasal, intraocular, sublingual, rectal, or topical) routes.

In some embodiments, the antibodies or antigen-binding fragments thereof provided herein may be administered alone or in combination with a therapeutically effective amount of a second therapeutic agent. For example, the antibodies or antigen-binding fragments thereof disclosed herein may be administered in combination with a second therapeutic agent, for example, a chemotherapeutic agent, an anti-cancer drug, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, or cytokines.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein that is administered in combination with one or more additional therapeutic agents may be administered simultaneously with the one or more additional therapeutic agents, and in certain of these embodiments the antibody or antigen-binding fragment thereof and the additional therapeutic agent(s) may be administered as part of the same pharmaceutical composition. However, an antibody or antigen-binding fragment thereof administered “in combination” with another therapeutic agent does not have to be administered simultaneously with or in the same composition as the agent. An antibody or antigen-binding fragment thereof administered prior to or after another agent is considered to be administered “in combination” with that agent as the phrase is used herein, even if the antibody or antigen-binding fragment and the second agent are administered via different routes. Where possible, additional therapeutic agents administered in combination with the antibodies or antigen-binding fragments thereof disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides methods of modulating CLDN18 (in particular, CLDN18.2) activity in CLDN18-positive cells, comprising exposing the CLDN18-positive cells to the antibodies or antigen-binding fragments thereof provided herein, and/or the pharmaceutical composition provided herein, and/or the chimeric antigen receptor provided herein. In some embodiments, the CLDN18-positive cell is an epithelial cell.

In another aspect, the present disclosure provides methods of detecting the presence or amount of CLDN18 (in particular, CLDN18.2) in a sample, comprising contacting the sample with the antibody or antigen-binding fragment thereof provided herein, and/or the pharmaceutical composition provided herein, and/or the chimeric antigen receptor provided herein, and determining the presence or the amount of CLDN18 (in particular, CLDN18.2) in the sample.

In another aspect, the present disclosure provides a method of diagnosing a CLDN18 (in particular, CLDN18.2) related disease, disorder or condition in a subject, comprising: a) contacting a sample obtained from the subject with the antibody or an antigen-binding fragment thereof provided herein and/or the pharmaceutical composition provided herein and/or the chimeric antigen receptor provided herein; b) determining the presence or amount of CLDN18 (in particular, CLDN18.2) in the sample; and c) correlating the presence or the amount of CLDN18 (in particular, CLDN18.2) to existence or status of the CLDN18 (in particular, CLDN18.2) related disease, disorder or condition in the subject.

In another aspect, the present disclosure provides kits comprising the antibody or antigen-binding fragment thereof provided herein and/or the pharmaceutical composition provided herein and/or the chimeric antigen receptor provided herein, optionally conjugated with a detectable moiety, which is useful in detecting CLDN18 (in particular, CLDN18.2). The kits may further comprise instructions for use.

In another aspect, the present disclosure also provides use of the antibody or antigen-binding fragment thereof provided herein and/or the pharmaceutical composition provided herein and/or the chimeric antigen receptor provided herein in the manufacture of a medicament for treating, preventing or alleviating a CLDN18 (in particular CLDN18.2) related disease, disorder or condition in a subject, in the manufacture of a diagnostic reagent for diagnosing a CLDN18 (in particular, CLDN18.2) related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention.

It will be understood that many variations can be made in the procedures herein described while still remaining within the bounds of the present invention. It is the intention of the inventors that such variations are included within the scope of the invention.

### EXAMPLES

### Example 1. Antibody Generation

### 1.1. Immunization

To generate antibodies against CLDN18.2, three different strategies of protein immunization using human CLDN18.2 (hCLDN18.2) stabilized protein, genetic immunization using hCLDN18.2 expression plasmid, and cell immunization using CHO-K1-hCLDN18.2 stable cell line were applied. The immunogens used in the Examples, i.e., hCLDN18.2 expression plasmid, CHO-K1-hCLDN18.2 stable cell line, hCLDN18.2 stabilized protein were commercially available, and were purchased from GenScript. IMAB362 (Zolbetuximab), which is a humanized anti-CLDN18.2 antibody, was used as a reference antibody in the Examples, and was purchased from GenScript. The description of each immunogen was provided in Table 17 below.

TABLE 17
Information of Each Immunogen

| Immunogen | Quantity | Purpose |
|---|---|---|
| hCLDN18.2 expression | 1 mg, transfection | Immunization |
| plasmid | grade | |
| CHO-K1-hCLDN18.2 stable | >1 × 10⁶/vial, 2 | Immunization |
| cell line | vials each | |
| hCLDN18.2 stabilized protein | 2 mg, purity >70% | Immunization |
| IMAB362 | 100 μg (>85% | Reference |
| | purity, >1 mg/ml) | antibody |

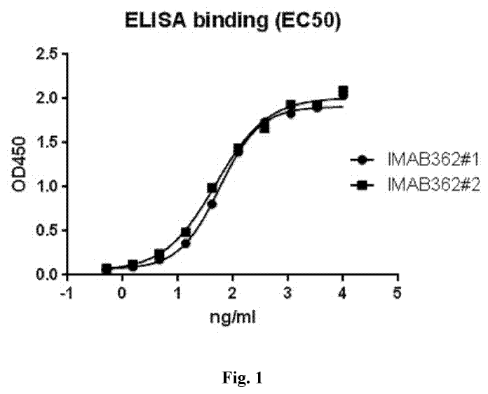

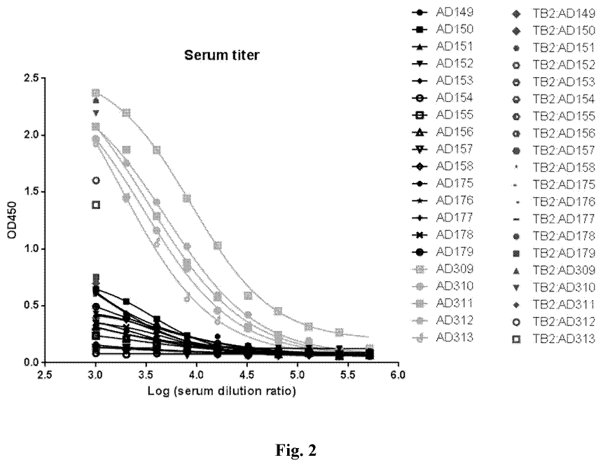

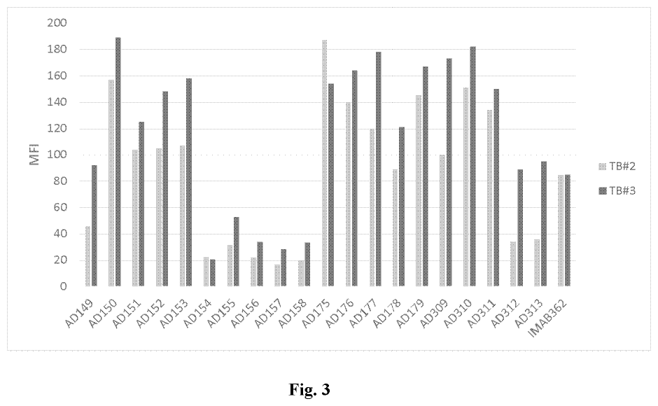

Specifically, five SJL mice were immunized with hCLDN18.2 stabilized protein, five BALB/C mice, five C57 mice, and five SJL mice were immunized with hCLDN18.2 expression plasmid and final boost with CHO-K1-hCLDN18.2 stable cell line, respectively. The immunization protocols were summarized in Tables 18, 19, 20 and 21 below. The primary immunization were followed by several boosts until animals developed satisfactory serum titers suitable for hybridoma development. ELISA assay against hCLDN18.2 stabilized protein with reference antibody IMAB362 (see FIG. 1), ELISA assay with hCLDN18.2 stabilized protein (see FIG. 2), and FACS assay with CHO-K1-hCLDN18.2 stable cell line (See FIG. 3) was used to detect serum titers of immunized mice. Mice with sufficient titers of anti-CLDN18.2 antibodies were used for cell fusions.

TABLE 18
Protocol of Protein Immunization

| Procedure | Schedule | Route | Dosage |
|---|---|---|---|
| Pre-Immune Bleed | T = −4 days | | |
| Primary | T = 0 days | s.c. injection | 50 μg target |
| Immunization | | | protein per animal |
| 1^{st} Boost | T = 14 days | i.p. injection | 25 μg target |
| | | | protein per animal |
| Test Bleed 1 | T = 21 days | | |
| 2^{nd} Boost | T = 28 days | s.c. injection | 25 μg target |
| | | | protein per animal |
| Test Bleed 2 | T = 35 days | | |
| Final Boost | T = 56 ± 7 | i.p. injection | 25 μg target |
| | days | | protein per animal |

TABLE 19
Protocol of Genetic Immunization

| Procedure | Schedule | Route | Dosage |
|---|---|---|---|
| Pre-Immune | T = −4 days | | |
| Bleed | | | |
| Primary | T = 0 days | Gene Gun to | 3 μg of DNA per |
| Immunization | | deliver the | animal |
| | | plasmid DNA | |
| 1^{st} Boost | T = 20 days | Gene Gun to | 3 μg of DNA per |
| | | deliver the | animal |
| | | plasmid DNA | |
| Test Bleed 1 | T = 27 days | | |
| 2^{nd} Boost | T = 40 days | i.p. injection | 5 × 10⁶ cells per |
| | | | animal |
| Test Bleed 2 | T = 47 days | | |
| 3^{rd} Boost | T = 54 days | Gene Gun to | 3 μg of DNA per |
| | | deliver the | animal |
| | | plasmid DNA | |
| Test Bleed 3 | T = 61 days | | |
| Final Boost | T = 69-76 days | i.p. injection | 5 × 10⁶ cells per |
| | | | animal |

TABLE 20
Protocol of Cell Immunization

| Procedure | Schedule | Route | Dosage |
|---|---|---|---|
| Pre-Immune | T = −4 days | | |
| Bleed | | | |
| Primary | T = 0 days | i.p. injection | 5 × 10⁶ cells per |
| Immunization | | | animal |
| 1^{st}Boost | T = 14 days | i.p. injection | 5 × 10⁶ cells per |
| | | | animal |
| Test Bleed 1 | T = 21 days | | |
| 2^{nd}Boost | T = 28 days | i.p. injection | 5 × 10⁶ cells per |
| | | | animal |
| Test Bleed 2 | T = 35 days | | |
| Final Boost | T = 56 ± 7 days | i.p. injection | 5 × 10⁶ cells per |
| | | | animal |

TABLE 21
Grouping of Animals

| Group | Immunogen | Species | Animal# |
|---|---|---|---|
| A | DNA + cell | BALB/C | AD149 |
| | | | AD150 |
| | | | AD151 |
| | | | AD152 |
| | | | AD153 |
| | | C57 | AD154 |
| | | | AD155 |
| | | | AD156 |
| | | | AD157 |
| | | | AD158 |
| | | SJL | AD175 |
| | | | AD176 |
| | | | AD177 |
| | | | AD178 |
| | | | AD179 |
| B | Protein | SJL | AD309 |
| | | | AD310 |
| | | | AD311 |
| | | | AD312 |
| | | | AD313 |

### 1.2. Hybridoma Generation and Screening

Cell fusions were performed at 4 days after the final boost for each immunization. Splenocytes and/or lymph node cells from immunized mice were isolated and fused to mouse myeloma cell line (SP2/0). FACS assay against HEK293-hCLDN18.2 cells was used for primary screening (see FIG. 3). Hybridoma clones specific to hCLDN18.2 were selected to do a counter screening using HEK293-hCLDN18.2 cells. Those specific clones against hCLDN18.2 were transferred to 24-well, supernatants were then collected and used for confirmatory by FACS and hybridoma clones that are specific to hCLDN18.2 were subcloned to get stable hybridoma clones. After 1-2 rounds of subcloning, hybridoma monoclones were expanded for antibody production and frozen as stock.

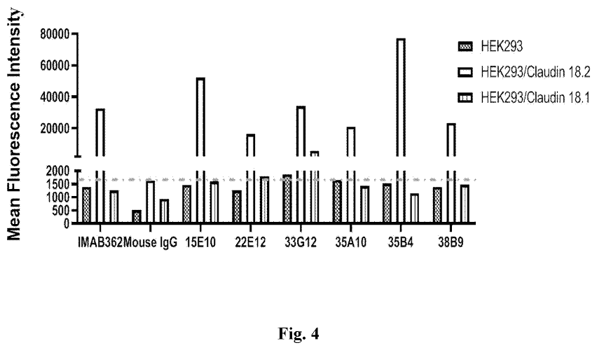

After around 10 days of culturing, the hybridoma cell culture medium were collected and purified by Protein A affinity chromatography column (GE). A total of 76 purified antibodies showed potent HEK293-hCLDN18.2 cells binding with an EC₅₀ value of about 1 nM. Among which, 60 antibodies showed unique sequences, 4 antibodies (i.e. clones 35B4, 33G12, 15E10, 40C1) showed HEK293-hCLDN18.2 cell death induction; 2 antibodies (i.e. clones 22E12 and 35A10) were identified with potent K_{D} value under pM by Octet assay; and more than 12 antibodies showed potent K_{D} values and good NK cell ADCC activation. The representative figure of hybridoma screening was shown in FIG. 4. As shown in FIG. 4, except for 33G12, all of the left tested antibodies (i.e. clones 15E10, 22E12, 35A10, 35B4, 38B9) showed specific binding to HEK-hCLDN18.2 cells.

### Example 2. Antibody Characterization

### 2.1. Antibodies

The hybridoma antibody clones 15E10, 22E12, 33G12, 35A10, 35B4, 38B9, 60F11, 97A9, and 99H8 were characterized in a series of binding and functional assays as described below.

### 2.2. Hybridoma Sequencing

Total RNA was isolated from the hybridoma cells following the technical manual of TRIzol® Reagent. Total RNA was then reverse transcribed into cDNA using isotype-specific anti-sense primers or universal primers following the technical manual of PrimeScript™ 1st Strand cDNA Synthesis Kit. The antibody fragments of VH and VL were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence of these clones was provided.

The variable region sequences of the hybridoma antibodies are provided herein in Table 3 above.

### 2.3. Antibody Binding Affinity, Cross-Reactivity and Selectivity Study

FACS assay was used to determine binding affinity of the antibodies to HEK293-hCLDN18.2 cells and SNU620 cells which naive CLDN18.2 was expressed, selectivity on HEK293-hCLDN18.1 cells, and cross-reactivity on HEK293-mouse CLDN18.2 cells. HEK293-hCLDN18.2 cells, HEK293-hCLDN18.1 cells, and HEK293-mouse CLDN18.2 (HEK293-mCLDN18.2) cells were maintained in culture medium with puromycin according to ATCC procedure. SNU620 cells were maintained in culture medium as KCLB procedure described. Cells were collected and re-suspended in blocking buffer at a density of 1×10⁶ cells/ml. Cells were transferred to 96 well FACS plates at 100 μl/well (1×10⁵ cells/well), the plates were centrifuged and washed twice with FACS buffer (PBS, 1% FBS, 0.05% Tween-20). 3-folds serial dilution of anti-CLDN18.2 antibodies were prepared in FACS buffer starting from 15 μg/ml. Reference antibody IMAB362, and mouse/human control IgG were used as positive and negative controls, respectively. Cells were re-suspended in 100 μL/well diluted antibodies, and the plates were incubated at 4° C. for 60 min. The plates were washed with FACS buffer, Alexa Fluor® 488-labeled secondary antibody (1:1000 in FACS buffer) were added to each well and incubated at 4° C. for 30 min. The plates were washed with FACS buffer, and cells were re-suspended in 100 μL/well of PBS. Cells were then analyzed with FACSCaliber™ and mean fluorescence intensity were determined. Full binding curves were generated on the hCLDN18.2 expressing cells by testing a range of antibody concentrations. Apparent affinity was determined for each antibody using Prism software.

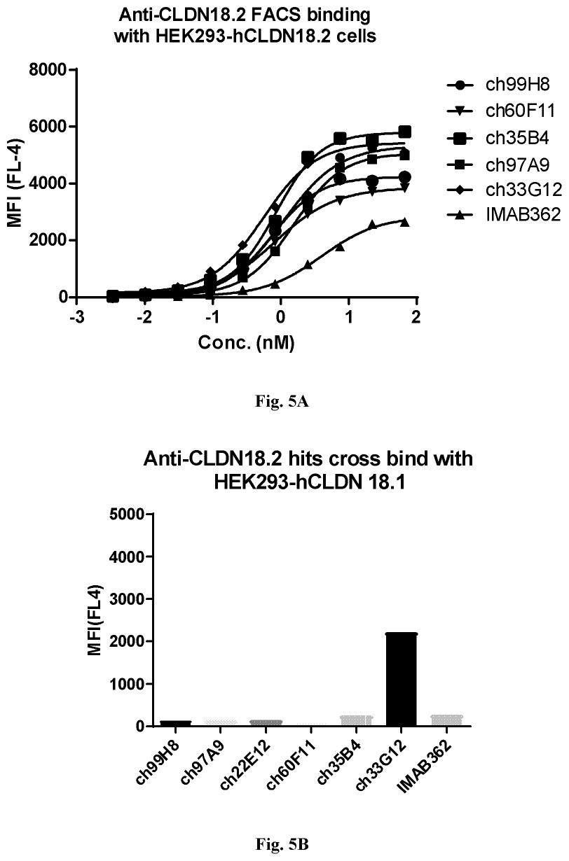

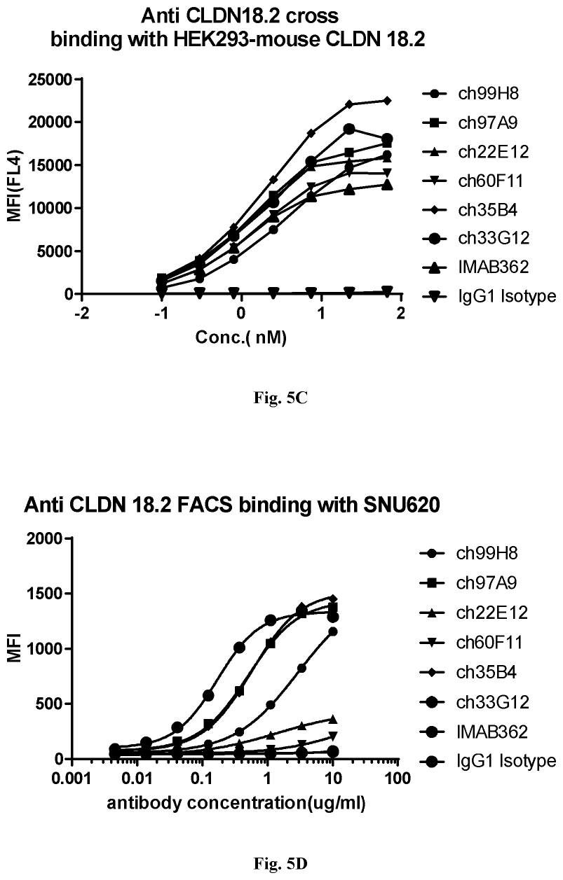

The binding affinities of the purified hybridoma antibodies to HEK293-hCLDN18.2 cells, HEK293-hCLDN18.1 cells, HEK293-mCLDN18.2 cells, and SNU620 cells were shown in FIG. 5A, FIG. 5B, FIG. 5C and FIG. 5D, respectively. As shown in FIGS. 5A-5D, all the tested hybridoma antibodies bound to human and mouse CLDN18.2 in a dose-dependent manner, only clone 33G12 bound to human CLDN18.1.

Cross reactivity and selectivity of the purified hybridoma antibodies against hCLDN18.2, mCLDN18.2, and hCLDN18.1 were determined by FACS assay using HEK293-hCLDN18.2 cells, HEK293-mCLDN18.2 cells, and HEK293-hCLDN18.1 cells, which stably expressing CLDN18.1 or CLDN18.2 protein. Briefly, the antibodies were incubated with target cells at 4° C. for 1 hour. After washing, fluorescence labeled anti-mouse or anti-human IgG 2nd antibody (Life Technologies) was added and incubated at 4° C. for 1 hour. Geometric median fluorescence intensity was detected and EC₅₀ was calculated. The cross reactivity property of 6 functional antibodies was summarized in Table 22 below. In particular, it is noted, in contrast to the other antibodies tested in the same experiment, 33G12 has recognized both hCLDN18.1 and hCLDN18.2.

### 2.4. Epitope Binning

Competitive ELISA assay was used for epitope binning with reference antibody. Briefly excessive competitor antibody and biotin labeled hCLDN18.2 were co-incubated with ELISA microplate coated reference antibody. After washing, HRP-SA was added and incubated at 37° C. for 1 hour. Then, 100 μl/well of TMB solution (Biotechnology) was added. After incubation for 15 minutes at room temperature, the reaction was stopped by the addition of 50 μl of 1N HCl. OD 450 nm was read. Competition ratio was calculated. The antibodies that can compete with reference antibody for binding to hCLDN18.2 have the similar binding epitope.

A total of 6 antibodies, as shown in Table 22 below, belong to three different epitope groups. Clones 97A9, 35B4 and 33G12 belong to the same epitope group as reference antibody IMAB362. 60F11 and 22E12 belong to the same epitope group, which is different from the reference antibody IMAB362.

Specifically, antibodies 97A9, 35B4 and 33G12 and reference antibody IMAB362 compete each other for binding to hCLDN18.2, indicating that they may bind to an identical or closely related epitope which is grouped into Group I as shown in Table 22. Antibodies 60F11 and 22E12 cannot be fully competed by reference antibody IMAB362, indicating that they may bind to a different epitope which is grouped into Group II as shown in Table 22.

TABLE 22
Anti-CLDN18.2 antibody characterization summary

| | CLDN18 binding & cross | | | | | | | |
|---|---|---|---|---|---|---|---|---|
| | reactivity (nM) | | | | | | Compete | PBMC VS |
| | | | | HEK293/ | | | with | HEK293- |
| Clone | HEK293/ | | | mCL | HEK293/ | Epitope | IMAB362 | CLDN 18.2 |
| No. | hCLDN18.2 | SNU620 | NUGC4 | DN18.2 | hCLDN18.1 | binning | (%) | ADCC |
| 99H8 | 1.1 | 19 | ++ | 3.5 | − | N.D. | N.D. | +++ |
| 97A9 | 1.6 | 3.3 | ++ | 1.4 | − | I | 92 | +++ |
| 60F11 | 0.81 | 3.5 | − | 1.5 | − | II | 40 | +++ |
| 35B4 | 0.85 | 3.8 | +++ | 1.9 | − | I | 81 | +++ |
| 22E12 | 0.86 | 2.1 | +++ | 0.9 | − | II | 49 | +++ |
| IMAB362 | 0.71 | − | − | 1.2 | − | I | 100 | +++ |
| 33G12 | 0.56 | 1.1 | +++ | 1.7 | + | I | 87 | +++ |

| | PBMC VS | | | | | | | |
|---|---|---|---|---|---|---|---|---|
| | NUGC4 | | | | | | | |
| | ADCC | CDC | | | | | | |
| Clone | (EC50 | human | Octet | | | Biacore | | |
| No. | nM) | serum | KD(M) | Kon | Koff | KD | Koff | Kon |
| 99H8 | 3.31 | +++ | 2.0E−09 | 1.1E+05 | 2.3E−04 | 1.75E−09 | 2.03E+05 | 3.55E−04 |
| 97A9 | 6.93 | +++ | 2.2E−09 | 1.8E+05 | 4.1E−04 | 1.15E−09 | 4.25E+05 | 4.89E−04 |
| 60F11 | 8.69 | +++ | 2.8E−09 | 2.2E+05 | 5.9E−04 | N.D. | N.D. | N.D. |
| 35B4 | 4.05 | +++ | 3.3E−09 | 9.0E+04 | 3.0E−04 | 1.57E−09 | 2.22E+05 | 3.50E−04 |
| 22E12 | 1.06 | +++ | <1.0E−12 | 1.2E+05 | <1.0E−07 | 2.6E−10 | 7.78E+05 | 2.02E−04 |
| IMAB362 | 25.14 | +++ | 1.6E−09 | 1.4E+05 | 2.2E−04 | 1.15E−07 | 1.81E+05 | 2.08E−02 |
| 33G12 | 0.4584 | +++ | 6.2E−09 | 1.4E+05 | 8.5E−04 | N.D. | N.D. | N.D. |

−: no response
+: weak response
++: medium response
+++: strong response
N.D.: not detect

### Example 3. Chimeric Antibody Generation and Characterization

### 3.1. Chimeric Antibody Generation and Production

DNA encoding variable regions of 6 selected hybridoma antibodies (i.e. clones 99H8, 97A9, 60F11, 35B4, 22E12, 33G12) were synthesized and subcloned into an expression vector where human IgG1 constant gene was included in advance. The vectors were transfected into mammalian cells for recombinant protein expression and the expressed antibody was purified using protein A affinity chromatography column. The resulting chimeric antibodies are referred to herein as ch99H8, ch97A9, ch60F11, ch35B4, ch22E12, ch33G12, where the prefix “ch” indicates “chimeric”, and the suffix indicates the hybridoma antibody clone, for example, “99H8” indicates that it is from the hybridoma antibody clone 99H8.

### 3.2. Chimeric Antibody Characterization: Antibody-Dependent Cellular Cytotoxicity (ADCC) Study

The purified 6 chimeric antibodies were tested for antibody-dependent cellular cytotoxicity (ADCC) activity. Briefly, the target cells, i.e. HEK293/hCLDN18.2 cells, were cultured as describe above, and collected and washed for twice with pre-warmed PBS before the study. The isolated cells were resuspended with PBS and labeled with CellTrace™ Violet and the cell density was adjusted to 4×10⁵ cells/ml and 25 μl per well was added into 96 well plates. The antibody concentration was diluted to 40 g/ml and 25 μl per well was added to reach a final concentration of 10 g/ml. Then the cells were incubated at 37° C. for 15 min and protected from light. The effector cells, i.e. human PBMC, was prepared to 5×10⁶/ml, and 501 per well was added, E/T=25:1. The cells mixture were incubated at 37° C. for 2 h, then stained with PI and live/dead cells were analyzed with FACS.

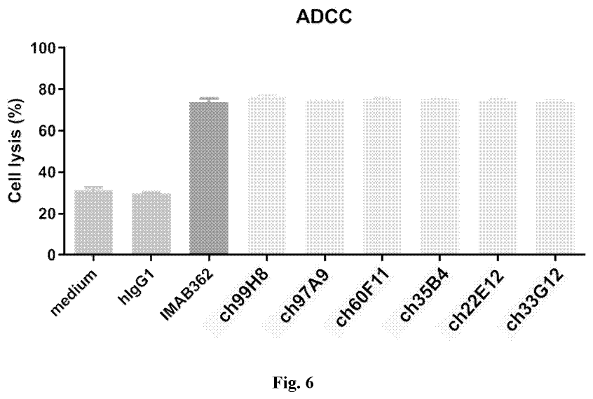

The ADCC study result was shown in Table 22 above and FIG. 6. As shown in FIG. 6, all of the tested chimeric antibodies showed good ADCC effect.

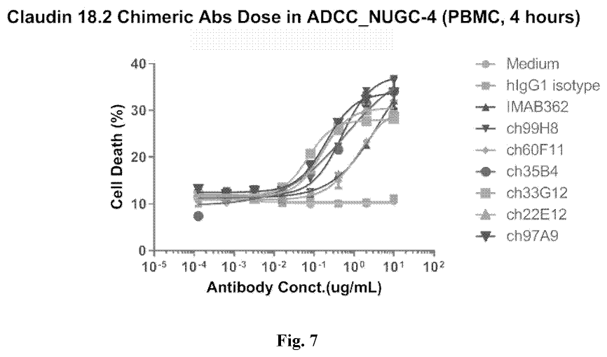

The dose response of the 6 chimeric antibodies (i.e., ch99H8, ch97A9, ch60F11, ch35B4, ch22E12, ch33G12) in ADCC with NUGC-4 cells as the target cells were performed with the procedures similar to those described above, and the result was shown in FIG. 7. As shown in FIG. 7, all of the 6 tested chimeric antibodies demonstrated activity in inducing NUGC-4 cell death.

The dose response of the 6 chimeric antibodies (i.e., ch99H8, ch97A9, ch60F11, ch35B4, ch22E12, ch33G12) in ADCC with HEK293-hCLDN18.1 cells as the target cells were performed with the procedures similar to those described above (except that the target cells are HEK293-hCLDN18.1 cells, the E/T=12.5:1, the incubation time is 3 h), and the result was shown in FIG. 8. As shown in FIG. 8, except for ch33G12, all of the other tested chimeric antibodies did not induce significantly increased HEK293 cell death compared to the reference antibody IMAB362.

### 3.3. Chimeric Antibody Characterization: Complement Dependent Cytotoxicity (CDC) Study

The capabilities of the 6 chimeric antibodies (i.e., ch99H8, ch97A9, ch60F11, ch35B4, ch22E12, ch33G12) to induce CDC activity were evaluated using the HEK293 cells overexpressing hCLDN18.2. The target cells, i.e. HEK293-hCLDN18.2 cells, were co-cultured with normal human serum (25%) for 2 h with or without antibodies, and then the cells were collected to stain live/dead using PI and analyze by FACS.

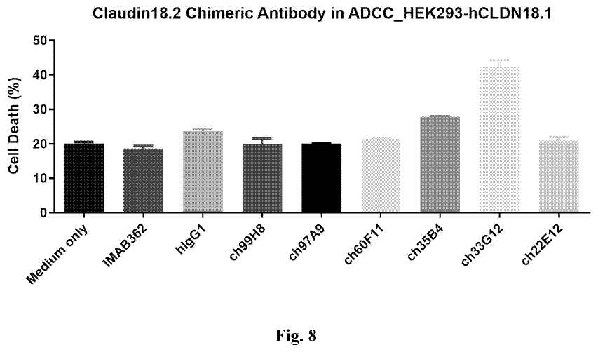

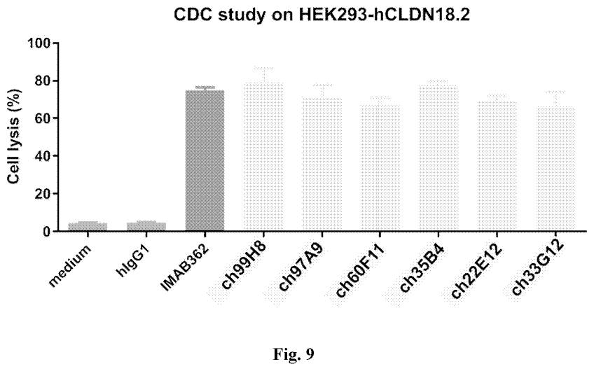

The CDC study result was shown in Table 22 above and FIG. 9. As shown in FIG. 9, all of the tested chimeric antibodies showed good CDC effect, which is comparable to the reference antibody IMAB362.

### Example 4. Antibody Humanization and Affinity Maturation

### 4.1. Humanization

The antibodies 22E12 and 35B4 were selected as the clones for humanization. Antibody sequences were subjected to profiling using sequences alignment, to identify best matched germline and then using the best fit model to identify those back mutation sites. The optimized mutants were synthesized and recombinant antibodies were produced for binding affinity determined by FCM.

After grafting and back mutation, the affinities of some 35B4 and 22E12 humanized antibodies were retained. Those best performance were subjected to functional evaluation by ADCC and/or CDC assay.

A total of 15 humanized antibody clones were obtained for clone 35B4, mixing and matching 3 variants of humanized 35B4 heavy chain variable regions (i.e. hu35B4.H1, hu35B4.H2, and hu35B4.H3) and 5 variants of humanized 35B4 light chain variable regions (i.e. hu35B4.L1, hu35B4.L2, hu35B4.L3, hu35B4.L4, and hu35B4.L1S92A). The 15 humanized antibody clones were designated as hu35B4.H1L1, hu35B4.H1L2, and so on, as shown in Table 23 below, where the prefix “hu” indicates “humanized”, and the suffix “H1L1”, for example, denotes the serial number of the humanized 35B4 antibody clone, having the hu35B4.H1 variant and the hu35B4.L1 variant variable regions.

TABLE 23
Heavy and light chain variable regions of humanized antibodies for 35B4.

| | hu35B4.H1 | hu35B4.H2 | hu35B4.H3 |
|---|---|---|---|
| | (SEQ ID NO: 311) | (SEQ ID NO: 312) | (SEQ ID NO: 313) |
| hu35B4.L1 | hu35B4.H1L1 | hu35B4.H2L1 | hu35B4.H3L1 |
| (SEQ ID NO: 314) | (SEQ ID NOs: | (SEQ ID NOs: | (SEQ ID NOs: |
| | 311/314) | 312/314) | 313/314) |
| hu35B4.L2 | hu35B4.H1L2 | hu35B4.H2L2 | hu35B4.H3L2 |
| (SEQ ID NO: 315) | (SEQ ID NOs: | (SEQ ID NOs: | (SEQ ID NOs: |
| | 311/315) | 312/315) | 313/315) |
| hu35B4.L3 | hu35B4.H1L3 | hu35B4.H2L3 | hu35B4.H3L3 |
| (SEQ ID NO: 316) | (SEQ ID NOs: | (SEQ ID NOs: | (SEQ ID NOs: |
| | 311/316) | 312/316) | 313/316) |
| hu35B4.L4 | hu35B4.H1L4 | hu35B4.H2L4 | hu35B4.H3L4 |
| (SEQ ID NO: 317) | (SEQ ID NOs: | (SEQ ID NOs: | (SEQ ID NOs: |
| | 311/317) | 312/317) | 313/317) |
| hu35B4.L1S92A | hu35B4.H1L1S92A | hu35B4.H2L1S92A | hu35B4.H3L1S92A |
| (SEQ ID NO: 402) | (SEQ ID NOs: | (SEQ ID NOs: | (SEQ ID NOs: |
| | 311/402) | 312/402) | 313/402) |

Similarly, a total of 12 humanized antibodies were obtained for clone 22E12, mixing and matching 4 variants of humanized 22E12 heavy chain variable regions (i.e. hu22E12.H1, hu22E12.H2, hu22E12.H3, hu22E12.H4) and 3 variants of humanized 22E12 light chain variable regions (i.e. hu22E12.L1, hu22E12.L2, hu22E12.L3). The 12 humanized antibody clones were designated as hu22E12.H1L1, hu22E12.H1L2, and so on, as shown in below Table 24, by the same token.

TABLE 24
Heavy and light chain variable regions of humanized antibodies for 22E12.

| | hu22E12.H1 | hu22E12.H2 | hu22E12.H3 | hu22E12.H4 |
|---|---|---|---|---|
| | (SEQ ID NO: | (SEQ ID NO: | (SEQ ID NO: | (SEQ ID NO: |
| | 318) | 319) | 320) | 321) |
| hu22E12.L1 | hu22E12.H1L1 | hu22E12.H2L1 | hu22E12.H3L1 | hu22E12.H4L1 |
| (SEQ ID NO: | (SEQ ID NOs: | (SEQ ID NOs: | (SEQ ID NOs: | (SEQ ID NOs: |
| 322) | 318/322) | 319/322) | 320/322) | 321/322) |
| hu22E12.L2 | hu22E12.H1L2 | hu22E12.H2L2 | hu22E12.H3L2 | hu22E12.H4L2 |
| (SEQ ID NO: | (SEQ ID NOs: | (SEQ ID NOs: | (SEQ ID NOs: | (SEQ ID NOs: |
| 323) | 318/323) | 319/323) | 320/323) | 321/323) |
| hu22E12.L3 | hu22E12.H1L3 | hu22E12.H2L3 | hu22E12.H3L3 | hu22E12.H4L3 |
| (SEQ ID NO: | (SEQ ID NOs: | (SEQ ID NOs: | (SEQ ID NOs: | (SEQ ID NOs: |
| 324) | 318/324) | 319/324) | 320/324) | 321/324) |

### 4.2. Humanized Antibody Characterization: Binding Affinity

The humanized antibodies in Tables 23 and 24 were recombinantly produced followed by testing for binding affinity, and were shown to be able to retain specific binding hCLDN18.2. The humanized antibodies for 22E12 and reference antibody IMAB362 were characterized for binding affinity against hCLDN18.2 by FACS assay using MFC cells over-expressing hCLDN18.2. The humanized antibodies for 35B4 and reference antibody IMAB362 were characterized for binding affinity against hCLDN18.2 by FACS assay using SNU620 cells over-expressing hCLDN18.2. Briefly, each of the humanized antibodies and reference antibody was diluted in 2% FBS to the top concentration (200 nM), then the 3-fold serial dilution were performed with 2% FBS. Cells were collected by centrifugation at 400 g for 5 min. The cells were then re-suspended in 2% FBS and plated in 96 well plates, 1×10⁵ cells/ml in 100 μl/well. 200 μl 2% FBS was added and centrifuge at 400 g for 5 min, then wash for once more. The cells were resuspended in 2% FBS containing test antibodies 100 μl/well. The cells were washed with 2% FBS for 2 times and centrifuged at 400 g for 5 min, then the supernatants were discarded. The cells was resuspended in 2% FBS containing secondary antibodies 100 μl/well, incubated at 4° C. for 60 min. The cells were washed with 2% FBS for 2 times and centrifuged at 1000 rpm for 5 min, then the supernatants were discarded. Finally, the cells were resuspended in 100 μl 2% FBS and analyzed by FACS.

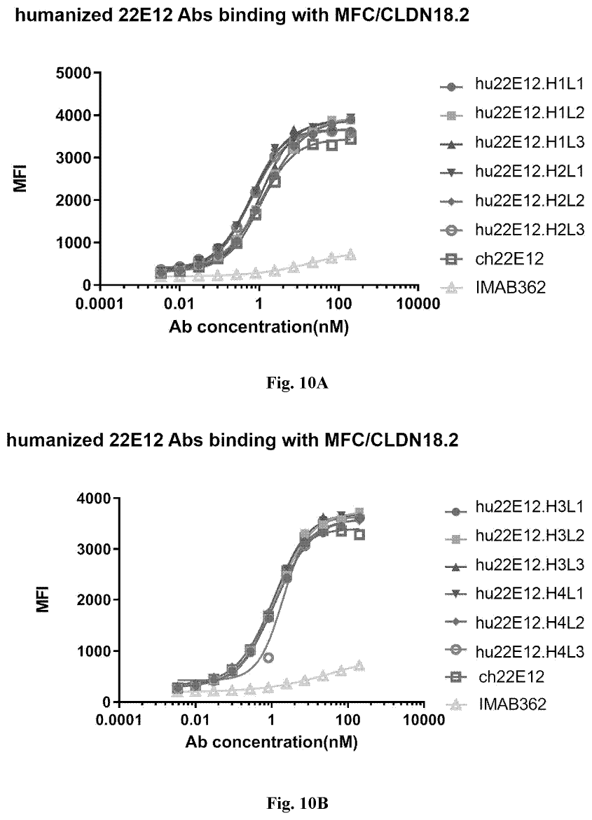

The humanized antibodies showing good binding affinity were shown in Tables 25-26 below and also shown in FIG. 10A, FIG. 10B, FIG. 11A and FIG. 11B. EC₅₀ value is the concentration of the indicated antibodies to reach 50% of the signal in this assay.

TABLE 25
Binding affinities of hu22E12 antibodies to MFC cells

| | hu22E12.H1L1 | hu22E12.H1L2 | hu22E12.H1L3 | hu22E12.H2L1 | hu22E12.H2L2 | hu22E12.H2L3 | ch22E12 | IMAB362 |
|---|---|---|---|---|---|---|---|---|
| EC₅₀ | 1.314 | 1.32 | 1.082 | 0.6562 | 0.5857 | 0.6418 | 1.055 | 23.52 |
| (nM) | | | | | | | | |
| | hu22E12.H3L1 | hu22E12.H3L2 | hu22E12.H3L3 | hu22E12.H4L1 | hu22E12.H4L2 | hu22E12.H4L3 | ch22E12 | IMAB362 |
| EC₅₀ | 1.233 | 1.25 | 1.191 | 1.156 | 1.054 | 1.999 | 0.9434 | 42.69 |
| (nM) | | | | | | | | |

TABLE 26
Binding affinities of hu35B4 antibodies to SNU620 cells

| | hu35B4.H1L1 | hu35B4.H1L2 | hu35B4.H1L3 | hu35B4.H1L4 | hu35B4.H2L1 | hu35B4.H2L2 | ch35B4 | IMAB362 | hIgG4 |
|---|---|---|---|---|---|---|---|---|---|
| EC₅₀ | 0.525 | 0.2526 | 0.3818 | 0.4738 | 0.3548 | 0.439 | 0.4192 | NA | NA |
| (nM) | | | | | | | | | |
| | hu35B4.H2L3 | hu35B4.H2L4 | hu35B4.H3L1 | hu35B4.H3L2 | hu35B4.H3L3 | hu35B4.H3L4 | ch35B4 | IMAB362 | hIgG4 |
| EC₅₀ | 0.4126 | 0.3667 | 0.2584 | 0.2967 | 0.3194 | 0.3502 | 0.4421 | NA | NA |
| (nM) | | | | | | | | | |

### 4.3. Humanized Antibody Characterization: ADCC Study

The humanized antibodies having relatively higher affinity (e.g. hu22E12.H1L2, hu35B4.H1L2) were further evaluated in functional assays including ADCC study. The ADCC study was performed in HEK293/hCLDN18.2 cells or NUGC4 cells by similar methods as described in Example 3.2 above (except that the effector cells are NK-CD16a cells).

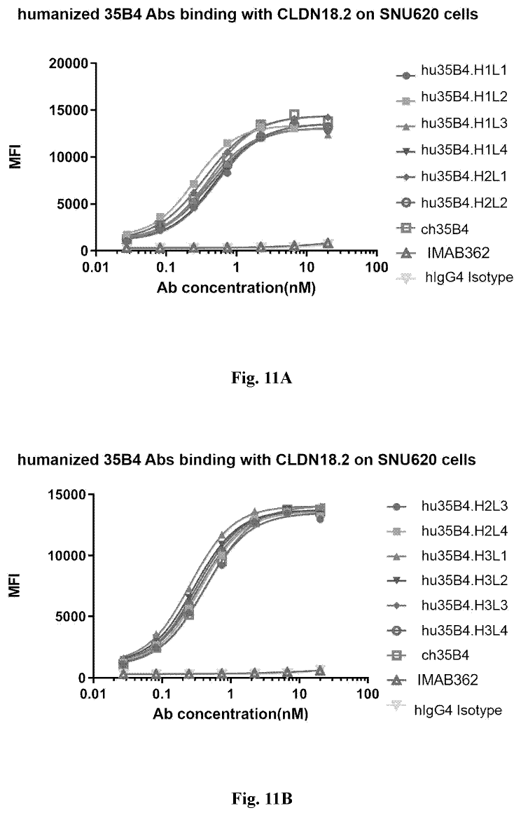

The ADCC study results were shown in FIG. 12A (HEK293/hCLDN18.2 cells) and FIG. 12B (NUGC4 cells), respectively. As shown in FIG. 12A and FIG. 12B, both of the tested humanized antibodies (i.e. hu22E12.H1L2, hu35B4.H1L2) showed similar good ADCC effect to the parental chimeric antibody and better efficacy compared to the reference antibody IMAB362.

### Example 5. ADCC Enhancement by Fc Engineering

### 5.1. Fc Engineering

Human IgG1 (hIgG1) Fc region was set as the template to make point mutation to modulate its binding to Fc gamma receptor and/or complement. Totally 6 engineered Fc regions were developed and shown in Table 27 below. The recombinant antibodies of ch99H8, ch22E12, humanized 22E12 and humanized 35B4 with those engineered Fc regions were produced as above described. And they were subjected to ADCC induction activity evaluation.

TABLE 27
Modification(s) to hIgG1 Fc region

| Mutation Name | Mutation Sites |
|---|---|
| ADE | G236A, S239D, I332E |
| DLE | S239D, A330L, I332E |
| DE | S239D, I332E |
| DFTE | S239D, H268F, S324T, I332E |
| LPLIL | F243L, R292P, Y300L, V305I, P396L |
| VLPLL | L235V, F243L, R292P, Y300L, P396L |

### 5.2. ADCC Activity of Fc Engineered Antibodies

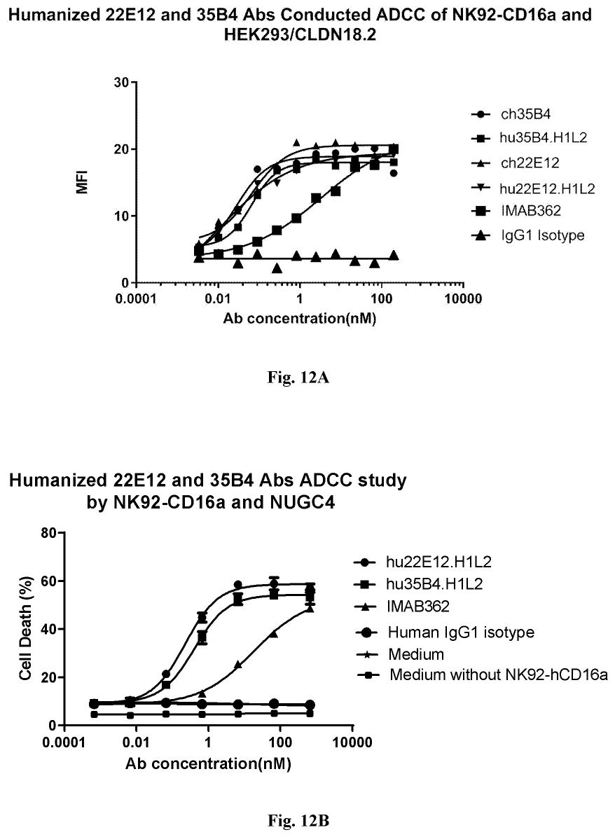

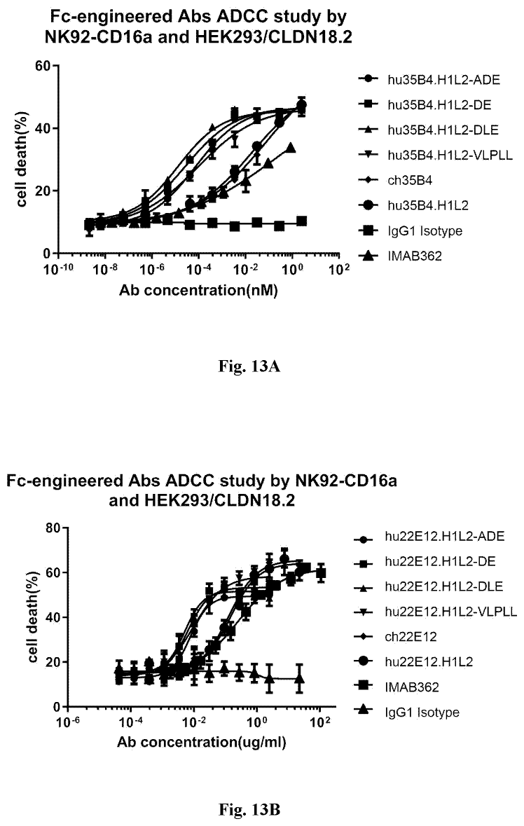

The capabilities of the Fe engineered antibodies to induce ADCC activity were evaluated using the HEK293 cells overexpressing hCLDN18.2. The ADCC study for Fc engineered antibodies were conducted by similar methods as described in Example 3.2 above (except that the effector cells are NK-CD16a cells). The ADCC study results were shown in FIG. 13A and FIG. 13B, and the EC₅₀ values were shown in Tables 28 and 29 below. As shown in Table 28, Table 29, FIG. 13A and FIG. 13B, all of the tested Fc engineered antibodies showed enhanced ADCC effect compared to the antibodies without Fc engineering.

TABLE 28
EC₅₀ values of Fc engineered humanized 35B4 antibodies

| Antibody ID | EC₅₀ (nM) |
|---|---|
| hu35B4.H1L2-ADE | 7.104E−05 |
| hu35B4.H1L2-DLE | 1.322E−05 |
| hu35B4.H1L2-DE | 2.751E−05 |
| hu35B4.H1L2-VLPLL | 9.433E−05 |
| ch35B4 | 0.08028 |
| hu35B4.H1L2 | 0.02002 |
| hIgG1 Isotype | N/A |
| IMAB362 | N/A |

TABLE 29
EC₅₀ values of Fc engineered humanized 22E12 antibodies

| Antibody ID | EC₅₀ (nM) |
|---|---|
| hu22E12.H1L2-ADE | 0.007649 |
| hu22E12.H1L2-DLE | 0.005027 |
| hu22E12.H1L2-DE | 0.006373 |
| hu22E12.H1L2-VLPLL | 0.009973 |
| ch22E12 | 0.1255 |
| hu22E12.H1L2 | 0.1444 |
| hIgG1 isotype | N/A |
| IMAB362 | 0.228 |

### Example 6. Immunohistochemistry (IHC) Staining

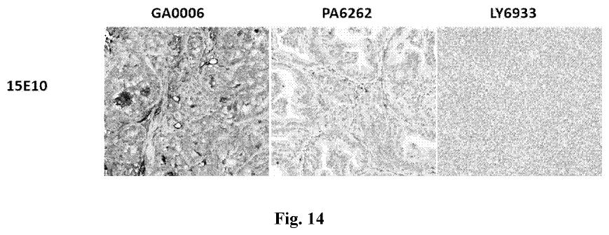

IHC staining was performed for several exemplary antibodies. GA006 is a patient-derived tumor xenograft (PDX) model (CLDN18.2 high expression) of gastric cancer, PA6262 is a PDX model (CLDN18.2 low expression) of pancreatic cancer, LY6933 is a PDX model (no claudin18.2 expression) of lyphoma. The PDX tumor samples were collected and paraffin-embedded sections were prepared. Anti-CLDN18.2 antibodies, 60F11, 40C1, 35B4, 35A10, 22E12, 33G12, 15E10, 97A9, 84F2, 38B9, 73E4 and 99H8 were then stained and it's binding were detected using HRP labeled anti-mouse IgG antibody. After development with DAB substrate, the colors of the antibody staining in the tissue sections were observed under microscopy. Representative images of IHC were shown in FIG. 14. As shown in FIG. 14, antibody 15E10 binds to GA0006 with the highest scores, and binds to LY6933 with the lowest scores, which suggested that antibody 15E10 is useful in diagnosing CLDN18 (especially CLDN18.2) related diseases. The results of the other tested antibodies were similar and were not shown herein.

### Example 7. Kinetics Study Using Surface Plasmon Resonance (SPR)

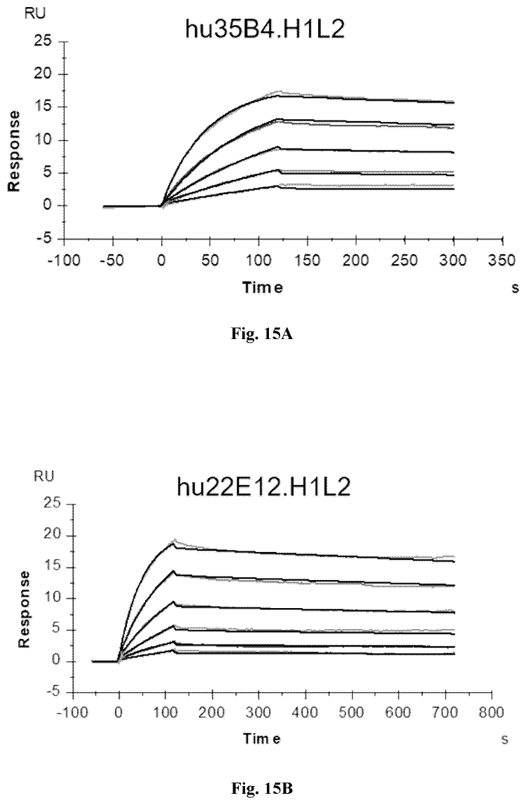

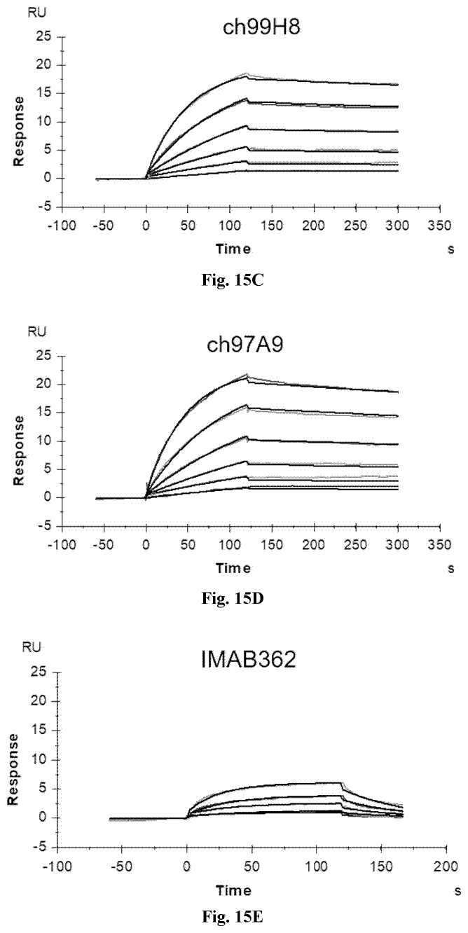

Kinetics study was performed for several exemplary antibodies. In particular, humanized antibodies hu35B4.H1L2 and hu22E12.H1L2, chimeric antibodies ch99H8 and ch97A9, and reference antibody IMAB362 were characterized for binding affinity against CLDN18.2 using Biacore (GE). Briefly, the recombinant CLDN18.2 protein was immobilized to CM5 chip (GE) using amine coupling kit (GE). The antigen of 6×His tagged human CLDN18.2 was diluted to 10 g/ml for immobilization (immobilization time: 120 s). The antibodies to be tested were serially diluted for multiple doses to the top concentration of 200 nM (hu35B4.H1L2), 25 nM (hu22E12.H1L2), 100 nM (ch99H8), 50 nM (ch97A9), and 100 nM (IMAB362), respectively. The association time for all tested antibodies is 120 s. The dissociation time is 600 s (for hu22E12.H1L2) or 180 s (for other tested antibodies). The kinetics analysis was performed by the Biacore T200 Analysis V10 using the 1:1 Global Fitting. The Ka/Kd/KD values for each antibody were calculated. The affinity data of the tested antibodies are summarized in Table 30 below and shown in FIGS. 15A to 15E. As shown in Table 30 and FIGS. 15A-15E, the tested humanized and chimeric antibodies showed good affinity compared to the reference antibody IMAB362, and they are ranking as hu22E12.H1L2>hu35B4.H1L2, ch99H8, ch97A9>IMAB362.

TABLE 30
Binding affinity of antibodies to CLDN18.2 as measured by SPR

| | | | | Rmax | Chi² | | |
|---|---|---|---|---|---|---|---|
| Antibody | ka (1/Ms) | kd (1/s) | KD (M) | (RU) | (RU²) | Ligand | Model |
| hu35B4.H1L2 | 2.22E+05 | 3.50E−04 | 1.57E−09 | 18.2 | 0.0992 | CLDN18.2 | 1:1 |
| | | | | | | | Binding |
| hu22E12.H1L2 | 7.78E+05 | 2.02E−04 | 2.6E−10 | 20.1 | 0.0641 | CLDN18.2 | 1:1 |
| | | | | | | | Binding |
| ch99H8 | 2.03E+05 | 3.55E−04 | 1.75E−09 | 19.6 | 0.0341 | CLDN18.2 | 1:1 |
| | | | | | | | Binding |
| ch97A9 | 4.25E+05 | 4.89E−04 | 1.15E−09 | 22.5 | 0.106 | CLDN18.2 | 1:1 |
| | | | | | | | Binding |
| IMAB362 | 1.81E+05 | 2.08E−02 | 1.15E−07 | 10.7 | 0.023 | CLDN18.2 | 1:1 |
| | | | | | | | Binding |

### Example 8. Antibody Induced Target Internalization on SNU620 Cells

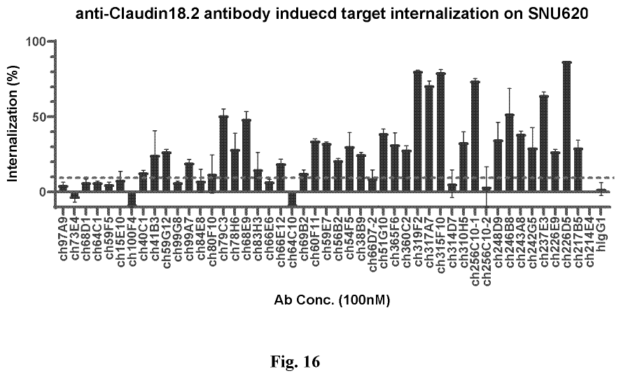

The internalization rates of several exemplary antibodies were assayed to evaluate their potency in antibody-drug conjugate area. Briefly, the tested antibodies were diluted to 200 nM and aliquoted to two plates in 50 μl/Ab/well, duplicated. SNU620 cells were cultured and collected as mononuclear cells and then 2×10⁵ cells in 50 μl FACS buffer were added into the antibody plates. Cell plates were incubated at 4° C. for half an hour for antibody binding and then the unbound antibody was removed by several cycles of spin-down and re-suspension using FACS buffer. 100 μl cell culture medium was added to suspend cells, one plate was incubated at 4 degree for 2 hours and the counter plates were incubated at 37° C. for 2 hrs. After several rounds wash, all cells were stained using fluorescence labeled anti-hIgG antibody at 4° C. for 1 hour after 3 repeats of washing step, and then read out using FACS. The internalization rate was calculated by using the following equation, and the results were shown in FIG. 16.

$$Internalization ( \% ) = ( MFI ( 4 ° C . ) - MFI ( 37 ° C . ) ) / MFI ( 4 ° C . )$$

As shown in FIG. 16, some tested anti-CLDN18.2 chimeric antibodies were efficiently internalized upon binding to CLDN18.2, such as ch319F2, ch317A7, ch315F10, ch256C10⁻¹, ch226D5, etc., suggesting that they are suitable for the further evaluation as antibody drug conjugates.

### Example 9. Combination Treatment of Anti-CLDN18.2 Antibody and Anti-SIRPα Antibody

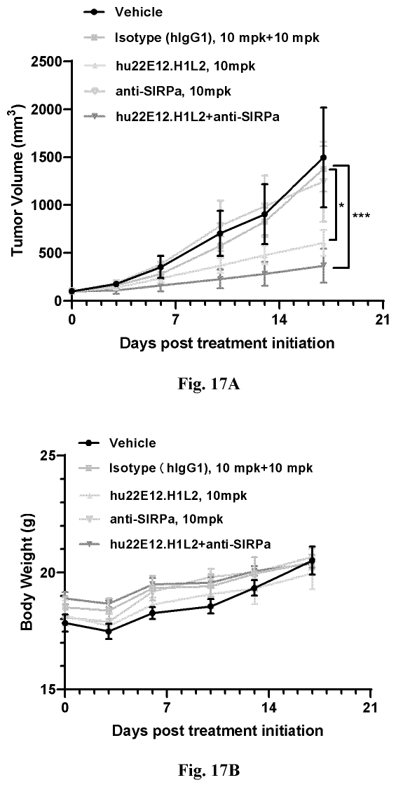

The in vivo efficacy of combination treatment of the anti-CLDN18.2 antibody of the present invention and anti-SIRP^{α} antibody was evaluated in this example. Briefly, hCD47/hCLDN18.2 overexpressing MC38 cells were inoculated into hCD47/hSIRPα double knock-in C57BL/6 mice, and the tumor size at DO (i.e. Day 0) was around 100 mm³. The mice were divided into five groups (seven mice each group), and the mice in each group were treated with vehicle, human IgG1 isotype, hu22E12.H1L2 alone, anti-SIRPα antibody (in-house prepared) alone, and hu22E12.H1L2+anti-SIRPα antibody combo, respectively, i.p., twice a week. The tumor size and body weight of each mouse were measured twice a week. The tumor volume changes and body weight changes in mice over the days post treatments were shown in FIG. 17A and FIG. 17B. As shown in FIG. 17A and FIG. 17B, the humanized antibody hu22E12.H1L2 significantly inhibited the tumor cells expressing CLDN18.2, and anti-SIRPα antibody significantly improved hu22E12.H1L2's in vivo efficacy.

1. An antibody or an antigen-binding fragment thereof capable of specifically binding to CLDN18, comprising a heavy chain variable region comprising HCDR1, HCDR2 and HCDR3 and/or a light chain variable region comprising LCDR1, LCDR2 and LCDR3, wherein
a) the HCDR1 comprises an amino acid sequence selected from the group consisting of SEQ ID NOs: 1-28, 201, 202, 332-337; or
b) the HCDR2 comprises an amino acid sequence selected from the group consisting of SEQ ID NOs: 29-67, 203, 338-343, 367; or
c) the HCDR3 comprises an amino acid sequence selected from the group consisting of SEQ ID NOs: 68-94, 344-346; or
d) the LCDR1 comprises an amino acid sequence selected from the group consisting of SEQ ID NOs: 95-113, 205, 347, 348; or
e) the LCDR2 comprises an amino acid sequence selected from the group consisting of SEQ ID NOs: 114-123, 349, 350; or
f) the LCDR3 comprises an amino acid sequence selected from the group consisting of SEQ ID NOs: 124-155, 204, 351-354.

2-3. (canceled)

4. The antibody or an antigen-binding fragment thereof of claim 1, wherein:
a) the HCDR1 comprises an amino acid sequence selected from the group consisting of SEQ ID NOs: 16, 19, 201, 202, and/or
b) the HCDR2 comprises an amino acid sequence selected from the group consisting of SEQ ID NOs: 47, 50, 203 and/or
c) the HCDR3 comprises an amino acid sequence selected from the group consisting of SEQ ID NO: 80, 83, and/or
d) the LCDR1 comprises an amino acid sequence selected from the group consisting of SEQ ID NOs: 96, 103, 205, and/or
e) the LCDR2 comprises an amino acid sequence selected from the group consisting of SEQ ID NO: 118, 120, and/or
f) the LCDR3 comprises an amino acid sequence selected from the group consisting of SEQ ID NOs: 138, 141, 204.

5. The antibody or an antigen-binding fragment thereof of claim 1, wherein the heavy chain variable region comprises:
(1) a HCDR1 comprising the sequence of SEQ ID NO: 1, a HCDR2 comprising the sequence of SEQ ID NO: 29, and a HCDR3 comprising the sequence of SEQ ID NO: 68; or
(2) a HCDR1 comprising the sequence of SEQ ID NO: 2, a HCDR2 comprising the sequence of SEQ ID NO: 30, and a HCDR3 comprising the sequence of SEQ ID NO: 69; or
(3) a HCDR1 comprising the sequence of SEQ ID NO: 3, a HCDR2 comprising the sequence of SEQ ID NO: 31, and a HCDR3 comprising the sequence of SEQ ID NO: 70; or
(4) a HCDR1 comprising the sequence of SEQ ID NO: 4, a HCDR2 comprising the sequence of SEQ ID NO: 32, and a HCDR3 comprising the sequence of SEQ ID NO: 69; or
(5) a HCDR1 comprising the sequence of SEQ ID NO: 5, a HCDR2 comprising the sequence of SEQ ID NO: 33, and a HCDR3 comprising the sequence of SEQ ID NO: 71; or
(6) a HCDR1 comprising the sequence of SEQ ID NO: 4, a HCDR2 comprising the sequence of SEQ ID NO: 34, and a HCDR3 comprising the sequence of SEQ ID NO: 69; or
(7) a HCDR1 comprising the sequence of SEQ ID NO: 6, a HCDR2 comprising the sequence of SEQ ID NO: 32, and a HCDR3 comprising the sequence of SEQ ID NO: 69; or
(8) a HCDR1 comprising the sequence of SEQ ID NO: 7, a HCDR2 comprising the sequence of SEQ ID NO: 35, and a HCDR3 comprising the sequence of SEQ ID NO: 72; or
(9) a HCDR1 comprising the sequence of SEQ ID NO: 8, a HCDR2 comprising the sequence of SEQ ID NO: 36, and a HCDR3 comprising the sequence of SEQ ID NO: 72; or
(10) a HCDR1 comprising the sequence of SEQ ID NO: 6, a HCDR2 comprising the sequence of SEQ ID NO: 37, and a HCDR3 comprising the sequence of SEQ ID NO: 69; or
(11) a HCDR1 comprising the sequence of SEQ ID NO: 5, a HCDR2 comprising the sequence of SEQ ID NO: 38, and a HCDR3 comprising the sequence of SEQ ID NO: 73; or
(12) a HCDR1 comprising the sequence of SEQ ID NO: 8, a HCDR2 comprising the sequence of SEQ ID NO: 35, and a HCDR3 comprising the sequence of SEQ ID NO: 74; or
(13) a HCDR1 comprising the sequence of SEQ ID NO: 2, a HCDR2 comprising the sequence of SEQ ID NO: 39, and a HCDR3 comprising the sequence of SEQ ID NO: 69; or
(14) a HCDR1 comprising the sequence of SEQ ID NO: 9, a HCDR2 comprising the sequence of SEQ ID NO: 40, and a HCDR3 comprising the sequence of SEQ ID NO: 75; or
(15) a HCDR1 comprising the sequence of SEQ ID NO: 9, a HCDR2 comprising the sequence of SEQ ID NO: 41, and a HCDR3 comprising the sequence of SEQ ID NO: 76; or
(16) a HCDR1 comprising the sequence of SEQ ID NO: 10, a HCDR2 comprising the sequence of SEQ ID NO: 42, and a HCDR3 comprising the sequence of SEQ ID NO: 77; or
(17) a HCDR1 comprising the sequence of SEQ ID NO: 11, a HCDR2 comprising the sequence of SEQ ID NO: 43, and a HCDR3 comprising the sequence of SEQ ID NO: 71; or
(18) a HCDR1 comprising the sequence of SEQ ID NO: 12, a HCDR2 comprising the sequence of SEQ ID NO: 44, and a HCDR3 comprising the sequence of SEQ ID NO: 75; or
(19) a HCDR1 comprising the sequence of SEQ ID NO: 13, a HCDR2 comprising the sequence of SEQ ID NO: 40, and a HCDR3 comprising the sequence of SEQ ID NO: 75; or
(20) a HCDR1 comprising the sequence of SEQ ID NO: 14, a HCDR2 comprising the sequence of SEQ ID NO: 45, and a HCDR3 comprising the sequence of SEQ ID NO: 78; or
(21) a HCDR1 comprising the sequence of SEQ ID NO: 8, a HCDR2 comprising the sequence of SEQ ID NO: 35, and a HCDR3 comprising the sequence of SEQ ID NO: 72; or
(22) a HCDR1 comprising the sequence of SEQ ID NO: 15, a HCDR2 comprising the sequence of SEQ ID NO: 46, and a HCDR3 comprising the sequence of SEQ ID NO: 79; or
(23) a HCDR1 comprising the sequence of SEQ ID NO: 16, a HCDR2 comprising the sequence of SEQ ID NO: 47, and a HCDR3 comprising the sequence of SEQ ID NO: 80; or
(24) a HCDR1 comprising the sequence of SEQ ID NO: 201, a HCDR2 comprising the sequence of SEQ ID NO: 47, and a HCDR3 comprising the sequence of SEQ ID NO: 80; or
(25) a HCDR1 comprising the sequence of SEQ ID NO: 17, a HCDR2 comprising the sequence of SEQ ID NO: 48, and a HCDR3 comprising the sequence of SEQ ID NO: 81; or
(26) a HCDR1 comprising the sequence of SEQ ID NO: 18, a HCDR2 comprising the sequence of SEQ ID NO: 49, and a HCDR3 comprising the sequence of SEQ ID NO: 82; or
(27) a HCDR1 comprising the sequence of SEQ ID NO: 19, a HCDR2 comprising the sequence of SEQ ID NO: 50, and a HCDR3 comprising the sequence of SEQ ID NO: 83; or
(28) a HCDR1 comprising the sequence of SEQ ID NO: 202, a HCDR2 comprising the sequence of SEQ ID NO: 50, and a HCDR3 comprising the sequence of SEQ ID NO: 83; or
(29) a HCDR1 comprising the sequence of SEQ ID NO: 202, a HCDR2 comprising the sequence of SEQ ID NO: 203, and a HCDR3 comprising the sequence of SEQ ID NO: 83; or
(30) a HCDR1 comprising the sequence of SEQ ID NO: 17, a HCDR2 comprising the sequence of SEQ ID NO: 51, and a HCDR3 comprising the sequence of SEQ ID NO: 84; or
(31) a HCDR1 comprising the sequence of SEQ ID NO: 2, a HCDR2 comprising the sequence of SEQ ID NO: 52, and a HCDR3 comprising the sequence of SEQ ID NO: 69; or
(32) a HCDR1 comprising the sequence of SEQ ID NO: 20, a HCDR2 comprising the sequence of SEQ ID NO: 53, and a HCDR3 comprising the sequence of SEQ ID NO: 85; or
(33) a HCDR1 comprising the sequence of SEQ ID NO: 21, a HCDR2 comprising the sequence of SEQ ID NO: 54, and a HCDR3 comprising the sequence of SEQ ID NO: 86; or
(34) a HCDR1 comprising the sequence of SEQ ID NO: 22, a HCDR2 comprising the sequence of SEQ ID NO: 55, and a HCDR3 comprising the sequence of SEQ ID NO: 87; or
(35) a HCDR1 comprising the sequence of SEQ ID NO: 2, a HCDR2 comprising the sequence of SEQ ID NO: 56, and a HCDR3 comprising the sequence of SEQ ID NO: 69; or
(36) a HCDR1 comprising the sequence of SEQ ID NO: 1, a HCDR2 comprising the sequence of SEQ ID NO: 57, and a HCDR3 comprising the sequence of SEQ ID NO: 69; or
(37) a HCDR1 comprising the sequence of SEQ ID NO: 11, a HCDR2 comprising the sequence of SEQ ID NO: 43, and a HCDR3 comprising the sequence of SEQ ID NO: 88; or
(38) a HCDR1 comprising the sequence of SEQ ID NO: 23, a HCDR2 comprising the sequence of SEQ ID NO: 58, and a HCDR3 comprising the sequence of SEQ ID NO: 89; or
(39) a HCDR1 comprising the sequence of SEQ ID NO: 24, a HCDR2 comprising the sequence of SEQ ID NO: 59, and a HCDR3 comprising the sequence of SEQ ID NO: 90; or
(40) a HCDR1 comprising the sequence of SEQ ID NO: 25, a HCDR2 comprising the sequence of SEQ ID NO: 60, and a HCDR3 comprising the sequence of SEQ ID NO: 90; or
(41) a HCDR1 comprising the sequence of SEQ ID NO: 26, a HCDR2 comprising the sequence of SEQ ID NO: 61, and a HCDR3 comprising the sequence of SEQ ID NO: 91; or
(42) a HCDR1 comprising the sequence of SEQ ID NO: 26, a HCDR2 comprising the sequence of SEQ ID NO: 62, and a HCDR3 comprising the sequence of SEQ ID NO: 92; or
(43) a HCDR1 comprising the sequence of SEQ ID NO: 27, a HCDR2 comprising the sequence of SEQ ID NO: 367, and a HCDR3 comprising the sequence of SEQ ID NO: 93; or
(44) a HCDR1 comprising the sequence of SEQ ID NO: 26, a HCDR2 comprising the sequence of SEQ ID NO: 63, and a HCDR3 comprising the sequence of SEQ ID NO: 92; or
(45) a HCDR1 comprising the sequence of SEQ ID NO: 26, a HCDR2 comprising the sequence of SEQ ID NO: 64, and a HCDR3 comprising the sequence of SEQ ID NO: 92; or
(46) a HCDR1 comprising the sequence of SEQ ID NO: 28, a HCDR2 comprising the sequence of SEQ ID NO: 65, and a HCDR3 comprising the sequence of SEQ ID NO: 85; or
(47) a HCDR1 comprising the sequence of SEQ ID NO: 28, a HCDR2 comprising the sequence of SEQ ID NO: 66, and a HCDR3 comprising the sequence of SEQ ID NO: 94; or
(48) a HCDR1 comprising the sequence of SEQ ID NO: 28, a HCDR2 comprising the sequence of SEQ ID NO: 66, and a HCDR3 comprising the sequence of SEQ ID NO: 85; or
(49) a HCDR1 comprising the sequence of SEQ ID NO: 26, a HCDR2 comprising the sequence of SEQ ID NO: 67, and a HCDR3 comprising the sequence of SEQ ID NO: 92; or
(50) a HCDR1 comprising the sequence of SEQ ID NO: 11, a HCDR2 comprising the sequence of SEQ ID NO: 61, and a HCDR3 comprising the sequence of SEQ ID NO: 91.

6. The antibody or an antigen-binding fragment thereof of claim 1, wherein the light chain variable region comprises:
(1) a LCDR1 comprising the sequence of SEQ ID NO: 95, a LCDR2 comprising the sequence of SEQ ID NO: 114, and a LCDR3 comprising the sequence of SEQ ID NO: 124; or
(2) a LCDR1 comprising the sequence of SEQ ID NO: 96, a LCDR2 comprising the sequence of SEQ ID NO: 114, and a LCDR3 comprising the sequence of SEQ ID NO: 125; or
(3) a LCDR1 comprising the sequence of SEQ ID NO: 96, a LCDR2 comprising the sequence of SEQ ID NO: 115, and a LCDR3 comprising the sequence of SEQ ID NO: 126; or
(4) a LCDR1 comprising the sequence of SEQ ID NO: 96, a LCDR2 comprising the sequence of SEQ ID NO: 115, and a LCDR3 comprising the sequence of SEQ ID NO: 127; or
(5) a LCDR1 comprising the sequence of SEQ ID NO: 96, a LCDR2 comprising the sequence of SEQ ID NO: 116, and a LCDR3 comprising the sequence of SEQ ID NO: 128; or
(6) a LCDR1 comprising the sequence of SEQ ID NO: 97, a LCDR2 comprising the sequence of SEQ ID NO: 115, and a LCDR3 comprising the sequence of SEQ ID NO: 129; or
(7) a LCDR1 comprising the sequence of SEQ ID NO: 96, a LCDR2 comprising the sequence of SEQ ID NO: 115, and a LCDR3 comprising the sequence of SEQ ID NO: 130; or
(8) a LCDR1 comprising the sequence of SEQ ID NO: 98, a LCDR2 comprising the sequence of SEQ ID NO: 115, and a LCDR3 comprising the sequence of SEQ ID NO: 127; or
(9) a LCDR1 comprising the sequence of SEQ ID NO: 96, a LCDR2 comprising the sequence of SEQ ID NO: 115, and a LCDR3 comprising the sequence of SEQ ID NO: 128; or
(10) a LCDR1 comprising the sequence of SEQ ID NO: 99, a LCDR2 comprising the sequence of SEQ ID NO: 115, and a LCDR3 comprising the sequence of SEQ ID NO: 131; or
(11) a LCDR1 comprising the sequence of SEQ ID NO: 99, a LCDR2 comprising the sequence of SEQ ID NO: 115, and a LCDR3 comprising the sequence of SEQ ID NO: 132; or
(12) a LCDR1 comprising the sequence of SEQ ID NO: 99, a LCDR2 comprising the sequence of SEQ ID NO: 115, and a LCDR3 comprising the sequence of SEQ ID NO: 133; or
(13) a LCDR1 comprising the sequence of SEQ ID NO: 100, a LCDR2 comprising the sequence of SEQ ID NO: 115, and a LCDR3 comprising the sequence of SEQ ID NO: 134; or
(14) a LCDR1 comprising the sequence of SEQ ID NO: 101, a LCDR2 comprising the sequence of SEQ ID NO: 115, and a LCDR3 comprising the sequence of SEQ ID NO: 135; or
(15) a LCDR1 comprising the sequence of SEQ ID NO: 96, a LCDR2 comprising the sequence of SEQ ID NO: 115, and a LCDR3 comprising the sequence of SEQ ID NO: 136; or
(16) a LCDR1 comprising the sequence of SEQ ID NO: 102, a LCDR2 comprising the sequence of SEQ ID NO: 117, and a LCDR3 comprising the sequence of SEQ ID NO: 137; or
(17) a LCDR1 comprising the sequence of SEQ ID NO: 103, a LCDR2 comprising the sequence of SEQ ID NO: 118, and a LCDR3 comprising the sequence of SEQ ID NO: 138; or
(18) a LCDR1 comprising the sequence of SEQ ID NO: 103, a LCDR2 comprising the sequence of SEQ ID NO: 118, and a LCDR3 comprising the sequence of SEQ ID NO: 204; or
(19) a LCDR1 comprising the sequence of SEQ ID NO: 96, a LCDR2 comprising the sequence of SEQ ID NO: 119, and a LCDR3 comprising the sequence of SEQ ID NO: 139; or
(20) a LCDR1 comprising the sequence of SEQ ID NO. 104, a LCDR2 comprising the sequence of SEQ ID NO: 118, and a LCDR3 comprising the sequence of SEQ ID NO: 140; or
(21) a LCDR1 comprising the sequence of SEQ ID NO: 96, a LCDR2 comprising the sequence of SEQ ID NO: 120, and a LCDR3 comprising the sequence of SEQ ID NO: 141; or
(22) a LCDR1 comprising the sequence of SEQ ID NO: 205, a LCDR2 comprising the sequence of SEQ ID NO: 120, and a LCDR3 comprising the sequence of SEQ ID NO: 141; or
(23) a LCDR1 comprising the sequence of SEQ ID NO: 96, a LCDR2 comprising the sequence of SEQ ID NO: 121, and a LCDR3 comprising the sequence of SEQ ID NO: 142; or
(24) a LCDR1 comprising the sequence of SEQ ID NO: 105, a LCDR2 comprising the sequence of SEQ ID NO: 115, and a LCDR3 comprising the sequence of SEQ ID NO: 143; or
(25) a LCDR1 comprising the sequence of SEQ ID NO: 106, a LCDR2 comprising the sequence of SEQ ID NO: 122, and a LCDR3 comprising the sequence of SEQ ID NO: 144; or
(26) a LCDR1 comprising the sequence of SEQ ID NO: 95, a LCDR2 comprising the sequence of SEQ ID NO: 115, and a LCDR3 comprising the sequence of SEQ ID NO: 128; or
(27) a LCDR1 comprising the sequence of SEQ ID NO: 98, a LCDR2 comprising the sequence of SEQ ID NO: 115, and a LCDR3 comprising the sequence of SEQ ID NO: 145; or
(28) a LCDR1 comprising the sequence of SEQ ID NO: 96, a LCDR2 comprising the sequence of SEQ ID NO: 115, and a LCDR3 comprising the sequence of SEQ ID NO: 146; or
(29) a LCDR1 comprising the sequence of SEQ ID NO: 96, a LCDR2 comprising the sequence of SEQ ID NO: 115, and a LCDR3 comprising the sequence of SEQ ID NO: 147; or
(30) a LCDR1 comprising the sequence of SEQ ID NO: 96, a LCDR2 comprising the sequence of SEQ ID NO: 115, and a LCDR3 comprising the sequence of SEQ ID NO: 148; or
(31) a LCDR1 comprising the sequence of SEQ ID NO: 107, a LCDR2 comprising the sequence of SEQ ID NO: 115, and a LCDR3 comprising the sequence of SEQ ID NO: 149; or
(32) a LCDR1 comprising the sequence of SEQ ID NO: 108, a LCDR2 comprising the sequence of SEQ ID NO: 115, and a LCDR3 comprising the sequence of SEQ ID NO: 150; or
(33) a LCDR1 comprising the sequence of SEQ ID NO: 108, a LCDR2 comprising the sequence of SEQ ID NO: 115, and a LCDR3 comprising the sequence of SEQ ID NO: 150; or
(34) a LCDR1 comprising the sequence of SEQ ID NO: 107, a LCDR2 comprising the sequence of SEQ ID NO: 115, and a LCDR3 comprising the sequence of SEQ ID NO: 149; or
(35) a LCDR1 comprising the sequence of SEQ ID NO: 109, a LCDR2 comprising the sequence of SEQ ID NO: 123, and a LCDR3 comprising the sequence of SEQ ID NO: 151; or
(36) a LCDR1 comprising the sequence of SEQ ID NO: 110, a LCDR2 comprising the sequence of SEQ ID NO: 115, and a LCDR3 comprising the sequence of SEQ ID NO: 150; or
(37) a LCDR1 comprising the sequence of SEQ ID NO: 111, a LCDR2 comprising the sequence of SEQ ID NO: 115, and a LCDR3 comprising the sequence of SEQ ID NO: 150; or
(38) a LCDR1 comprising the sequence of SEQ ID NO: 101, a LCDR2 comprising the sequence of SEQ ID NO: 120, and a LCDR3 comprising the sequence of SEQ ID NO: 152; or
(39) a LCDR1 comprising the sequence of SEQ ID NO: 101, a LCDR2 comprising the sequence of SEQ ID NO: 120, and a LCDR3 comprising the sequence of SEQ ID NO: 153; or
(40) a LCDR1 comprising the sequence of SEQ ID NO: 112, a LCDR2 comprising the sequence of SEQ ID NO: 120, and a LCDR3 comprising the sequence of SEQ ID NO: 152; or
(41) a LCDR1 comprising the sequence of SEQ ID NO: 101, a LCDR2 comprising the sequence of SEQ ID NO: 120, and a LCDR3 comprising the sequence of SEQ ID NO: 153; or
(42) a LCDR1 comprising the sequence of SEQ ID NO: 107, a LCDR2 comprising the sequence of SEQ ID NO: 115, and a LCDR3 comprising the sequence of SEQ ID NO: 149; or
(43) a LCDR1 comprising the sequence of SEQ ID NO: 101, a LCDR2 comprising the sequence of SEQ ID NO: 120, and a LCDR3 comprising the sequence of SEQ ID NO: 152; or
(44) a LCDR1 comprising the sequence of SEQ ID NO: 101, a LCDR2 comprising the sequence of SEQ ID NO: 120, and a LCDR3 comprising the sequence of SEQ ID NO: 154; or
(45) a LCDR1 comprising the sequence of SEQ ID NO: 96, a LCDR2 comprising the sequence of SEQ ID NO: 115, and a LCDR3 comprising the sequence of SEQ ID NO: 155; or
(46) a LCDR1 comprising the sequence of SEQ ID NO: 108, a LCDR2 comprising the sequence of SEQ ID NO: 115, and a LCDR3 comprising the sequence of SEQ ID NO: 150; or
(47) a LCDR1 comprising the sequence of SEQ ID NO: 107, a LCDR2 comprising the sequence of SEQ ID NO: 115, and a LCDR3 comprising the sequence of SEQ ID NO: 149; or
(48) a LCDR1 comprising the sequence of SEQ ID NO: 113, a LCDR2 comprising the sequence of SEQ ID NO: 115, and a LCDR3 comprising the sequence of SEQ ID NO: 149; or
(49) a LCDR1 comprising the sequence of SEQ ID NO: 108, a LCDR2 comprising the sequence of SEQ ID NO: 115, and a LCDR3 comprising the sequence of SEQ ID NO: 150; or
(50) a LCDR1 comprising the sequence of SEQ ID NO: 107, a LCDR2 comprising the sequence of SEQ ID NO: 115, and a LCDR3 comprising the sequence of SEQ ID NO: 149; or
(51) a LCDR1 comprising the sequence of SEQ ID NO: 101, a LCDR2 comprising the sequence of SEQ ID NO: 120, and a LCDR3 comprising the sequence of SEQ ID NO: 153.

7. The antibody or an antigen-binding fragment thereof of claim 1, wherein:
(1) the HCDR1 comprises the sequence of SEQ ID NO: 1, the HCDR2 comprises the sequence of SEQ ID NO: 29, the HCDR3 comprises the sequence of SEQ ID NO: 68; the LCDR1 comprises the sequence of SEQ ID NO: 95, the LCDR2 comprises the sequence of SEQ ID NO: 114, and the LCDR3 comprises the sequence of SEQ ID NO: 124; or
(2) the HCDR1 comprises the sequence of SEQ ID NO: 2, the HCDR2 comprises the sequence of SEQ ID NO: 30, the HCDR3 comprises the sequence of SEQ ID NO: 69; the LCDR1 comprises the sequence of SEQ ID NO: 96, the LCDR2 comprises the sequence of SEQ ID NO: 114, and the LCDR3 comprises the sequence of SEQ ID NO: 125; or
(3) the HCDR1 comprises the sequence of SEQ ID NO: 3, the HCDR2 comprises the sequence of SEQ ID NO: 31, the HCDR3 comprises the sequence of SEQ ID NO: 70; the LCDR1 comprises the sequence of SEQ ID NO: 96, the LCDR2 comprises the sequence of SEQ ID NO: 115, and the LCDR3 comprises the sequence of SEQ ID NO: 126; or
(4) the HCDR1 comprises the sequence of SEQ ID NO: 4, the HCDR2 comprises the sequence of SEQ ID NO: 32, the HCDR3 comprises the sequence of SEQ ID NO: 69; the LCDR1 comprises the sequence of SEQ ID NO: 96, the LCDR2 comprises the sequence of SEQ ID NO: 115, and the LCDR3 comprises the sequence of SEQ ID NO: 127; or
(5) the HCDR1 comprises the sequence of SEQ ID NO: 5, the HCDR2 comprises the sequence of SEQ ID NO: 33, the HCDR3 comprises the sequence of SEQ ID NO: 71; the LCDR1 comprises the sequence of SEQ ID NO: 96, the LCDR2 comprises the sequence of SEQ ID NO: 116, and the LCDR3 comprises the sequence of SEQ ID NO: 128; or
(6) the HCDR1 comprises the sequence of SEQ ID NO: 4, the HCDR2 comprises the sequence of SEQ ID NO: 34, the HCDR3 comprises the sequence of SEQ ID NO: 69; the LCDR1 comprises the sequence of SEQ ID NO: 97, the LCDR2 comprises the sequence of SEQ ID NO: 115, and the LCDR3 comprises the sequence of SEQ ID NO: 129; or
(7) the HCDR1 comprises the sequence of SEQ ID NO: 6, the HCDR2 comprises the sequence of SEQ ID NO: 32, the HCDR3 comprises the sequence of SEQ ID NO: 69; the LCDR1 comprises the sequence of SEQ ID NO: 96, the LCDR2 comprises the sequence of SEQ ID NO: 115, and the LCDR3 comprises the sequence of SEQ ID NO: 127; or
(8) the HCDR11 comprises the sequence of SEQ ID NO: 7, the HCDR2 comprises the sequence of SEQ ID NO: 35, the HCDR3 comprises the sequence of SEQ ID NO: 72; the LCDR1 comprises the sequence of SEQ ID NO: 96, the LCDR2 comprises the sequence of SEQ ID NO: 115, and the LCDR3 comprises the sequence of SEQ ID NO: 130; or
(9) the HCDR1 comprises the sequence of SEQ ID NO: 8, the HCDR2 comprises the sequence of SEQ ID NO: 36, the HCDR3 comprises the sequence of SEQ ID NO: 72: the LCDR11 comprises the sequence of SEQ ID NO: 96, the LCDR2 comprises the sequence of SEQ ID NO: 115, and the LCDR3 comprises the sequence of SEQ ID NO: 130; or
(10) the HCDR1 comprises the sequence of SEQ ID NO: 6, the HCDR2 comprises the sequence of SEQ ID NO: 37, the HCDR3 comprises the sequence of SEQ ID NO: 69; the LCDR1 comprises the sequence of SEQ ID NO: 98, the LCDR2 comprises the sequence of SEQ ID NO: 115, and the LCDR3 comprises the sequence of SEQ ID NO: 127; or
(11) the HCDR1 comprises the sequence of SEQ ID NO: 5, the HCDR2 comprises the sequence of SEQ ID NO: 38, the HCDR3 comprises the sequence of SEQ ID NO: 73; the LCDR1 comprises the sequence of SEQ ID NO: 96, the LCDR2 comprises the sequence of SEQ ID NO: 115, and the LCDR3 comprises the sequence of SEQ ID NO: 128; or
(12) the HCDR1 comprises the sequence of SEQ ID NO: 8, the HCDR2 comprises the sequence of SEQ ID NO: 35, the HCDR3 comprises the sequence of SEQ ID NO: 74; the LCDR1 comprises the sequence of SEQ ID NO: 96, the LCDR2 comprises the sequence of SEQ ID NO: 115, and the LCDR3 comprises the sequence of SEQ ID NO: 130; or
(13) the HCDR1 comprises the sequence of SEQ ID NO: 6, the HCDR2 comprises the sequence of SEQ ID NO: 32, the HCDR3 comprises the sequence of SEQ ID NO: 69; the LCDR1 comprises the sequence of SEQ ID NO: 96, the LCDR2 comprises the sequence of SEQ ID NO: 115, and the LCDR3 comprises the sequence of SEQ ID NO 127; or
(14) the HCDR1 comprises the sequence of SEQ ID NO: 2, the HCDR2 comprises the sequence of SEQ ID NO: 39, the HCDR3 comprises the sequence of SEQ ID NO: 69; the LCDR1 comprises the sequence of SEQ ID NO: 99, the LCDR2 comprises the sequence of SEQ ID NO: 115, and the LCDR3 comprises the sequence of SEQ ID NO: 131; or
(15) the HCDR1 comprises the sequence of SEQ ID NO: 5, the HCDR2 comprises the sequence of SEQ ID NO: 33, the HCDR3 comprises the sequence of SEQ ID NO: 71; the LCDR1 comprises the sequence of SEQ ID NO: 96, the LCDR2 comprises the sequence of SEQ ID NO: 115, and the LCDR3 comprises the sequence of SEQ ID NO: 128; or
(16) the HCDR11 comprises the sequence of SEQ ID NO: 9, the HCDR2 comprises the sequence of SEQ ID NO: 40, the HCDR3 comprises the sequence of SEQ ID NO: 75; the LCDR1 comprises the sequence of SEQ ID NO: 99, the LCDR2 comprises the sequence of SEQ ID NO: 115, and the LCDR3 comprises the sequence of SEQ ID NO: 132; or
(17) the HCDR1 comprises the sequence of SEQ ID NO: 9, the HCDR2 comprises the sequence of SEQ ID NO: 41, the HCDR3 comprises the sequence of SEQ ID NO: 76; the LCDR1 comprises the sequence of SEQ ID NO: 99, the LCDR2 comprises the sequence of SEQ ID NO: 115, and the LCDR3 comprises the sequence of SEQ ID NO: 133; or
(18) the HCDR1 comprises the sequence of SEQ ID NO: 10, the HCDR2 comprises the sequence of SEQ ID NO: 42, the HCDR3 comprises the sequence of SEQ ID NO: 77; the LCDR1 comprises the sequence of SEQ ID NO: 100, the LCDR2 comprises the sequence of SEQ ID NO: 115, and the LCDR3 comprises the sequence of SEQ ID NO: 134; or
(19) the HCDR1 comprises the sequence of SEQ ID NO: 11, the HCDR2 comprises the sequence of SEQ ID NO: 43, the HCDR3 comprises the sequence of SEQ ID NO: 71; the LCDR1 comprises the sequence of SEQ ID NO: 101, the LCDR2 comprises the sequence of SEQ ID NO: 115, and the LCDR3 comprises the sequence of SEQ ID NO: 135; or
(20) the HCDR1 comprises the sequence of SEQ ID NO: 12, the HCDR2 comprises the sequence of SEQ ID NO: 44, the HCDR3 comprises the sequence of SEQ ID NO: 75; the LCDR1 comprises the sequence of SEQ ID NO: 99, the LCDR2 comprises the sequence of SEQ ID NO: 115, and the LCDR3 comprises the sequence of SEQ ID NO: 132; or
(21) the HCDR1 comprises the sequence of SEQ ID NO: 13, the HCDR2 comprises the sequence of SEQ ID NO: 40, the HCDR3 comprises the sequence of SEQ ID NO: 75; the LCDR1 comprises the sequence of SEQ ID NO: 99, the LCDR2 comprises the sequence of SEQ ID NO: 115, and the LCDR3 comprises the sequence of SEQ ID NO: 132; or
(22) the HCDR1 comprises the sequence of SEQ ID NO: 12, the HCDR2 comprises the sequence of SEQ ID NO: 44, the HCDR3 comprises the sequence of SEQ ID NO: 75; the LCDR1 comprises the sequence of SEQ ID NO: 99, the LCDR2 comprises the sequence of SEQ ID NO: 115, and the LCDR3 comprises the sequence of SEQ ID NO: 132; or
(23) the HCDR1 comprises the sequence of SEQ ID NO: 14, the HCDR2 comprises the sequence of SEQ ID NO: 45, the HCDR3 comprises the sequence of SEQ ID NO: 78; the LCDR1 comprises the sequence of SEQ ID NO: 96, the LCDR2 comprises the sequence of SEQ ID NO: 115, and the LCDR3 comprises the sequence of SEQ ID NO: 136; or
(24) the HCDR1 comprises the sequence of SEQ ID NO: 8, the HCDR2 comprises the sequence of SEQ ID NO: 35, the HCDR3 comprises the sequence of SEQ ID NO: 72; the LCDR1 comprises the sequence of SEQ ID NO: 96, the LCDR2 comprises the sequence of SEQ ID NO: 115, and the LCDR3 comprises the sequence of SEQ ID NO: 130; or
(25) the HCDR1 comprises the sequence of SEQ ID NO: 15, the HCDR2 comprises the sequence of SEQ ID NO: 46, the HCDR3 comprises the sequence of SEQ ID NO: 79; the LCDR1 comprises the sequence of SEQ ID NO: 102, the LCDR2 comprises the sequence of SEQ ID NO: 117, and the LCDR3 comprises the sequence of SEQ ID NO: 137; or
(26) the HCDR1 comprises the sequence of SEQ ID NO: 16, the HCDR2 comprises the sequence of SEQ ID NO: 47, the HCDR3 comprises the sequence of SEQ ID NO: 80; the LCDR1 comprises the sequence of SEQ ID NO: 103, the LCDR2 comprises the sequence of SEQ ID NO: 118, and the LCDR3 comprises the sequence of SEQ ID NO: 138; or
(27) the HCDR1 comprises the sequence of SEQ ID NO: 17, the H-CDR2 comprises the sequence of SEQ ID NO: 48, the HCDR3 comprises the sequence of SEQ ID NO: 81; the LCDR1 comprises the sequence of SEQ ID NO: 96, the LCDR2 comprises the sequence of SEQ ID NO: 119, and the LCDR3 comprises the sequence of SEQ ID NO: 139; or
(28) the HCDR1 comprises the sequence of SEQ ID NO: 18, the HCDR2 comprises the sequence of SEQ ID NO: 49, the HCDR3 comprises the sequence of SEQ ID NO: 82; the LCDR1 comprises the sequence of SEQ ID NO: 104, the LCDR2 comprises the sequence of SEQ ID NO: 118, and the LCDR3 comprises the sequence of SEQ ID NO: 140; or
(29) the HCDR1 comprises the sequence of SEQ ID NO: 19, the HCDR2 comprises the sequence of SEQ ID NO: 50, the HCDR3 comprises the sequence of SEQ ID NO: 83; the LCDR1 comprises the sequence of SEQ ID NO: 96, the LCDR2 comprises the sequence of SEQ ID NO: 120, and the LCDR3 comprises the sequence of SEQ ID NO: 141; or
(30) the HCDR1 comprises the sequence of SEQ ID NO: 17, the HCDR2 comprises the sequence of SEQ ID NO: 51, the HCDR3 comprises the sequence of SEQ ID NO: 84; the LCDR1 comprises the sequence of SEQ ID NO: 96, the LCDR2 comprises the sequence of SEQ ID NO: 121, and the LCDR3 comprises the sequence of SEQ ID NO: 142; or
(31) the HCDR1 comprises the sequence of SEQ ID NO: 2, the HCDR2 comprises the sequence of SEQ ID NO: 52, the HCDR3 comprises the sequence of SEQ ID NO: 69; the LCDR1 comprises the sequence of SEQ ID NO: 105, the LCDR2 comprises the sequence of SEQ ID NO: 115, and the LCDR3 comprises the sequence of SEQ ID NO: 143; or
(32) the HCDR1 comprises the sequence of SEQ ID NO: 20, the HCDR2 comprises the sequence of SEQ ID NO: 53, the HCDR3 comprises the sequence of SEQ ID NO: 85; the LCDR1 comprises the sequence of SEQ ID NO: 106, the LCDR2 comprises the sequence of SEQ ID NO: 122, and the LCDR3 comprises the sequence of SEQ ID NO: 144; or
(33) the HCDR1 comprises the sequence of SEQ ID NO: 21, the HCDR2 comprises the sequence of SEQ ID NO: 54, the HCDR3 comprises the sequence of SEQ ID NO: 86; the LCDR1 comprises the sequence of SEQ ID NO: 95, the LCDR2 comprises the sequence of SEQ ID NO: 115, and the LCDR3 comprises the sequence of SEQ ID NO: 128; or
(34) the HCDR1 comprises the sequence of SEQ ID NO: 22, the HCDR2 comprises the sequence of SEQ ID NO: 55, the HCDR3 comprises the sequence of SEQ ID NO: 87; the LCDR1 comprises the sequence of SEQ ID NO: 98, the LCDR2 comprises the sequence of SEQ ID NO: 115, and the LCDR3 comprises the sequence of SEQ ID NO: 145; or
(35) the HCDR1 comprises the sequence of SEQ ID NO: 2, the HCDR2 comprises the sequence of SEQ ID NO: 56, the HCDR3 comprises the sequence of SEQ ID NO: 69; the LCDR1 comprises the sequence of SEQ ID NO: 98, the LCDR2 comprises the sequence of SEQ ID NO: 115, and the LCDR3 comprises the sequence of SEQ ID NO: 127; or
(36) the HCDR1 comprises the sequence of SEQ ID NO: 1, the HCDR2 comprises the sequence of SEQ ID NO: 57, the HCDR3 comprises the sequence of SEQ ID NO: 69; the LCDR1 comprises the sequence of SEQ ID NO: 98, the LCDR2 comprises the sequence of SEQ ID NO: 115, and the LCDR3 comprises the sequence of SEQ ID NO: 127; or
(37) the HCDR1 comprises the sequence of SEQ ID NO: 11, the HCDR2 comprises the sequence of SEQ ID NO: 43, the HCDR3 comprises the sequence of SEQ ID NO: 88; the LCDR1 comprises the sequence of SEQ ID NO: 96, the LCDR2 comprises the sequence of SEQ ID NO: 115, and the LCDR3 comprises the sequence of SEQ ID NO: 146; or
(38) the HCDR1 comprises the sequence of SEQ ID NO: 23, the HCDR2 comprises the sequence of SEQ ID NO: 58, the HCDR3 comprises the sequence of SEQ ID NO: 89; the LCDR1 comprises the sequence of SEQ ID NO: 96, the LCDR2 comprises the sequence of SEQ ID NO: 115, and the LCDR3 comprises the sequence of SEQ ID NO: 147; or
(39) the HCDR1 comprises the sequence of SEQ ID NO: 22, the HCDR2 comprises the sequence of SEQ ID NO: 55, the HCDR3 comprises the sequence of SEQ ID NO: 87; the LCDR1 comprises the sequence of SEQ ID NO: 96, the LCDR2 comprises the sequence of SEQ ID NO: 115, and the LCDR3 comprises the sequence of SEQ ID NO: 130; or
(40) the HCDR1 comprises the sequence of SEQ ID NO: 24, the HCDR2 comprises the sequence of SEQ ID NO: 59, the HCDR3 comprises the sequence of SEQ ID NO: 90; the LCDR1 comprises the sequence of SEQ ID NO: 96, the LCDR2 comprises the sequence of SEQ ID NO: 115, and the LCDR3 comprises the sequence of SEQ ID NO: 148; or
(41) the HCDR1 comprises the sequence of SEQ ID NO: 25, the HCDR2 comprises the sequence of SEQ ID NO: 60, the HCDR3 comprises the sequence of SEQ ID NO: 90; the LCDR1 comprises the sequence of SEQ ID NO: 96, the LCDR2 comprises the sequence of SEQ ID NO: 115, and the LCDR3 comprises the sequence of SEQ ID NO: 118; or
(42) the HCDR1 comprises the sequence of SEQ ID NO: 26, the HCDR2 comprises the sequence of SEQ ID NO: 61, the HCDR3 comprises the sequence of SEQ ID NO: 91; the LCDR1 comprises the sequence of SEQ ID NO: 107, the LCDR2 comprises the sequence of SEQ ID NO: 115, and the LCDR3 comprises the sequence of SEQ ID NO: 149; or
(43) the HCDR11 comprises the sequence of SEQ ID NO: 26, the HCDR2 comprises the sequence of SEQ ID NO: 62, the HCDR3 comprises the sequence of SEQ ID NO: 92; the LCDR1 comprises the sequence of SEQ ID NO: 108, the LCDR2 comprises the sequence of SEQ ID NO: 115, and the LCDR3 comprises the sequence of SEQ ID NO: 150; or
(44) the HCDR11 comprises the sequence of SEQ ID NO: 24, the HCDR2 comprises the sequence of SEQ ID NO: 59, the HCDR3 comprises the sequence of SEQ ID NO: 90; the LCDR1 comprises the sequence of SEQ ID NO: 96, the LCDR2 comprises the sequence of SEQ ID NO: 115, and the LCDR3 comprises the sequence of SEQ ID NO: 148; or
(45) the HCDR1 comprises the sequence of SEQ ID NO: 26, the HCDR2 comprises the sequence of SEQ ID NO: 61, the HCDR3 comprises the sequence of SEQ ID NO: 91; the LCDR1 comprises the sequence of SEQ ID NO: 107, the LCDR2 comprises the sequence of SEQ ID NO: 115, and the LCDR3 comprises the sequence of SEQ ID NO: 149; or
(46) the HCDR1 comprises the sequence of SEQ ID NO: 25, the HCDR2 comprises the sequence of SEQ ID NO: 60, the HCDR3 comprises the sequence of SEQ ID NO: 90; the LCDR1 comprises the sequence of SEQ ID NO: 109, the LCDR2 comprises the sequence of SEQ ID NO: 123, and the LCDR3 comprises the sequence of SEQ ID NO: 151; or
(47) the HCDR1 comprises the sequence of SEQ ID NO: 27, the HCDR2 comprises the sequence of SEQ ID NO: 367, the HCDR3 comprises the sequence of SEQ ID NO: 93; the LCDR1 comprises the sequence of SEQ ID NO: 109, the LCDR2 comprises the sequence of SEQ ID NO.: 123, and the LCDR3 comprises the sequence of SEQ ID NO: 151; or
(48) the HCDR1 comprises the sequence of SEQ ID NO: 26, the HCDR2 comprises the sequence of SEQ ID NO: 63, the HCDR3 comprises the sequence of SEQ ID NO: 92; the LCDR1 comprises the sequence of SEQ ID NO: 110, the LCDR2 comprises the sequence of SEQ ID NO: 115, and the LCDR3 comprises the sequence of SEQ ID NO: 150; or
(49) the HCDR1 comprises the sequence of SEQ ID NO: 26, the HCDR2 comprises the sequence of SEQ ID NO: 64, the HCDR3 comprises the sequence of SEQ ID NO: 92; the LCDR1 comprises the sequence of SEQ ID NO: 111, the LCDR2 comprises the sequence of SEQ ID NO: 115, and the LCDR3 comprises the sequence of SEQ ID NO: 150; or
(50) the HCDR1 comprises the sequence of SEQ ID NO: 28, the HCDR2 comprises the sequence of SEQ ID NO: 65, the HCDR3 comprises the sequence of SEQ ID NO: 85; the LCDR1 comprises the sequence of SEQ ID NO: 101, the LCDR2 comprises the sequence of SEQ ID NO: 120, and the LCDR3 comprises the sequence of SEQ ID NO: 152; or
(51) the HCDR1 comprises the sequence of SEQ ID NO: 28, the HCDR2 comprises the sequence of SEQ ID NO: 66, the HCDR3 comprises the sequence of SEQ ID NO: 94; the LCDR1 comprises the sequence of SEQ ID NO: 101, the LCDR2 comprises the sequence of SEQ ID NO: 120, and the LCDR3 comprises the sequence of SEQ ID NO: 153; or
(52) the HCDR1 comprises the sequence of SEQ ID NO: 28, the HCDR2 comprises the sequence of SEQ ID NO: 65, the HCDR3 comprises the sequence of SEQ ID NO: 85; the LCDR1 comprises the sequence of SEQ ID NO: 112, the LCDR2 comprises the sequence of SEQ ID NO: 120, and the LCDR3 comprises the sequence of SEQ ID NO: 152; or
(53) the HCDR1 comprises the sequence of SEQ ID NO: 28, the HCDR2 comprises the sequence of SEQ ID NO: 66, the HCDR3 comprises the sequence of SEQ ID NO: 85; the LCDR1 comprises the sequence of SEQ ID NO: 101, the LCDR2 comprises the sequence of SEQ ID NO: 120, and the LCDR3 comprises the sequence of SEQ ID NO: 154; or
(54) the HCDR1 comprises the sequence of SEQ ID NO: 26, the H-CDR2 comprises the sequence of SEQ ID NO: 67, the HCDR3 comprises the sequence of SEQ ID NO: 92; the LCDR1 comprises the sequence of SEQ ID NO: 96, the LCDR2 comprises the sequence of SEQ ID NO: 115, and the LCDR3 comprises the sequence of SEQ ID NO: 155; or
(55) the HCDR1 comprises the sequence of SEQ ID NO: 26, the HCDR2 comprises the sequence of SEQ ID NO: 63, the HCDR3 comprises the sequence of SEQ ID NO: 92; the LCDR1 comprises the sequence of SEQ ID NO: 108, the LCDR2 comprises the sequence of SEQ ID NO: 115, and the LCDR3 comprises the sequence of SEQ ID NO: 150; or
(56) the HCDR1 comprises the sequence of SEQ ID NO: 26, the HCDR2 comprises the sequence of SEQ ID NO: 61, the HCDR3 comprises the sequence of SEQ ID NO: 91; the LCDR1 comprises the sequence of SEQ ID NO: 107, the LCDR2 comprises the sequence of SEQ ID NO: 115, and the LCDR3 comprises the sequence of SEQ ID NO: 149; or
(57) the HCDR1 comprises the sequence of SEQ ID NO: 11, the HCDR2 comprises the sequence of SEQ ID NO: 61, the HCDR3 comprises the sequence of SEQ ID NO: 91; the LCDR1 comprises the sequence of SEQ ID NO: 113, the LCDR2 comprises the sequence of SEQ ID NO: 115, and the LCDR3 comprises the sequence of SEQ ID NO: 149; or
(58) the HCDR1 comprises the sequence of SEQ ID NO: 26, the HCDR2 comprises the sequence of SEQ ID NO: 61, the HCDR3 comprises the sequence of SEQ ID NO: 91; the LCDR1 comprises the sequence of SEQ ID NO: 107, the LCDR2 comprises the sequence of SEQ ID NO: 115, and the LCDR3 comprises the sequence of SEQ ID NO: 149; or
(59) the HCDR1 comprises the sequence of SEQ ID NO: 11, the HCDR2 comprises the sequence of SEQ ID NO: 61, the HCDR3 comprises the sequence of SEQ ID NO: 91; the LCDR1 comprises the sequence of SEQ ID NO: 96, the LCDR2 comprises the sequence of SEQ ID NO: 115, and the LCDR3 comprises the sequence of SEQ ID NO: 148; or
(60) the HCDR1 comprises the sequence of SEQ ID NO: 28, the HCDR2 comprises the sequence of SEQ ID NO: 66, the HCDR3 comprises the sequence of SEQ ID NO: 85; the LCDR1 comprises the sequence of SEQ ID NO: 101, the LCDR2 comprises the sequence of SEQ ID NO: 120, and the LCDR3 comprises the sequence of SEQ ID NO: 153; or
(61) the HCDR1 comprises the sequence of SEQ ID NO: 16, the HCDR2 comprises the sequence of SEQ ID NO: 47, the HCDR3 comprises the sequence of SEQ ID NO: 80, the LCDR1 comprises the sequence of SEQ ID NO: 103, the LCDR2 comprises the sequence of SEQ ID NO: 118, and the LCDR3 comprises the sequence of SEQ ID NO: 204; or
(62) the HCDR1 comprises the sequence of SEQ ID NO: 201, the HCDR2 comprises the sequence of SEQ ID NO: 47, the HCDR3 comprises the sequence of SEQ ID NO: 80, the LCDR1 comprises the sequence of SEQ ID NO: 103, the LCDR2 comprises the sequence of SEQ ID NO: 118, and the LCDR3 comprises the sequence of SEQ ID NO: 138; or
(63) the HCDR1 comprises the sequence of SEQ ID NO: 201, the HCDR2 comprises the sequence of SEQ ID NO: 47, the HCDR3 comprises the sequence of SEQ ID NO: 80, the LCDR1 comprises the sequence of SEQ ID NO: 103, the LCDR2 comprises the sequence of SEQ ID NO: 118, and the LCDR3 comprises the sequence of SEQ ID NO: 204; or
(64) the HCDR11 comprises the sequence of SEQ ID NO: 202, the HCDR2 comprises the sequence of SEQ ID NO: 203, the HCDR3 comprises the sequence of SEQ ID NO: 83, the LCDR1 comprises the sequence of SEQ ID NO: 96, the LCDR2 comprises the sequence of SEQ ID NO: 120, and the LCDR3 comprises the sequence of SEQ ID NO: 141; or
(65) the HCDR1 comprises the sequence of SEQ ID NO: 202, the HCDR2 comprises the sequence of SEQ ID NO: 203, the HCDR3 comprises the sequence of SEQ ID NO: 83, the LCDR1 comprises the sequence of SEQ ID NO: 205, the LCDR2 comprises the sequence of SEQ ID NO: 120, and the LCDR3 comprises the sequence of SEQ ID NO: 141; or
(66) the HDR1 comprises the sequence of SEQ ID NO: 19, the HCDR2 comprises the sequence of SEQ ID NO: 50, the HCDR3 comprises the sequence of SEQ ID NO: 83, the LCDR1 comprises the sequence of SEQ ID NO: 205, the LCDR2 comprises the sequence of SEQ ID NO: 120, and the LCDR3 comprises the sequence of SEQ ID NO: 141.

8-11. (canceled)

12. The antibody or an antigen-binding fragment thereof of claim 1, comprising a heavy chain variable region comprising a sequence selected from the group consisting of SEQ ID Nos: 206-259, 311-313, 318-321, and a homologous sequence thereof having at least 80% sequence identity yet retaining specific binding affinity to CLDN18, and
a light chain variable region comprising a sequence selected from the group consisting of SEQ ID Nos: 260-310, 314-317, 322-324, and a homologous sequence thereof having at least 80% sequence identity yet retaining specific binding affinity to CLDN18.

13. (canceled)

14. The antibody or an antigen-binding fragment thereof of claim 1, comprising:
(1) a heavy chain variable region comprising the sequence of SEQ ID NO: 249 and a light chain variable region comprising the sequence of SEQ ID NO: 294; or
(2) a heavy chain variable region comprising the sequence of SEQ ID NO: 208 and a light chain variable region comprising the sequence of SEQ ID NO: 278; or
(3) a heavy chain variable region comprising the sequence of SEQ ID NO: 225 and a light chain variable region comprising the sequence of SEQ ID NO: 296; or
(4) a heavy chain variable region comprising the sequence of SEQ ID NO: 242 and a light chain variable region comprising the sequence of SEQ ID NO: 289; or
(5) a heavy chain variable region comprising the sequence of SEQ ID NO: 244 and a light chain variable region comprising the sequence of SEQ ID NO: 265; or
(6) a heavy chain variable region comprising the sequence of SEQ ID NO: 242 and a light chain variable region comprising the sequence of SEQ ID NO: 295; or
(7) a heavy chain variable region comprising the sequence of SEQ ID NO: 243 and a light chain variable region comprising the sequence of SEQ ID NO: 267; or
(8) a heavy chain variable region comprising the sequence of SEQ ID NO: 237 and a light chain variable region comprising the sequence of SEQ ID NO: 304; or
(9) a heavy chain variable region comprising the sequence of SEQ ID NO: 239 and a light chain variable region comprising the sequence of SEQ ID NO: 277; or
(10) a heavy chain variable region comprising the sequence of SEQ ID NO: 225 and a light chain variable region comprising the sequence of SEQ ID NO: 310; or
(11) a heavy chain variable region comprising the sequence of SEQ ID NO: 246 and a light chain variable region comprising the sequence of SEQ ID NO: 273; or
(12) a heavy chain variable region comprising the sequence of SEQ ID NO: 226 and a light chain variable region comprising the sequence of SEQ ID NO: 298; or
(13) a heavy chain variable region comprising the sequence of SEQ ID NO: 224 and a light chain variable region comprising the sequence of SEQ ID NO: 296; or
(14) a heavy chain variable region comprising the sequence of SEQ ID NO: 226 and a light chain variable region comprising the sequence of SEQ ID NO: 297; or
(15) a heavy chain variable region comprising the sequence of SEQ ID NO: 240 and a light chain variable region comprising the sequence of SEQ ID NO: 276; or
(16) a heavy chain variable region comprising the sequence of SEQ ID NO: 238 and a light chain variable region comprising the sequence of SEQ ID NO: 275; or
(17) a heavy chain variable region comprising the sequence of SEQ ID NO: 258 and a light chain variable region comprising the sequence of SEQ ID NO: 301; or
(18) a heavy chain variable region comprising the sequence of SEQ ID NO: 209 and a light chain variable region comprising the sequence of SEQ ID NO: 301; or
(19) a heavy chain variable region comprising the sequence of SEQ ID NO: 244 and a light chain variable region comprising the sequence of SEQ ID NO: 266; or
(20) a heavy chain variable region comprising the sequence of SEQ ID NO: 247 and a light chain variable region comprising the sequence of SEQ ID NO: 289; or
(21) a heavy chain variable region comprising the sequence of SEQ ID NO: 228 and a light chain variable region comprising the sequence of SEQ ID NO: 300; or
(22) a heavy chain variable region comprising the sequence of SEQ ID NO: 245 and a light chain variable region comprising the sequence of SEQ ID NO: 266; or
(23) a heavy chain variable region comprising the sequence of SEQ ID NO: 258 and a light chain variable region comprising the sequence of SEQ ID NO: 289; or
(24) a heavy chain variable region comprising the sequence of SEQ ID NO: 248 and a light chain variable region comprising the sequence of SEQ ID NO: 289; or
(25) a heavy chain variable region comprising the sequence of SEQ ID NO: 230 and a light chain variable region comprising the sequence of SEQ ID NO: 303; or
(26) a heavy chain variable region comprising the sequence of SEQ ID NO: 212 and a light chain variable region comprising the sequence of SEQ ID NO: 309; or
(27) a heavy chain variable region comprising the sequence of SEQ ID NO: 207 and a light chain variable region comprising the sequence of SEQ ID NO: 280; or
(28) a heavy chain variable region comprising the sequence of SEQ ID NO: 210 and a light chain variable region comprising the sequence of SEQ ID NO: 307; or
(29) a heavy chain variable region comprising the sequence of SEQ ID NO: 231 and a light chain variable region comprising the sequence of SEQ ID NO: 306; or
(30) a heavy chain variable region comprising the sequence of SEQ ID NO: 241 and a light chain variable region comprising the sequence of SEQ ID NO: 283; or
(31) a heavy chain variable region comprising the sequence of SEQ ID NO: 213 and a light chain variable region comprising the sequence of SEQ ID NO: 308; or
(32) a heavy chain variable region comprising the sequence of SEQ ID NO: 214 and a light chain variable region comprising the sequence of SEQ ID NO: 269; or
(33) a heavy chain variable region comprising the sequence of SEQ ID NO: 206 and a light chain variable region comprising the sequence of SEQ ID NO: 305; or
(34) a heavy chain variable region comprising the sequence of SEQ ID NO: 259 and a light chain variable region comprising the sequence of SEQ ID NO: 271; or
(35) a heavy chain variable region comprising the sequence of SEQ ID NO: 221 and a light chain variable region comprising the sequence of SEQ ID NO: 281; or
(36) a heavy chain variable region comprising the sequence of SEQ ID NO: 227 and a light chain variable region comprising the sequence of SEQ ID NO: 286; or
(37) a heavy chain variable region comprising the sequence of SEQ ID NO: 215 and a light chain variable region comprising the sequence of SEQ ID NO: 263; or
(38) a heavy chain variable region comprising the sequence of SEQ ID NO: 218 and a light chain variable region comprising the sequence of SEQ ID NO: 262; or
(39) a heavy chain variable region comprising the sequence of SEQ ID NO: 216 and a light chain variable region comprising the sequence of SEQ ID NO: 263; or
(40) a heavy chain variable region comprising the sequence of SEQ ID NO: 234 and a light chain variable region comprising the sequence of SEQ ID NO: 274; or
(41) a heavy chain variable region comprising the sequence of SEQ ID NO: 211 and a light chain variable region comprising the sequence of SEQ ID NO: 261; or
(42) a heavy chain variable region comprising the sequence of SEQ ID NO: 217 and a light chain variable region comprising the sequence of SEQ ID NO: 262; or
(43) a heavy chain variable region comprising the sequence of SEQ ID NO: 219 and a light chain variable region comprising the sequence of SEQ ID NO: 264; or
(44) a heavy chain variable region comprising the sequence of SEQ ID NO: 230 and a light chain variable region comprising the sequence of SEQ ID NO: 279; or
(45) a heavy chain variable region comprising the sequence of SEQ ID NO: 257 and a light chain variable region comprising the sequence of SEQ ID NO: 271; or
(46) a heavy chain variable region comprising the sequence of SEQ ID NO: 233 and a light chain variable region comprising the sequence of SEQ ID NO: 292; or
(47) a heavy chain variable region comprising the sequence of SEQ ID NO: 255 and a light chain variable region comprising the sequence of SEQ ID NO: 299; or
(48) a heavy chain variable region comprising the sequence of SEQ ID NO: 252 and a light chain variable region comprising the sequence of SEQ ID NO: 272; or
(49) a heavy chain variable region comprising the sequence of SEQ ID NO: 223 and a light chain variable region comprising the sequence of SEQ ID NO: 285; or
(50) a heavy chain variable region comprising the sequence of SEQ ID NO: 232 and a light chain variable region comprising the sequence of SEQ ID NO: 287; or
(51) a heavy chain variable region comprising the sequence of SEQ ID NO. 253 and a light chain variable region comprising the sequence of SEQ ID NO: 270; or
(52) a heavy chain variable region comprising the sequence of SEQ ID NO: 222 and a light chain variable region comprising the sequence of SEQ ID NO: 260; or
(53) a heavy chain variable region comprising the sequence of SEQ ID NO: 220 and a light chain variable region comprising the sequence of SEQ ID NO: 284; or
(54) a heavy chain variable region comprising the sequence of SEQ ID NO: 251 and a light chain variable region comprising the sequence of SEQ ID NO: 291; or
(55) a heavy chain variable region comprising the sequence of SEQ ID NO: 256 and a light chain variable region comprising the sequence of SEQ ID NO: 268; or
(56) a heavy chain variable region comprising the sequence of SEQ ID NO: 236 and a light chain variable region comprising the sequence of SEQ ID NO: 293; or
(57) a heavy chain variable region comprising the sequence of SEQ ID NO: 250 and a light chain variable region comprising the sequence of SEQ ID NO: 290; or
(58) a heavy chain variable region comprising the sequence of SEQ ID NO: 235 and a light chain variable region comprising the sequence of SEQ ID NO: 288; or
(59) a heavy chain variable region comprising the sequence of SEQ ID NO: 229 and a light chain variable region comprising the sequence of SEQ ID NO: 282; or
(60) a heavy chain variable region comprising the sequence of SEQ ID NO: 254 and a light chain variable region comprising the sequence of SEQ ID NO: 302; or
(61) a heavy chain variable region comprising the sequence of SEQ ID NO: 311 and a light chain variable region comprising the sequence of SEQ ID NO: 314; or
(62) a heavy chain variable region comprising the sequence of SEQ ID NO: 311 and a light chain variable region comprising the sequence of SEQ ID NO: 315; or
(63) a heavy chain variable region comprising the sequence of SEQ ID NO: 311 and a light chain variable region comprising the sequence of SEQ ID NO: 316; or
(64) a heavy chain variable region comprising the sequence of SEQ ID NO: 311 and a light chain variable region comprising the sequence of SEQ ID NO: 317; or
(65) a heavy chain variable region comprising the sequence of SEQ ID NO: 311 and a light chain variable region comprising the sequence of SEQ ID NO: 402; or
(66) a heavy chain variable region comprising the sequence of SEQ ID NO: 312 and a light chain variable region comprising the sequence of SEQ ID NO: 314; or
(67) a heavy chain variable region comprising the sequence of SEQ ID NO: 312 and a light chain variable region comprising the sequence of SEQ ID NO: 315; or
(68) a heavy chain variable region comprising the sequence of SEQ ID NO: 312 and a light chain variable region comprising the sequence of SEQ ID NO: 316; or
(69) a heavy chain variable region comprising the sequence of SEQ ID NO: 312 and a light chain variable region comprising the sequence of SEQ ID NO: 317; or
(70) a heavy chain variable region comprising the sequence of SEQ ID NO: 312 and a light chain variable region comprising the sequence of SEQ ID NO: 402; or
(71) a heavy chain variable region comprising the sequence of SEQ ID NO: 313 and a light chain variable region comprising the sequence of SEQ ID NO: 314; or
(72) a heavy chain variable region comprising the sequence of SEQ ID NO: 313 and a light chain variable region comprising the sequence of SEQ ID NO: 315; or
(73) a heavy chain variable region comprising the sequence of SEQ ID NO: 313 and a light chain variable region comprising the sequence of SEQ ID NO: 316; or
(74) a heavy chain variable region comprising the sequence of SEQ ID NO: 313 and a light chain variable region comprising the sequence of SEQ ID NO: 317; or
(75) a heavy chain variable region comprising the sequence of SEQ ID NO: 313 and a light chain variable region comprising the sequence of SEQ ID NO: 402; or
(76) a heavy chain variable region comprising the sequence of SEQ ID NO: 318 and a light chain variable region comprising the sequence of SEQ ID NO: 322; or
(77) a heavy chain variable region comprising the sequence of SEQ ID NO: 318 and a light chain variable region comprising the sequence of SEQ ID NO: 323; or
(78) a heavy chain variable region comprising the sequence of SEQ ID NO: 318 and a light chain variable region comprising the sequence of SEQ ID NO: 324; or
(79) a heavy chain variable region comprising the sequence of SEQ ID NO: 319 and a light chain variable region comprising the sequence of SEQ ID NO: 322; or
(80) a heavy chain variable region comprising the sequence of SEQ ID NO: 319 and a light chain variable region comprising the sequence of SEQ ID NO: 323; or
(81) a heavy chain variable region comprising the sequence of SEQ ID NO: 319 and a light chain variable region comprising the sequence of SEQ ID NO: 324; or
(82) a heavy chain variable region comprising the sequence of SEQ ID NO: 320 and a light chain variable region comprising the sequence of SEQ ID NO: 322; or
(83) a heavy chain variable region comprising the sequence of SEQ ID NO: 320 and a light chain variable region comprising the sequence of SEQ ID NO: 323; or
(84) a heavy chain variable region comprising the sequence of SEQ ID NO: 320 and a light chain variable region comprising the sequence of SEQ ID NO: 324; or
(85) a heavy chain variable region comprising the sequence of SEQ ID NO: 321 and a light chain variable region comprising the sequence of SEQ ID NO: 322; or
(86) a heavy chain variable region comprising the sequence of SEQ ID NO: 321 and a light chain variable region comprising the sequence of SEQ ID NO: 323; or
(87) a heavy chain variable region comprising the sequence of SEQ ID NO: 321 and a light chain variable region comprising the sequence of SEQ ID NO: 324.

15. The antibody or an antigen-binding fragment thereof of claim 1, further comprising one or more amino acid residue substitutions or modifications yet retains specific binding affinity to CLDN18; optionally wherein at least one of the substitutions or modifications is in one or more of the CDR sequences, and/or in one or more of the non-CDR sequences of the heavy chain variable region or light chain variable region.

16. (canceled)

17. The antibody or an antigen-binding fragment thereof of claim 1, further comprising an Fc region, optionally an Fc region of human immunoglobulin (Ig), or optionally an Fc region of human IgG, or optionally an Fc region derived from human IgG1, IgG2, IgG3, IgG4, IgA1, IgA2 or IgM; optionally wherein the Fc region derived from human IgG1 comprises one or more mutations selected from the group consisting of L235V, G236A, S239D, F243L, H268F, R292P, Y300L, V305I, S324T, A330L, I332E, and P396L; optionally wherein the Fe region derived from human IgG1 comprises a mutation selected from the group consisting of: (1) G236A, S239D and I332E; (2) S239D, A330L and I332E, S239D and I332E; (4) S239D, H268F, S324T and I332E; (5) F243L, R292P, Y300L, V305I and P396L; (6) L235V, F243L, R292P, Y300L and P396L.

18-21. (canceled)

22. The antibody or an antigen-binding fragment thereof of claim 1, which is humanized; optionally which is a monoclonal antibody, a bispecific antibody, a multi-specific antibody, a recombinant antibody, a chimeric antibody, a labeled antibody, a bivalent antibody, an anti-idiotypic antibody or a fusion protein; optionally which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

23-34. (canceled)

35. The antibody or an antigen-binding fragment thereof of claim 1, which is linked to one or more conjugate moieties.

36-37. (canceled)

38. A pharmaceutical composition comprising the antibody or an antigen-binding fragment thereof of claim 1, and one or more pharmaceutically acceptable carriers.

39. A chimeric antigen receptor, comprising the antibody or an antigen-binding fragment thereof of claim 1, a transmembrane region and an intracellular signal region.

40-42. (canceled)

43. An isolated polynucleotide encoding the antibody or an antigen-binding fragment thereof of claim 1, and/or a chimeric antigen receptor comprising the antibody or an antigen-binding fragment thereof, a transmembrane region and an intracellular signal region.

44. A vector comprising the isolated polynucleotide of claim 43.

45. A host cell comprising the vector of claim 44.

46. (canceled)

47. A method of expressing the antibody or antigen-binding fragment thereof of claim 1 or a chimeric antigen receptor comprising the antibody or antigen-binding fragment thereof, a transmembrane region and an intracellular signal region, comprising culturing a host cell comprising a vector encoding the antibody or antigen-binding fragment thereof under the condition at which the vector is expressed.

48. A method of treating, preventing or alleviating a CLDN18 related disease, disorder or condition in a subject, or a method of treating, preventing or alleviating a disease, disorder or condition in a subject that would benefit from modulation of CLDN18 activity, comprising administering to the subject a therapeutically effective amount of the antibody or an antigen-binding fragment thereof of claim 1 and/or a pharmaceutical composition comprising the antibody or antigen-binding fragment thereof and one or more pharmaceutically acceptable carriers, and/or a chimeric antigen receptor comprising the antibody or an antigen-binding fragment thereof, a transmembrane region and an intracellular signal region.

49-55. (canceled)

56. The method of claim 48, further comprising administering a therapeutically effective amount of a second therapeutic agent.

57. (canceled)

58. A method of modulating CLDN18 activity in a CLDN18-positive cell, comprising exposing the CLDN18-positive cell to the anti body or antigen-binding fragment thereof of claim 1, and/or a pharmaceutical composition comprising the antibody or antigen-binding fragment thereof and one or more pharmaceutically acceptable carriers, and/or a chimeric antigen receptor comprising the antibody or antigen-binding fragment thereof, a transmembrane region and an intracellular signal region.

59. (canceled)

60. A method of diagnosing a CLDN18 related disease, disorder or condition in a subject, comprising: a) contacting a sample obtained from the subject with the antibody or an antigen-binding fragment thereof of claim 1, and/or a pharmaceutical composition comprising the antibody or antigen-binding fragment thereof and one or more pharmaceutically acceptable carriers, and/or a chimeric antigen receptor comprising the antibody or an antigen-binding fragment thereof, a transmembrane region and an intracellular signal region; b) determining the presence or amount of CLDN18 in the sample; and c) correlating the presence or the amount of CLDN18 to existence or status of the CLDN18 related disease, disorder or condition in the subject.

61-65. (canceled)


SEQUENCE LISTING (508 sequences)

SEQ ID NO: 1 - protein, 5 aa, Artificial Sequence
  Synthetic
NFDIN

SEQ ID NO: 2 - protein, 5 aa, Artificial Sequence
  Synthetic
NYDIN

SEQ ID NO: 3 - protein, 5 aa, Artificial Sequence
  Synthetic
RYWIQ

SEQ ID NO: 4 - protein, 5 aa, Artificial Sequence
  Synthetic
RNDIN

SEQ ID NO: 5 - protein, 5 aa, Artificial Sequence
  Synthetic
SYWIP

SEQ ID NO: 6 - protein, 5 aa, Artificial Sequence
  Synthetic
SYDIN

SEQ ID NO: 7 - protein, 5 aa, Artificial Sequence
  Synthetic
DYNIH

SEQ ID NO: 8 - protein, 5 aa, Artificial Sequence
  Synthetic
DYNMH

SEQ ID NO: 9 - protein, 5 aa, Artificial Sequence
  Synthetic
DYTIH

SEQ ID NO: 10 - protein, 5 aa, Artificial Sequence
  Synthetic
DYYMA

SEQ ID NO: 11 - protein, 5 aa, Artificial Sequence
  Synthetic
SYWMH

SEQ ID NO: 12 - protein, 5 aa, Artificial Sequence
  Synthetic
DYSMH

SEQ ID NO: 13 - protein, 5 aa, Artificial Sequence
  Synthetic
DYTMH

SEQ ID NO: 14 - protein, 5 aa, Artificial Sequence
  Synthetic
DYGMH

SEQ ID NO: 15 - protein, 5 aa, Artificial Sequence
  Synthetic
SYAMS

SEQ ID NO: 16 - protein, 5 aa, Artificial Sequence
  Synthetic
SYALS

SEQ ID NO: 17 - protein, 5 aa, Artificial Sequence
  Synthetic
SFGMH

SEQ ID NO: 18 - protein, 5 aa, Artificial Sequence
  Synthetic
RYAMS

SEQ ID NO: 19 - protein, 4 aa, Artificial Sequence
  Synthetic
NWVH

SEQ ID NO: 20 - protein, 5 aa, Artificial Sequence
  Synthetic
DYNMD

SEQ ID NO: 21 - protein, 6 aa, Artificial Sequence
  Synthetic
SGYLWN

SEQ ID NO: 22 - protein, 5 aa, Artificial Sequence
  Synthetic
SYWIH

SEQ ID NO: 23 - protein, 5 aa, Artificial Sequence
  Synthetic
TYGIS

SEQ ID NO: 24 - protein, 5 aa, Artificial Sequence
  Synthetic
NYWMN

SEQ ID NO: 25 - protein, 5 aa, Artificial Sequence
  Synthetic
SYWMN

SEQ ID NO: 26 - protein, 5 aa, Artificial Sequence
  Synthetic
TYWMH

SEQ ID NO: 27 - protein, 5 aa, Artificial Sequence
  Synthetic
NFGMY

SEQ ID NO: 28 - protein, 5 aa, Artificial Sequence
  Synthetic
DYYMN

SEQ ID NO: 29 - protein, 17 aa, Artificial Sequence
  Synthetic
RLYPRDGTTTYNEKFKG

SEQ ID NO: 30 - protein, 17 aa, Artificial Sequence
  Synthetic
RIYPRDDSTTYNEKFKG

SEQ ID NO: 31 - protein, 17 aa, Artificial Sequence
  Synthetic
MIHPNSGSTNYNEKFKK

SEQ ID NO: 32 - protein, 17 aa, Artificial Sequence
  Synthetic
LSYPRDGTTQYNGKFKG

SEQ ID NO: 33 - protein, 17 aa, Artificial Sequence
  Synthetic
MIHPNSGSTNYNEKFKR

SEQ ID NO: 34 - protein, 17 aa, Artificial Sequence
  Synthetic
RIYPRDGGTNYNEKFKG

SEQ ID NO: 35 - protein, 17 aa, Artificial Sequence
  Synthetic
YINPKNGGTRYNQKFKG

SEQ ID NO: 36 - protein, 17 aa, Artificial Sequence
  Synthetic
YINPNNGGTTYNQKFKG

SEQ ID NO: 37 - protein, 17 aa, Artificial Sequence
  Synthetic
LSYPRDSSTQYNGRFRG

SEQ ID NO: 38 - protein, 17 aa, Artificial Sequence
  Synthetic
MIHPNSDSTNYNEKFKS

SEQ ID NO: 39 - protein, 17 aa, Artificial Sequence
  Synthetic
LIYPRDKNTNYNGKFKG

SEQ ID NO: 40 - protein, 17 aa, Artificial Sequence
  Synthetic
YINPYNSGTRYNQKFKG

SEQ ID NO: 41 - protein, 17 aa, Artificial Sequence
  Synthetic
SINPYNPGTRYNQKFEG

SEQ ID NO: 42 - protein, 17 aa, Artificial Sequence
  Synthetic
NINYDGSSTFYLDSLKS

SEQ ID NO: 43 - protein, 17 aa, Artificial Sequence
  Synthetic
MIHPNSGSTNYNEKFKS

SEQ ID NO: 44 - protein, 17 aa, Artificial Sequence
  Synthetic
FINPYSGSTTYNQKFKG

SEQ ID NO: 45 - protein, 17 aa, Artificial Sequence
  Synthetic
YISSGSSNIYYADTVKG

SEQ ID NO: 46 - protein, 17 aa, Artificial Sequence
  Synthetic
YISNLGGSTYYPDTVKG

SEQ ID NO: 47 - protein, 17 aa, Artificial Sequence
  Synthetic
YISNLGGSTFYPDTVKG

SEQ ID NO: 48 - protein, 17 aa, Artificial Sequence
  Synthetic
YISSGSSSFYYADTVKG

SEQ ID NO: 49 - protein, 16 aa, Artificial Sequence
  Synthetic
YISIGGTTYYPDTIKG

SEQ ID NO: 50 - protein, 17 aa, Artificial Sequence
  Synthetic
EINPTNGRSNYNEKFKK

SEQ ID NO: 51 - protein, 17 aa, Artificial Sequence
  Synthetic
YISSGSSSIYYVDTVKG

SEQ ID NO: 52 - protein, 17 aa, Artificial Sequence
  Synthetic
GIHPRDGNTKYNEKFKD

SEQ ID NO: 53 - protein, 17 aa, Artificial Sequence
  Synthetic
DVNPNYSTTRYNQKFKD

SEQ ID NO: 54 - protein, 16 aa, Artificial Sequence
  Synthetic
HITYDGSNNYNPSLKN

SEQ ID NO: 55 - protein, 17 aa, Artificial Sequence
  Synthetic
RIRPSDSDSTYNQNFKG

SEQ ID NO: 56 - protein, 17 aa, Artificial Sequence
  Synthetic
WIFPRDGSTKYNEKFRG

SEQ ID NO: 57 - protein, 17 aa, Artificial Sequence
  Synthetic
LSYPRDSTTQYNGKFRG

SEQ ID NO: 58 - protein, 16 aa, Artificial Sequence
  Synthetic
VIWGDGSTHYHSALIS

SEQ ID NO: 59 - protein, 17 aa, Artificial Sequence
  Synthetic
QIYPGNGNTNYNGGFKG

SEQ ID NO: 60 - protein, 17 aa, Artificial Sequence
  Synthetic
QIYPGDGDTNYNGGFRG

SEQ ID NO: 61 - protein, 17 aa, Artificial Sequence
  Synthetic
RIRPSDSDSNYNQKFKG

SEQ ID NO: 62 - protein, 17 aa, Artificial Sequence
  Synthetic
GIRPSDSNTNYNHKFKG

SEQ ID NO: 63 - protein, 17 aa, Artificial Sequence
  Synthetic
GIRPFDSNTNYNHKFKG

SEQ ID NO: 64 - protein, 17 aa, Artificial Sequence
  Synthetic
GIRPSDSNNNYNHKFKG

SEQ ID NO: 65 - protein, 17 aa, Artificial Sequence
  Synthetic
DINPKNGGSRYNQKFRG

SEQ ID NO: 66 - protein, 17 aa, Artificial Sequence
  Synthetic
DINPKNGGSRYNQKFKG

SEQ ID NO: 67 - protein, 17 aa, Artificial Sequence
  Synthetic
RIRPSDTATNYNQKFKG

SEQ ID NO: 68 - protein, 9 aa, Artificial Sequence
  Synthetic
GNYGNSFAY

SEQ ID NO: 69 - protein, 9 aa, Artificial Sequence
  Synthetic
GYYGNSFAY

SEQ ID NO: 70 - protein, 9 aa, Artificial Sequence
  Synthetic
MGLGNAMDF

SEQ ID NO: 71 - protein, 9 aa, Artificial Sequence
  Synthetic
MGLGNAMDY

SEQ ID NO: 72 - protein, 9 aa, Artificial Sequence
  Synthetic
IYYGNSFDY

SEQ ID NO: 73 - protein, 9 aa, Artificial Sequence
  Synthetic
MGLGNALDY

SEQ ID NO: 74 - protein, 9 aa, Artificial Sequence
  Synthetic
LYYGNSFDY

SEQ ID NO: 75 - protein, 9 aa, Artificial Sequence
  Synthetic
IFYGNSFDY

SEQ ID NO: 76 - protein, 9 aa, Artificial Sequence
  Synthetic
VFYGNSFDY

SEQ ID NO: 77 - protein, 10 aa, Artificial Sequence
  Synthetic
QVGYYDPMDY

SEQ ID NO: 78 - protein, 9 aa, Artificial Sequence
  Synthetic
FYYGNSFAY

SEQ ID NO: 79 - protein, 10 aa, Artificial Sequence
  Synthetic
HLYHYDAFAY

SEQ ID NO: 80 - protein, 10 aa, Artificial Sequence
  Synthetic
HLYNYDAFAS

SEQ ID NO: 81 - protein, 10 aa, Artificial Sequence
  Synthetic
NAYYGNALDY

SEQ ID NO: 82 - protein, 10 aa, Artificial Sequence
  Synthetic
HYYGHDVMDY

SEQ ID NO: 83 - protein, 9 aa, Artificial Sequence
  Synthetic
IYYGNSFAH

SEQ ID NO: 84 - protein, 10 aa, Artificial Sequence
  Synthetic
NAYYGNAFDY

SEQ ID NO: 85 - protein, 9 aa, Artificial Sequence
  Synthetic
LYYGNSFAY

SEQ ID NO: 86 - protein, 9 aa, Artificial Sequence
  Synthetic
GRYGNNRDY

SEQ ID NO: 87 - protein, 10 aa, Artificial Sequence
  Synthetic
GAYYSNSFGY

SEQ ID NO: 88 - protein, 10 aa, Artificial Sequence
  Synthetic
NGYYGNAMDY

SEQ ID NO: 89 - protein, 9 aa, Artificial Sequence
  Synthetic
PYYSNAMDY

SEQ ID NO: 90 - protein, 9 aa, Artificial Sequence
  Synthetic
WGTGNTMDY

SEQ ID NO: 91 - protein, 10 aa, Artificial Sequence
  Synthetic
GAYFSNSFAY

SEQ ID NO: 92 - protein, 10 aa, Artificial Sequence
  Synthetic
GAYYSNSFAY

SEQ ID NO: 93 - protein, 9 aa, Artificial Sequence
  Synthetic
TGYGNAMDY

SEQ ID NO: 94 - protein, 9 aa, Artificial Sequence
  Synthetic
LYFGNSFAY

SEQ ID NO: 95 - protein, 17 aa, Artificial Sequence
  Synthetic
KSSQSLFNSGNQKNYLT

SEQ ID NO: 96 - protein, 17 aa, Artificial Sequence
  Synthetic
KSSQSLLNSGNQKNYLT

SEQ ID NO: 97 - protein, 17 aa, Artificial Sequence
  Synthetic
KSSQSLLNDGNQKNYLT

SEQ ID NO: 98 - protein, 17 aa, Artificial Sequence
  Synthetic
KSSQSLLNGGNQKNYLT

SEQ ID NO: 99 - protein, 17 aa, Artificial Sequence
  Synthetic
KSSQSLLNSGNLKNYLT

SEQ ID NO: 100 - protein, 17 aa, Artificial Sequence
  Synthetic
KSSQSLLYSSNQKNYLA

SEQ ID NO: 101 - protein, 17 aa, Artificial Sequence
  Synthetic
KSSQSLLNSGNQRNYLT

SEQ ID NO: 102 - protein, 10 aa, Artificial Sequence
  Synthetic
RASSSLSYMH

SEQ ID NO: 103 - protein, 10 aa, Artificial Sequence
  Synthetic
RASSSVNYIH

SEQ ID NO: 104 - protein, 10 aa, Artificial Sequence
  Synthetic
RATSSVSYMH

SEQ ID NO: 105 - protein, 17 aa, Artificial Sequence
  Synthetic
KSGQSLLNSGNQRNYLT

SEQ ID NO: 106 - protein, 17 aa, Artificial Sequence
  Synthetic
RSSQILLNSGNQKNYLT

SEQ ID NO: 107 - protein, 17 aa, Artificial Sequence
  Synthetic
KSSQTLLNRGNQKNYLT

SEQ ID NO: 108 - protein, 17 aa, Artificial Sequence
  Synthetic
KSSQSLLNSGNQKNYVT

SEQ ID NO: 109 - protein, 17 aa, Artificial Sequence
  Synthetic
KSSQSLFNSGNQKNYLS

SEQ ID NO: 110 - protein, 17 aa, Artificial Sequence
  Synthetic
KSNQSLLNSGNQKNYVT

SEQ ID NO: 111 - protein, 17 aa, Artificial Sequence
  Synthetic
KSSQSLLNRGNQKNYVT

SEQ ID NO: 112 - protein, 17 aa, Artificial Sequence
  Synthetic
KSSQSLLNSGNRRNYLT

SEQ ID NO: 113 - protein, 17 aa, Artificial Sequence
  Synthetic
KSSQTLLNRGNQKNYVT

SEQ ID NO: 114 - protein, 7 aa, Artificial Sequence
  Synthetic
WASTRQS

SEQ ID NO: 115 - protein, 7 aa, Artificial Sequence
  Synthetic
WASTRES

SEQ ID NO: 116 - protein, 7 aa, Artificial Sequence
  Synthetic
WASTRAS

SEQ ID NO: 117 - protein, 7 aa, Artificial Sequence
  Synthetic
GTSNLAS

SEQ ID NO: 118 - protein, 7 aa, Artificial Sequence
  Synthetic
ATSNLAS

SEQ ID NO: 119 - protein, 7 aa, Artificial Sequence
  Synthetic
WASTRRS

SEQ ID NO: 120 - protein, 7 aa, Artificial Sequence
  Synthetic
WSSTRES

SEQ ID NO: 121 - protein, 7 aa, Artificial Sequence
  Synthetic
WASTRTS

SEQ ID NO: 122 - protein, 7 aa, Artificial Sequence
  Synthetic
WASTRDY

SEQ ID NO: 123 - protein, 7 aa, Artificial Sequence
  Synthetic
WASTRKS

SEQ ID NO: 124 - protein, 9 aa, Artificial Sequence
  Synthetic
QNGFSFPYT

SEQ ID NO: 125 - protein, 9 aa, Artificial Sequence
  Synthetic
QNDFGFPYT

SEQ ID NO: 126 - protein, 9 aa, Artificial Sequence
  Synthetic
QNNYVYPLT

SEQ ID NO: 127 - protein, 9 aa, Artificial Sequence
  Synthetic
QNDYYFPYT

SEQ ID NO: 128 - protein, 9 aa, Artificial Sequence
  Synthetic
QNDYYYPLT

SEQ ID NO: 129 - protein, 9 aa, Artificial Sequence
  Synthetic
QNGYSFPYT

SEQ ID NO: 130 - protein, 9 aa, Artificial Sequence
  Synthetic
QNDYSFPFT

SEQ ID NO: 131 - protein, 9 aa, Artificial Sequence
  Synthetic
QNDYYYPYT

SEQ ID NO: 132 - protein, 9 aa, Artificial Sequence
  Synthetic
QNDYFYPFT

SEQ ID NO: 133 - protein, 9 aa, Artificial Sequence
  Synthetic
QNNYFYPFT

SEQ ID NO: 134 - protein, 9 aa, Artificial Sequence
  Synthetic
QQYYTYPLT

SEQ ID NO: 135 - protein, 9 aa, Artificial Sequence
  Synthetic
QNAYSYPLT

SEQ ID NO: 136 - protein, 9 aa, Artificial Sequence
  Synthetic
QNAYSFPFT

SEQ ID NO: 137 - protein, 9 aa, Artificial Sequence
  Synthetic
QQWSSNPLT

SEQ ID NO: 138 - protein, 9 aa, Artificial Sequence
  Synthetic
QQWNSNPLT

SEQ ID NO: 139 - protein, 9 aa, Artificial Sequence
  Synthetic
QNVYVYPLT

SEQ ID NO: 140 - protein, 9 aa, Artificial Sequence
  Synthetic
QQWSRNPLT

SEQ ID NO: 141 - protein, 9 aa, Artificial Sequence
  Synthetic
QNNYYYPLT

SEQ ID NO: 142 - protein, 9 aa, Artificial Sequence
  Synthetic
QNGYTYPLT

SEQ ID NO: 143 - protein, 9 aa, Artificial Sequence
  Synthetic
QNAYFYPYT

SEQ ID NO: 144 - protein, 9 aa, Artificial Sequence
  Synthetic
QNAYFYPFT

SEQ ID NO: 145 - protein, 9 aa, Artificial Sequence
  Synthetic
QNDYFFPYT

SEQ ID NO: 146 - protein, 9 aa, Artificial Sequence
  Synthetic
QNNYNYPVT

SEQ ID NO: 147 - protein, 9 aa, Artificial Sequence
  Synthetic
QNVYSYPLT

SEQ ID NO: 148 - protein, 9 aa, Artificial Sequence
  Synthetic
QNVYSYPIT

SEQ ID NO: 149 - protein, 9 aa, Artificial Sequence
  Synthetic
QNDYFFPFT

SEQ ID NO: 150 - protein, 9 aa, Artificial Sequence
  Synthetic
QNDYVYPFT

SEQ ID NO: 151 - protein, 9 aa, Artificial Sequence
  Synthetic
QNNYFYPLT

SEQ ID NO: 152 - protein, 9 aa, Artificial Sequence
  Synthetic
QNDYTYPLT

SEQ ID NO: 153 - protein, 9 aa, Artificial Sequence
  Synthetic
QNDYSYPLT

SEQ ID NO: 154 - protein, 9 aa, Artificial Sequence
  Synthetic
QNDYNYPLT

SEQ ID NO: 155 - protein, 9 aa, Artificial Sequence
  Synthetic
QNDYSYPFM

SEQ ID NO: 156 - protein, 24 aa, Artificial Sequence
  Synthetic murine FR
EAKLVESGGDFMQPGGSLKLSCAA

SEQ ID NO: 157 - protein, 24 aa, Artificial Sequence
  Synthetic humanized FR for humanized antibody of 35B4
EVQLLESGGGLVQPGGSLRLSCAA

SEQ ID NO: 158 - protein, 25 aa, Artificial Sequence
  Synthetic murine FR
QVQLQQPGTELVKTGTSVKLSCKAS

SEQ ID NO: 159 - protein, 25 aa, Artificial Sequence [sequence omitted]

SEQ ID NO: 160 - protein, 25 aa, Artificial Sequence
  Synthetic humanized FR for humanized antibody of 22E12
QVQLVQSGAEVVKPGASVKVSCKAS

SEQ ID NO: 161 - protein, 25 aa, Artificial Sequence
  Synthetic humanized FR for humanized antibody of 22E12
QVQLVQSGAEVVKPGASVKLSCKAS

SEQ ID NO: 162 - protein, 14 aa, Artificial Sequence
  Synthetic murine FR
WVRQTPEKRLEWVA

SEQ ID NO: 163 - protein, 14 aa, Artificial Sequence
  Synthetic humanized FR for humanized antibody of 35B4
WVRQAPGKGLEWVS

SEQ ID NO: 164 - protein, 14 aa, Artificial Sequence
  Synthetic humanized FR for humanized antibody of 35B4
WVRQAPGKGLEWVA

SEQ ID NO: 165 - protein, 14 aa, Artificial Sequence
  Synthetic murine FR
WVIQRPGQGLEWIG

SEQ ID NO: 166 - protein, 14 aa, Artificial Sequence
  Synthetic humanized FR for humanized antibody of 22E12
WVRQAPGQGLEWMG

SEQ ID NO: 167 - protein, 14 aa, Artificial Sequence
  Synthetic humanized FR for humanized antibody of 22E12
WVIQAPGQGLEWMG

SEQ ID NO: 168 - protein, 14 aa, Artificial Sequence
  Synthetic humanized FR for humanized antibody of 22E12
WVIQAPGQGLEWIG

SEQ ID NO: 169 - protein, 32 aa, Artificial Sequence
  Synthetic murine FR
RFTISRDNARNTLFLQMSSLQSEDTAIYYCAT

SEQ ID NO: 170 - protein, 32 aa, Artificial Sequence
  Synthetic humanized FR for humanized antibody of 35B4
RFTISRDNSKNTLYLQMNSLRAEDTAVYYCAK

SEQ ID NO: 171 - protein, 32 aa, Artificial Sequence
  Synthetic humanized FR for humanized antibody of 35B4
RFTISRDNSKNTLYLQMNSLRAEDTAVYYCAT

SEQ ID NO: 172 - protein, 32 aa, Artificial Sequence
  Synthetic murine FR
KATLTLDRSSTTAYMQLSSLTSEDSAVYFCAG

SEQ ID NO: 173 - protein, 32 aa, Artificial Sequence
  Synthetic humanized FR for humanized antibody of 22E12
RVTMTRDTSTSTVYMELSSLRSEDTAVYYCAR

SEQ ID NO: 174 - protein, 32 aa, Artificial Sequence
  Synthetic humanized FR for humanized antibody of 22E12
RVTMTRDRSTSTVYMELSSLRSEDTAVYFCAG

SEQ ID NO: 175 - protein, 32 aa, Artificial Sequence
  Synthetic humanized FR for humanized antibody of 22E12
RVTMTLDRSTSTVYMELSSLRSEDTAVYFCAG

SEQ ID NO: 176 - protein, 32 aa, Artificial Sequence
  Synthetic humanized FR for humanized antibody of 22E12
RVTLTLDRSTSTVYMELSSLRSEDTAVYFCAG

SEQ ID NO: 177 - protein, 11 aa, Artificial Sequence
  Synthetic murine FR
WGQGTLVTVSA

SEQ ID NO: 178 - protein, 11 aa, Artificial Sequence
  Synthetic humanized FR for humanized antibody of 22E12
WGQGTLVTVSS

SEQ ID NO: 179 - protein, 23 aa, Artificial Sequence
  Synthetic murine FR
QIVLSQSPAILSASPGEKVTMTC

SEQ ID NO: 180 - protein, 23 aa, Artificial Sequence
  Synthetic humanized FR for humanized antibody of 35B4
DIQLTQSPSFLSASVGDRVTITC

SEQ ID NO: 181 - protein, 23 aa, Artificial Sequence
  Synthetic humanized FR for humanized antibody of 35B4
DIQLTQSPSFLSASVGDRVTMTC

SEQ ID NO: 182 - protein, 23 aa, Artificial Sequence
  Synthetic murine FR
DIVMTQSPSSLTVTAGEKVTLSC

SEQ ID NO: 183 - protein, 23 aa, Artificial Sequence
  Synthetic humanized FR for humanized antibody of 22E12
DIVMTQSPDSLAVSLGERATINC

SEQ ID NO: 184 - protein, 23 aa, Artificial Sequence
  Synthetic humanized FR for humanized antibody of 22E12
DIVMTQSPDSLAVSLGERVTLNC

SEQ ID NO: 185 - protein, 15 aa, Artificial Sequence
  Synthetic murine FR
WYQQKPGSSPKAWIY

SEQ ID NO: 186 - protein, 15 aa, Artificial Sequence
  Synthetic humanized FR for humanized antibody of 35B4
WYQQKPGKAPKLLIY

SEQ ID NO: 187 - protein, 15 aa, Artificial Sequence
  Synthetic humanized FR for humanized antibody of 35B4
WYQQKPGKAPKALIY

SEQ ID NO: 188 - protein, 15 aa, Artificial Sequence
  Synthetic humanized FR for humanized antibody of 35B4
WYQQKPGKSPKALIY

SEQ ID NO: 189 - protein, 15 aa, Artificial Sequence
  Synthetic humanized FR for humanized antibody of 22E12
WYQQKPGQPPKLLIY

SEQ ID NO: 190 - protein, 32 aa, Artificial Sequence
  Synthetic murine FR
GVPTRFSGSGSGTSYSLTIDRVEAEDAATYYC

SEQ ID NO: 191 - protein, 32 aa, Artificial Sequence
  Synthetic humanized FR for humanized antibody of 35B4
GVPSRFSGSGSGTEFTLTISSLQPEDFATYYC

SEQ ID NO: 192 - protein, 32 aa, Artificial Sequence
  Synthetic humanized FR for humanized antibody of 35B4
GVPSRFSGSGSGTEYTLTISSLQPEDFATYYC

SEQ ID NO: 193 - protein, 32 aa, Artificial Sequence
  Synthetic humanized FR for humanized antibody of 35B4
GVPSRFSGSGSGTEYTLTISSVQPEDFATYYC

SEQ ID NO: 194 - protein, 32 aa, Artificial Sequence
  Synthetic murine FR
GVPDRFTGSGSGTDFTLTISSVQAEDLAVYHC

SEQ ID NO: 195 - protein, 32 aa, Artificial Sequence
  Synthetic humanized FR for humanized antibody of 22E12
GVPDRFSGSGSGTDFTLTISSLQAEDVAVYYC

SEQ ID NO: 196 - protein, 32 aa, Artificial Sequence
  Synthetic humanized FR for humanized antibody of 22E12
GVPDRFSGSGSGTDFTLTISSLQAEDVAVYHC

SEQ ID NO: 197 - protein, 32 aa, Artificial Sequence
  Synthetic humanized FR for humanized antibody of 22E12
GVPDRFSGSGSGTDFTLTISSVQAEDVAVYHC

SEQ ID NO: 198 - protein, 10 aa, Artificial Sequence
  Synthetic murine FR
FGAGTKLELK

SEQ ID NO: 199 - protein, 10 aa, Artificial Sequence
  Synthetic humanized FR for humanized antibody of 35B4
FGQGTKLEIK

SEQ ID NO: 200 - protein, 10 aa, Artificial Sequence
  Synthetic humanized FR for humanized antibody of 22E12
FGGGTKLEIK

SEQ ID NO: 201 - protein, 11 aa, Artificial Sequence
  Synthetic
SGFTLSSYALS

SEQ ID NO: 202 - protein, 9 aa, Artificial Sequence
  Synthetic
GYTFTNWVH

SEQ ID NO: 203 - protein, 17 aa, Artificial Sequence
  Synthetic
EINPTNARSNYNEKFKK

SEQ ID NO: 204 - protein, 9 aa, Artificial Sequence
  Synthetic
QQWNANPLT

SEQ ID NO: 205 - protein, 17 aa, Artificial Sequence
  Synthetic
KSSQSLLNAGNQKNYLT

SEQ ID NO: 206 - protein, 118 aa, Artificial Sequence
  Synthetic
DVQLQESGPGLVKPSQSLSLTCSVTGYSITSGYLWNWIRQSPGNKLEWMGHITYDGSNNYNPSLKNRISITRDTSKNQFFLKLNSVTTEDTATYFCSRGRYGNNRDYWGQGTTLTVSS

SEQ ID NO: 207 - protein, 119 aa, Artificial Sequence
  Synthetic
DVQLVESGGGLVQPGGSRKLSCAASGFTFSSFGMHWVRQAPEKGLEWVAYISSGSSSFYYADTVKGRFTISRDNPKNTLFLQMTSLRSEDTAMYYCARNAYYGNALDYWGQGTTLTVSS

SEQ ID NO: 208 - protein, 119 aa, Artificial Sequence
  Synthetic
DVQLVESGGGLVQPGGSRKLSCAASGFTFSSFGMHWVRQAPEKGLEWVAYISSGSSSIYYVDTVKGRFTISRDNPKNTLFLQMTSLRSEDTAMYYCARNAYYGNAFDYWGQGTTLTVSS

SEQ ID NO: 209 - protein, 118 aa, Artificial Sequence
  Synthetic
DVQLVESGGGLVQPGGSRRLSCAASGFSFSNFGMYWVRQAPEKGLEWVAFITSDSTSIYYVDTVKGRFTVSRDNPKNTLFLQMTSLRSEDTAMYYCGRTGYGNAMDYWGQGTSVTVSS

SEQ ID NO: 210 - protein, 119 aa, Artificial Sequence
  Synthetic
EAKLVESGGDFMQPGGSLKLSCAASGFTLSSYALSWVRQTPEKRLEWVAYISNLGGSTFYPDTVKGRFTISRDNARNTLFLQMSSLQSEDTAIYYCATHLYNYDAFASWGQGTLVTVSA

SEQ ID NO: 211 - protein, 119 aa, Artificial Sequence
  Synthetic
EVKLVESEGGLVQPGSSMKLSCTASGFTFSDYYMAWVRQVPEKGLEWVANINYDGSSTFYLDSLKSRFIISRDNARNILYLQMTSLKSEDTATYFCGRQVGYYDPMDYWGQGTSVTVAS

SEQ ID NO: 212 - protein, 118 aa, Artificial Sequence
  Synthetic
EVKLVESGGGLVQPGGSLKLSCAASGFTFSRYAMSWVRQTPEKRLEWVAYISIGGTTYYPDTIKGRFTISRDNAKNTLYLQMSSLKSEDTAMYYCTRHYYGHDVMDYWGQGTSVTVSS

SEQ ID NO: 213 - protein, 119 aa, Artificial Sequence
  Synthetic
EVNLVESGGGFVQPGGSLKLSCVASGFTFSSYAMSWVRQTPDTRLEWVAYISNLGGSTYYPDTVKGRFTISRDNARNTLFLQMSSLQSEDTAMYYCTGHLYHYDAFAYWGQGTLVTVSA

SEQ ID NO: 214 - protein, 118 aa, Artificial Sequence
  Synthetic
EVQLQQFGAELVKPGTSVKISCKASGYTFTDYNMDWVKQSHGKSLEWIGDVNPNYSTTRYNQKFKDKATLTVDKSSSTAYMELRSLTSEDTAVYYCARLYYGNSFAYWGQGTLVTVSA

SEQ ID NO: 215 - protein, 118 aa, Artificial Sequence
  Synthetic
EVQLQQSGPDLLKPGASVKMSCKASGYTFTDYSMHWVRQSHGKRLEWIGFINPYSGSTTYNQKFKGKATLTVNKSSSTVYMEVRSLTSDDSAVYYCTRIFYGNSFDYWGQGTTLTVSS

SEQ ID NO: 216 - protein, 118 aa, Artificial Sequence
  Synthetic
EVQLQQSGPELLKPGASVKMSCKASGYTFTDYSMHWVRQSHGKRLEWIGFINPYSGSTTYNQKFKGKATLTVNKSSSTVYMEVRSLTSDDSAVYYCTRIFYGNSFDYWGQGTTLTVSS

SEQ ID NO: 217 - protein, 118 aa, Artificial Sequence
  Synthetic
EVQLQQSGPELLKPGASVKMSCKASGYTFTDYTIHWVKQSHGESLEWIGYINPYNSGTRYNQKFKGKATLTVNKSSSTAYMEVRSLTSEDSAVYFCTRIFYGNSFDYWGQGTTLTVSS

SEQ ID NO: 218 - protein, 118 aa, Artificial Sequence
  Synthetic
EVQLQQSGPELLKPGASVKMSCKASGYTFTDYTMHWVKQSHGKSLEWIGYINPYNSGTRYNQKFKGKATLTVNKSSNTAYMEVRSLTSEDSAVYYCTRIFYGNSFDYWGQGTTLTVSS

SEQ ID NO: 219 - protein, 118 aa, Artificial Sequence
  Synthetic
EVQLQQSGPELLKPGASVTMSCKASGYTFTDYTIHWVKQSHGKSLEWIGSINPYNPGTRYNQKFEGKATLTVNKSSNTAYMEFRSLTSEDSAVYYCTRVFYGNSFDYWGQGTTLTVSS

SEQ ID NO: 220 - protein, 118 aa, Artificial Sequence
  Synthetic
EVQLQQSGPELVKPGASVKMSCKASGYTFTDYNIHWLKQSPGKSLEWIGYINPKNGGTRYNQKFKGKATLTVNKSSSTAYMELRSLTSEDSAVYYCSRIYYGNSFDYWGQGTTLTVSS

SEQ ID NO: 221 - protein, 118 aa, Artificial Sequence
  Synthetic
EVQLQQSGPELVKPGASVKMSCKASGYTFTDYNMHWLKQSHGKSLEWIGYINPKNGGTRYNQKFKGKATLTVNKSSSTAYMELRSLTSEDSAVYYCARIYYGNSFDYWGQGTTLTVSS

SEQ ID NO: 222 - protein, 118 aa, Artificial Sequence
  Synthetic
EVQLQQSGPELVKPGASVKMSCKASGYTFTDYNMHWVKQSHGKSLEWIGYINPNNGGTTYNQKFKGKATLTVNKSSSTAYMELRGLTSEDSAIYYCARIYYGNSFDYWGQGTTLTVSS

SEQ ID NO: 223 - protein, 118 aa, Artificial Sequence
  Synthetic
EVQLQQSGPELVKPGSSVKMSCKASGYTFTDYNMHWLKQSHGKSLEWIGYINPKNGGTRYNQKFKGKATLTVNKSSSTAYMELRSLTSEDSAVYYCARLYYGNSFDYWGQGTTLTVSS

SEQ ID NO: 224 - protein, 118 aa, Artificial Sequence
  Synthetic
EVQLQQSGPEVVKPGTSVKISCKASGYTFTDYYMNWVKQSHGKSLEWIGDINPKNGGSRYNQKFKGKATLTADKSSNTAYMELRSLTSEDSAVYYCTRLYFGNSFAYWGQGTLVTVSA

SEQ ID NO: 225 - protein, 118 aa, Artificial Sequence
  Synthetic
EVQLQQSGPEVVKPGTSVKISCKASGYTFTDYYMNWVKQSHGKSLEWIGDINPKNGGSRYNQKFKGKATLTVDKSSNTAYMELRSLTSEDSAVYYCARLYYGNSFAYWGQGTLVTVSA

SEQ ID NO: 226 - protein, 118 aa, Artificial Sequence
  Synthetic
EVQLQQSGPEVVKPGTSVKISCKASGYTFTDYYMNWVKQSHGKSLEWIGDINPKNGGSRYNQKFRGKATLTVDKSSNTAFMELRSLTSEDSAVYYCARLYYGNSFAYWGQGTLVTVSA

SEQ ID NO: 227 - protein, 118 aa, Artificial Sequence
  Synthetic
EVQLVESGGGLVKPGGSLKLSCAASGFTFSDYGMHWVRQAPEKGLEWVAYISSGSSNIYYADTVKGRFTISRDNAKNTLFLQMTSLRSEDTAMYYCARFYYGNSFAYWGQGTLVTVSA

SEQ ID NO: 228 - protein, 119 aa, Artificial Sequence
  Synthetic
LVQLQQPGAELVKPGASVKVSCKASGSTFTTYWMHWVKKRPGQGLEWIGGIRPSDSNTNYNHKFKGKATLTVDKASSTAYMQLSSLTSEDSAVYYCAMGAYYSNSFAYWAQGTVVTVSA

SEQ ID NO: 229 - protein, 118 aa, Artificial Sequence
  Synthetic
QVHLQQSGPELVKPGASVKVSCKASGYSFRNYDINWVKQRPGQGLEWIGRIYPRDDSTTYNEKFKGKASLTVDTSSSTAYMEFHSLTSEDSAVYFCARGYYGNSFAYWGQGTLVTVSA

SEQ ID NO: 230 - protein, 119 aa, Artificial Sequence
  Synthetic
QVLLQQPGTELVKPGASVKVSCKASAYTFTSYWIHWVKQRPGQGLEWIGRIRPSDSDSTYNQNFKGKATLTVDKSSDTAYMQLTSLTSEDSAVYYCSMGAYYSNSFGYWGQGSLVTVSA

SEQ ID NO: 231 - protein, 117 aa, Artificial Sequence
  Synthetic
QVQLKESGPGLVAPSQSLSITCIVSGFSLTTYGISWVRQPPGKGLEWLGVIWGDGSTHYHSALISRLSISKDNSKSQVFLKLNSLQTDDTATYYCAKPYYSNAMDYWGQGTSVTVSS

SEQ ID NO: 232 - protein, 118 aa, Artificial Sequence
  Synthetic
QVQLQQPGAELIKPGASVKLSCKASGYTFTSYWIPWVKQRPGQGLEWIGMIHPNSDSTNYNEKFKSKATLTVDKSSSTAYIQLSSLTSDDSAVYYCARMGLGNALDYWGQGTSVTVSS

SEQ ID NO: 233 - protein, 118 aa, Artificial Sequence
  Synthetic
QVQLQQPGAELVKPGASVKLSCKASGYTFSSYWIPWVKQRPGQGLEWIGMIHPNSGSTNYNEKFKRKAILIVDKSSNTAYMQLSSLTSDDSAVYYCGRMGLGNAMDYWGQGTSVTVSS

SEQ ID NO: 234 - protein, 118 aa, Artificial Sequence
  Synthetic
QVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARMGLGNAMDYWGQGTSVTVSS

SEQ ID NO: 235 - protein, 118 aa, Artificial Sequence
  Synthetic
QVQLQQPGAELVKPGASVKLSCKASGYTVTRYWIQWVKQRPGQGLEWIGMIHPNSGSTNYNEKFKKKAALTLDKSSSTAYMQLSSPTSEDSAVYYCVRMGLGNAMDFWGQGTSVTVSS

SEQ ID NO: 236 - protein, 118 aa, Artificial Sequence
  Synthetic
QVQLQQPGAELVKPGASVKLSCKPSGYTFSSYWIPWVKQRPGQGLEWIGMIHPNSGSTNYNEKFKRKAILTVDKSSSTAYMQLSSLTSDDSAVYYCGRMGLGNAMDYWGQGTSVTVSS

SEQ ID NO: 237 - protein, 119 aa, Artificial Sequence
  Synthetic
QVQLQQPGAELVKPGASVKVSCKASGSTFTTYWMHWVKKRPGQGLEWIGGIRPFDSNTNYNHKFKGKATLTVDKASNTAYMQLSSLTSEDSAVYYCAMGAYYSNSFAYWGQGTVVTVSA

SEQ ID NO: 238 - protein, 119 aa, Artificial Sequence
  Synthetic
QVQLQQPGAELVKPGASVKVSCKASGSTFTTYWMHWVKKRPGQGLEWIGGIRPFDSNTNYNHKFKGKATLTVDKASSTAYMQLSSLTSEDSAVYYCAMGAYYSNSFAYWGQGTVVTVSA

SEQ ID NO: 239 - protein, 119 aa, Artificial Sequence
  Synthetic
QVQLQQPGAELVKPGASVKVSCKASGYTFTTYWMHWVRQRPGQGLEWIGRIRPSDTATNYNQKFKGKATLTVNKSSSTAYMQFSSLTSEDSAVFYCAMGAYYSNSFAYWGQGTLVTVSA

SEQ ID NO: 240 - protein, 119 aa, Artificial Sequence
  Synthetic
QVQLQQPGAELVNPGASVKVSCKASGSTFTTYWMHWVKKRPGQGLEWIGGIRPSDSNNNYNHKFKGKATLTVDKASSTAYLQLSSLTSEDSAVYYCAMGAYYSNSFAYWGQGTVVTVSA

SEQ ID NO: 241 - protein, 119 aa, Artificial Sequence
  Synthetic
QVQLQQPGPELVKPGASVKLSCKASGYTFTSYWMHWVRQRPGQGLEWIGMIHPNSGSTNYNEKFKSKATLTVDKSSSTAYMQLSSLTSEDSAVYFCARNGYYGNAMDYWGQGTSVTVSS

SEQ ID NO: 242 - protein, 119 aa, Artificial Sequence
  Synthetic
QVQLQQPGTELVKPGASVKVSCKASGYTFTSYWMHWVKQRPGQGLEWIGRIRPSDSDSNYNQKFKGKATLTVDKSSDTAYMQLNSLPSEDSAVYYCAMGAYFSNSFAYWGQGTLVTVSA

SEQ ID NO: 243 - protein, 119 aa, Artificial Sequence
  Synthetic
QVQLQQPGTELVKPGASVKVSCKASGYTFTTYWMHWVKQRPGQGLEWIGRIRPSDSDSNYNQKFKGKATLTVDKSSDTAYMQLNGLTSEDSAVYYCAMGAYFSNSFAYWGQGTLVTVSA

SEQ ID NO: 244 - protein, 119 aa, Artificial Sequence
  Synthetic
QVQLQQPGTELVKPGASVKVSCKASGYTFTTYWMHWVKQRPGQGLEWIGRIRPSDSDSNYNQKFKGKATLTVDKSSDTAYMQLNSLTSEDSAVYYCAMGAYFSNSFAYWGQGTLVTVSA

SEQ ID NO: 245 - protein, 119 aa, Artificial Sequence
  Synthetic
QVQLQQPGTELVKPGASVKVSCKASGYTFTTYWMHWVTQRPGQGLEWIGRIRPSDSDSNYNQKFKGKATLTVDKSSDTAYMQLNSLTSEDSAVYYCAMGAYFSNSFAYWGQGTLVTVSA

SEQ ID NO: 246 - protein, 117 aa, Artificial Sequence
  Synthetic
QVQLQQPGTELVKTGTSVKLSCKASGYTFTNWVHWVIQRPGQGLEWIGEINPTNGRSNYNEKFKKKATLTLDRSSTTAYMQLSSLTSEDSAVYFCAGIYYGNSFAHWGQGTLVTVSA

SEQ ID NO: 247 - protein, 118 aa, Artificial Sequence
  Synthetic
QVQLQQSGAELVKPGASVKISCKASGYAFSNYWMNWVKQRPGKGLEWIGQIYPGNGNTNYNGGFKGKATLTADKSSSTAYMHLNSLTSEDSAVYFCARWGTGNTMDYWGQGTSVTVSS

SEQ ID NO: 248 - protein, 118 aa, Artificial Sequence
  Synthetic
QVQLQQSGAELVKPGASVKISCKASGYAFSNYWMNWVNQRPGKGLEWIGQIYPGNGNTNYNGGFKGKATLTADKSSSTAYMHLNSLTSEDSAVYFCARWGTGNTMDYWGQGTSVTVSS

SEQ ID NO: 249 - protein, 118 aa, Artificial Sequence
  Synthetic
QVQLQQSGPDLVKPGASVKLSCKASGYTFTNYDINWVKQRPGQGLEWIGGIHPRDGNTKYNEKFKDKATLTIDTSANTAYMEFHSLTSEDSAVYFCARGYYGNSFAYWGQGTLVTVSA

SEQ ID NO: 250 - protein, 118 aa, Artificial Sequence
  Synthetic
QVQLQQSGPELVKPGASMKLSCKASGYSFTRNDINWVKQRPGQGLEWIGLSYPRDGTTQYNGKFKGKATLTVDTSSSTAYMELRSLTSEDSAVYFCARGYYGNSFAYWGQGTLVTVSA

SEQ ID NO: 251 - protein, 118 aa, Artificial Sequence
  Synthetic
QVQLQQSGPELVKPGASMKLSCKASGYTFTSYDINWVKQRPGQGLEWIGLSYPRDGTTQYNGKFKGEATLTVDRSSSTAYMELRSLTSEDSAVYFCARGYYGNSFAYWGQGTLVTVSA

SEQ ID NO: 252 - protein, 118 aa, Artificial Sequence
  Synthetic
QVQLQQSGPELVKPGASMKLSCKASGYTFTSYDINWVKQRPGQGLEWIGLSYPRDGTTQYNGKFKGKATLTVDTSSSTAYMELRSLTSEDSAVYFCARGYYGNSFAYWGQGTLVTVSA

SEQ ID NO: 253 - protein, 118 aa, Artificial Sequence
  Synthetic
QVQLQQSGPELVKPGASMKLSCKASGYTFTSYDINWVKQRPGQGPEWIGLSYPRDSSTQYNGRFRGKATLTVDTSSTTAYMELRSLTSEDSAVYFCARGYYGNSFAYWGQGTLVTVSA

SEQ ID NO: 254 - protein, 118 aa, Artificial Sequence
  Synthetic
QVQLQQSGPELVKPGASVKLSCKASGYSFTNFDINWVKQRPGQGLQWIGRLYPRDGTTTYNEKFKGKASLTVDTSSTTSYMDLHSLTSEDSAVYFCVRGNYGNSFAYWGQGTLVTVSA

SEQ ID NO: 255 - protein, 118 aa, Artificial Sequence
  Synthetic
QVQLQQSGPELVKPGASVKLSCKASGYTFTNYDINWVKQRPGQGLEWIGLIYPRDKNTNYNGKFKGKATLTVDTSSSTAYMELHSLTSEDSAVYFCARGYYGNSFAYWGQGTLVTVFA

SEQ ID NO: 256 - protein, 118 aa, Artificial Sequence
  Synthetic
QVQLQQSGPELVKPGSSVKLSCKASGYTFTRNDINWVKQRPGQGLEWIGRIYPRDGGTNYNEKFKGKATLTVDTLSSTAYMELHSLTSEDSAVHFCARGYYGNSFAYWGQGTLVTVSA

SEQ ID NO: 257 - protein, 118 aa, Artificial Sequence
  Synthetic
QVQLQQSGPELVKPGTSVKLSCKASGYTFINYDINWVKQRPGQGLEWIAWIFPRDGSTKYNEKFRGEATLTVDTSSSTAYLGLHSLTSEDSAVYFCARGYYGNSFAYWGQGTLVTVSA

SEQ ID NO: 258 - protein, 118 aa, Artificial Sequence
  Synthetic
QVQLQQSGTELVKPGASVKISCKASGYAFNSYWMNWLKQRPGKGLEWIGQIYPGDGDTNYNGGFRGKATLTADKSSRTAYMHLNSLTSEDSAVYFCARWGTGNTMDYWGQGTSVTVSS

SEQ ID NO: 259 - protein, 118 aa, Artificial Sequence
  Synthetic
RVQLQQSGPELVKPGASMKLSCKTSGYTFTNFDINWVKQRPGQGLEWIGLSYPRDSTTQYNGKFRGKATLTVDTSSTTAYMELRSLTSEDSAVYFCARGYYGNSFAYWGQGTLVTVSA

SEQ ID NO: 260 - protein, 113 aa, Artificial Sequence
  Synthetic
DIMMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSFPFTFGSGTKLEIK

SEQ ID NO: 261 - protein, 113 aa, Artificial Sequence
  Synthetic
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYTYPLTFGAGTKLELK

SEQ ID NO: 262 - protein, 113 aa, Artificial Sequence
  Synthetic
DIVMTQSPSFLTVTAGEKVTMSCKSSQSLLNSGNLKNYLTWYQQKPGQPPKLLIYWASTRESGVPDRFTGSGSGSDFTLTISSVQAEDLAVYYCQNDYFYPFTFGSGTRLEIK

SEQ ID NO: 263 - protein, 113 aa, Artificial Sequence
  Synthetic
DIVMTQSPSFLTVTAGEKVTMSCKSSQSLLNSGNLKNYLTWYQQKPGQPPKLLIYWASTRESGVPDRFTGSGSGSDFTLTISSVQAEDLAVYYCQNDYFYPFTFGSGTRLEMK

SEQ ID NO: 264 - protein, 113 aa, Artificial Sequence
  Synthetic
DIVMTQSPSFLTVTAGEKVTMSCKSSQSLLNSGNLKNYLTWYQQKPGQPPKLLIYWASTRESGVPDRFTGSGSGSDFTLTISSVQAEDLAVYYCQNNYFYPFTFGSGTRLEIK

SEQ ID NO: 265 - protein, 113 aa, Artificial Sequence
  Synthetic
DIVMTQSPSPLTVIVGEKVTMTCKSSQTLLNRGNQKNYLTWYQQKPGQPPKLLIYWASTRESGVPDRFTGSGSGTDFTLTINSVQAEDLAVYYCQNDYFFPFTFGSGTKLEIR

SEQ ID NO: 266 - protein, 113 aa, Artificial Sequence
  Synthetic
DIVMTQSPSPLTVTAGEKVTMTCKSSQTLLNRGNQKNYLTWYQQKPGQPPKLLIYWASTRESGVPDRFTGSGSGTDFTLTINSVQAEDLAVYYCQNDYFFPFTFGSGTKLEIR

SEQ ID NO: 267 - protein, 113 aa, Artificial Sequence
  Synthetic
DIVMTQSPSPLTVTAGEKVTMTCKSSQTLLNRGNQKNYLTWYQQKPGQPPKLLIYWASTRESGVPDRFTGSGSGTDFTLTINSVQAEDLAVYYCQNDYFFPFTFGSGTKLETR

SEQ ID NO: 268 - protein, 113 aa, Artificial Sequence
  Synthetic
DIVMTQSPSSLAVTPGEKVTMNCKSSQSLLNDGNQKNYLTWYQQKPGQPPKLLIYWASTRESGVPDRFAGSGSGTSFTLTINSVQAEDLAVYYCQNGYSFPYTFGGGTNLEIK

SEQ ID NO: 269 - protein, 113 aa, Artificial Sequence
  Synthetic
DIVMTQSPSSLPVTTGERVTMSCRSSQILLNSGNQKNYLTWYQQKPGQPPKLLIYWASTRDYGVPDRFTGSGSGTDFTLTISSVQAEDLAVYYCQNAYFYPFTFGAGTKLELK

SEQ ID NO: 270 - protein, 113 aa, Artificial Sequence
  Synthetic
DIVMTQSPSSLTVTAGEKATMSCKSSQSLLNGGNQKNYLTWYQQKPGQPPKLLIYWASTRESGVPDRFTGSGSGTYFTLTISSVQAEDLAVYYCQNDYYFPYTFGGGTKLEIK

SEQ ID NO: 271 - protein, 113 aa, Artificial Sequence
  Synthetic
DIVMTQSPSSLTVTAGEKATMSCKSSQSLLNGGNQKNYLTWYQQKPGQPPTLLIYWASTRESGVPDRFTGSGSGTYFTLTISSVQAEDLAVYYCQNDYYFPYTFGGGTKLEIK

SEQ ID NO: 272 - protein, 113 aa, Artificial Sequence
  Synthetic
DIVMTQSPSSLTVTAGEKVTLSCKSSQSLLNSGNQKNYLTWYQQKPGQPPKLLIYWASTRESGVPDRFTGSGSGTYFTLTISSVQAEDLAVYYCQNDYYFPYTFGGGTKLEIK

SEQ ID NO: 273 - protein, 113 aa, Artificial Sequence
  Synthetic
DIVMTQSPSSLTVTAGEKVTLSCKSSQSLLNSGNQKNYLTWYQQKPGQPPKLLIYWSSTRESGVPDRFTGSGSGTDFTLTISSVQAEDLAVYHCQNNYYYPLTFGAGTKLELK

SEQ ID NO: 274 - protein, 113 aa, Artificial Sequence
  Synthetic
DIVMTQSPSSLTVTAGEKVTLSCKSSQSLLNSGNQRNYLTWYQQKPGQPPKLLIYWASTRESGVPDRFTGSGSGTDFTLTISSVQAEDLAVYYCQNAYSYPLTFGAGTKLELK

SEQ ID NO: 275 - protein, 113 aa, Artificial Sequence
  Synthetic
DIVMTQSPSSLTVTAGEKVTMNCKSNQSLLNSGNQKNYVTWYQQKPGQPPKLLIFWASTRESGVPDRFTGSGSETDFTLIISSVQAEDLAVYYCQNDYVYPFTFGSGTKLEIK

SEQ ID NO: 276 - protein, 113 aa, Artificial Sequence
  Synthetic
DIVMTQSPSSLTVTAGEKVTMNCKSSQSLLNRGNQKNYVTWYQQKPGQPPKLLIFWASTRESGVPDRFTGSGSGTDFTLIISSVQAEDLAVYYCQNDYVYPFTFGSGTKLEIK

SEQ ID NO: 277 - protein, 113 aa, Artificial Sequence
  Synthetic
DIVMTQSPSSLTVTAGEKVTMNCKSSQSLLNSGNQKNYLTWYQQKPGQPPKLLIYWASTRESGVPDRFTGSGSGTDFTLTINSVQAEDLAVYYCQNDYSYPFMFGSGTKLEIK

SEQ ID NO: 278 - protein, 113 aa, Artificial Sequence
  Synthetic
DIVMTQSPSSLTVTAGEKVTMNCKSSQSLLNSGNQKNYLTWYQQKPGQPPKLLIYWASTRTSGVPDRFTGSGSGTDFTLTISSVQAEDLAVYCCQNGYTYPLTFGAGTKLELK

SEQ ID NO: 279 - protein, 113 aa, Artificial Sequence
  Synthetic
DIVMTQSPSSLTVTAGEKVTMSCKSSQSLLNGGNQKNYLTWYQQKPGQPPKLLIYWASTRESGVPDRFTGSGSGTDFTLTISTMQAEDLAVYYCQNDYFFPYTFGGGTKLEIK

SEQ ID NO: 280 - protein, 113 aa, Artificial Sequence
  Synthetic
DIVMTQSPSSLTVTAGEKVTMSCKSSQSLLNSGNQKNYLTWYQHKPGQPPKLLIYWASTRRSGVPDRFTGSGSGTDFTLTITSVQAEDLAVYCCQNVYVYPLTFGAGTKLELK

SEQ ID NO: 281 - protein, 113 aa, Artificial Sequence
  Synthetic
DIVMTQSPSSLTVTAGEKVTMSCKSSQSLLNSGNQKNYLTWYQKKPGQPPKLLIYWASTRESGVPDRFTGSGSGTDFTLTISSVQAEDLAVYFCQNDYSFPFTFGSGTKLEIK

SEQ ID NO: 282 - protein, 113 aa, Artificial Sequence
  Synthetic
DIVMTQSPSSLTVTAGEKVTMSCKSSQSLLNSGNQKNYLTWYQQKPGQAPKLLIYWASTRQSGVPDRFTGSGFGTDFTLIITTVQTEDLAVYFCQNDFGFPYTFGGGTKLEMN

SEQ ID NO: 283 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGRGSGTDFTLTISSVQAEDLAVYYCQNNYNYPVTFGAGTKLELK

SEQ ID NO: 284 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAADLAVYFCQNDYSFPFTFGSGTKLEIK

SEQ ID NO: 285 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYFCQNDYSFPFTFGSGTKLEIK

SEQ ID NO: 286 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNAYSFPFTFGSGTQLEIR

SEQ ID NO: 287 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYYYPLTFGAGTKLELK

SEQ ID NO: 288 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNNYVYPLTFGAGTKLELR

SEQ ID NO: 289 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNVYSYPITFGSGTKLEIK

SEQ ID NO: 290 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTYFTLTISSVQAEDLAVYFCQNDYYFPYTFGGGTKLEIK

SEQ ID NO: 291 - protein, 113 aa, Artificial Sequence
  Synthetic
DIVMTQSPSSLTVTAGEKVTMSCKSSQSLLNSGNQKNYLTWYQQKPGQPPKLLMYWASTRESGVPDRFTGSGSGTYFTLTISSVQAEDLAVYYCQNDYYFPYTFGGGTKLEIK

SEQ ID NO: 292 - protein, 113 aa, Artificial Sequence
  Synthetic
DIVMTQSPSSLTVTAGEKVTMSCKSSQSLLNSGNQKNYLTWYQQKPGQPPKMLIYWASTRESGVPDRFTGSGSGTDFTLTLSSVKAEDLAVYYCQNDYYYPLTFGAGTKLELR

SEQ ID NO: 293 - protein, 113 aa, Artificial Sequence
  Synthetic
DIVMTQSPSSLTVTAGEKVTMSCKSSQSLLNSGNQKNYLTWYQQKPGQSPKMLIYWASTRASGVPDRFTGSGSGTDFTLTLSSVKAEDLAVYYCQNDYYYPLTFGAGTKLELR

SEQ ID NO: 294 - protein, 113 aa, Artificial Sequence
  Synthetic
DIVMTQSPSSLTVTAGEKVTMTCKSGQSLLNSGNQRNYLTWYQQKPGQSPKLLIYWASTRESGVPDRFTGSGSGADFTLTISSVQAEDLALYYCQNAYFYPYTFGGGTKLEIK

SEQ ID NO: 295 - protein, 113 aa, Artificial Sequence
  Synthetic
DIVMTQSPSSLTVTAGEKVTMTCKSSQTLLNRGNQKNYVTWYQQKPGQPPKLLIYWASTRESGVPDRFTGSGSGTDFTLTISSVQAEDLAVYYCQNDYFFPFTFGSGTKLEIR

SEQ ID NO: 296 - protein, 113 aa, Artificial Sequence
  Synthetic
DIVMTQSPSSLTVTAGEMVTMNCKSSQSLLNSGNQRNYLTWYQQKPGQPPKLLIYWSSTRESGVPDRFTGSGSGTDFTLTISSVQAEDLAVYYCQNDYSYPLTFGAGTKLELK

SEQ ID NO: 297 - protein, 113 aa, Artificial Sequence
  Synthetic
DIVMTQSPSSLTVTAGEMVTMNCKSSQSLLNSGNQRNYLTWYQQKPGQPPKLLIYWSSTRESGVPDRFTGSGSGTDFTLTISSVQAEDLAVYYCQNDYTYPLTFGAGTKLELK

SEQ ID NO: 298 - protein, 113 aa, Artificial Sequence
  Synthetic
DIVMTQSPSSLTVTAGEMVTMNCKSSQSLLNSGNRRNYLTWYQQKPGQPPKLLIYWSSTRESGVPDRFAGSGSGTDFTLTISSVQAEDLAVYYCQNDYTYPLTFGAGTKLELK

SEQ ID NO: 299 - protein, 113 aa, Artificial Sequence
  Synthetic
DIVMTQSPSSLTVTAGERVTMSCKSSQSLLNSGNLKNYLTWYQQKPGQPPKLLIYWASTRESGVPDRFTGSGSGTYFTLTISSVQAEDLAVYYCQNDYYYPYTFGGGTKLEIK

SEQ ID NO: 300 - protein, 113 aa, Artificial Sequence
  Synthetic
DIVMTQSPSSLTVTAGKKVTMNCKSSQSLLNSGNQKNYVTWYQQKPGQPPKLLIFWASTRESGVPDRFTGSGSGTDFSLIISTVQAEDLAVYYCQNDYVYPFTFGSGTKLEIK

SEQ ID NO: 301 - protein, 113 aa, Artificial Sequence
  Synthetic
DIVMTQSPSSLTVTAREKVIMNCKSSQSLFNSGNQKNYLSWYQQKPGQPPKLLIYWASTRKSGVPDRFTGSGSGTGFTLTISSVQAEDLAVYYCQNNYFYPLTFGAGTKLELN

SEQ ID NO: 302 - protein, 113 aa, Artificial Sequence
  Synthetic
DIVMTQSPSSLTVTAREKVIMNCKSSQSLFNSGNQKNYLTWYQQKPGQSPKLLIYWASTRQSGVPDRFTGSGSGTDFTLTISTVQAEDLAVYFCQNGFSFPYTFGGGTKLEMN

SEQ ID NO: 303 - protein, 113 aa, Artificial Sequence
  Synthetic
DIVMTQSPSSLTVTAREKVTMNCKSSQSLLNSGNQKNYLTWYQQKPGQPPKMLIYWASTRESGVPDRFTGSGSGTYFTLTISSVQAEDLAVYYCQNDYSFPFTFGSGTKLEIK

SEQ ID NO: 304 - protein, 113 aa, Artificial Sequence
  Synthetic
DIVMTQSPSSLTVTPGEKVTMNCKSSQSLLNSGNQKNYVTWYQQKPGQPPKLLMFWASTRESGVPDRFTGSGSGTDFTLIISSVQAEDLAVYHCQNDYVYPFTFGSGTKLEIK

SEQ ID NO: 305 - protein, 113 aa, Artificial Sequence
  Synthetic
DIVMTQSPSSLTVTSGEKVTMSCKSSQSLFNSGNQKNYLTWYQQKLGQPPKLLIFWASTRESGVPDRFTGSGSGTDFTLTISSVQTEDLAVYYCQNDYYYPLTFGAGTKLELK

SEQ ID NO: 306 - protein, 113 aa, Artificial Sequence
  Synthetic
DIVMTQSPSSLTVTVGEKVTMSCKSSQSLLNSGNQKNYLTWYRQKPGQPPELLIYWASTRESGVPDRFTGSGSGTDFTLTISSVQAEDLAVYYCQNVYSYPLTFGAGTKLELK

SEQ ID NO: 307 - protein, 106 aa, Artificial Sequence
  Synthetic
QIVLSQSPAILSASPGEKVTMTCRASSSVNYIHWYQQKPGSSPKAWIYATSNLASGVPTRFSGSGSGTSYSLTIDRVEAEDAATYYCQQWNSNPLTFGAGTKLELK

SEQ ID NO: 308 - protein, 106 aa, Artificial Sequence
  Synthetic
QIVLSQSPAILSASPGEKVTVTCRASSSLSYMHWYQQRPGSSPKPWIYGTSNLASGVPARFSGSGSGTSYSLTISRVEAEDAATYYCQQWSSNPLTFGAGTKLELK

SEQ ID NO: 309 - protein, 106 aa, Artificial Sequence
  Synthetic
QIVLSQSPTILSASPGEKVTMTCRATSSVSYMHWFQQKPGSSPKPWIYATSNLASGVPARFSGSGSGTSYSLTISRVEAEDAATYYCQQWSRNPLTFGAGTKLELK

SEQ ID NO: 310 - protein, 113 aa, Artificial Sequence
  Synthetic
DIVMTQSPSSLTVTAGEMVTMNCKSSQSLLNSGNQRNYLTWYQQKPGQPPKLLIYWSSTRESGVPDRFTGSGSGTDFTLTISSVQAEDLAVYYCQNDYNYPLTFGAGTKLELK

SEQ ID NO: 311 - protein, 119 aa, Artificial Sequence
  Synthetic
EVQLLESGGGLVQPGGSLRLSCAASGFTLSSYALSWVRQAPGKGLEWVSYISNLGGSTFYPDTVKGRFTISRDNSKNTLYLQMNSLRAEDTAVYYCAKHLYNYDAFASWGQGTLVTVSS

SEQ ID NO: 312 - protein, 119 aa, Artificial Sequence
  Synthetic
EVQLLESGGGLVQPGGSLRLSCAASGFTLSSYALSWVRQAPGKGLEWVSYISNLGGSTFYPDTVKGRFTISRDNSKNTLYLQMNSLRAEDTAVYYCATHLYNYDAFASWGQGTLVTVSS

SEQ ID NO: 313 - protein, 119 aa, Artificial Sequence
  Synthetic
EVQLLESGGGLVQPGGSLRLSCAASGFTLSSYALSWVRQAPGKGLEWVAYISNLGGSTFYPDTVKGRFTISRDNSKNTLYLQMNSLRAEDTAVYYCATHLYNYDAFASWGQGTLVTVSS

SEQ ID NO: 314 - protein, 106 aa, Artificial Sequence
  Synthetic
DIQLTQSPSFLSASVGDRVTITCRASSSVNYIHWYQQKPGKAPKLLIYATSNLASGVPSRFSGSGSGTEFTLTISSLQPEDFATYYCQQWNSNPLTFGQGTKLEIK

SEQ ID NO: 315 - protein, 106 aa, Artificial Sequence
  Synthetic
DIQLTQSPSFLSASVGDRVTITCRASSSVNYIHWYQQKPGKAPKALIYATSNLASGVPSRFSGSGSGTEYTLTISSLQPEDFATYYCQQWNSNPLTFGQGTKLEIK

SEQ ID NO: 316 - protein, 106 aa, Artificial Sequence
  Synthetic
DIQLTQSPSFLSASVGDRVTITCRASSSVNYIHWYQQKPGKSPKALIYATSNLASGVPSRFSGSGSGTEYTLTISSLQPEDFATYYCQQWNSNPLTFGQGTKLEIK

SEQ ID NO: 317 - protein, 106 aa, Artificial Sequence
  Synthetic
DIQLTQSPSFLSASVGDRVTMTCRASSSVNYIHWYQQKPGKSPKALIYATSNLASGVPSRFSGSGSGTEYTLTISSVQPEDFATYYCQQWNSNPLTFGQGTKLEIK

SEQ ID NO: 318 - protein, 117 aa, Artificial Sequence
  Synthetic
QVQLVQSGAEVKKPGASVKVSCKASGYTFTNWVHWVRQAPGQGLEWMGEINPTNARSNYNEKFKKRVTMTRDTSTSTVYMELSSLRSEDTAVYYCARIYYGNSFAHWGQGTLVTVSS

SEQ ID NO: 319 - protein, 117 aa, Artificial Sequence
  Synthetic
QVQLVQSGAEVKKPGASVKVSCKASGYTFTNWVHWVRQAPGQGLEWMGEINPTNARSNYNEKFKKRVTMTRDRSTSTVYMELSSLRSEDTAVYFCAGIYYGNSFAHWGQGTLVTVSS

SEQ ID NO: 320 - protein, 117 aa, Artificial Sequence
  Synthetic
QVQLVQSGAEVVKPGASVKVSCKASGYTFTNWVHWVIQAPGQGLEWMGEINPTNARSNYNEKFKKRVTMTLDRSTSTVYMELSSLRSEDTAVYFCAGIYYGNSFAHWGQGTLVTVSS

SEQ ID NO: 321 - protein, 117 aa, Artificial Sequence
  Synthetic
QVQLVQSGAEVVKPGASVKLSCKASGYTFTNWVHWVIQAPGQGLEWIGEINPTNARSNYNEKFKKRVTLTLDRSTSTVYMELSSLRSEDTAVYFCAGIYYGNSFAHWGQGTLVTVSS

SEQ ID NO: 322 - protein, 113 aa, Artificial Sequence
  Synthetic
DIVMTQSPDSLAVSLGERATINCKSSQSLLNAGNQKNYLTWYQQKPGQPPKLLIYWSSTRESGVPDRFSGSGSGTDFTLTISSLQAEDVAVYYCQNNYYYPLTFGGGTKLEIK

SEQ ID NO: 323 - protein, 113 aa, Artificial Sequence
  Synthetic
DIVMTQSPDSLAVSLGERATINCKSSQSLLNAGNQKNYLTWYQQKPGQPPKLLIYWSSTRESGVPDRFSGSGSGTDFTLTISSLQAEDVAVYHCQNNYYYPLTFGGGTKLEIK

SEQ ID NO: 324 - protein, 113 aa, Artificial Sequence
  Synthetic
DIVMTQSPDSLAVSLGERVTLNCKSSQSLLNAGNQKNYLTWYQQKPGQPPKLLIYWSSTRESGVPDRFSGSGSGTDFTLTISSVQAEDVAVYHCQNNYYYPLTFGGGTKLEIK

SEQ ID NO: 325 - protein, 330 aa, Artificial Sequence
  Wild type human IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 329 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 331 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 332 - protein, 5 aa, Artificial Sequence
  Synthetic
  MISC_FEATURE (1)..(1): Xaa=N,S or R
  MISC_FEATURE (2)..(2): Xaa=F, Y, or N
XXDIN

SEQ ID NO: 333 - protein, 5 aa, Artificial Sequence
  Synthetic
  MISC_FEATURE (1)..(1): Xaa=D, S or T
  MISC_FEATURE (3)..(3): Xaa=N, W, S or T
XYXMH

SEQ ID NO: 334 - protein, 5 aa, Artificial Sequence
  Synthetic
  MISC_FEATURE (1)..(1): Xaa=S or R
  MISC_FEATURE (4)..(4): Xaa=M or L
XYAXS

SEQ ID NO: 335 - protein, 5 aa, Artificial Sequence
  Synthetic
  MISC_FEATURE (1)..(1): Xaa=N, D or S
  MISC_FEATURE (3)..(3): Xaa=Y or W
XYXMN

SEQ ID NO: 336 - protein, 5 aa, Artificial Sequence
  Synthetic
  MISC_FEATURE (1)..(1): Xaa=D or S
  MISC_FEATURE (3)..(3): Xaa=N, T or W
XYXIH

SEQ ID NO: 337 - protein, 5 aa, Artificial Sequence
  Synthetic
  MISC_FEATURE (1)..(1): Xaa=D or S
  MISC_FEATURE (2)..(2): Xaa=D or S
  MISC_FEATURE (2)..(2): Xaa=Y or F
XXGMH

SEQ ID NO: 338 - protein, 17 aa, Artificial Sequence
  Synthetic
  MISC_FEATURE (1)..(1): Xaa=R or L
  MISC_FEATURE (2)..(2): Xaa=L, I, or S
  MISC_FEATURE (7)..(7): Xaa=G, D, or K
  MISC_FEATURE (8)..(8): Xaa=T, S, G, or N
  MISC_FEATURE (10)..(10): Xaa=T, N, or Q
  MISC_FEATURE (13)..(13): Xaa=E or G
XXYPRDXXTXYNXKFKG

SEQ ID NO: 339 - protein, 17 aa, Artificial Sequence
  Synthetic
  MISC_FEATURE (5)..(5): Xaa=K, N or Y
  MISC_FEATURE (7)..(7): Xaa=G or S
  MISC_FEATURE (10)..(10): Xaa=R or T
YINPXNXGTXYNQKFKG

SEQ ID NO: 340 - protein, 17 aa, Artificial Sequence
  Synthetic
  MISC_FEATURE (7)..(7): Xaa=G or D
  MISC_FEATURE (17)..(17): Xaa=K, R or S
MIHPNSXSTNYNEKFKX

SEQ ID NO: 341 - protein, 17 aa, Artificial Sequence
  Synthetic
  MISC_FEATURE (10)..(10): Xaa=Y OR F
YISNLGGSTXYPDTVKG

SEQ ID NO: 342 - protein, 17 aa, Artificial Sequence
  Synthetic
  MISC_FEATURE (4)..(4): Xaa=S or I
  MISC_FEATURE (6)..(6): Xaa=S or G
  MISC_FEATURE (7)..(7): Xaa=S or T
  MISC_FEATURE (8)..(8): Xaa=S or T
  MISC_FEATURE (9)..(9): Xaa=F, I or absent
  MISC_FEATURE (12)..(12): Xaa=A, P or V
  MISC_FEATURE (15)..(15): Xaa=V or I
YISXGXXXXYYXDTXKG

SEQ ID NO: 343 - protein, 17 aa, Artificial Sequence
  Synthetic
  MISC_FEATURE (7)..(7): Xaa=G or A
EINPTNXRSNYNEKFKK

SEQ ID NO: 344 - protein, 9 aa, Artificial Sequence
  Synthetic
  MISC_FEATURE (1)..(1): Xaa=G, F, L or I
  MISC_FEATURE (2)..(2): Xaa=N or Y
  MISC_FEATURE (3)..(3): Xaa=Y or F
  MISC_FEATURE (9)..(9): Xaa=Y or H
XXXGNSFAX

SEQ ID NO: 345 - protein, 10 aa, Artificial Sequence
  Synthetic
  MISC_FEATURE (4)..(4): Xaa=H or N
  MISC_FEATURE (10)..(10): Xaa=Y or S
HLYXYDAFAX

SEQ ID NO: 346 - protein, 10 aa, Artificial Sequence
  Synthetic
  MISC_FEATURE (8)..(8): Xaa=L or F
NAYYGNAXDY

SEQ ID NO: 347 - protein, 17 aa, Artificial Sequence
  Synthetic
  MISC_FEATURE (1)..(1): Xaa=K or R
  MISC_FEATURE (5)..(5): Xaa=S or I
  MISC_FEATURE (7)..(7): Xaa=L or F
  MISC_FEATURE (9)..(9): Xaa=S or A
  MISC_FEATURE (13)..(13): Xaa=K or R
XSSQXLXNXGNQXNYLT

SEQ ID NO: 348 - protein, 10 aa, Artificial Sequence
  Synthetic
  MISC_FEATURE (3)..(3): Xaa=S or T
  MISC_FEATURE (6)..(6): Xaa=L or V
  MISC_FEATURE (7)..(7): Xaa=S or N
  MISC_FEATURE (9)..(9): Xaa=M or I
RAXSSXXYXH

SEQ ID NO: 349 - protein, 7 aa, Artificial Sequence
  Synthetic
  MISC_FEATURE (2)..(2): Xaa=A or S
  MISC_FEATURE (6)..(6): Xaa=Q, R, T, K, D or E
  MISC_FEATURE (7)..(7): Xaa=S or Y
WXSTRXX

SEQ ID NO: 350 - protein, 7 aa, Artificial Sequence
  Synthetic
  MISC_FEATURE (1)..(1): Xaa=G or A
XTSNLAS

SEQ ID NO: 351 - protein, 9 aa, Artificial Sequence
  Synthetic
  MISC_FEATURE (3)..(3): Xaa=G or D
  MISC_FEATURE (4)..(4): Xaa=F or Y
  MISC_FEATURE (5)..(5): Xaa=S or Y
QNXXXFPYT

SEQ ID NO: 352 - protein, 9 aa, Artificial Sequence
  Synthetic
  MISC_FEATURE (5)..(5): Xaa=S or F
  MISC_FEATURE (8)..(8): Xaa=L or F
QNAYXYPXT

SEQ ID NO: 353 - protein, 9 aa, Artificial Sequence
  Synthetic
  MISC_FEATURE (3)..(3): Xaa=N, V or G
  MISC_FEATURE (5)..(5): Xaa=Y, V or T
QNXYXYPLT

SEQ ID NO: 354 - protein, 9 aa, Artificial Sequence
  Synthetic
  MISC_FEATURE (4)..(4): Xaa=S or N
  MISC_FEATURE (5)..(5): Xaa=S, A or R
QQWXXNPLT

SEQ ID NO: 355 - protein, 24 aa, Artificial Sequence
  Synthetic
  MISC_FEATURE (2)..(2): Xaa=A or V
  MISC_FEATURE (3)..(3): Xaa=K or Q
  MISC_FEATURE (5)..(5): Xaa=V or L
  MISC_FEATURE (10)..(10): Xaa=D or G
  MISC_FEATURE (11)..(11): Xaa=F or L
  MISC_FEATURE (12)..(12): Xaa=M or V
  MISC_FEATURE (19)..(19): Xaa=K or R
EXXLXESGGXXXQPGGSLXLSCAA

SEQ ID NO: 356 - protein, 25 aa, Artificial Sequence
  Synthetic
  MISC_FEATURE (5)..(5): Xaa=Q or V
  MISC_FEATURE (7)..(7): Xaa=P or S
  MISC_FEATURE (9)..(9): Xaa=T or A
  MISC_FEATURE (11)..(11): Xaa=L or V
  MISC_FEATURE (12)..(12): Xaa=V or K
  MISC_FEATURE (14)..(14): Xaa=T or P
  MISC_FEATURE (16)..(16): Xaa=T or A
  MISC_FEATURE (20)..(20): Xaa=L or V
QVQLXQXGXEXXKXGXSVKXSCKAS

SEQ ID NO: 357 - protein, 14 aa, Artificial Sequence
  Synthetic
  MISC_FEATURE (5)..(5): Xaa=T or A
  MISC_FEATURE (7)..(7): Xaa=E or G
  MISC_FEATURE (9)..(9): Xaa=R or G
  MISC_FEATURE (14)..(14): Xaa=A or S
WVRQXPXKXLEWVX

SEQ ID NO: 358 - protein, 14 aa, Artificial Sequence
  Synthetic
  MISC_FEATURE (3)..(3): Xaa=I or R
  MISC_FEATURE (5)..(5): Xaa=R or A
  MISC_FEATURE (13)..(13): Xaa=I or M
WVXQXPGQGLEWXG

SEQ ID NO: 359 - protein, 32 aa, Artificial Sequence
  Synthetic
  MISC_FEATURE (9)..(9): Xaa=A or S
  MISC_FEATURE (10)..(10): Xaa=R or K
  MISC_FEATURE (14)..(14): Xaa=F or Y
  MISC_FEATURE (18)..(18): Xaa=S or N
  MISC_FEATURE (21)..(21): Xaa=Q or R
  MISC_FEATURE (22)..(22): Xaa=S or A
  MISC_FEATURE (27)..(27): Xaa=I or V
  MISC_FEATURE (32)..(32): Xaa=T or K
RFTISRDNXXNTLXLQMXSLXXEDTAXYYCAX

SEQ ID NO: 360 - protein, 32 aa, Artificial Sequence
  Synthetic
  MISC_FEATURE (1)..(1): Xaa=K or R
  MISC_FEATURE (2)..(2): Xaa=A or V
  MISC_FEATURE (4)..(4): Xaa=L or M
  MISC_FEATURE (6)..(6): Xaa=L or R
  MISC_FEATURE (8)..(8): Xaa=R or T
  MISC_FEATURE (10)..(10): Xaa=S or T
  MISC_FEATURE (11)..(11): Xaa=T or S
  MISC_FEATURE (13)..(13): Xaa=A or V
  MISC_FEATURE (16)..(16): Xaa=Q or E
  MISC_FEATURE (21)..(21): Xaa=T or R
  MISC_FEATURE (25)..(25): Xaa=S or T
  MISC_FEATURE (29)..(29): Xaa=F or Y
  MISC_FEATURE (32)..(32): Xaa=G or R
XXTXTXDXSXXTXYMXLSSLXSEDXAVYXCAX

SEQ ID NO: 361 - protein, 11 aa, Artificial Sequence
  Synthetic
  MISC_FEATURE (11)..(11): Xaa=A or S
WGQGTLVTVSX

SEQ ID NO: 362 - protein, 11 aa, Artificial Sequence
  Synthetic
  MISC_FEATURE (11)..(11): Xaa=A or S
WGQGTLVTVSX

SEQ ID NO: 363 - protein, 23 aa, Artificial Sequence
  Synthetic
  MISC_FEATURE (1)..(1): Xaa=Q or D
  MISC_FEATURE (3)..(3): Xaa=V or Q
  MISC_FEATURE (4)..(4): Xaa=L or M
  MISC_FEATURE (5)..(5): Xaa=S or T
  MISC_FEATURE (9)..(9): Xaa=A or S or D
  MISC_FEATURE (10)..(10): Xaa=I or F or S
  MISC_FEATURE (12)..(12): Xaa=S or T or A
  MISC_FEATURE (13)..(13): Xaa=A or V
  MISC_FEATURE (14)..(14): Xaa=S or T
  MISC_FEATURE (15)..(15): Xaa=P or V or A or L
  MISC_FEATURE (17)..(17): Xaa=E or D
  MISC_FEATURE (18)..(18): Xaa=K or R
  MISC_FEATURE (19)..(19): Xaa=V or A
  MISC_FEATURE (21)..(21): Xaa=M or I or L
  MISC_FEATURE (22)..(22): Xaa=T or S or N
XIXXXQSPXXLXXXXGXXXTXXC

SEQ ID NO: 364 - protein, 15 aa, Artificial Sequence
  Synthetic
  MISC_FEATURE (8)..(8): Xaa=S or K or Q
  MISC_FEATURE (9)..(9): Xaa=S or A or P
  MISC_FEATURE (12)..(12): Xaa=A or L
  MISC_FEATURE (13)..(13): Xaa=W or L
WYQQKPGXXPKXXIY

SEQ ID NO: 365 - protein, 32 aa, Artificial Sequence
  Synthetic
  MISC_FEATURE (4)..(4): Xaa=T or S or D
  MISC_FEATURE (7)..(7): Xaa=S or T
  MISC_FEATURE (14)..(14): Xaa=S or E or D
  MISC_FEATURE (15)..(15): Xaa=Y or F
  MISC_FEATURE (16)..(16): Xaa=S or T
  MISC_FEATURE (20)..(20): Xaa=D or S
  MISC_FEATURE (21)..(21): Xaa=R or S
  MISC_FEATURE (22)..(22): Xaa=V or L
  MISC_FEATURE (23)..(23): Xaa=E or Q
  MISC_FEATURE (24)..(24): Xaa=A or P
  MISC_FEATURE (27)..(27): Xaa=A or F or L or V
  MISC_FEATURE (29)..(29): Xaa=T or V
  MISC_FEATURE (31)..(31): Xaa=Y or H
GVPXRFXGSGSGTXXXLTIXXXXXEDXAXYXC

SEQ ID NO: 366 - protein, 10 aa, Artificial Sequence
  Synthetic
  MISC_FEATURE (3)..(3): Xaa=A or Q or A or G
  MISC_FEATURE (9)..(9): Xaa=L or I
FGXGTKLEXK

SEQ ID NO: 367 - protein, 17 aa, Artificial Sequence
  Synthetic
FITSDSTSIYYVDTVKG

SEQ ID NO: 368 - protein, 19 aa, Artificial Sequence
  Synthetic
MGWNWIFLFLLSGTAGVHS

SEQ ID NO: 369 - protein, 19 aa, Artificial Sequence
  Synthetic
MDSRLNLVFLVLILKGVQC

SEQ ID NO: 370 - protein, 19 aa, Artificial Sequence
  Synthetic
MGWSWIFLFLLSGTAGVLS

SEQ ID NO: 371 - protein, 19 aa, Artificial Sequence
  Synthetic
MRWSCIILFLVATATGVHS

SEQ ID NO: 372 - protein, 19 aa, Artificial Sequence
  Synthetic
MRWSCIIFLFIATATGVHS

SEQ ID NO: 373 - protein, 19 aa, Artificial Sequence
  Synthetic
MEWPLIFLFLLSGTAGVQS

SEQ ID NO: 374 - protein, 19 aa, Artificial Sequence
  Synthetic
MRWSCIILFLVASATGVHS

SEQ ID NO: 375 - protein, 19 aa, Artificial Sequence
  Synthetic
MGWSYIILFLVATATDVHS

SEQ ID NO: 376 - protein, 19 aa, Artificial Sequence
  Synthetic
MRWSCIIFLFIATATGIHS

SEQ ID NO: 377 - protein, 19 aa, Artificial Sequence
  Synthetic
MNFGLSLIFLVLVLKGVLC

SEQ ID NO: 378 - protein, 19 aa, Artificial Sequence
  Synthetic
MAVLALLLCLVTFPSCVLS

SEQ ID NO: 379 - protein, 19 aa, Artificial Sequence
  Synthetic
MGWSYIILFLVATATGVHS

SEQ ID NO: 380 - protein, 19 aa, Artificial Sequence
  Synthetic
MEWSWIFLFLLSGTASVLS

SEQ ID NO: 381 - protein, 18 aa, Artificial Sequence
  Synthetic
MKVLSLLYLLTAIPGILS

SEQ ID NO: 382 - protein, 19 aa, Artificial Sequence
  Synthetic
MGWSWIFLFLLSRTAGVHS

SEQ ID NO: 383 - protein, 19 aa, Artificial Sequence
  Synthetic
MGWSWIFLFLLSETAGVLS

SEQ ID NO: 384 - protein, 19 aa, Artificial Sequence
  Synthetic
MYFRLSSVFLLLILKGVQC

SEQ ID NO: 385 - protein, 19 aa, Artificial Sequence
  Synthetic
MGWSWIFLFLLSEAAGVLS

SEQ ID NO: 386 - protein, 19 aa, Artificial Sequence
  Synthetic
MGWSWIFLFLLSGTAGVHS

SEQ ID NO: 387 - protein, 19 aa, Artificial Sequence
  Synthetic
MGWYWIFLFLLSGTAGVHS

SEQ ID NO: 388 - protein, 19 aa, Artificial Sequence
  Synthetic
MGWSWIFLFLLSGTAGVRS

SEQ ID NO: 389 - protein, 20 aa, Artificial Sequence
  Synthetic
MESQTQVLMSLLFWVSGTYG

SEQ ID NO: 390 - protein, 20 aa, Artificial Sequence
  Synthetic
MESQTQVLMSLLFWVSGTCG

SEQ ID NO: 391 - protein, 20 aa, Artificial Sequence
  Synthetic
MESQTQVLMSLLFWVSGSCG

SEQ ID NO: 392 - protein, 22 aa, Artificial Sequence
  Synthetic
MDFQVQIFSFLLISASVIMSRG

SEQ ID NO: 393 - protein, 22 aa, Artificial Sequence
  Synthetic
MDFQVQIFSLLLISASVILSRG

SEQ ID NO: 394 - protein, 22 aa, Artificial Sequence
  Synthetic
MDFQVQIFSFLLISASVMMSRG

SEQ ID NO: 395 - protein, 20 aa, Artificial Sequence
  Synthetic
MESQTQVLMSLLFWVSGICG

SEQ ID NO: 396 - protein, 20 aa, Artificial Sequence
  Synthetic
MDSQAQVLMLLLLWVSGTCG

SEQ ID NO: 397 - protein, 118 aa, Artificial Sequence
  Synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 398 - protein, 113 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 399 - protein, 448 aa, Artificial Sequence
  Synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 400 - protein, 220 aa, Artificial Sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 401 - protein, 261 aa, Artificial Sequence
  Synthetic
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 402 - protein, 106 aa, Artificial Sequence
  Synthetic
DIQLTQSPSFLSASVGDRVTITCRASSSVNYIHWYQQKPGKAPKALIYATSNLASGVPSRFSGSGSGTEYTLTISSLQPEDFATYYCQQWNANPLTFGQGTKLEIK

SEQ ID NO: 403 - protein, 136 aa, Artificial Sequence
  Synthetic
MAVLALLLCLVTFPSCVLSQVQLKESGPGLVAPSQSLSITCIVSGFSLTTYGISWVRQPPGKGLEWLGVIWGDGSTHYHSALISRLSISKDNSKSQVFLKLNSLQTDDTATYYCAKPYYSNAMDYWGQGTSVTVSS

SEQ ID NO: 404 - protein, 138 aa, Artificial Sequence
  Synthetic
MDSRLNLVFLVLILKGVQCDVQLVESGGGLVQPGGSRKLSCAASGFTFSSFGMHWVRQAPEKGLEWVAYISSGSSSFYYADTVKGRFTISRDNPKNTLFLQMTSLRSEDTAMYYCARNAYYGNALDYWGQGTTLTVSS

SEQ ID NO: 405 - protein, 138 aa, Artificial Sequence
  Synthetic
MDSRLNLVFLVLILKGVQCDVQLVESGGGLVQPGGSRKLSCAASGFTFSSFGMHWVRQAPEKGLEWVAYISSGSSSIYYVDTVKGRFTISRDNPKNTLFLQMTSLRSEDTAMYYCARNAYYGNAFDYWGQGTTLTVSS

SEQ ID NO: 406 - protein, 137 aa, Artificial Sequence
  Synthetic
MDSRLNLVFLVLILKGVQCDVQLVESGGGLVQPGGSRRLSCAASGFSFSNFGMYWVRQAPEKGLEWVAFITSDSTSIYYVDTVKGRFTVSRDNPKNTLFLQMTSLRSEDTAMYYCGRTGYGNAMDYWGQGTSVTVSS

SEQ ID NO: 407 - protein, 137 aa, Artificial Sequence
  Synthetic
MDSRLNLVFLVLILKGVQCEVQLVESGGGLVKPGGSLKLSCAASGFTFSDYGMHWVRQAPEKGLEWVAYISSGSSNIYYADTVKGRFTISRDNAKNTLFLQMTSLRSEDTAMYYCARFYYGNSFAYWGQGTLVTVSA

SEQ ID NO: 408 - protein, 137 aa, Artificial Sequence
  Synthetic
MEWPLIFLFLLSGTAGVQSQVQLQQSGAELVKPGASVKISCKASGYAFSNYWMNWVKQRPGKGLEWIGQIYPGNGNTNYNGGFKGKATLTADKSSSTAYMHLNSLTSEDSAVYFCARWGTGNTMDYWGQGTSVTVSS

SEQ ID NO: 409 - protein, 137 aa, Artificial Sequence
  Synthetic
MEWPLIFLFLLSGTAGVQSQVQLQQSGAELVKPGASVKISCKASGYAFSNYWMNWVNQRPGKGLEWIGQIYPGNGNTNYNGGFKGKATLTADKSSSTAYMHLNSLTSEDSAVYFCARWGTGNTMDYWGQGTSVTVSS

SEQ ID NO: 410 - protein, 137 aa, Artificial Sequence
  Synthetic
MEWPLIFLFLLSGTAGVQSQVQLQQSGTELVKPGASVKISCKASGYAFNSYWMNWLKQRPGKGLEWIGQIYPGDGDTNYNGGFRGKATLTADKSSRTAYMHLNSLTSEDSAVYFCARWGTGNTMDYWGQGTSVTVSS

SEQ ID NO: 411 - protein, 137 aa, Artificial Sequence
  Synthetic
MEWSWIFLFLLSGTASVLSEVQLQQFGAELVKPGTSVKISCKASGYTFTDYNMDWVKQSHGKSLEWIGDVNPNYSTTRYNQKFKDKATLTVDKSSSTAYMELRSLTSEDTAVYYCARLYYGNSFAYWGQGTLVTVSA

SEQ ID NO: 412 - protein, 137 aa, Artificial Sequence
  Synthetic
MGWNWIFLFLLSGTAGVHSQVQLQQSGPDLVKPGASVKLSCKASGYTFTNYDINWVKQRPGQGLEWIGGIHPRDGNTKYNEKFKDKATLTIDTSANTAYMEFHSLTSEDSAVYFCARGYYGNSFAYWGQGTLVTVSA

SEQ ID NO: 413 - protein, 137 aa, Artificial Sequence
  Synthetic
MGWSWIFLFLLSEAAGVLSEVQLQQSGPELLKPGASVKMSCKASGYTFTDYTIHWVKQSHGESLEWIGYINPYNSGTRYNQKFKGKATLTVNKSSSTAYMEVRSLTSEDSAVYFCTRIFYGNSFDYWGQGTTLTVSS

SEQ ID NO: 414 - protein, 137 aa, Artificial Sequence
  Synthetic
MGWSWIFLFLLSETAGVLSEVQLQQSGPDLLKPGASVKMSCKASGYTFTDYSMHWVRQSHGKRLEWIGFINPYSGSTTYNQKFKGKATLTVNKSSSTVYMEVRSLTSDDSAVYYCTRIFYGNSFDYWGQGTTLTVSS

SEQ ID NO: 415 - protein, 137 aa, Artificial Sequence
  Synthetic
MGWSWIFLFLLSETAGVLSEVQLQQSGPELLKPGASVKMSCKASGYTFTDYSMHWVRQSHGKRLEWIGFINPYSGSTTYNQKFKGKATLTVNKSSSTVYMEVRSLTSDDSAVYYCTRIFYGNSFDYWGQGTTLTVSS

SEQ ID NO: 416 - protein, 137 aa, Artificial Sequence
  Synthetic
MGWSWIFLFLLSETAGVLSEVQLQQSGPELLKPGASVKMSCKASGYTFTDYTMHWVKQSHGKSLEWIGYINPYNSGTRYNQKFKGKATLTVNKSSNTAYMEVRSLTSEDSAVYYCTRIFYGNSFDYWGQGTTLTVSS

SEQ ID NO: 417 - protein, 137 aa, Artificial Sequence
  Synthetic
MGWSWIFLFLLSETAGVLSEVQLQQSGPELLKPGASVTMSCKASGYTFTDYTIHWVKQSHGKSLEWIGSINPYNPGTRYNQKFEGKATLTVNKSSNTAYMEFRSLTSEDSAVYYCTRVFYGNSFDYWGQGTTLTVSS

SEQ ID NO: 418 - protein, 137 aa, Artificial Sequence
  Synthetic
MGWSWIFLFLLSETAGVLSEVQLQQSGPELVKPGASVKMSCKASGYTFTDYNIHWLKQSPGKSLEWIGYINPKNGGTRYNQKFKGKATLTVNKSSSTAYMELRSLTSEDSAVYYCSRIYYGNSFDYWGQGTTLTVSS

SEQ ID NO: 419 - protein, 137 aa, Artificial Sequence
  Synthetic
MGWSWIFLFLLSETAGVLSEVQLQQSGPELVKPGASVKMSCKASGYTFTDYNMHWLKQSHGKSLEWIGYINPKNGGTRYNQKFKGKATLTVNKSSSTAYMELRSLTSEDSAVYYCARIYYGNSFDYWGQGTTLTVSS

SEQ ID NO: 420 - protein, 137 aa, Artificial Sequence
  Synthetic
MGWSWIFLFLLSETAGVLSEVQLQQSGPELVKPGASVKMSCKASGYTFTDYNMHWVKQSHGKSLEWIGYINPNNGGTTYNQKFKGKATLTVNKSSSTAYMELRGLTSEDSAIYYCARIYYGNSFDYWGQGTTLTVSS

SEQ ID NO: 421 - protein, 137 aa, Artificial Sequence
  Synthetic
MGWSWIFLFLLSETAGVLSEVQLQQSGPELVKPGSSVKMSCKASGYTFTDYNMHWLKQSHGKSLEWIGYINPKNGGTRYNQKFKGKATLTVNKSSSTAYMELRSLTSEDSAVYYCARLYYGNSFDYWGQGTTLTVSS

SEQ ID NO: 422 - protein, 137 aa, Artificial Sequence
  Synthetic
MGWSWIFLFLLSGTAGVHSQVQLQQSGPELVKPGASMKLSCKASGYSFTRNDINWVKQRPGQGLEWIGLSYPRDGTTQYNGKFKGKATLTVDTSSSTAYMELRSLTSEDSAVYFCARGYYGNSFAYWGQGTLVTVSA

SEQ ID NO: 423 - protein, 137 aa, Artificial Sequence
  Synthetic
MGWSWIFLFLLSGTAGVHSQVQLQQSGPELVKPGASMKLSCKASGYTFTSYDINWVKQRPGQGLEWIGLSYPRDGTTQYNGKFKGEATLTVDRSSSTAYMELRSLTSEDSAVYFCARGYYGNSFAYWGQGTLVTVSA

SEQ ID NO: 424 - protein, 137 aa, Artificial Sequence
  Synthetic
MGWSWIFLFLLSGTAGVHSQVQLQQSGPELVKPGASMKLSCKASGYTFTSYDINWVKQRPGQGLEWIGLSYPRDGTTQYNGKFKGKATLTVDTSSSTAYMELRSLTSEDSAVYFCARGYYGNSFAYWGQGTLVTVSA

SEQ ID NO: 425 - protein, 137 aa, Artificial Sequence
  Synthetic
MGWSWIFLFLLSGTAGVHSQVQLQQSGPELVKPGASVKLSCKASGYTFTNYDINWVKQRPGQGLEWIGLIYPRDKNTNYNGKFKGKATLTVDTSSSTAYMELHSLTSEDSAVYFCARGYYGNSFAYWGQGTLVTVFA

SEQ ID NO: 426 - protein, 137 aa, Artificial Sequence
  Synthetic
MGWSWIFLFLLSGTAGVHSQVQLQQSGPELVKPGSSVKLSCKASGYTFTRNDINWVKQRPGQGLEWIGRIYPRDGGTNYNEKFKGKATLTVDTLSSTAYMELHSLTSEDSAVHFCARGYYGNSFAYWGQGTLVTVSA

SEQ ID NO: 427 - protein, 137 aa, Artificial Sequence
  Synthetic
MGWSWIFLFLLSGTAGVHSQVQLQQSGPELVKPGTSVKLSCKASGYTFINYDINWVKQRPGQGLEWIAWIFPRDGSTKYNEKFRGEATLTVDTSSSTAYLGLHSLTSEDSAVYFCARGYYGNSFAYWGQGTLVTVSA

SEQ ID NO: 428 - protein, 137 aa, Artificial Sequence
  Synthetic
MGWSWIFLFLLSGTAGVLSEVQLQQSGPEVVKPGTSVKISCKASGYTFTDYYMNWVKQSHGKSLEWIGDINPKNGGSRYNQKFKGKATLTADKSSNTAYMELRSLTSEDSAVYYCTRLYFGNSFAYWGQGTLVTVSA

SEQ ID NO: 429 - protein, 137 aa, Artificial Sequence
  Synthetic
MGWSWIFLFLLSGTAGVLSEVQLQQSGPEVVKPGTSVKISCKASGYTFTDYYMNWVKQSHGKSLEWIGDINPKNGGSRYNQKFKGKATLTVDKSSNTAYMELRSLTSEDSAVYYCARLYYGNSFAYWGQGTLVTVSA

SEQ ID NO: 430 - protein, 137 aa, Artificial Sequence
  Synthetic
MGWSWIFLFLLSGTAGVLSEVQLQQSGPEVVKPGTSVKISCKASGYTFTDYYMNWVKQSHGKSLEWIGDINPKNGGSRYNQKFRGKATLTVDKSSNTAFMELRSLTSEDSAVYYCARLYYGNSFAYWGQGTLVTVSA

SEQ ID NO: 431 - protein, 137 aa, Artificial Sequence
  Synthetic
MGWSWIFLFLLSGTAGVRSQVQLQQSGPELVKPGASVKLSCKASGYSFTNFDINWVKQRPGQGLQWIGRLYPRDGTTTYNEKFKGKASLTVDTSSTTSYMDLHSLTSEDSAVYFCVRGNYGNSFAYWGQGTLVTVSA

SEQ ID NO: 432 - protein, 137 aa, Artificial Sequence
  Synthetic
MGWSWIFLFLLSRTAGVHSQVQLQQSGPELVKPGASMKLSCKASGYTFTSYDINWVKQRPGQGPEWIGLSYPRDSSTQYNGRFRGKATLTVDTSSTTAYMELRSLTSEDSAVYFCARGYYGNSFAYWGQGTLVTVSA

SEQ ID NO: 433 - protein, 137 aa, Artificial Sequence
  Synthetic
MGWSWIFLFLLSRTAGVHSRVQLQQSGPELVKPGASMKLSCKTSGYTFTNFDINWVKQRPGQGLEWIGLSYPRDSTTQYNGKFRGKATLTVDTSSTTAYMELRSLTSEDSAVYFCARGYYGNSFAYWGQGTLVTVSA

SEQ ID NO: 434 - protein, 136 aa, Artificial Sequence
  Synthetic
MGWSYIILFLVATATDVHSQVQLQQPGTELVKTGTSVKLSCKASGYTFTNWVHWVIQRPGQGLEWIGEINPTNGRSNYNEKFKKKATLTLDRSSTTAYMQLSSLTSEDSAVYFCAGIYYGNSFAHWGQGTLVTVSA

SEQ ID NO: 435 - protein, 137 aa, Artificial Sequence
  Synthetic
MGWSYIILFLVATATGVHSQVQLQQPGAELIKPGASVKLSCKASGYTFTSYWIPWVKQRPGQGLEWIGMIHPNSDSTNYNEKFKSKATLTVDKSSSTAYIQLSSLTSDDSAVYYCARMGLGNALDYWGQGTSVTVSS

SEQ ID NO: 436 - protein, 137 aa, Artificial Sequence
  Synthetic
MGWSYIILFLVATATGVHSQVQLQQPGAELVKPGASVKLSCKASGYTFSSYWIPWVKQRPGQGLEWIGMIHPNSGSTNYNEKFKRKAILIVDKSSNTAYMQLSSLTSDDSAVYYCGRMGLGNAMDYWGQGTSVTVSS

SEQ ID NO: 437 - protein, 137 aa, Artificial Sequence
  Synthetic
MGWSYIILFLVATATGVHSQVQLQQPGAELVKPGASVKLSCKASGYTFTSYWMHWVKQRPGQGLEWIGMIHPNSGSTNYNEKFKSKATLTVDKSSSTAYMQLSSLTSEDSAVYYCARMGLGNAMDYWGQGTSVTVSS

SEQ ID NO: 438 - protein, 137 aa, Artificial Sequence
  Synthetic
MGWSYIILFLVATATGVHSQVQLQQPGAELVKPGASVKLSCKASGYTVTRYWIQWVKQRPGQGLEWIGMIHPNSGSTNYNEKFKKKAALTLDKSSSTAYMQLSSPTSEDSAVYYCVRMGLGNAMDFWGQGTSVTVSS

SEQ ID NO: 439 - protein, 137 aa, Artificial Sequence
  Synthetic
MGWSYIILFLVATATGVHSQVQLQQPGAELVKPGASVKLSCKPSGYTFSSYWIPWVKQRPGQGLEWIGMIHPNSGSTNYNEKFKRKAILTVDKSSSTAYMQLSSLTSDDSAVYYCGRMGLGNAMDYWGQGTSVTVSS

SEQ ID NO: 440 - protein, 138 aa, Artificial Sequence
  Synthetic
MGWSYIILFLVATATGVHSQVQLQQPGPELVKPGASVKLSCKASGYTFTSYWMHWVRQRPGQGLEWIGMIHPNSGSTNYNEKFKSKATLTVDKSSSTAYMQLSSLTSEDSAVYFCARNGYYGNAMDYWGQGTSVTVSS

SEQ ID NO: 441 - protein, 137 aa, Artificial Sequence
  Synthetic
MGWYWIFLFLLSGTAGVHSQVHLQQSGPELVKPGASVKVSCKASGYSFRNYDINWVKQRPGQGLEWIGRIYPRDDSTTYNEKFKGKASLTVDTSSSTAYMEFHSLTSEDSAVYFCARGYYGNSFAYWGQGTLVTVSA

SEQ ID NO: 442 - protein, 136 aa, Artificial Sequence
  Synthetic
MKVLSLLYLLTAIPGILSDVQLQESGPGLVKPSQSLSLTCSVTGYSITSGYLWNWIRQSPGNKLEWMGHITYDGSNNYNPSLKNRISITRDTSKNQFFLKLNSVTTEDTATYFCSRGRYGNNRDYWGQGTTLTVSS

SEQ ID NO: 443 - protein, 138 aa, Artificial Sequence
  Synthetic
MNFGLSLIFLVLVLKGVLCEAKLVESGGDFMQPGGSLKLSCAASGFTLSSYALSWVRQTPEKRLEWVAYISNLGGSTFYPDTVKGRFTISRDNARNTLFLQMSSLQSEDTAIYYCATHLYNYDAFASWGQGTLVTVSA

SEQ ID NO: 444 - protein, 137 aa, Artificial Sequence
  Synthetic
MNFGLSLIFLVLVLKGVLCEVKLVESGGGLVQPGGSLKLSCAASGFTFSRYAMSWVRQTPEKRLEWVAYISIGGTTYYPDTIKGRFTISRDNAKNTLYLQMSSLKSEDTAMYYCTRHYYGHDVMDYWGQGTSVTVSS

SEQ ID NO: 445 - protein, 138 aa, Artificial Sequence
  Synthetic
MNFGLSLIFLVLVLKGVLCEVNLVESGGGFVQPGGSLKLSCVASGFTFSSYAMSWVRQTPDTRLEWVAYISNLGGSTYYPDTVKGRFTISRDNARNTLFLQMSSLQSEDTAMYYCTGHLYHYDAFAYWGQGTLVTVSA

SEQ ID NO: 446 - protein, 138 aa, Artificial Sequence
  Synthetic
MRWSCIIFLFIATATGIHSLVQLQQPGAELVKPGASVKVSCKASGSTFTTYWMHWVKKRPGQGLEWIGGIRPSDSNTNYNHKFKGKATLTVDKASSTAYMQLSSLTSEDSAVYYCAMGAYYSNSFAYWAQGTVVTVSA

SEQ ID NO: 447 - protein, 138 aa, Artificial Sequence
  Synthetic
MRWSCIIFLFIATATGVHSQVQLQQPGAELVKPGASVKVSCKASGSTFTTYWMHWVKKRPGQGLEWIGGIRPFDSNTNYNHKFKGKATLTVDKASNTAYMQLSSLTSEDSAVYYCAMGAYYSNSFAYWGQGTVVTVSA

SEQ ID NO: 448 - protein, 138 aa, Artificial Sequence
  Synthetic
MRWSCIIFLFIATATGVHSQVQLQQPGAELVKPGASVKVSCKASGSTFTTYWMHWVKKRPGQGLEWIGGIRPFDSNTNYNHKFKGKATLTVDKASSTAYMQLSSLTSEDSAVYYCAMGAYYSNSFAYWGQGTVVTVSA

SEQ ID NO: 449 - protein, 138 aa, Artificial Sequence
  Synthetic
MRWSCIIFLFIATATGVHSQVQLQQPGAELVNPGASVKVSCKASGSTFTTYWMHWVKKRPGQGLEWIGGIRPSDSNNNYNHKFKGKATLTVDKASSTAYLQLSSLTSEDSAVYYCAMGAYYSNSFAYWGQGTVVTVSA

SEQ ID NO: 450 - protein, 138 aa, Artificial Sequence
  Synthetic
MRWSCIILFLVASATGVHSQVQLQQPGTELVKPGASVKVSCKASGYTFTTYWMHWVKQRPGQGLEWIGRIRPSDSDSNYNQKFKGKATLTVDKSSDTAYMQLNGLTSEDSAVYYCAMGAYFSNSFAYWGQGTLVTVSA

SEQ ID NO: 451 - protein, 138 aa, Artificial Sequence
  Synthetic
MRWSCIILFLVASATGVHSQVQLQQPGTELVKPGASVKVSCKASGYTFTTYWMHWVKQRPGQGLEWIGRIRPSDSDSNYNQKFKGKATLTVDKSSDTAYMQLNSLTSEDSAVYYCAMGAYFSNSFAYWGQGTLVTVSA

SEQ ID NO: 452 - protein, 138 aa, Artificial Sequence
  Synthetic
MRWSCIILFLVATATGVHSQVLLQQPGTELVKPGASVKVSCKASAYTFTSYWIHWVKQRPGQGLEWIGRIRPSDSDSTYNQNFKGKATLTVDKSSDTAYMQLTSLTSEDSAVYYCSMGAYYSNSFGYWGQGSLVTVSA

SEQ ID NO: 453 - protein, 138 aa, Artificial Sequence
  Synthetic
MRWSCIILFLVATATGVHSQVQLQQPGAELVKPGASVKVSCKASGYTFTTYWMHWVRQRPGQGLEWIGRIRPSDTATNYNQKFKGKATLTVNKSSSTAYMQFSSLTSEDSAVFYCAMGAYYSNSFAYWGQGTLVTVSA

SEQ ID NO: 454 - protein, 138 aa, Artificial Sequence
  Synthetic
MRWSCIILFLVATATGVHSQVQLQQPGTELVKPGASVKVSCKASGYTFTSYWMHWVKQRPGQGLEWIGRIRPSDSDSNYNQKFKGKATLTVDKSSDTAYMQLNSLPSEDSAVYYCAMGAYFSNSFAYWGQGTLVTVSA

SEQ ID NO: 455 - protein, 138 aa, Artificial Sequence
  Synthetic
MRWSCIILFLVATATGVHSQVQLQQPGTELVKPGASVKVSCKASGYTFTTYWMHWVKQRPGQGLEWIGRIRPSDSDSNYNQKFKGKATLTVDKSSDTAYMQLNSLTSEDSAVYYCAMGAYFSNSFAYWGQGTLVTVSA

SEQ ID NO: 456 - protein, 138 aa, Artificial Sequence
  Synthetic
MRWSCIILFLVATATGVHSQVQLQQPGTELVKPGASVKVSCKASGYTFTTYWMHWVTQRPGQGLEWIGRIRPSDSDSNYNQKFKGKATLTVDKSSDTAYMQLNSLTSEDSAVYYCAMGAYFSNSFAYWGQGTLVTVSA

SEQ ID NO: 457 - protein, 138 aa, Artificial Sequence
  Synthetic
MYFRLSSVFLLLILKGVQCEVKLVESEGGLVQPGSSMKLSCTASGFTFSDYYMAWVRQVPEKGLEWVANINYDGSSTFYLDSLKSRFIISRDNARNILYLQMTSLKSEDTATYFCGRQVGYYDPMDYWGQGTSVTVAS

SEQ ID NO: 458 - protein, 128 aa, Artificial Sequence
  Synthetic
MDFQVQIFSFLLISASVIMSRGQIVLSQSPTILSASPGEKVTMTCRATSSVSYMHWFQQKPGSSPKPWIYATSNLASGVPARFSGSGSGTSYSLTISRVEAEDAATYYCQQWSRNPLTFGAGTKLELK

SEQ ID NO: 459 - protein, 128 aa, Artificial Sequence
  Synthetic
MDFQVQIFSFLLISASVMMSRGQIVLSQSPAILSASPGEKVTVTCRASSSLSYMHWYQQRPGSSPKPWIYGTSNLASGVPARFSGSGSGTSYSLTISRVEAEDAATYYCQQWSSNPLTFGAGTKLELK

SEQ ID NO: 460 - protein, 128 aa, Artificial Sequence
  Synthetic
MDFQVQIFSLLLISASVILSRGQIVLSQSPAILSASPGEKVTMTCRASSSVNYIHWYQQKPGSSPKAWIYATSNLASGVPTRFSGSGSGTSYSLTIDRVEAEDAATYYCQQWNSNPLTFGAGTKLELK

SEQ ID NO: 461 - protein, 133 aa, Artificial Sequence
  Synthetic
MDSQAQVLMLLLLWVSGTCGDIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYTYPLTFGAGTKLELK

SEQ ID NO: 462 - protein, 133 aa, Artificial Sequence
  Synthetic
MESQTQVLMSLLFWVSGICGDIVMTQSPSSLTVTAGEKATMSCKSSQSLLNGGNQKNYLTWYQQKPGQPPKLLIYWASTRESGVPDRFTGSGSGTYFTLTISSVQAEDLAVYYCQNDYYFPYTFGGGTKLEIK

SEQ ID NO: 463 - protein, 133 aa, Artificial Sequence
  Synthetic
MESQTQVLMSLLFWVSGICGDIVMTQSPSSLTVTAGEKATMSCKSSQSLLNGGNQKNYLTWYQQKPGQPPTLLIYWASTRESGVPDRFTGSGSGTYFTLTISSVQAEDLAVYYCQNDYYFPYTFGGGTKLEIK

SEQ ID NO: 464 - protein, 133 aa, Artificial Sequence
  Synthetic
MESQTQVLMSLLFWVSGICGDIVMTQSPSSLTVTAGEKVTLSCKSSQSLLNSGNQKNYLTWYQQKPGQPPKLLIYWASTRESGVPDRFTGSGSGTYFTLTISSVQAEDLAVYYCQNDYYFPYTFGGGTKLEIK

SEQ ID NO: 465 - protein, 133 aa, Artificial Sequence
  Synthetic
MESQTQVLMSLLFWVSGICGDIVMTQSPSSLTVTAGEKVTMSCKSSQSLLNSGNQKNYLTWYQQKPGQPPKLLIYWASTRESGVPDRFTGSGSGTYFTLTISSVQAEDLAVYFCQNDYYFPYTFGGGTKLEIK

SEQ ID NO: 466 - protein, 133 aa, Artificial Sequence
  Synthetic
MESQTQVLMSLLFWVSGICGDIVMTQSPSSLTVTAGEKVTMSCKSSQSLLNSGNQKNYLTWYQQKPGQPPKLLMYWASTRESGVPDRFTGSGSGTYFTLTISSVQAEDLAVYYCQNDYYFPYTFGGGTKLEIK

SEQ ID NO: 467 - protein, 133 aa, Artificial Sequence
  Synthetic
MESQTQVLMSLLFWVSGICGDIVMTQSPSSLTVTAGERVTMSCKSSQSLLNSGNLKNYLTWYQQKPGQPPKLLIYWASTRESGVPDRFTGSGSGTYFTLTISSVQAEDLAVYYCQNDYYYPYTFGGGTKLEIK

SEQ ID NO: 468 - protein, 133 aa, Artificial Sequence
  Synthetic
MESQTQVLMSLLFWVSGSCGDIVMTQSPSSLTVTAGEKVTMTCKSSQTLLNRGNQKNYVTWYQQKPGQPPKLLIYWASTRESGVPDRFTGSGSGTDFTLTISSVQAEDLAVYYCQNDYFFPFTFGSGTKLEIR

SEQ ID NO: 469 - protein, 133 aa, Artificial Sequence
  Synthetic
MESQTQVLMSLLFWVSGTCGDIMMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSFPFTFGSGTKLEIK

SEQ ID NO: 470 - protein, 133 aa, Artificial Sequence
  Synthetic
MESQTQVLMSLLFWVSGTCGDIVMTQSPSFLTVTAGEKVTMSCKSSQSLLNSGNLKNYLTWYQQKPGQPPKLLIYWASTRESGVPDRFTGSGSGSDFTLTISSVQAEDLAVYYCQNDYFYPFTFGSGTRLEIK

SEQ ID NO: 471 - protein, 133 aa, Artificial Sequence
  Synthetic
MESQTQVLMSLLFWVSGTCGDIVMTQSPSFLTVTAGEKVTMSCKSSQSLLNSGNLKNYLTWYQQKPGQPPKLLIYWASTRESGVPDRFTGSGSGSDFTLTISSVQAEDLAVYYCQNDYFYPFTFGSGTRLEMK

SEQ ID NO: 472 - protein, 133 aa, Artificial Sequence
  Synthetic
MESQTQVLMSLLFWVSGTCGDIVMTQSPSFLTVTAGEKVTMSCKSSQSLLNSGNLKNYLTWYQQKPGQPPKLLIYWASTRESGVPDRFTGSGSGSDFTLTISSVQAEDLAVYYCQNNYFYPFTFGSGTRLEIK

SEQ ID NO: 473 - protein, 133 aa, Artificial Sequence
  Synthetic
MESQTQVLMSLLFWVSGTCGDIVMTQSPSPLTVIVGEKVTMTCKSSQTLLNRGNQKNYLTWYQQKPGQPPKLLIYWASTRESGVPDRFTGSGSGTDFTLTINSVQAEDLAVYYCQNDYFFPFTFGSGTKLEIR

SEQ ID NO: 474 - protein, 133 aa, Artificial Sequence
  Synthetic
MESQTQVLMSLLFWVSGTCGDIVMTQSPSPLTVTAGEKVTMTCKSSQTLLNRGNQKNYLTWYQQKPGQPPKLLIYWASTRESGVPDRFTGSGSGTDFTLTINSVQAEDLAVYYCQNDYFFPFTFGSGTKLEIR

SEQ ID NO: 475 - protein, 133 aa, Artificial Sequence
  Synthetic
MESQTQVLMSLLFWVSGTCGDIVMTQSPSPLTVTAGEKVTMTCKSSQTLLNRGNQKNYLTWYQQKPGQPPKLLIYWASTRESGVPDRFTGSGSGTDFTLTINSVQAEDLAVYYCQNDYFFPFTFGSGTKLETR

SEQ ID NO: 476 - protein, 133 aa, Artificial Sequence
  Synthetic
MESQTQVLMSLLFWVSGTCGDIVMTQSPSSLAVTPGEKVTMNCKSSQSLLNDGNQKNYLTWYQQKPGQPPKLLIYWASTRESGVPDRFAGSGSGTSFTLTINSVQAEDLAVYYCQNGYSFPYTFGGGTNLEIK

SEQ ID NO: 477 - protein, 133 aa, Artificial Sequence
  Synthetic
MESQTQVLMSLLFWVSGTCGDIVMTQSPSSLPVTTGERVTMSCRSSQILLNSGNQKNYLTWYQQKPGQPPKLLIYWASTRDYGVPDRFTGSGSGTDFTLTISSVQAEDLAVYYCQNAYFYPFTFGAGTKLELK

SEQ ID NO: 478 - protein, 133 aa, Artificial Sequence
  Synthetic
MESQTQVLMSLLFWVSGTCGDIVMTQSPSSLTVTAGEKVTLSCKSSQSLLNSGNQKNYLTWYQQKPGQPPKLLIYWSSTRESGVPDRFTGSGSGTDFTLTISSVQAEDLAVYHCQNNYYYPLTFGAGTKLELK

SEQ ID NO: 479 - protein, 133 aa, Artificial Sequence
  Synthetic
MESQTQVLMSLLFWVSGTCGDIVMTQSPSSLTVTAGEKVTLSCKSSQSLLNSGNQRNYLTWYQQKPGQPPKLLIYWASTRESGVPDRFTGSGSGTDFTLTISSVQAEDLAVYYCQNAYSYPLTFGAGTKLELK

SEQ ID NO: 480 - protein, 133 aa, Artificial Sequence
  Synthetic
MESQTQVLMSLLFWVSGTCGDIVMTQSPSSLTVTAGEKVTMNCKSNQSLLNSGNQKNYVTWYQQKPGQPPKLLIFWASTRESGVPDRFTGSGSETDFTLIISSVQAEDLAVYYCQNDYVYPFTFGSGTKLEIK

SEQ ID NO: 481 - protein, 133 aa, Artificial Sequence
  Synthetic
MESQTQVLMSLLFWVSGTCGDIVMTQSPSSLTVTAGEKVTMNCKSSQSLLNRGNQKNYVTWYQQKPGQPPKLLIFWASTRESGVPDRFTGSGSGTDFTLIISSVQAEDLAVYYCQNDYVYPFTFGSGTKLEIK

SEQ ID NO: 482 - protein, 133 aa, Artificial Sequence
  Synthetic
MESQTQVLMSLLFWVSGTCGDIVMTQSPSSLTVTAGEKVTMNCKSSQSLLNSGNQKNYLTWYQQKPGQPPKLLIYWASTRESGVPDRFTGSGSGTDFTLTINSVQAEDLAVYYCQNDYSYPFMFGSGTKLEIK

SEQ ID NO: 483 - protein, 133 aa, Artificial Sequence
  Synthetic
MESQTQVLMSLLFWVSGTCGDIVMTQSPSSLTVTAGEKVTMNCKSSQSLLNSGNQKNYLTWYQQKPGQPPKLLIYWASTRTSGVPDRFTGSGSGTDFTLTISSVQAEDLAVYCCQNGYTYPLTFGAGTKLELK

SEQ ID NO: 484 - protein, 133 aa, Artificial Sequence
  Synthetic
MESQTQVLMSLLFWVSGTCGDIVMTQSPSSLTVTAGEKVTMSCKSSQSLLNGGNQKNYLTWYQQKPGQPPKLLIYWASTRESGVPDRFTGSGSGTDFTLTISTMQAEDLAVYYCQNDYFFPYTFGGGTKLEIK

SEQ ID NO: 485 - protein, 133 aa, Artificial Sequence
  Synthetic
MESQTQVLMSLLFWVSGTCGDIVMTQSPSSLTVTAGEKVTMSCKSSQSLLNSGNQKNYLTWYQHKPGQPPKLLIYWASTRRSGVPDRFTGSGSGTDFTLTITSVQAEDLAVYCCQNVYVYPLTFGAGTKLELK

SEQ ID NO: 486 - protein, 133 aa, Artificial Sequence
  Synthetic
MESQTQVLMSLLFWVSGTCGDIVMTQSPSSLTVTAGEKVTMSCKSSQSLLNSGNQKNYLTWYQKKPGQPPKLLIYWASTRESGVPDRFTGSGSGTDFTLTISSVQAEDLAVYFCQNDYSFPFTFGSGTKLEIK

SEQ ID NO: 487 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQAPKLLIYWASTRQSGVPDRFTGSGFGTDFTLIITTVQTEDLAVYFCQNDFGFPYTFGGGTKLEMN

SEQ ID NO: 488 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGRGSGTDFTLTISSVQAEDLAVYYCQNNYNYPVTFGAGTKLELK

SEQ ID NO: 489 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAADLAVYFCQNDYSFPFTFGSGTKLEIK

SEQ ID NO: 490 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYFCQNDYSFPFTFGSGTKLEIK

SEQ ID NO: 491 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNAYSFPFTFGSGTQLEIR

SEQ ID NO: 492 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYYYPLTFGAGTKLELK

SEQ ID NO: 493 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNNYVYPLTFGAGTKLELR

SEQ ID NO: 494 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNVYSYPITFGSGTKLEIK

SEQ ID NO: 495 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQPPKMLIYWASTRESGVPDRFTGSGSGTDFTLTLSSVKAEDLAVYYCQNDYYYPLTFGAGTKLELR

SEQ ID NO: 496 - protein, 133 aa, Artificial Sequence
  Synthetic
MESQTQVLMSLLFWVSGTCGDIVMTQSPSSLTVTAGEKVTMSCKSSQSLLNSGNQKNYLTWYQQKPGQSPKMLIYWASTRASGVPDRFTGSGSGTDFTLTLSSVKAEDLAVYYCQNDYYYPLTFGAGTKLELR

SEQ ID NO: 497 - protein, 133 aa, Artificial Sequence
  Synthetic
MESQTQVLMSLLFWVSGTCGDIVMTQSPSSLTVTAGEMVTMNCKSSQSLLNSGNQRNYLTWYQQKPGQPPKLLIYWSSTRESGVPDRFTGSGSGTDFTLTISSVQAEDLAVYYCQNDYNYPLTFGAGTKLELK

SEQ ID NO: 498 - protein, 133 aa, Artificial Sequence
  Synthetic
MESQTQVLMSLLFWVSGTCGDIVMTQSPSSLTVTAGEMVTMNCKSSQSLLNSGNQRNYLTWYQQKPGQPPKLLIYWSSTRESGVPDRFTGSGSGTDFTLTISSVQAEDLAVYYCQNDYSYPLTFGAGTKLELK

SEQ ID NO: 499 - protein, 133 aa, Artificial Sequence
  Synthetic
MESQTQVLMSLLFWVSGTCGDIVMTQSPSSLTVTAGEMVTMNCKSSQSLLNSGNQRNYLTWYQQKPGQPPKLLIYWSSTRESGVPDRFTGSGSGTDFTLTISSVQAEDLAVYYCQNDYTYPLTFGAGTKLELK

SEQ ID NO: 500 - protein, 133 aa, Artificial Sequence
  Synthetic
MESQTQVLMSLLFWVSGTCGDIVMTQSPSSLTVTAGEMVTMNCKSSQSLLNSGNRRNYLTWYQQKPGQPPKLLIYWSSTRESGVPDRFAGSGSGTDFTLTISSVQAEDLAVYYCQNDYTYPLTFGAGTKLELK

SEQ ID NO: 501 - protein, 133 aa, Artificial Sequence
  Synthetic
MESQTQVLMSLLFWVSGTCGDIVMTQSPSSLTVTAGKKVTMNCKSSQSLLNSGNQKNYVTWYQQKPGQPPKLLIFWASTRESGVPDRFTGSGSGTDFSLIISTVQAEDLAVYYCQNDYVYPFTFGSGTKLEIK

SEQ ID NO: 502 - protein, 133 aa, Artificial Sequence
  Synthetic
MESQTQVLMSLLFWVSGTCGDIVMTQSPSSLTVTAREKVIMNCKSSQSLFNSGNQKNYLSWYQQKPGQPPKLLIYWASTRKSGVPDRFTGSGSGTGFTLTISSVQAEDLAVYYCQNNYFYPLTFGAGTKLELN

SEQ ID NO: 503 - protein, 133 aa, Artificial Sequence
  Synthetic
MESQTQVLMSLLFWVSGTCGDIVMTQSPSSLTVTAREKVIMNCKSSQSLFNSGNQKNYLTWYQQKPGQSPKLLIYWASTRQSGVPDRFTGSGSGTDFTLTISTVQAEDLAVYFCQNGFSFPYTFGGGTKLEMN

SEQ ID NO: 504 - protein, 133 aa, Artificial Sequence
  Synthetic
MESQTQVLMSLLFWVSGTCGDIVMTQSPSSLTVTAREKVTMNCKSSQSLLNSGNQKNYLTWYQQKPGQPPKMLIYWASTRESGVPDRFTGSGSGTYFTLTISSVQAEDLAVYYCQNDYSFPFTFGSGTKLEIK

SEQ ID NO: 505 - protein, 133 aa, Artificial Sequence
  Synthetic
MESQTQVLMSLLFWVSGTCGDIVMTQSPSSLTVTPGEKVTMNCKSSQSLLNSGNQKNYVTWYQQKPGQPPKLLMFWASTRESGVPDRFTGSGSGTDFTLIISSVQAEDLAVYHCQNDYVYPFTFGSGTKLEIK

SEQ ID NO: 506 - protein, 133 aa, Artificial Sequence
  Synthetic
MESQTQVLMSLLFWVSGTCGDIVMTQSPSSLTVTSGEKVTMSCKSSQSLFNSGNQKNYLTWYQQKLGQPPKLLIFWASTRESGVPDRFTGSGSGTDFTLTISSVQTEDLAVYYCQNDYYYPLTFGAGTKLELK

SEQ ID NO: 507 - protein, 133 aa, Artificial Sequence
  Synthetic
MESQTQVLMSLLFWVSGTCGDIVMTQSPSSLTVTVGEKVTMSCKSSQSLLNSGNQKNYLTWYRQKPGQPPELLIYWASTRESGVPDRFTGSGSGTDFTLTISSVQAEDLAVYYCQNVYSYPLTFGAGTKLELK

SEQ ID NO: 508 - protein, 133 aa, Artificial Sequence
  Synthetic
MESQTQVLMSLLFWVSGTYGDIVMTQSPSSLTVTAGEKVTMTCKSGQSLLNSGNQRNYLTWYQQKPGQSPKLLIYWASTRESGVPDRFTGSGSGADFTLTISSVQAEDLALYYCQNAYFYPYTFGGGTKLEIK